(19) United States
(12) Patent Application Publication
(10) Pub. No.: US 2017/0073337 A1
(43) Pub. Date: Mar. 16, 2017

(54) DIHYDROTHIAZINE AND DIHYDROOXAZINE DERIVATIVES HAVING BACE1 INHIBITORY ACTIVITY

(71) Applicant: Shionogi & Co., Ltd., Osaka (JP)

(72) Inventors: Ken-ichi KUSAKABE, Osaka (JP); Genta TADANO, Osaka (JP); Kazuo KOMANO, Osaka (JP); Kouki FUCHINO, Osaka (JP); Kenji NAKAHARA, Osaka (JP)

(21) Appl. No.: 15/302,892

(22) PCT Filed: Apr. 10, 2015
(86) PCT No.: PCT/JP2015/062314

§ 371 Date: Oct. 7, 2016

(30) Foreign Application Priority Data
Apr. 11, 2014 (JP) 2014-081524

(51) Int. Cl. C07D 413/12; C07D 417/12; C07D 498/04; C07D 417/14

(52) U.S. Cl. CPC C07D 413/12; C07D 417/14; C07D 417/12; C07D 498/04

(57) ABSTRACT

The present invention provides a compound which has an effect of inhibiting amyloid β production, especially an effect of inhibiting BACE1, and which is useful as a therapeutic or prophylactic agent, for diseases induced by production, secretion and/or deposition of amyloid β proteins.

A compound of the formula (I) wherein X is —S— or —O—,
R^{3a} is alkyl, haloalkyl or the like,
R^{2a} is H, halogen, alkyloxy, haloalkyloxy or the like,
R^{2b} is H or the like,
R^{3b} is H or alkyl,
ring A and ring B is each independently a substituted or unsubstituted aromatic carbocycle, a substituted or unsubstituted aromatic heterocycle or the like, and
R¹ is substituted or unsubstituted alkyl or the like,
or a pharmaceutically acceptable salt thereof.

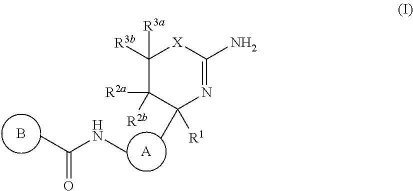

### TECHNICAL FIELD

The present invention relates to a compound which has amyloid β production inhibitory activity, and is useful as an agent for treating or preventing disease induced by production, secretion and/or deposition of amyloid β proteins.

### BACKGROUND ART

In the brain of Alzheimer's patient, the peptide composed of about 40 amino acids residue as is called amyloid β protein, that accumulates to form insoluble specks (senile specks) outside nerve cells is widely observed. It is concerned that these senile specks kill nerve cells to cause Alzheimer's disease, so the therapeutic agents for Alzheimer's disease, such as decomposition agents of amyloid β protein and amyloid vaccine, are under investigation.

Secretase is an enzyme which cleaves a protein called amyloid β precursor protein (APP) in cell and produces amyloid β protein. The enzyme which controls the production of N terminus of amyloid β protein is called as β-secretase (beta-site APP-cleaving enzyme 1, BACE1). It is thought that inhibition of this enzyme leads to reduction of producing amyloid β protein and that the therapeutic or prophylactic agent for Alzheimer's disease will be created due to the inhibition.

Patent Literatures 1 to 39 and Non-Patent laterature 1 disclose compounds having a structure similar to those of the compounds of the present invention. Each of these documents discloses each compound is useful as therapeutic agent for Alzheimer's disease, Alzheimer's relating symptoms, diabetes or the like, but each of substantially disclosed compounds has a structure different from the compounds of the present invention.

### CITATION LIST

### Patent Literature

### [PTL 1]

### WO2007/049532

### [PTL 2]

### WO2008/133273

### [PTL 3]

### WO2008/133274

### [PTL 4]

### WO2009/151098

### [PTL 5]

### WO2010/047372

### [PTL 6]

### WO2010/113848

### [PTL 7]

### WO2011/071057

### [PTL 8]

### WO2011/058763

### [PTL 9]

### WO2011/070781

### [PTL 10]

### WO2011/077726

### [PTL 11]

### WO2011/071135

### [PTL 12]

### WO2011/071109

### [PTL 13]

### WO2012/057247

### [PTL 14]

### WO2012/057248

### [PTL 15]

### WO2012/147762

### [PTL 16]

### WO2012/147763

### [PTL 17]

### JP2012/250933A

### [PTL 18]

### WO2014/010748

### [PTL 19]

### JP2014/101354A

### [PTL 20]

### WO2014/065434

### [PTL 21]

### JP2014/101353A

### [PTL 22]

### WO2013/110622

### [PTL 23]

### WO2014/001228

### [PTL 24]

### WO2013/041499

### [PTL 25]

### WO2012/107371

### [PTL 26]

### WO2011/069934

### [PTL 27]

### WO2011/070029

### [PTL 28]

### WO2012/139993

### [PTL 29]

### WO2012/168164

### [PTL 30]

### WO2012/168175

### [PTL 31]

### WO2012/156284

### [PTL 32]

### WO2014/166906

### [PTL 33]

### WO2014/114532

### [PTL 34]

### WO2013/027188

### [PTL 35]

### WO2014/134341

### [PTL 36]

### WO2008/103351

### [PTL 37]

### US2006/0200445

### [PTL 38]

### US2006/0287294

### [PTL 39]

### WO2014/098831

### Non Patent Literature

### [NPL 1]

### Journal of Medicinal Chemistry, 2013, 56(10), pp 3980-3995

### SUMMARY OF INVENTION

### Technical Problem

The present invention provider compounds which have reducing effects to produce amyloid β protein, especially BACE1 inhibitory activity, and are useful as an agent for treating disease induced by production, secretion and/or deposition of amyloid β protein.

### Solution to Problem

The present invention, for example, provides the inventions described in the following items.

(1) A compound of formula (1):

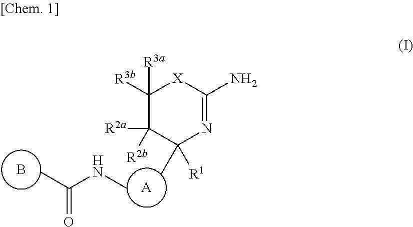

wherein
X is —S— or —O—,
(i) when X is —S—, then
   R^{3a} is alkyl, haloalkyl, hydroxyalkyl or alkyloxyalkyl,
   R^{2a} is halogen, alkyloxy or haloalkyloxy and
   R^{2a} may be alkyl when R^{3a} is haloalkyl,
   R^{2b} is H,
   R^{2a} and R^{2b} together with the carbon atom to which they are attached may form substituted cycloalkane,
   R^{3a} may be H when R^{2a} and R^{2b} together with the carbon atom to which they are attached may form substituted cycloalkane,
(ii) when X is —O—, then
   R^{3a} is haloalkyl optionally substituted with one or more selected from alkyloxy and cycloalkyl, or cycloalkyl substituted with one or more selected from halogen,
   R^{2a} is H, halogen, alkyl, atkyloxy or haloalkyloxy,
   R^{2b} is H,
   R^{2a} and R^{2b} together with the carbon atom to which they are attacbed may form substituted cycloalkane,
R^{3a} may be H or alkyl when R^{2a} and R^{2b} together with the carbon atom to which they are attached may form substituted cycloalkane,
R^{3b} is H or alkyl,

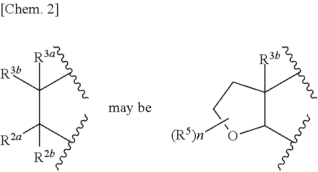

ring A is a substituted or unsubstituted aromatic carbocycle, a substituted or unsubstituted non-aromatic carbocycle, a substituted or unsubstituted aromatic heterocycle or a substituted or unsubstituted non-aromatic heterocycle,
ring B is a substituted or unsubstituted aromatic carbocycle, a substituted or unsubstituted non-aromatic carbocycle, a substituted or unsubstituted aromatic heterocycle or a substituted or unsubstituted non-aromatic heterocycle,
R¹ is substituted or unsubstituted alkyl, substituted or unsubstituted alkenyl, substituted or unsubstituted alkynyl or substituted or unsubstituted cycloalkyl,
R⁵ is halogen or substituted or unsubstituted alkyl,
n is an integer of 0 to 2,
   provided that the following compounds are excluded:
   (1) a compound wherein X is —O—, R^{3a} is CH₂F or CF₃, R^{3b} is H, R^{2a} is H or F, and R^{2b} is H,
   (2) a compound wherein X is —O—, R^{3a} is CHF₂, R^{3b} is H, R^{2a} is OMe and R^{2b} is H, and
   (3) the following compound:

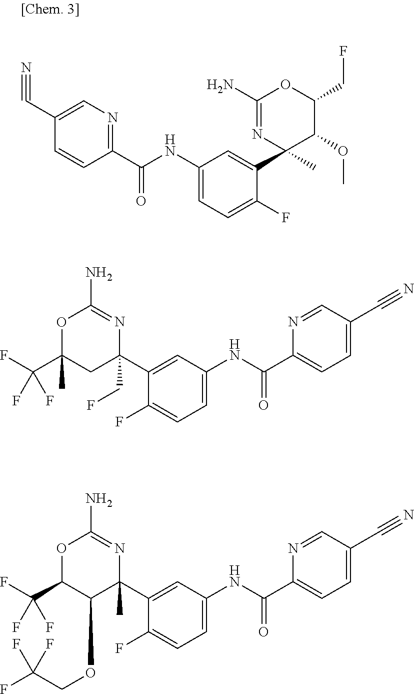 [SMILES: CO[C@H]1[C@@H](CF)OC(N)=N[C@]1(C)c1cc(NC(=O)c2ccc(C#N)cn2)ccc1F ; C[C@]1(C(F)(F)F)C[C@](CF)(c2cc(NC(=O)c3ccc(C#N)cn3)ccc2F)N=C(N)O1 ; C[C@]1(c2cc(NC(=O)c3ccc(C#N)cn3)ccc2F)N=C(N)O[C@H](C(F)(F)F)[C@@H]1OCC(F)(F)F]

or a pharmaceutically acceptable salt thereof.
(1-1) A compoond of formula (I):

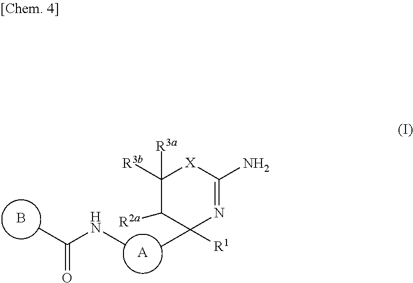

wherein
X is —O— or —S—,
(i) when X is —O—, then
   R^{3a} is haloalkyl,
   R^{2a} is H, halogen, alkyl, alkyloxy or haloalkyloxy,
(ii) when X is —S—, then
   R^{3a} is alkyl or haloalkyl,
   R^{2a} halogen, alkyloxy or haloalkyloxy and
   R^{2a} may be alkyl when R^{3a} is haloalkyl,
R^{3b} is is H or alkyl, and

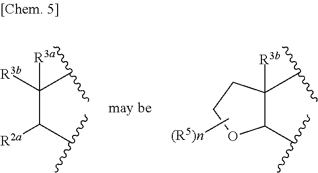

ring A is a substituted or unsubstituted aromatic carbocycle, a substituted or unsubstituted non-aromatic carbocycle, a substituted or unsubstituted aromatic heterocycle or a substituted or unsubstituted non-aromatic heterocycle,
ring B is a substituted or unsubstituted aromatic carbocycle, a substituted or unsubstituted non-aromatic carbocycle, a substituted or unsubstituted aromatic heterocycle or a substituted or unsubstituted non-aromatic heterocycle,
R¹ is substituted or unsubstituted alkyl substituted or unsubstituted alkenyl, substituted or unsubstituted alkynyl or substituted or unsubstituted cycloalkyl,
R⁵ is halogen or substituted or unsubstituted alkyl,
n is an integer of 0 to 2,
provided that the following compounds are excluded:
   (1) a compound wherein X is —O—, R^{3a} is CH₂F or CF₃, R^{3b} is H, and R^{2a} is H or F, and
   (2) the following compound:

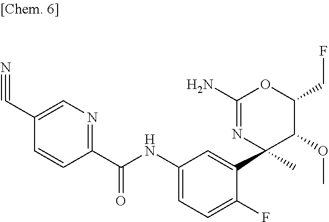 [SMILES: CO[C@H]1[C@@H](CF)OC(N)=N[C@]1(C)c1cc(NC(=O)c2ccc(C#N)cn2)ccc1F]

or a pharmaceuticlly acceptable salt thereof.
(1-2) A compound according to the item (1)
provided that the following compounds are excluded:
   (1) a compound wherein X is —O—, R^{3a} is CH₂F or CF₃, R^{3b} is H, and R^{2a} is H, F or OMe, and
   (3) a compound wherein X is —O—, R^{3a} is CHF₂, R^{3b} is H, and R^{2a} is OMe,
or a pharmaceutically acceptable salt thereof.
(2) The compound according to the item (1), (1-1) or (1-2) wherein X is —O—, or a pharmaceutically acceptable salt thereof.
(3) The compound according the item (2) wherein R^{3a} is CH₂F, CHF₂, CF₃, CH(F)CH₃ or CF₂CH₃, and R^{3b} in H or CH₃, or a pharmacoutlcally acceptable salt thereof.
(4) The compound according to the item (2) or (3) wherein R^{2a} is H, F, CH₃, OCH₃ or OCH₂CF₃, or a pharmaceutically acceptable salt thereof.
(5) The compound according to the item (2) or (3) wherein R^{2a} is H, halogen or alkyl, R^{2b} is H, and R^{3a} is CHF₂, CH(F)CH₃ or CF₂CH₃, or a pharmaceutically acceptable salt thereof.
(5-1) The compound according to any one of the items (2) to (4) wherein R^{2a} is H or halogen, and R^{3a} is CHF₂, CH(F)CH₃ or CF₂CH₃, or a pharmaceutically acceptable salt thereof.
(6) The compound according to item (2) wherein R^{2a} is H or halogen, R^{2b} is H, R^{3a} is CH₂F or CF₃, R^{3b} is alkyl, and R¹ is unsubstituted alkyl, or a pharmaceutically acceptable salt thereof.
(6-1) The compound according to any one of the items (2) to (4) wherein R^{2a} is H or halogen, R^{3a} is CH₂F or CF₃, and R^{3b} is alkyl, or a pharmaceutically acceptable salt thereof.
(7) The compound according to any one of the items (2) to (4) wherein R^{2a} is alkyl, alkyloxy or haloalkyloxy, or a pharmaceutically acceptable salt thereof.
(8) The compound according to item (2) wherein

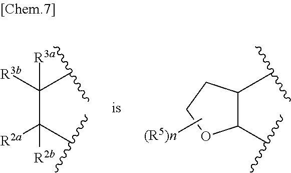

R⁵ is halogen and n is 1 or 2, or a pharmaceutically acceptable salt thereof.
(9) The compound according to item (2) or (4) wherein R^{3a} is haloalkyl substituted with alkyloxy or cycloalkyl, or a pharmaceutically acceptable salt thereof.
(10) The compound according to any one of items (1), (1-1) and (1-2) wherein X is —S—, R^{2a} in halogen or alkyloxy, R^{2b} is H, R^{3a} is alkyl, haloalkyl, hydroxyalkyl or alkyloxyalkyl, and R^{3b} H, or a pharmaceutically acceptable salt thereof.
(11) The compound according to the item (1) wherein X is —S—, R^{2a} is F, R^{2b} is H, R^{3a} is CH₃ or CH₃F, and R^{3b} is H, or a pharmaceutically acceptable salt thereof.
(12) The compound according to any one of items (1), (1-1) and (1-2) wherein R^{2a} and R^{2b} together with the carbon atom to which they are attached form cycloalkane substituted with halogen, R^{3a} is H or alkyl, or a pharmaceutically accceptable salt thereof.
(13) The compound according to any one of the items (1), (1-1), (1-2), (2) to (5), (5-1), (6), (6-1), and (7) to (12) wherein R¹ is alkyl, or a pharmaceutically acceptable salt thereof.
(14) The compound according to any one of the item (1), (1-1), (1-2), (2) (5), (5-1), (6), (6-1), and (7) to (13) wherein ring A is

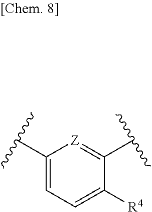

wherein R⁴ is H or halogen, and —Z═ is —CH═ or —N═, or a pharmaceutically acceptable salt thereof.
(15) The compound according to item (14) wherein R⁴ is halogen and —Z═ is —CH═, or a pharmaceutically acceptable salt thereof.
(16) The compound according to any one of items (1), (1-1), (1-2), (2) to (5), (5-1), (6), (6-1), and (7) to (15) to (15) wherein ring B is substituted or unsubstituted pyridine, substituted or unsubstituted pyrazine, substituted or unsubstituted pyrimidine, substituted or unsubstituted pyridazine or substituted or unsubstituted oxazole, or a pharmaceutically acceptable salt thereof.
(16-1) The compound according to any one of the items (1), (1-1), (1-2), (2) to (5), (5-1), (6), (6-1), and (7) to (15) wherein ring B is substituted or unsubstituted pyridine, substituted or unsubstituted pyrazine, or substituted or unsubstituted oxazole, or a pharmaceutically acceptable salt thereof.
(17) A pharmaceutical composition comprising the compound according to any one of the items (1), (1-1), (1-2), (2) to (5), (5-1), (6), (6-1), (7) to (16) and (16-1), or a pharmaceutically acceptable salt thereof.
(18) A pharmaceutical composition having BACE1 inhibitory activity comprising the compound according to any one of the items (1), (1-1), (1-2), (2) to (5), (5-1), (6), (6-1), (7) to (16) and (16-1), or a pharmaceutically acceptable salt thereof.
(19) A method for inhibiting BACE1 activity comprising administering the compound according to any one of items (1), (1-1), (1-2), (2) to (5), (5-1), (6), (6-1), (7) to (16) and (16-1), or a pharmaceutically acceptable salt thereof.
(20) The compound according to any one of items (1), (1-1), (1-2), (2) to (5), (5-1), (6), (6-1), (7) to (16) and (16-1), or a pharmaceutically acceptable salt thereof for use in a method for inhibiting BACE1 activity.
(21) The pharmaceutical composition according to item (17) or (18) for treating or preventing Alzheimer dementia, mild cognitive impairment or prodromal Alzheimer's disease, for preventing the progression of Alzheimer dementia, mild cognitive impairment, or prodromal Alzheimer's disease, or for preventing the progression in a patient asymptomatic at risk for Alzheimer dementia.
(22) A method for treating or preventing Alzheimer dementia, mild cognitive impairment or prodromal Alzheimer's dissaae, for preventing the progression of Alzheimer dementia, mild cognitive impairment, or prodromal Alzheimer's disease, or for preventing the progression in a patient asymptomatic at risk for Alzheimer dementia comprising administering the compound according to any one of items (1), (1-1), (1-2), (2) to (5), (5-1), (6), (6-1), (7) to (16) and (16-1), or a pharmaceutically acceptable salt thereof.
(23) A compound according to any one of items (1), (1-1), (1-2), (2) to (5), (5-1), (6), (6-1), (7) to (16) and (16-1), or a pharmaceutically acceptable salt thereof for use in treating or preventing Alzheimer dementia, mild cognitive impairment or prodromal Alzheimer's disease, for use in preventing the progression of Alzheimer dementia, mild cognitive impairment or prodromal Alzheimer's disease, or for use in preventing the progression in a patient asymptomatic at risk for Alzheimer dementia.
(24) Use of the compound according to any one of items (1), (1-1), (1-2), (2) to (5), (5-1), (6), (6-1), (7) to (16) and (16-1), or a pharmaceutically acceptable salt thereof for manufacturing a medicament for inhibiting BACE1 activity.
(25) The pharmaceutical composition according to the item (17) or (18) for treating for preventing a disease induced by production, secretion or deposition of amyloid β proteins.
(26) A method tor treating or preventing diseases induced by production, secretion or deposition of amyloid β proteins comprising administering the compound according to any one of items (1), (1-1), (1-2), (2) to (5), (5-1), (6), (6-1), (7) to (16) and (16-1) or a pharmaceutically acceptable salt thereof.
(27) A compound according to any one of items (1), (1-1), (1-2), (2) to (5), (5-1), (6), (6-1), (7) to (16) and (16-1), or a pharmaceutically acceptable salt thereof for use in treating or preventing diseases induced by production, secretion or deposition of amyloid β proteins.
(28) Use of the compound according to any one of items (1), (1-1), (1-2), (2) to (5), (5-1), (6), (6-1), (7) to (16) and (16-1), or a pharmaeeuticaliy acceptable salt thereof for manufacturing a medicament for treating or preventing diseases induced by production, secretion or deposition of amyloid β proteins.
(29) The pharmaceutical composition according to the item (17) or (18) for treating or preventing Alzheimer dementia.
(30) A method for treating or preventing Alzheimer dementia comprising administering the compound according to any one of items (1), (1-1), (1-2), (2) to (5), (5-1), (6), (6-1), (7) to (16) and (16-1), or a pharmaceutically acceptable salt thereof.
(31) A compound according to any one of items (1), (1-1), (1-2), (2) to (5), (5-1), (6), (6-1), (7) to (16) and (16-1), or a pharmaceutically acceptable salt thereof for use in treating or preventing Alzheimer dementia.
(32) Use of the compound according to any one of items (1), (1-1), (1-2), (2) to (5), (5-1), (6), (6-1), (7) to (16) and (16-1), or a pharmaceutically acceptable salt thereof for manufacturing a medicament for treating or preventing Alzheimer dementia.

### Advantageous Effects of Invention

The compound of the present invention has BACE1 inhibitory activity and is useful as an agent for treating and/or preventing dUcase induced by production, secretion or deposition of amyloid β proteins such as Alzheimer dementia.
(33) A pharmaceutical composition comprising the compound of any one of items (1), (1-1), (1-2), (2) to (5), (5-1), (6), (6-1), (7) to (16) and (16-1), or a pharmaceutically acceptable salt thereof, for oral administration.
(34) The pharmaceutical composition of (33), which is a tablet, powder, granule, capsule, pill, film, suspension, emulsion, elixir, syrup, lemonade, spirit, aromatic water, extract, decoction or tincture.
(35) The pharmaceutical composition of (34), which is a sugar-coated tablet, film-coated tablet, enteric-coated tablet, sustained-release tablet, troche tablet, sublingual tablet, buccal tablet, chewable tablet, orally disintegrated tablet, dry syrup, soft, capsule, micro capsule or sustained-release capsule.
(36) A pharmaceutical composition comprising the compound of any one of items (1), (1-1), (1-2), (2) to (5), (5-1), (6), (6-1), (7) to (16) and (16-1), or a pharmaceutically acceptable salt thereof, for parental administration.
(37) The pharmaceutical composition of (36), for dermal, subcutaneous, intravenous, intraarterial, intramuscular, intraperitoneal, tranamucosal, inhalation, transnasal, ophthalmic, inner ear or vaginal administration.
(38) The pharmaceutical composition of (36) or (37), which is injection, infusion, eye drop, nose drop, ear drop, aerosol, inhalation, lotion, impregnation, liniment, mouthwash, enema, ointment, planter, jelly, cream, patch, cataplasm, external powder or suppository.
(39) A pharmaceutical composition con prising the compound of any one of items (1), (1-1), (1-2), (2) to (5), (5-1), (6), (6-1), (7) to (16) and (16-1), or a pharmaceutically acceptable salt thereof, for a pediatric or geriatric patient.
(40) A pharmaceutical composition consisting of a combination of the compound of any one of items (1), (1-1), (1-2), (2) to (5), (5-1), (6), (6-1), (7) to (16) and (16-1) or a pharmaceutically acceptable salt thereof and acetylcholinesterase inhibitor, NMDA antagonist, or other medicament tor Alzheimer dementia.
(41) A pharmaceutical composition comprising the compound of any one of items (1), (1-1), (1-2), (2) to (5), (5-1), (6), (6-1), (7) to (16) and (16-1), or a pharmaceutically acceptable salt thereof, for a combination therapy with acetylcholinesterase inhibitor, NMDA antagonist, or other medicament for Alzheimer dementia.

### DESCRIPTION OF EMBODIMENTS

Each meaning of terms used herein is described below. Both when used alone and in combination unless otherwise noted, each term is used in the same meaning.

In the specification, the term of “consisting of” means having only components.

In the specification, the term of “comprising” means not restricting with components and not excluding undescribed factors.

In the specification, the “halogen” includes fluorine, chlorine, bromine, and iodine. Fluorine and chlorine are preferable.

In the specification, the “alkyl” includes linear or branched alkyl of a carbon number of 1 to 15, for example, a carbon number of 1 to 10, for example, a carbon number of 1 to 6, and for example, a carbon number of 1 to 4. Examples include methyl, ethyl, n-propyl, leopropyl, n-butyl, isobutyl, sec-butyl, tert-butyl, n-pentyl, isopentyl, neopentyl, hexyl, isohexyl, n-heptyl, isoheptyl, n-octytl, isotyl, n-nonyl and n-decyl. Examples are methyl, ethyl, n-propyl, isopropyl, n-butyl, isobutyl, secbutyl, tertbutyl and n-pentyl.

In one embodiment, “alkyl” is methyl, ethyl, n-propyl, isopropyl or tert-butyl.

The term “alkenyl” includes linear or branched alkenyl of a carbon number or 2 to 15, for example, a carbon number of 2 to 10, for example, a carbon number of 2 to 6, and for example, a carbon number of 2 to 4, having one or more double bonds at any available positions. Examples include vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, ortenyl, nonenyl, decenyl, undecenyl, dodecenyl, tridecenyl, tetradecenyl and pentadecenyl. Examples are vinyl, allyl, propenyl, isopropenyl and butenyl.

The term “alkynyl” includes a linear or branched alkynyl of a carbon number of 2 to 15, for example, a carbon number of 2 to 10, for example, a carbon number of 2 to 8, for example, a carbon number of 2 to 6, and for example, a carbon number of 2 to 4 having one or more triple bonds at optionally positions.

Specific examples are ethynyl, propynyl, butynyl, pentynyl, hexynyl, heptynyl, octynyl, nonynyl and decynyl. These may have further a double bond at any available position. Examples are ethynyl, propynyl, butynyl and pentynyl.

The term “alkylene” include a linear or branched divalent carbon chain of a carbon number of 1 to 15, for example, a carbon number of 1 to 10, for example, a carbon number of 1 to 6, and for example a carbon number of 1 to 4. Examples are methylene, dimethylene, trimethylene, tetramethylene, pentamethylene and hexamethylene.

Alkylene portion in “alkylenedioxy” in the same as the above “alkylene”. Examples are methylenedioxy and dimethylenedioxy.

The term of “aromatic carbocyclyl” includes an aromatic hydrocarbon group which is monocyclic or which consists of two or more rings. Examples are an aromatic hydrocarbon group of a carbon number at 6 to 14, and specific examples are phenyl, naphthyl, anthryl and penanthryl.

In one embodiment, “aromatic carbocyclyl is phenyl.

The term of “non-aromatic carbocyclyl” includes saturated carbocyclyl or unsaturated non-aromatic carbocyclyl which is monocyclic or which consists of two or more rings. A “non-aromatic carbocyclyl” of two or more rings includes a fused cyclic group wherein a non-aromatic monocyclic carbocyde or a non-aromatic carbocycle of two or more rings is fused with a ring of the above “aromatic carbocyclyl”.

In addition, the “non-aromatic carbocycly” also includes a cyclic group having a bridge or a cyclic group to form a spiro ring as follows:

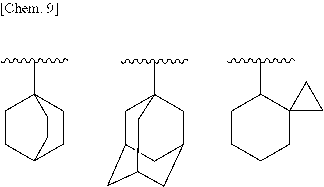 [SMILES: *C12CCC(CC1)CC2 ; *C12CC3CC(CC(C3)C1)C2 ; *C1CCCCC12CC2]

The term “non-aromatic monocyclic carbocycle” includes a group having 3 to 16 carbon atoms, for example, 3 to 12 carbon atoms, for example, 3 to 8 carbon atoms, and for example, 3 to 5 carbon atoms. Examples are cyclopropane, cyclobutane, cyclopentane, cyclohexane, cycloheptane, cyclooctane, cyclononane, cyclodecane, cyclopropene, cyclobutene, cyclopentene, cyclohexene cycloheptene and cyclohexadiene.

Examples of non-aromatic carbocyclyl consisting of two or more rings include a group having 6 to 14 carbon atoms, and examples are indanyl, indenyl, acenaphthyl, tetrahydronaphthyl and fluorenyl.

The term “cycloalkyl” includes a carbocyclic group of a carbon number of 3 to 10, for example, a carbon number of 3 to 8, and for example, a carbon number 4 to 8. Examples are cyclopropyl, cyclobutyl, cyclopentyl, cyclohexyl, cycloheptyl, cyclooctyl, cyclononyl and cyclodecyl.

The term “cycloalkane” includes a carbocycle of a carbon number of 3 to 10, for example, a carbon number of 3 to 8, for example, a carbon number 3 to 5. Examples are cyclopropane, cyclobutane, cyclopentane, cyclohexane, cycloheptane, cyclooctane, cyclononane and cyclodecane.

Cycloalkyl portion in “cycloalkylalkyl”, “cycloalkylamino” and “cycloalkylalkyloxy” are the same as the above “cycloalkane”.

The term of “aromatic heterocyclyl” includes an aromatic group which is monocyclic, or which consists of two or more rings, containing one or more of heteroatoms selected independently from oxygen, sulfur and nitrogen atoms.

An “aromatic heterocyclyl” of two or more rings includes a fused cyclic group wherein aromatic monocyclic heterocyclyl or non-aromatic heterocyclyl consisting of two or more rings is fused with a ring of the above “aromatic carbocyclyl”.

The term “aromatic monocyclic heterocyclyl” includes a 5- to 8-membered group, and for example, 5- to 6-membered group. Examples are pyrrolyl, imidazolyl, pyrazolyl, pyridyl, pyridazinyl, pyrimidinyl, pyrazinyl, triazolyl, triazinyl, tetrazolyl, furyl, thienyl, isoxazolyl, oxazolyl, oxadiaxotyl, isothiazolyl, thiazolyl and thiadiazolyl.

Examples of aromatic bicyclic heterocyclyl includes a 9- to 10-membered group, and examples are indolinyl, isoindolinyl, indazolinyl, iodolizinyl, quinolinyl, isoquinolinyl, cinnolinyl, phthalazinyl, quinazolinnyl, naphthyridinyl, quinoxalinyl, purinyl, pteridinyl, benzimidazolyl, benzisoxazolyl, benzoxazolyl, benzoxadiazolyl, benzisothiazolyl, benzothiazolyl, benzothiadiazoly, benzofuryl, isobenzofuryl, benzothienyl, benzotriadiazolyl, imidazopyridyl, triazolopyridyl, imidazothiazolyl, pyrazinopyridazinyl, oxazolopyridyl and thiazolopyridyl.

Examples of aromatic heterocyclyl of three or more rings includes a 13 to 14-membered group, and examples are carbazolyl, acridinyl, xanthenyl, phenothiazinyl, phenoxathiinyl, phenoxazinyl and dibenzofuryl.

The term of “non-aromatic heterocyclyl” includes a non-aromatic group which is monocyclic, or which consists of two or more rings, containing one or more of heteroatoms selected independently from oxygen, sulfur and nitrogen atoms.

A “non-aromatic heterocyclyl” of two or more rings includes a fused cyclic group wherein non-aromatic monocyclic heterocyclyl or non-aromatic heterocyclyl of two or more rings is fused with a ring of the above “aromatic carbocyclyl”, “non-aromatic, carbocyclyl” and/or “aromatic heterocyclyl”.

In addition, the “non-aromatic heterocyclyl” also includes a cyclic group having a bridge or a cyclic group to form a spiro ring as follows:

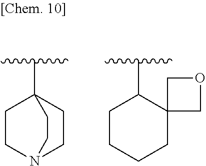

The term “non-aromatic monocyclic heterocyclyl” includes a 3- to 8-membered ring, and for example, 4-, 5- or 6-membered ring. Examples are dioxanyl, thiiranyl, oxiranyl, oxetanyl, oxathiolanyl, azetidinyl, thianyl, thiazolidinyl, pyrrolidinyl, pyrrolinyl, imidazolidioyl, imidazolinyl, pyrazolidinyl, pyrazolinyl, piperidyl, piperazinyl, morpholinyl, morpholino, thiomorpholinyl, thiomorpholino, dihydropyridyl, tetrahydropyridyl, tetrahydrofuryl, tetrahydropyranyl, dihydrothiazolyl, tetrahydrothiazolyl, tetrahydroisothiazolyl, dihydrooxazinyl, hexahydonazepinyl, tetrahydrodiazepinyl, tetrahydropyridazinyl, hexahydropyrimidinyl, dioxolanyl, dioxazinyl, aziridinyl, dioxolinyl, oxepanyl, thiolanyl, thiinyl and thinzinyl.

Examples of non-aromatic heterocyclyl of two or more rings includes a 9 to 14-membered group, and examples are indolinyl, isoindolinyl, chromanyl and isochromanyl.

The term of “alkyloxy” includes a group wherein an oxygen atom is substituted with the show “alkyl”. Examples are methyloxy, ethyloxy, n-propyloxy, isopropyloxy, n-butyloxy, tert-butyloxy, isobutyloxy, sec-butyloxy, pentyloxy, isopentyloxy and hexyloxy.

In one embodiment, “alkyloxy” is metbyloxy, ethyloxy, n-propyloxy, isopropyloxy or tert-butyloxy.

The term of “alkenyloxy” induces a group wherein an oxygen atom is substituted with the above “alkenyl”. Examples are vinyloxy, allyloxy, 1-propenyloxy, 2-butenyloxy, 2-pentenyloxy, 2-hexenyloxy, 2-heptenyloxy and 2-octenyloxy.

The term of “alkynytoxy” induces a group wherein an oxygen atom is substituted with the above “alkynyl”. Examples are ethynyloxy, 1-propynyloxy, 2-propynyloxy, 2-butynyloxy, 2-pentynyloxy, 2-hexynyloxy, 2-heptynyloxy and 2-octynyloxy.

The term of “haloalkyl” includes a group wherein one or more hydrogen atoms attached to one or more carbon atoms of the above “alkyl” are replaced with one or more above “halogen”. Examples are monofluoromethyl, monofluoroethyl, monofluoropropyl, difluoromethyl, difluoroethyl, difluoropropyl, trifluoromethyl, trifluoroothyl, trifluoropropyl, pentafluoropropyl, monochloromethyl, monochloroethyl, monochloropropyl, dichloromethyl, dichloroethyl, dichloropropyl, trichloromethyl, trichloroethyl, trichloropropyl, pentachloropropyl, 1-fluoromethyl, 2-fluoroethyl, 1,1-difluoroethyl, 2,2-difluoroethyl, 2,2,2′-trifluoroethyl, 1-chloroethyl, 2-chloroethyl, 1,1-dichloroethyl, 2,2-dichloroethyl, 2,2,2-trichloroethyl, 1,2-dibromoethyl, 1,1,1-trifluoropropan-2-yl and 2,2,3,3,3-pentafluoropropyl. Examples are monofluoromethyl, difluoromethyl, trifluoromethyl, 1-fluoroethyl, 1,1-difluoroethyl, and 2,2-difluoroethyl. Examples are monofluoromethyl, difluoromethyl, 1-fluoroehtyl, 1,1-difluoroethyl and 2,2-difluoroethyl.

The term of “haloalkenyl” includes a group wherein one or more hydrogen atoms attached to one or more carbon atoms of the above “alkenyl” are replaced with one or more above “halogen”. Examples are monofluorovinyl, monofluoroallyl, monofluoropropenyl, difluorovinyl, difluoroallyl and difluoropropenyl.

The term of “haloalkynyl” includes a group wherein one or more hydrogen atoms attached to one or more carbon atoms of the above “alkynyl” are replaced with one or more above “halogen”. Examples are fluoroethynyl, monofluoropropynyl, difluoropropynyl, monofluorobutynyl, chloroethynyl, monochloropropynyl, monochlorobutynyl and dichloropropynyl.

The term of “haloalkyloxy” includes a group wherein an oxygen atom is substituted with the above “haloalkyl”. Examples are monofluoromethyloxy, monofluorethyloxy, difluoromethyloxy, 1,1-difluoroethyloxy, 2,2-difluoroethyloxy, trifluoromethyloxy, trichloromethyloxy, 2,2,2-trifluoroethyloxy and trichloroethyloxy.

In one embodiment, “haloalkyloxy” is difluoromethyloxy, 2,2,2-difluoroethyloxy, trifluoromethyloxy, 2,2,2-trifluoroethyloxy, or trichloromethyloxy.

The term of “cyanoalkyloxy” includes a group wherein the above “alkyloxy” is substituted with a cyano group. Examples are cyanomethyloxy and cyanoethyloxy.

The term of “alkyloxyalkyl” includes a group wherein the above “alkyl” is substituted with the above “alkyloxy”. Examples are methoxymethyl, methoxyethyl and ethoxymethyl.

The term of “alkyloxyalkyloxy” includes a group wherein the above “alkyloxy” is substituted with the above “alkyloxy”. Examples are methyloxymethyloxy, methyloxyethyloxy, ethyloxymethyloxy and ethyloxyethyloxy.

The term of “cycloalkylalkyloxy” includes a group wherein the above “alkyloxy” is substituted with the above “cycloalkyl”. Examples are cyclopropylmethyloxy, cyclopropylethyloxy, cyclobutytmethyloxy and cyclobutylethyloxy.

The term of “alkylcarbonyl” includes a group wherein a carbonyl group is substituted with the above “alkyl”. Examples are methylcarbonyl, ethylcarbonyl, n-propylcarbonyl, isopropylcarbonyl, tertbutylcarbonyl, isobutylcarbonyl, sec-butylcarbonyl, pentylcarbonyl, isopentylcarbonyl and hexylcarbonyl. Examples are methylcarbonyl, ethylcarbonyl and n-propylcarbonyl.

The term of “alkenylcarbonyl” includes a group wherein a carbonyl group is substituted with the above “alkenyl”. Examples are ethylenylcarbonyl, propenylcarbonyl and butenylcarbonyl.

The term of “alkynylcarbonyl” includes a group wherein a carbonyl group is substituted with the above “alkynyl”. Examples are ethynylcarbonyl, propynyl carbonyl and butynylcarbonyl.

The term of “monoalkylamino” includes a group wherein a hydrogen atom attached to a nitrogen atom of an amino group is replaced with the above “alkyl”. Examples are methylamino, ethylamino and isopropylamino. In one embodiment, “monoalkylamino” is methylamino or ethylamino.

The term of “dialkylamino” includes a group wherein two hydrogen atoms attached to a nitrogen atom of an amino group are replaced with two above “alkyl”. These two alkyl groups may be the same or different. Examples are dimethylamino, diethylamide, N,N-diisopropylamino, N-methyl-N-ethylamino and K-isopropyl-N-ethylamino.

In one embodiment, “dialkylamino” is dimethylamino or diethylamino.

The term of “alkylsulfonyl” includes a group wherein a aulfonyl group is substituted with the above “alkyl”. Examples are methylsulfonyl, ethylsulfonyl, propylsulfonyl, isopropylsulfonyl, tert-butylsulfonyl, isobutylsulfonyl and sec-butylsulfonyl.

In one embodiment, “alkylsulfonyl” is methylsulfonyl or ethylsulfonyl.

The term of “alkenylsulfonyl” includes a group wherein a sulfonyl group is substituted with the above “alkenyl”. Examples are ethylenylsulfonyl, propenyl sulfonyl and butenylsulfonyl.

The term of “alkynylsulfonyl” includes a group wherein a sulfonyl group is substituted with the above “alkynyl”. Examples are ethynylsulfonyl, propynylsulfonyl and butynylsulfonyl.

The term of “monoalkylcarbonylamino” includes a group wherein a hydrogen atom attached to a nitrogen atom of an amino group is replaced with the above “alkylcarbonyl”. Examples are methylcarbonylamino, ethylcarbonylamino, propylcarbonylamino, isopropylcarbonylamino, tert-butylcarbonylamino, isobutylcarbonylamino and sec-butylcarbonylamino.

In one embodiment, “monoalkylcarbonylamino” is methycarbonylamino or ethylcarbonylamino.

The term of “dialkylcarbonylamino” includes a group wherein two hydrogen atoms attached to a nitrogen atom of an amino group are replaced with two ahove “alkylcarbonyl”. These two alkylcarbonyl groups may be the same or different. Examples are dimethylcarbonylamino, diethylcarbonylamino and N,N-diisopropylcarbonylamino. In one embodiment, “dialkylcarbonylamino” is dimethylcarbonylamino or diethylcarbonylamino.

The term of “monoalkylsulfonylamino” includes a group wherein a hydrogen atom attached to a nitrogen atom of an amino group is replaced with the above “alkylsulfonyl”. Examples are methylsulfonylamino, ethylsulfonylamino, propylsulfonylamino, isopropylsulfonylamino, tert-butylsulfonylamino, isobutylsulfonylamino and sec-butylsulfonylamino. In one embodiment, monoalkylsulfonylamino” is methylsulfonylamino or ethylsulfonylamino.

The term of “dialkylsulfonylamino” includes a group wherein two hydrogen atoms attached to a nitrogen atom of an amino group are replaced with two above “alkylsulfonyl”. These two alkylsulfonyl groups may be the same or different. Examples are dimethylsulfonylamino, diethylsulfonylamino and N,N-diisopropylsulfonylamino. In one embodiment, “dialkylsulfonylamino” is dimethylsulfonylamino or diethylsulfonylamino.

The term of “alkylimino” includes a group wherein a hydrogen atom attached to a nitrogen atom of an imino group is replaced with the above “alkyl”. Examples are methylimino, ethylimino, n-propylimino and isopropylimino.

The term of “alkenylimino” includes a group wherein a hydrogen atom attached to a nitrogen atom of an imino group is replaced with the above “alkenyl”. Examples are ethylenylimino and propenylimino.

The term of “alkynylimino” includes a group wherein a hydrogen atom attached to a nitrogen atom of an imino group is replaced with the above “alkynyl”. Examples are ethynylimino and propynylimino.

The term of “alkylcarbonylimino” includes a group wherein a hydrogen atom attached to a nitrogen atom of an imino group is replaced with the above “alkylcarbonyl”. Examples are methycarbonylimino, ethylcarbonylimino, n-propylcarbonylimino and isopropylcarbonylimino.

The term of “alkenylcarbonylimino” includes a group wherein a hydrogen atom attached to a nitrogen atom of an imino group is replaced with the above “alkenylcarbonyl”. Examples are ethylenylcarbonylimino and propenylcarbonylimino.

The term of “alkynylcarbonylimino” includes a group wherein a hydrogen atom attached to a nitrogen atom of an imino group is replaced with the above “alkynylcarbonyl”. Examples are ethynylcarbonylimino and propynylcarbonylimino.

The term of “alkyloxyimino” includes a group wherein a hydrogen atom attached to a nitrogen atom of an imino group is replaced with the above “alkyloxy”. Examples are methyloxyimino, ethyloxyimino, n-propyloxyimino and isopropyloxyimino.

The term of “alkenyloxyimino” includes a group wherein a hydrogen atom attached to a nitrogen atom of an imino group is replaced with the above “alkynyloxy”. Examples are ethylenyloxyimino and propenyloxyimino.

The term of “alkynyloxyimino” includes a group wherein a hydrogen atom attached to a nitrogen atom of an imino group is replaced with the above “alkynyloxy”. Examples are ethynyloxyimino and propynyloxyimino.

The term of “alkynylcarbonyloxy” includes a group wherein an oxygen atom is substituted with the above “alkylcarbonyl”. Examples are methylcarbonyloxy, ethylcarbonyloxy, propylcarbonyloxy, isopropylcarbonyloxy, tert-butylcarbonyloxy, isobutylcarbonyloxy and sec-butylcarbonyloxy. In one embodiment, “alkylcarbonyloxy” is methylcarbonyloxy or ethylcarbonyloxy.

The term of “alkenylcarbonyloxy” includes a group wherein an oxygen atom is substituted with the above “alkylcarbonyl”. Examples are ethylenylcarbonyloxy and propenylcarbonyloxy

The term of “alkynylcarbonyloxy” includes a group wherein an oxygen atom is substituted with the above “alkynylcarbonyl”. Examples are ethynylcarbonyloxy and propynylcarbonyloxy.

The term of “alkyloxycarbonyl” includes a group wherein a carbonyl group is substituted with the above “alkyloxy”. Examples are methyloxycarbonyl, ethyloxycarbonyl, propyloxycarbonyl, isopropyloxycarbonyl, tert-butyloxycarbonyl, isobutyloxycarbonyl, secbutyloxycarbonyl, pentyloxycarbonyl, isopentyloxycarbonyl and hexyloxycarbonyl. In one embodiment, “alkyloxycarbonyl” is methyloxycarbonyl, ethyloxycarbonyl or propyloxycarbonyl.

The term of “alkenyloxycarbonyl” includes a group wherein a carbonyl group is substituted with the above “alkenyloxy”. Examples are ethylenylcarbonyl, propenyloxycarbonyl and butenyloxycarbonyl.

The term of “alkynyloxycarbonyl” includes a group wherein a carbonyl group is substituted with the above, “alkynyloxy”. Examples are ethynyloxycarbonyl, propynyloxycarbonyl and butynyloxyarbonyl.

The term of “alkylsulfanyl” includes a group wherein a hydrogen atom attached to a sulfur atom of a sulfanyl group is replaced with the above “alkyl”. Examples are methylsulfanyl, ethylsulfanyl, n-propylsulfanyl, isopropylsulfanyl, tert-butylsulfanyl and isubotylsulfanyl.

The term “cyanoalkylsulfanyl” includes a group wherein the above “alkylsulfanyl” is substituted with a cyano group. Examples are cyanomethylsulfanyl, cyanoethylsulfanyl and cyanopropylsulfanyl.

The term of “alkenylsulfanyl” includes a group wherein a hydrogen atom attached to a sulfur atom of a sulfanyl group is replaced with the above “alkenyl”. Examples are ethylethylsulfanyl, propenylsulfanyl and butenylsulfanyl.

The term of “alkynylsulfanyl” includes a group wherein a hydrogen atom attached to a sulfur atom of a sulfanyl group is replaced with the above “alkynyl”. Examples are ethynylsulfanyl, propynylsulfanyl and butynylsulfanyl.

The term of “alkylfulfanyl” includes a group wherein a sulfinyl group is substituted with the above “alkyl”. Examples are methylsulfinyl ethylsulfinyl, n-propylsulfinyl and isopropylsulfinyl.

The term of “alkenysulfinyl” includes a group wherein a sulfonyl group is substituted with the above “alkenyl”. Examples are ethylenylsulfinyl, propenylsulfinyl and butenylsulfinyl.

The term of “alkynylsulfinyl” includes a group wherein a sulfinyl group is substituted with the above “alkynyl”. Examples are ethynylsulfinyl, propynylsulfinyl and butynylsulfinyl.

The term of “monoalkylcarbamoyl” includes a group wherein a hydrogen atom attached to a nitrogen atom of a carbamoyl group is replaced with the above “alkyl”. Examples are methylcarbamoyl, ethylcarbamoyl, n-propylcarbamoyl and isopropylcarbamoyl.

The term of “dialkylcarbamoyl” includes a group wherein two hydrogen atom attached to a nitrogen atom of a carbamoyl group are replaced with two above “alkyl”. These two alkyl groups may be the same or different. Examples are dimethycarbamoyl, diethlcarbamoyl and N-methyl-N-ethylcarbamoyl.

The term of “monoalkylsulfamoyl” includes a group wherein a hydrogen atom attached to a nitrogen atom of a sulfamoyl group is replaced with the above “alkyl”. Examples are methylsulfamoyl, ethylsulfamoyl, n-propylsulfumoyl and isopropylsulfamoyl.

The term of “dialkylsulfamoyl” includes a group wherein two hydrogen atoms attached to a nitrogen atom of a sulfamoyl group are replaced with two above “alkyl”. These two alkyl groups may be the same or different. Examples are dimethylsulfamoyl, diethylsulfamoyl and N-methyl-N-ethylsulfamoyl.

The term of “trialkylsilyl” includes a group wherein a silicon atom is substituted with three above “alkyl”. These three alkyl groups may be the same or different. Examples are trimethylsilyl, triethylsilyl and tertbutyldimethylsilyl.

The term of “alkylidene” includes a divalent group derived from alkane by removing two hydrogen atoms from the same carbon atom. Examples are methylidene, ethylidene, propylidene, isopropylidene, butylidene, pentylidene and hexylidene.

The alkenyl portion of “alkenylcarbonylamino”, “alkyloxyalkenyloxy”, “alkenylsulfanyl” and “alkenylamino” means the above “alkenyl”.

The alkynyl portion of “alkynylcarbonylamino”, “alkyloxyalkynyloxy”, “alkynylsulfanyl” and “alkynylamino” means the above “alkynyl”.

The alkyl portion of “hydroxyalkyl”, “hydroxyalkyloxy”, “monoalkylcarbonylamino”, “dialkylcarbonylamino”, “monoalkylamino”, “dialkylamino”, “aminoalkyl”, “alkyloxyalkenyloxy”, “alkyloxyalkynyloxy”, “alkylcarbonyl”, “monoalkylcarbamoyl”. “dialkylcarbamoyl”, “hydroxyalkylcarbamoyl”, “alkyloxyamino”, “alkylsulfanyl”, “monoalkylsulfonylamino”, “dialkylsulfonylamino”, “alkylsulfonylalkylamino”, “alkylsulfonylimino”, “alkylsulfinyl”, “alkylsulfinylamino”, “alkylsulfinylalkylamino”, “alkylsulfinylimino”, “monoalkylsulfamoyl”, “dialkylsulfamoyl”, “aromatic carbocyclylalkyl”, “non-aromatic carbocyclylalkyl”, “aromatic hetorocyclylalkyl” and “non-aromatic heterocyclylalkyl”, “aromatic carbocyclylalkyloxy”, “non-aromatic carbocyclylalkyloxy”, “aromatic heterocyclylalkyloxy” and “non-aromatic heterocyclylalkyloxy”, “aromatic carbocyclylalkyloxycarbonyl”, “non-aromatic carbocyclylalkyloxycarbonyl”, “aromatic heterocyclylalkyloxycarbonyl” and “non-aromatic hetarocyclylalkyloxycarbonyl”, “aromatic carbocyclylalkyloxyalkyl”, “non-aromatic carbocyclylalkyloxyalkyl”, “aromatic hetecoryclylalkyloxyalkyl” and “non-aromatic heterocyclylalkyloxyalkyl”, “aromatic carbocyclylalkylamino”, “non-aromatic carbocyclylalkylamino”, “aromatic heterocyclylalkylamino”, “non-aromatic, heterocyclylalkylamino”, “aromatic carbocyclylalkylcarbamoyl”, “non-aromatic carbocyclylalkylcarbamoyl”, “aromatic heterocyclylalkylcarbamoyl” and “non-aromatic heterocyclylalkylcarbamoyl”, and “cycloalkylalkyl” means the above “alkyl”.

The term of “aromatic carbocyclylalkyl” includes alkyl substituted with one or more above “aromatic carbocyclyl”. Examples are benzyl, phenethyl, phenylpropyl, benzhydryl, trityl, naphthylmethyl and a group of the formula of

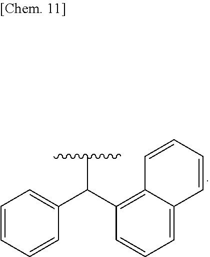 [SMILES: *C(c1ccccc1)c1cccc2ccccc12]

In one embodiment, “aromatic carbocyclylalkyl” is benzyl, phenethyl or benzhydryl.

The term of “non-aromatic carbocyclylalkyl” includes alkyl substituted with one or more above “non-aromatic carbocyclyl”. Also, “non-aromatic carbocyclylalkyl” includes a “non-aromatic carbocyclyl alkyl” wherein the alkyl portion thereof is substituted with one or more above “aromatic carbocyclyl”. Examples are cyclopropylmethyl, cyclobutylmethyl, cyclopentylmethyl, cyclohexylmethyl and a group of the formula of

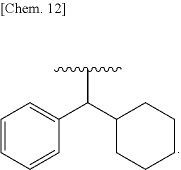 [SMILES: *C(c1ccccc1)C1CCCCC1]

The term of “aromatic heterocylylalkyl” includes alkyl substituted with one or more above “aromatic heterocyclyl”. Also, “aromatic heterocyclylalkyl” includes “aromatic heterocyclyl alkyl” wherein the alkyl portion thereof is substituted with one or more above “aromatic carbocyclyl”, and/or “non-aromatic carbocyclyl”. Examples are pyridylmethyl, furanylmethyl, imidaaolylmethyl, indolylmethyl, benzothiophenylmethyl, oxazolylmethyl, isoxazolylmethyl, thiazolylmethyl, isothiazolylmethyl, pyrazolylmethyl, isopyrazolylmethyl, pyrrolidinylmethyl, benzoxazolylmethyl and groups of the formula of

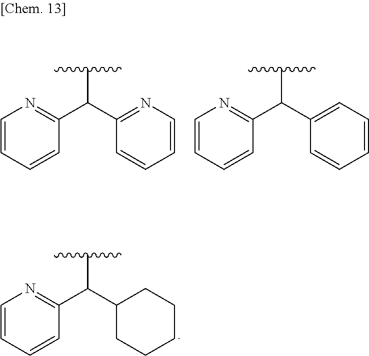

The term of “non-aromatic hetorocyclylalkyl” includes alkyl substituted with one or more above “non-aromatic heterocyclyl”. Also, “non-aromatic heterocyclylalkyl” includes a “non-aromatic heterocyclylalky” wherein the alkyl portion thereof is substituted with one or more above “aromatic carbocyclyl”, “non-aromatic carbocyclyl” and/or “aromatic heterocyclyl”. Examples are tetrahydropyronylmethyl, morpholinylmethyl, morpholinylethyl, pipendinylmethyl, piperazinylmethyl and groups of the formula of

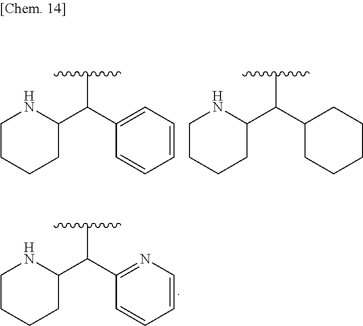

The term of “aromatic carbocyclylalkyloxy” includes alkyloxy substituted with one or more above “aromatic carbocyclyl”. Examples are benzyloxy, phenethyloxy, phenylpropyloxy, benzhydryloxy, triyloxy, naphthylmethyloxy and a group of the formula of

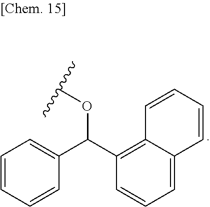 [SMILES: *OC(c1ccccc1)c1cccc2ccccc12]

The term of non-aromatic carbocyclylalkyloxyl includes alkyloxy substituted with one or more above “non-aromatic carbocyclyl”. Also, “non-aromatic carbocyclylalkyloxy” includes a “non-aromatic carbocyclylalkyloxy” wherein the alkyl portion thereof is substituted with one or more above “aromatic carbocyclyl”. Examples are cyclopropylmethyloxy, cyclobutylmethyloxy, cyclopentylmethyloxy, cyclohexylmethyloxy and a group of the formula of

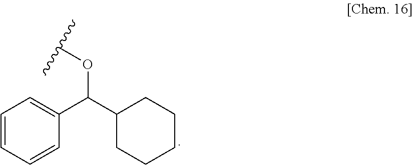 [SMILES: *OC(c1ccccc1)C1CCCCC1]

The term of “aromatic heterocyclylalkyloxy” includes alkyloxy substituted with one or more above “aromatic heterocyclyl”. Also, “aromatic hetcrocyclylalkyloxy” includes “aromatic heterocyclylalkyloxy” wherein the alkyl portion thereof is substituted with one or more above “aromatic carbocyclyl”, and/or “non-aromatic carbocyclyl”. Examples are pyridylmethyloxy, furanylmethyloxy, imidazolylmethyloxy, indolylmethyloxy, benzothiophenylmethyloxy, oxazolylmethyloxy, isoxazolylmethyloxy, thiazolylmethyloxy, isothiazolylmothyloxy, pyrazolylmethyloxy, isopyrazolylmethyloxy, pyrrolidinylmethyloxy, benzoxazolylmethyloxy and groups of the formula of

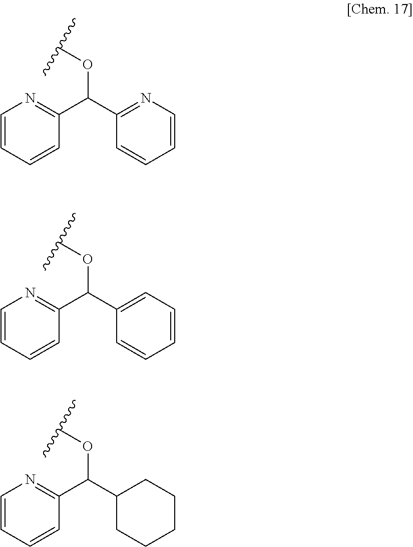 [SMILES: *OC(c1ccccn1)c1ccccn1 ; *OC(c1ccccc1)c1ccccn1 ; *OC(c1ccccn1)C1CCCCC1]

The term of “non-aromatic heterocyclylalkyloxy” includes alkyloxy substituted with one or more above “non-aromatic heterocyclyl”. Also, “non-aromatic heterocyclylalkyloxy” includes a “non-aromatic heteroyclylalkyloxy” wherein the alkyl portion thereof is substituted with one or more above “aromatic carbocyclyl”, “non-aromatic carbocyclyl” and/or “aromatic heterocyclyl”. Examples are tetrahydropyranylmethyloxy, morpholinylmethyloxy, morpholinylethyloxy, piperidinylmethyloxy, piperazinylmethyloxy and groups of the formula of

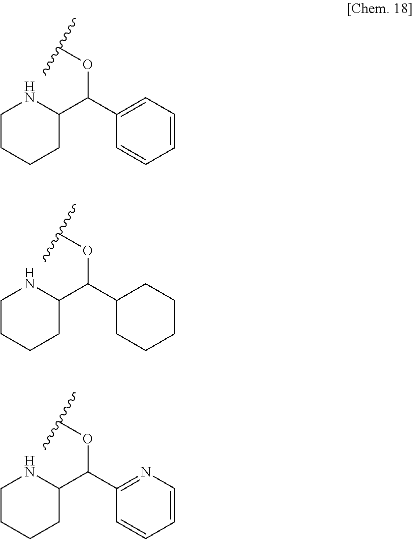 [SMILES: *OC(c1ccccc1)C1CCCCN1 ; *OC(C1CCCCC1)C1CCCCN1 ; *OC(c1ccccn1)C1CCCCN1]

The term of “aromatic carbocyclyl alkyloxycarbonyl” includes alkyloxycarbonyl substituted with one or more above “aromatic carbocyclyl”. Examples are benxyloxycarbonyl, phenethyloxycarbonyl, phenylpropyloxycarbonyl, benzhydryloxycarbonyl, trityloxycarbonyl, naphthylmethyloxycarbonyl and a group of the formula of

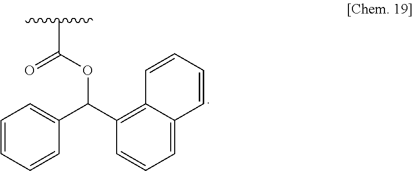 [SMILES: *C(=O)OC(c1ccccc1)c1cccc2ccccc12]

The term of “non-aromatic carbocyclylalkyloxycarbonyl” includes alkyloxycarbonyl substituted with one or more above “non-aromatic carbocyclyl”. Also, “non-aromatic carbocyclylalkyloxycarbonyl” includes “non-aromatic carbocyclylalkyloxycarbonyl” wherein the alkyl portion thereof is substituted with one or more above “aromatic carboryclyl”. Examples are cyclopropylmethyloxycarbonyl, cyclobutylmethoxycarbonyl, cyclopentylmethyloxycarbonyl, cyclohexylmethyloxycarbonyl and a group of the formula of

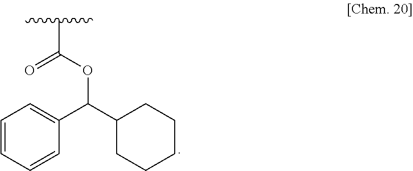 [SMILES: *C(=O)OC(c1ccccc1)C1CCCCC1]

The term of “aromatic heterocycyl alkyloxycarbonyl” includes alkyloxycarbonyl substituted with one or more above “aromatic heterocyclyl”. Also, “aromatic heterocyclyl alkyloxycarbonyl” includes “aromatic heterocyclyl alkyloxycarbonyl” wherein the alkyl portion thereof is substituted with one or more above “aromatic carbocyclyl”, and/or “non-aromatic carbocyclyl”. Examples are pyridylmethyloxycarbonyl, furanylmethyloxycarbonyl, imidazolylmethyloxycarbonyl, indolylmethyloxycarbonyl, benzothiophenylmethyloxycarbonyl, oxazolylmethyloxycarbonyl, isoxazolylmethyloxycarbonyl, thiazolylmethyloxycarbonyl, isothiazolylmethyloxycarbonyl, pyrazolylmethyloxycarbonyl, isopyrazolylmethyloxycarbonyl, pyrrolidinylmethyloxycarbonyl, benzoxazolylmethyloxycarbonyl and groups of the formula of

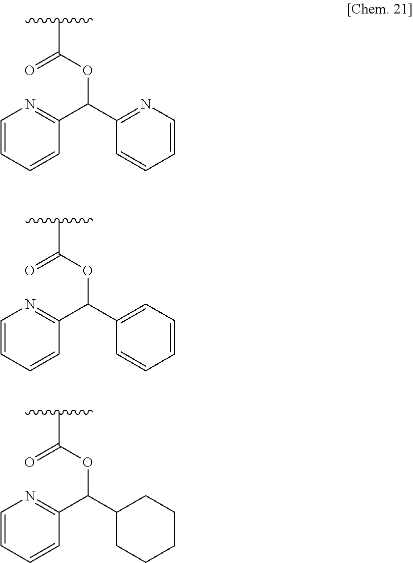 [SMILES: *C(=O)OC(c1ccccn1)c1ccccn1 ; *C(=O)OC(c1ccccc1)c1ccccn1 ; *C(=O)OC(c1ccccn1)C1CCCCC1]

The term of “non-aromatic heterocylyl alkyloxycarbonyl” includes alkyloxy carbonyl substituted with one or more above “non-aromatic heterocyclyl”. Also, “non-aromatic heterocyclyl alkyloxycarbonyl” includes “non-aromatic heteroryclyl alkyloxycarbonyl” wherein the alkyl portion thereof is substituted with one or more above “aromatic carbocyclyl”, “non-aromatic carbocyclyl” and/or “aromatic hetorocyclyl”. Examples are tetrahydropyranylmethyloxycarbonyl, morpholinylmethyloxycarbonyl, morpholinylethyloxycarbonyl, piperidinylmethyloxycarbonyl, piperazinylmethyloxycarbonyl and groups of the formula of

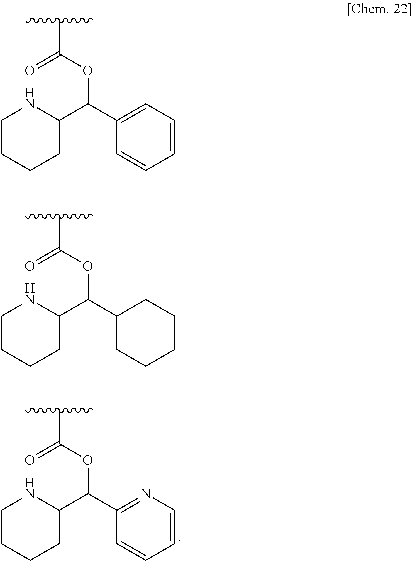 [SMILES: *C(=O)OC(c1ccccc1)C1CCCCN1 ; *C(=O)OC(C1CCCCC1)C1CCCCN1 ; *C(=O)OC(c1ccccn1)C1CCCCN1]

The term of “aromatic carbocyclylalkyloxyalkyl” includes alkyloxyalkyl substituted with one or more above “aromatic carbocyclyl”. Examples are benzyloxymethyl, phenethyloxymethyl, phenylpropyloxymethyl, benzhydryloxymethyl, trityloxymethyl, naphthylmethyloxymethyl and a group of the formula of

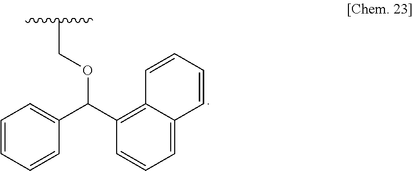 [SMILES: *COC(c1ccccc1)c1cccc2ccccc12]

The term of “non-aromatic carbocyclylalkyloxyalkyl” includes alkyloxyalkyl substituted with one or more above “non-aromatic carbocyclyl”. Also, “non-aromatic carbocyclylalkyloxyalkyl” includes a “non-aromatic carbocyclylalkyloxyalkyl” wherein the alkyl portion attached to non-aromatic carbocyclyl is substituted with one or more above “aromatic carbocyclyl”. Examples are cyclopropylmethyloxymethyl, cyclobutylmethyloxymethyl, cyclopentylmethyloxymethyl, cyclohexylmethyloxymethyl and a group of the formula of

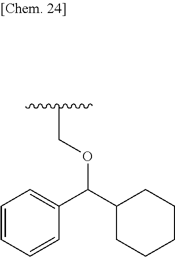 [SMILES: *COC(c1ccccc1)C1CCCCC1]

The term of “aromatic heterocyclylalkyloxyalkyl” includes alkyloxyalkyl substituted with one or more above “aromatic heterocyclyl”. Also, “aromatic heterocyclylalkyloxyalkyl” includes “aromatic heterocyclylalkyloxyalkyl” wherein the alkyl portion attached to aromatic heterocyclyl is substituted with one or more above “aromatic carbocyclyl” and/or “non-aromatic carbocyclyl”. Examples are pyridylmethyloxymethyl, furanylmethyloxymethyl, imidazolylmethyloxymethyl, indolylmethyloxymethyl, benzothiophenylmethyloxymethyl, oxazolylmethyloxymethyl, isoxazolylmethyloxymethyl, thiazolylmethyloxymethyl, isothiazolylmethyloxymethyl, pyrazolylmethyloxymethyl, isopyrazolylmethyloxymethyl, pyrrolidinylmethyloxymethyl, benzoxazolylmethyloxymethyl and groups of the formula of

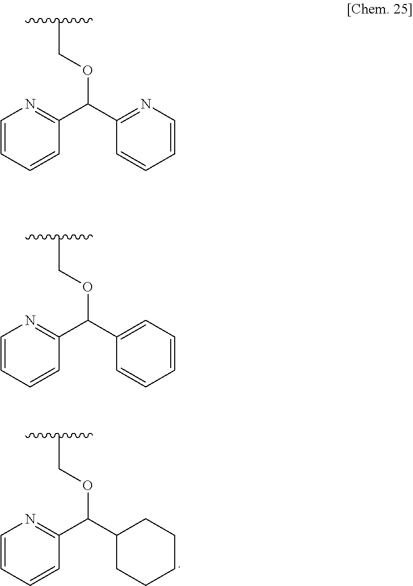 [SMILES: *COC(c1ccccn1)c1ccccn1 ; *COC(c1ccccc1)c1ccccn1 ; *COC(c1ccccn1)C1CCCCC1]

The term of “non-aromatic heterocyclylalkyloxyalkyl” includes alkyloxyalkyl substituted with one or more above “non-aromatic heterocyclyl”. Also, “non-aromatic heterocyclylalkyloxyalkyl” includes “non-aromatic heterocyclylalkyloxyalkyl” wherein the alkyl portion attached to non-aromatic heterocyclyl is substituted with one or more above “aromatic carbocyclyl”, “non-aromatic carbocyclyl” and/or “aromatic heterocyclyl”. Examples are tetrahydropyranylmethyloxymethyl, morpholinylmethyloxymethyl, morpholinylethyloxymethyl, piperidinylmethyloxymethyl, piperazinylmethyloxymethyl and groups of the formula of

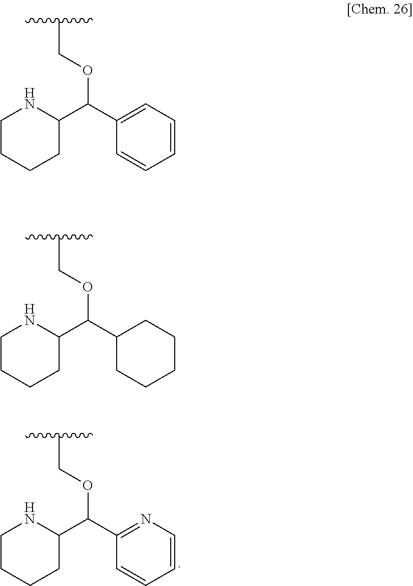 [SMILES: *COC(c1ccccc1)C1CCCCN1 ; *COC(C1CCCCC1)C1CCCCN1 ; *COC(c1ccccn1)C1CCCCN1]

The term of “aromatic carbocyclylalkylamino” includes a group wherein one or two hydrogen atoms attached to a nitrogen atom of an amino group is replaced with the above “aromatic carbocyclylalkyl”. Examples are benzylamino, phemethylamino, phenylpropylamino, benzhydrylamino, tritylamino, naphthylmethylamino and dibenzylamino.

The term of “non-aromatic carbocyclylalkylamino” includes a group wherein one or two hydrogen atoms attached to a nitrogen atom of an amino group is replaced with the above “non-aromatic carbocyclylalkyl”. Examples are cyclopropylmethylamino, cyclobutylmethylamino, cyclopentylmethylamino and cyclohexylmethylamino.

The term of “aromatic heterocyclylalkylamino” includes a group wherein one or two hydrogen atoms attached to a nitrogen atom of an amino group is replaced with the above “aromatic heterocyclylylkyl”. Examples are pyridylmethylamino, furanylmethylamino, imidazolylmethylamino, indolylmethylamino, benzothiophenylmethylamino, oxazolylmethylamino, isoxazolylmethylamino, thiazolylmethylamino, isothiaxolylmethylamino, pyrazolylmethylamino, isopyrazolylmethylamino, pyrrolidinylmethylamino and benzoxazolylmethylamino.

The term of “non-aromatic heterocyclylalkylamino” includes a group wherein one or two hydrogen atoms attached to a nitrogen atom of an amino group is replaced with the above “non-aromatic heterocyclylalkyl”. Examples are tetrahydropyranylmethylamino, morpholinylethylamino, piperadinylmethylamino and piperazinylmethylamino.

The term of “aromatic carbocyclylalkylcarbamoyl” includes a group wherein one or two hydrogen atoms attached to a nitrogen atom of a carbamoyl group is replaced with the above “aromatic carbocycylalkyl”. Examples are benzylcarbamoyl, phenethylcarbamoyl, phenylpropylcarbamoyl, benzhydrylcarbamoyl, tritylcarbamoyl, naphthylmethylcarbamoyl and dibenzylcarbamoyl.

The term of “non-aromatic carbocyclylalkylcarbamoyl” includes a group wherein one or two hydrogen atoms attached to a nitrogen atom of a carbamoyl group is replaced with the above “non-aromatic carbocyclylalkyl”. Examples are cyclopropylmethylcarbamoyl, cyclobutylmethylcarbamoyl, cyclopentylmethylcarbamoyl and cyclohexylmethylcarbamoyl.

The term of “aromatic heterocycylalkylcarbamoyl” includes a group wherein one or two hydrogen atoms attached to a nitrogen atom of a carbamoyl group is replaced with the above “aromatic heterocyclylalkyl”. Examples are pyridylmethylcarbamoyl, furanylmethylcarbamoyl, imidazolylmethylcarbamoyl, indolylmethylcarbamoyl, benzothiophenylmethylcarbamoyl, oxazolylmethylcarbamoyl, isoxazolylmethylcarbamoyl, thiazolylmethylcarbamoyl, isothiazolylmethylcarbamoyl, pyrazolylmethylcarbamoyl, isopyrazolylmethylcarbamoyl, pyrrolidinylmethylcarbamoyl and benzoxazolylmethylcarbamoyl.

The term of “non-aromatic heterocyclylalkylcarbamoyl” includes a group wherein one or two hydrogen atoms attached to a nitrogen atom of a carbamoyl group is replaced with the above “non-aromatic heterocyclyl alkyl”. Examples are tetrahydropyranylmethylcarbamoyl, morpholinylethylcarbamoyl, piperidinylmethylcarbamoyl and piperazinylmethycarbamoyl.

The “aromatic carbocycle” portion of “aromatic carborcycle”, “aromatic carbocyclyloxy”, “aromatic carbocyclylcarbonyl”, “aromatic carbocyclylcarbonyloxy”, “aromatic carbocyclyloxycarbonyl”, “aromatic carbocyclylcarbonylamino”, “aromatic carbocyclylamino”, “aromatic carbocyclylsulfanyl” and “aromatic carbocyclyl sulfonyl”, “aromatic carbocyclylsulfamoyl” and “aromatic carbocyclylcarbamoyl” means the above “aromatic carbocyclyl”.

The term of “aromatic carbocyclyloxy” includes a group wherein an oxygen atom is substituted with the above “aromatic carbocyclyl”. Examples are phenyloxy and naphthyloxy.

The term of “aromatic carboryclylcarbonyl” includes a group whereto a carbonyl group is substituted with the above “aromatic carbocyclyl”. Examples are phenylcarbonyl and naphthylcarbony.

The term of “aromatic carbocyclylcarbonyloxy” includes a group wherein a carbonyloxy group is substituted with the above “aromatic carbocyclyl”. Examples are phenylcarbonyloxy and naphthylcarbonyloxy.

The term of “aromatic carbocyclyloxycarbonyl” includes a group wherein a carbonyl group is substituted with the above “aromatic carbocyclyloxy”. Examples are phenyloxycarbonyl and naphthyloxycarbonyl.

The term of “aromatic carbocyclylcarbonylamino” includes a group wherein one or two hydrogen atoms attached to a nitrogen atom of an amino group is replacad with the above “aromatic carbocyclylcarbonyl”. Examples are benzoylamino and naphthylcarbonylamino.

The term of “aromatic carbocyclylamino” includes a group wherein one or two hydrogen atoms attached to a nitrogen atom of an amino group is replaced with the above “aromatic carbocyclyl”. Examples are phenylamino and naphthylamino.

The term of “aromatic carbocylylsulfanyl” includes a group wherein a hydrogen atom attached to a sulfur atoms of sulfanyl is replaced with the above “aromatic carbocyclyl”. Examples are phenylsulfanyl and naphthylsulfanyl.

The term of “aromatic carbocyclylsulfonyl” includes a group wherein a sulfonyl group is substituted with the above “aromatic carbocyclyl”. Examples are phenylsulfonyl and naphthylsulfonyl.

The term of “aromatic carbocyclylsulfamoyl” includes a group wherein one or two hydrogen atoms attached to a nitrogen atom of a sulfamoyl group is replaced with the above “aromatic carbocyclyl”. Examples are phenylsulfamoyl and naphthylsulfamoyl.

The term of “aromatic carbocyclylcarbamoyl” includes a group wherein one or two hydrogen atoms attached to a nitrogen atom of a carbamoyl group is replaced with the above “aromatic carbocyclyl”. Examples are phenylcarbamoyl and naphthylcarbamoyl.

The “non-aromatic carbocycle” portion of “non-aromatic carbocycle”, “non-aromatic carbocyclyloxy”, “non-aromatic carbocyclylcarbonyloxy”, “non-aromatic carbocyclylcarbonyl”, “non-aromatic carbocyclyloxycarbonyl”, “non-aromatic carbocyclylcarbonylamino”, “non-aromatic carbocyclylamino”, “non-aromatic carbocyclylsulfanyl”, “non-aromatic carbocyclylsulfonyl”, “non-aromatic carbocyclylsulfamoyl” and “non-aromatic carbocyclylcarbamoyl” means the above “non-aromatic carbocyclyl”.

The term of “non-aromatic carboxyclyloxy” includes a group wherein an oxygen atom is substituted with the above “non-aromatic carbocyclyl”. Examples are cyclopropyloxy, cyclohexytoxy and cyclohexenyloxy.

The term of “non-aromalic carbocyclylcarbonyl” includes a group wherein a carbonyl group is substituted with the above “non-aromntic carbocyclyl”. Examples are cyclopropylcarbonyl, cyclohexylcarbonyl and cyclohexenylcarbonyl.

The term of “non-aromatic carbocyclycarbonyloxy” includes a group wherein a carbonyloxy group is substituted with the above “non-aromatic carbocyclyl”. Examples are cyclopropylcarbonyloxy, cyclohexylcarbonyloxy and cyclohexenylcarbonyloxy.

The term of “non-aromatic carbocycyloxycarbonyl” includes a group where in a carbonyl group is substituted with the above “non-aromatic carbocyclyloxy”. Examples are cyclopropyloxycarbonyl, cyclohexyloxycarbonyl and cyclohexenyloxycarbonyl.

The term of “non-aromatic carbocyclylcarbonylamino” includes a group wherein one or two hydrogen atoms attached to a nitrogen atom of an amino group is replaced with the above “non-aromatic carbocyclylcarbonyl”. Examples are cyclopropylcarbonylamino, cyclohexylcarbonylamino and cyclohexenylcarbonylamino.

The term of “non-aromatic carbocyclylamino” includes a group wherein one or two hydrogen atoms attached to a nitrogen atom of an amino group is replaced with the above “non-aromatic carbocyclyl”. Examples are cyclopropylamino, cyclohexylamino and cyclohexenylamino.

The term of “non-aromatic carbocyclylsulfanyl” includes a group wherein a hydrogen atom attached to a sulfur atom of a sulfanyl is replaced with the above “non aromatic carbocyclyl”. Examples are cyclopropylsulfanyl, cyclohexylsulfanyl and cyclohexenylsulfanyl.

The term of “non-aromatic carbocyclylsulfonyl” includes a group wherein a sulfonyl group is substituted with the above “non-aromatic carbocyclyl”. Examples are cyclopropylsulfonyl, cyclohexylsulfonyl and cyclohexenylsulfonyl.

The term of “non-aromatic carbocyclylsulfamoyl” includes a group wherein one or two hydrogen atoms attached to a nitrogen atom of a sulfamoyl group is replaced with the above “non-aromatic carbocyclyl”. Examples are cyclopropylsulfamoyl, cyclohexylsulfamoyl and cyclohexenylsulfamoyl.

The term of “non-aromatic carbocyclylcarbamoyl” includes a group wherein one or two hydrogen atoms attached to a nitrogen atom of a carbamoyl group is replaced with the above “non-aromatic carbocyclyl”. Examples are cyclopropycarbamoyl, cyclohexylcarbamoyl and cyclohexenylcarbamoyl.

The “aromatic heterocycle” portion of “aromatic heterocycle”, “aromatic heterocyclyloxy”, “aromatic heterocylylcarbonyl”, “aromatic heterocyclylcarbonyloxy”, “aromatic heterocyclyloxycarbonyl”, “aromatic heterocyclylcarbonylamino”, “aromatic heterocyclylamino”, “aromatic heterocycylsulfanyl”, “aromatic heterocyclylsulfonyl”, “aromatic heterocyclysulfamoyl” and “aromatic heterocycylcarbamoyl” means the above “aromatic heterocyclyl”.

Examples of “aromatic heterocycle” in ring B is pyridine, pyrazine, pyrimidine, pyridazine and oxazole.

The term of “aromatic heterocyclyloxy” includes a group wherein an oxygen atom is substituted with the above “aromatic heterocyclyl”. Examples are pyridyloxy and oxaxolyloxy.

The term of “aromatic heterocylylcarbonyl” includes a group wherein a carbonyl group is substituted with the above “aromatic heterocyclyl”. Examples are pyridylcarbonyl and oxaxolylcarbonyl.

The term of “aromatic heterocyclylcarbonyloxy” includes a group wherein a carbonyloxy group is substituted with the above “aromatic heterocyclyl”. Examples are pyridylcarbonyloxy and oxazolylcarbonyloxy.

The term of “aromatic heterocyclyloxycarbonyl” includes a group wherein a carbonyl group is substituted with the above “aromatic heterocyclyloxy”. Examples are pyridyloxycarbonyl and oxazolyloxycarbonyl.

The term of “aromatic heterocyclylcarbonylamino” includes a group wherein one or two hydrogen atoms attached to a nitrogen atom of an amino group is replaced with the above “aromatic heterocyclylcarbonyl”. Examples are pyridylcarbonylamino and oxazolylcarbonylamino.

The term of “aromatic heterocyclylamino” includes a group wherein one or two hydrogen atoms attached to a nitrogen atom of an amino group is replaced with the above “aromatic heterocyclyl”. Examples are pyridylamino and oxazolylamino.

The term of “aromatic heterocyclylsulfanyl” includes a group wherein a hydrogen atom attached to a sulfur atom of sulfanyl is replaced with the above “aromatic heterocyclyl”. Examples are pyridylsulfanyl and oxazolylsulfanyl.

The term of “aromatic heterocyclylsulfonyl” includes a group wherein a sulfonyl group is substituted with the above “aromatic heterocyclyl”. Examples are pyridylsulfonyl and oxazolylsulfonyl.

The term of “aromatic heterocyclylsulfamoyl” includes a group wherein one or two hydrogen atoms attached to a nitrogen atom of a sulfamoyl group is replaced with the above “aromatic heterocyclyl”. Examples are pyridylsulfamoyl and oxazolylsulfamoyl.

The term of “aromatic heterocyclylcarbamoyl” includes a group wherein one or two hydrogen atoms attached to a nitrogen atom of a carbamoyl group is replaced with the above “aromatic heterocyclyl”. Examples are pyridylcarbamoyl and oxazolylcarbamoyl.

The “non-aromatic heterocycle” portion of “non-aromatic heterocyclyl”, “non-aromatic heterocyclyloxy”, “non-aromatic heterocyclylcarbonyl”, “non-aromatic heterocyclylcarbonyloxy”, “non-aromatic heterocyclyloxycarbonyl”, “non-aromatic heterocyclylcarbonylamino”, “non-aromatic heterocyclylamino”, “non-aromatic heterocyclylsulfanyl”, “non-aromatic heterocyclylsulfonyl”, “non-aromatic heterocyclylsulfamoyl” and “non-aromatic heterocyclylcarbamoyl” means the above “non-aromatic heterocyclyl”.

The term of “non-aromatic hetorocyclyloxy” includes a group wherein an oxygen atom substituted with the above “non-aromatic heterocyclyl”. Examples are piperidinyloxy and tetrahydrofuryloxy.

The term of “non-aromatic heterocyclylcarbonyl” includes a group wherein a carbonyl group is substituted with the above “non-aromatic heterocyclyl”. Examples are piperidinylcarbonyl and tetrahydrofurylcarbonyl.

The term of “non-aromatic heterocyclylcarbonyloxy” includes a group wherein a carbonyloxy group is substituted with the above “non-aromatic heterocyclyl”. Examples are piperidinylcarbonyloxy and tetrahydrofurylcarbonyloxy.

The term of “non-aromatic heterocyclyloxycarbonyl” includes a group wherein a carbonyl group is substituted with the above “non-aromatic heterocyclyloxy”. Examples are piperidinyloxycarbonyl and tetrahydrofuryloxycarbonyl.

The term of “non-aromatic heterocyclylcarbonylamino” includes a group wherein one or two hydrogen atoms attached to a nitrogen atom of an amino group is replaced with the above “non-aromatic heterocyclylcarbonyl”. Examples are pipieidinylcarbonylamino and tetrahydrofurylcarbonylamino.

The term of “non-aromatic heterocyclylamino” includes a group wherein one or two hydrogen atoms attached to a nitrogen atom of an amino group is replaced with the above “non-aromatic heterocyclyl”. Examples are piperidinylamino and tetrahydrofurylamino.

The term of “non-aromatic heterocyclylsulfanyl” includes a group wherein a hydrogen atom attached to a sulfur atom of sulfanyl is replaced with the above “non-aromatic heterocyclyl”. Examples are piperidinylsulfanyl and tetrahydrofurylsulfanyl.

The term of “non-aromatic heterocyclysulfonyl” includes a group wherein a sulfonyl group in substituted with the above “non-aromatic heterocyclyl”. Examples are piperidinylsulfonyl and tetrahydrofurysulfonyl.

The term of “non-aromatic heterocyclylsulfamoyl” includes a group wherein one or two hydrogen atoms attached to a nitrogen atom of a sulfamoyl group is replaced with the above “non-aromatic heterocyclyl”. Examples are piperidinylsulfamoyl and tetrahydrofurylsulfamoyl.

The term of “non-aromatic heterocyclylcarbamoyl” includes a group wherein one or two hydrogen atoms attached to a nitrogen atom of a carbamoyl group is replaced with the above “non-aromatic heterocyclyl”. Examples are piperidinyl carbamoyl and tetrahydrofurylcarbamoyl.

The term “R^{2a} and R^{2b} together with the carbon atom to which they are attached may form substituted cycloalkane” includes

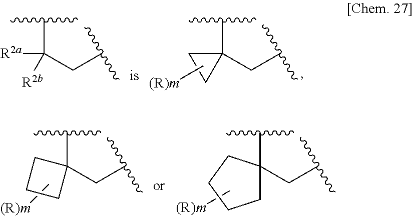

wherein R is halogen or substituted or unsubstituted alkyl, and
m is an integer of 1 or 2.

Examples of substituents of “substituted or unsubstituted alkyl”, “substituted or unsubstituted alkenyl”, and “substituted or unsubstituted alkynyl”, are the group as follows. A carbon atom at any possible position(s) can be substituted with one or more substituents selected from the following groups.

Substituent: halogen, hydroxy, carboxy, amino, imino, hydroxyamino, hydroxyimino, formyl, formyloxy, carbamoyl, sulfamoyl, sulfanyl, sulfino, sulfo, thioformyl, thiocarboxy, dithiocarboxy, thiocarbamoyl, cyano, nitro, nitroso, azide, hydrazino, ureido, amidino, guanidino, trialkylsilyl, alkyloxy, alkenyloxy, alkynylnxy, haloalkyloxy, alkylcarbonyl, alkenylcarbonyl, alkynylcarbonyl, monoalkylamino, dialkylamino,
alkylsulfonyl, alkenylsulfonyl, alkynylsulfonyl, monoalkylcarbonylamino, dialkylcarbonylamino, monoalkylsulfonylamino, dialkylsulfonylamino, alkylimino, alkenylimino, alkynylimino, alkylcarbonylimino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynylcarbonyl, alkylsulfanyl, alkenylsulfanyl, alkynylsulfanyl, alkylsulfinyl, alkenylsulfinyl, alkynylsulfinyl, monoalkylcarbamoyl, dialkylcarbamoyl, monoalkylsulfamoyl, dialkylsulfamoyl, aromatic carbocyclyl, non-aromatic carbocyclyl, aromatic heterocyclyl, non-aromatic heterocyclyl, aromatic carbocyclyloxy, non-aromatic carbocyclyloxy, aromatic heterocyclyloxy, non-aromatic heterocyclyloxy, aromatic carbocyclylcarbonyl, non-aromatic carbocyclycarbonyl, aromatic heterocyclylcarbonyl, non-aromatic heterocyclylcarbonyl, aromatic carbocyclyloxycarbonyl, non-aromatic carbocyclyloxycarbonyl, aromatic heterocyclyloxycarbonyl, non-aromatic heterocyclyloxycarbonyl, aromatic carbocyclyl alkloxy, non-aromatic carbocyclyl alkyloxy, aromatic heterocyclyl alkyloxy, non-aromatic heterocyclyl alkyloxy,
aromatic carbocyclyl alkyloxycarbonyl, non-aromatic carbocyclyl alkyloxycarbonyl, aromatic heterocyclyl alkyloxycarbonyl, non-aromatic heterocyclylalkyloxycarbonyl, aromatic carbocyclyl alkylamino, non-aromatic carbocyclyl alkylamino, aromatic heterocyclyl alkylamino, non-aromatic heterocyclyl alkylamino,
aromatic carbocyclylsulfanyl, non-aromatic carbocyclylsulfanyl, aromatic heteroryclylsulfonyl, non-aromatic heterocyclylsulfonyl,
aromatic carbocyclylsulfonyl, non-aromatic carbocyclylsulfonyl, aromatic heterocyclylsulfonyl, and non-aromatic heterocyclylsulfonyl.

Examples of substituents of “substituted or unsubstituted alkyl” are one or more groups selected from the following substituent group α.

The substituent group α is a group consisting of halogen, hydroxy, alkyloxy, haloalkyloxy, hydroxyalkyloxy, alkyloxyalkyloxy, formyl, alkylcarbonyl, alkenylcarbonyl, alkynylcarbonyl, aromatic carbocyclylcarbonyl, non-aromatic carbocyclylcarbonyl, aromatic heterocyclylcarbonyl, non-aromatic heterocyclylcarbonyl, alkylcarbonyloxy, alkenylcarbonyloxy, aromatic carbocyclylcarbonyloxy, non-aromatic carbocyclylcarbonyloxy, aromatic heterocyclylcarbonyloxy, non-aromatic heterocyclylcarbonyloxy, carboxy, alkyloxycarbonyl, amino, monoalkylcarbonylamino, dialkylcarbonylamino, alkenylcarbonylamino, alkynylcarbonylamino, aromatic carbocyclylcarbonylamino, non-aromatic carbocyclylcarbonylamino, aromatic heterocyclylcarbonylamimo, non-aromatic heterocyclylcarbonylamino, monoalkylamino, dialkylamino, imino, hydroxyimino, alkyloxyamino, alkylsulfanyl, carbamoyl, monoalkylcarbamoyl, dialkylcarbamoyl, hydroxyalkylcarbamoyl, sulfamoyl, monoalkylsulfamoyl, dialkylsulfamoyl, alkylsulfonyl, monoalkylsulfonylamino, dialkylsulfonylamino, alkylsulfonylalkylamino, alkylsulfonylimino, alkylsulfinyl, alkylsulfonylamino, alkylsulfinylalkylamino, alkylsulfinylimino, cyano, nitro, aromatic carbocyclyl, nonaromatic carbocyclyl, aromatic heterocyclyl and non-aromatic heterocyclyl (each of aromatic carbocyclo, non-aromatic carbocycle, aromatic hetercycle and non-aromatic heterocycle is optionally substituted with one or more selected from halogen, alkyl, hydroxy and alkyloxy).

The substituents of “substituted or unsubstituted alkyl” are, for example, halogen, hydroxy and the like.

Examples of substituents of “substituted or unsubstited alkyloxy”, “substituted or unsubstituted alkenyl” and “substituted or unsubstituted alkynyl” are one or more selected from the above substituent group α. Specific examples are halogen, hydroxy and the like.

Examples of substituents of “substituted or unsubstituted amino” are one or two selected from alkyl, alkylcarbonyl, alkenylcarbonyl, alkynylcarbonyl, aromatic carbocyclylcarbonyl, non-aromatic carbocyclylcarbonyl, aromatic heterocylylcarbonyl, non-aromatic heterocyclylcarbonyl, hydroxy, alkyloxy, alkyloxycarbonyl, aromatic carbocyclyl, non-aromatic carbocyclyl, aromatic heterocyclyl and non-aromtic heterocyclyl and the like. Specific examples are alkyl, alkylcarbonyl and the like.

Examples of substituents on “aromatic carbocycle”, “non-aromatic carbocycle”, “cycloalkyl”, “aromatic heterocycle” and “non-aromatic heterocycle” of “substituted or unsubstituted aromatic carbocyclyl”, “substituted or unsubstited non-aromatic carbocyclyl”, “substituted or unsubstituted cycloalkyl”, “substituted or unsubstituted aromatic heterocyclyl”, and “substituted or unsubstituted non-aromatic heterocyclyl” include the group as follows. One or more atoms at any possible position(s) on each ring can be substituted with one or more substituents selected from the following group.

Substituents halogen, hydroxy, carboxy, amino, imino, hydroxyamino, hydroxyimino, formyl, formyloxy, carbamoyl, sulfamoyl, sulfonyl, sulfino, sulfo, thioformyl, thiocarboxy, dithiocarboxy, thiocarbamoyl, cyano, nitro, nitroso, azide, hydrazine, ureido, amidino, guanidino, trialkylsilyl, alkyl, alkenyl, alkynyl, haloalkyl, alkyloxy, alkenyloxy, olkynyloxy, haloalkyloxy, alkyloxyalkyl, alkyloxyalkyloxy, alkylcarbonyl, alkenylcarbonyl, alkynylcarbonyl, monoalkylamino, dialkylamino, alkylsulfonyl, alkenylsulfonyl, alkynylsulfonyl, monoalkylcarbonylamino, dialkylcarbonylamino, monoalkylsulfonylamino, dialkylsulfonylamino, alkylimino, alkenylimino, alkynylimino, alkylcarbonylimino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynyloxycarbonyl, alkylsulfanyl, alkenylsulfanyl, alkynylsulfanyl, alkylsulfinyl, alkenylsulfinyl, alkynylsulfinyl, monoalkylcarbamoyl, dialkylcarbamoyl, monoalkylsulfamoyl, dialkylsulfamoyl, aromatic carbocyclyl, non-aromatic carbocyclyl, aromatic heterocyclyl, non-aromatic heterocyclyl, aromatic carbocyclyloxy, nonaromatic carbocyclyloxy, aromatic heteroryclyloxy, non-aromatic heterocyclyloxy, aromatic carbocyclylcarbonyl, non-aromatic carbocyclylcarbonyl, aromatic heterocyclylcarbonyl, non-aromatic heterocyclylcarbonyl, aromatic carbocyclyloxycarbonyl, non-aromatic carbocyclyloxycarbonyl, aromatic heterocyclyloxycarbonyl, non-aromatic heterocyclyloxycarbonyl, aromatic carbocyclylalkyl, non aromatic carbocyclylalkyl, aromatic heterocyclylalkyl, non-aromatic heterocyclylalkyl,
aromatic carbocyclylalkyloxy, non-aromatic carbocyclylalkyloxy, aromatic heterocyclylalkyloxy, non-aromatic heterocyclylalkyloxy,
aromatic carbocycylalkyloxycarbonyl, non-aromatic carbocyclylalkyloxycarbonyl, aromatic heterocyclylalkyloxycarboxyl, non-aromatic heterocyclylalkyloxycarbonyl, aromatic carbocyclylalkyloxyalkyl, non-aromatic carbocyclylalkyloxyalkyl, aromatic heterocyclylalkyloxyalkyl, non-aromatic heterocyclylalkyloxyalkyl,
aromatic carbocyclylalkylamino, non-aromatic carbocyclylalkylamino, aromatic heterocyclylalkylamino, non-aromatic heterocyclylalkylamino,
aromatic carbocyclylsulfanyl, non-aromatic carbocyclylsulfanyl, aromatic heterocyclylsulfanyl, non-aromatic heterocyclylsulfanyl,
aromatic carbocyclylsulfonyl, non-aromatic carbocyclylsulfonyl, aromatic heterocyclylsulfonyl, and non-aromatic heterocyclylsulfonyl.

A “substituted or unsubstituted non-aromatic carbocyclyl” and “substituted or unsubstituted non-aromatic heterocyclyl” can be substituted with “oxo”. A group wherein two hydrogen atoms attached to the same carbon atom are replaced with oxo as follows is included:

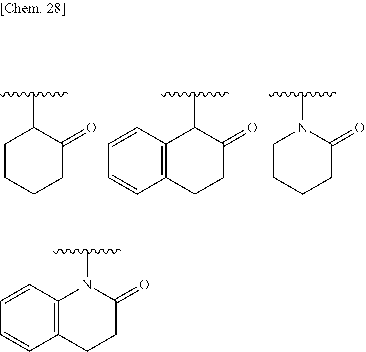

Examples of the substituent of “substituted or unsubstituted aromatic carbocycle”, “substituted or unsubstituted non-aromatic carbocycle”, “substituted or unsubstituted aromatic heterocycle”, “substituted or unsubstituted non-aromatic heterocycle”, “substituted or unsubstituted benzene”, “substituted or unsubstituted pyridine”, “substituted or unsubstituted pyrazine”, “substituted or unsubstituted oxazole”, “substituted or unsubstituted pyrimidine” or “substituted or unsubstituted pyridazine” in ring A and ring B include

(a) a group selected from the substituted group α, for example, halogen, hydroxy, alkyloxy, formyl, alkylcarbonyl, alkenylcarbonyl, alkynylcarbonyl, aromatic carbocyclylcarbonyl, non-aromatic carbocyclylcarbonyl, aromatic heterocyclylcarbonyl, non-aromatic heterocyclylcarbonyl, formyloxy, alkylcarbonyloxy, alkenylcarbonyloxy, alkynylcarbonyloxy, aromatic carbocyclycarbonyloxy, non-aromatic carbocyclylcarbonyloxy, aromatic heterocyclylcarbonyloxy, non-aromatic heterocyclylcarbonyloxy, carboxy, alkyloxycarbonyl, carbamoyl, amino, cyano, monoalkylamino, dialkylamino and/or alkylsulfanyl;
(b) unsubstituted alkyl or alkyl substituted with one or more groups selected from the substituent group α, hydroxyimino and alkyloxyimino;
(c) aminoalkyl substituted with one or more groups selected from the substituent group α;
(d) unsubstituted alkenyl or alkenyl substituted with one or more substituents selected from the substituent group α;
(e) unsubstituted alkynyl or alkynyl substituted with one or more substituent selected from the substituent group α;
(f) alkyloxy substituted with one or more substituted selected from the substituent group α;
(g) alkyloxyalkyloxy substituted with one or more substituents selected from the substituent group α;
(h) unsubstituted alkenyloxy or alkenyloxy substituted with one or more substituent selected from the substituent group α;
(i) alkyloxyalkenyloxy substituted with one or more substituents selected from the substituent group α;
(j) unsubstituated alkynyloxy or alkynyloxy substituted with one or more substituents from the substituent group α;
(k) alkyloxyalkynyloxy substituted with one or more groups selected from the substituent group α;
(l) unsubstituted alkylsulfanyl or alkylsulfanyl substituted with one or more substituents selected from the substituent group α;
(m) unsubstituted alkenylsulfanyl or alkenylsulfanyl substituted with one or more substituents selected from the substituent group α;
(n) unsubstituted alkynylsulfanyl or alkynylsulfanyl substituted with one or more substituents selected from the substituent group α;
(o) monoalkylamino substituted with one or more substituents selected from the substituent group α;
(p) dialkylamino substituted with one or more substituents selected from the substituent group α;
(q) alkenylamino substituted with one or more substituents selected from the substituent group α;
(r) alkynylamino substituted with one or more substituents selected from the substituent group α;
(s) unsubstituted aminooxy or aminooxy substituted with one or more substituents selected from the substituent group α and alkylidene;
(t) alkylcarbonyl substituted with one or more substituents selected from the substituent group α;
(u) alkenylcarbonyl substituted with one or more substituents selected from the substituent group α;
(v) alkynylcarbonyl substituted with one or more substituents selected from the substituent group α;
(w) aromatic carbocyclylcarbonyl substituted with one or more substituents selected from the substituent group α;
(x) non-aromatic carbocyclylcarbonyl substituted with one or more substituents selected from the substituent group α;
(y) aromatic heterocyclylcarbonyl substituted with one or more substituents selected from the substituent group α;
(z) non-aromatic heterocyclylcarbonyl substituted with one or more substituents selected from the substituent group α;
(aa) monoalkylcarbamoyl substituted with one or more substituents selected from the substituent group α;
(ab) dialkylcarbamoyl substituted with one or more substituents selected from the substituent group α;
(ac) alkyloxycarbonyl substituted with one or more substituents selected from the substituent group α;
(ad) unsubstituted alkylsulfonyl or alkylsulfonyl substituted with one or more substituents selected from the substituent group α;
(ae) unsubstituted alkylsulfonyl or alkylsulfonyl substituted with one or more substituents selected from the substituent group α;
(af) monoalkylsulfamoyl substituted with one or more substituents selected from the substituent group α;
(ag) dialkylsulfamoyl substituted with one or more substituents selected from the substituent group α;
(ah) aromatic carbocyclyl substituted with one or more substituents selected from the substituent group α, azide, alkyl and haloalkyl;
(ai) non-aromatic carbocyclyl substituted with one or more substituents selected from the substituent group α, oxide, alkyl and haloalkyl;
(aj) aromatic heterocyclyl substituted with one or more substituents selected from the substituent group α, azide, alkyl and haloalkyl;
(ak) non-aromatic heterocyclyl substituted with one or more substituents selected from the substituent group α, azide, alkyl and haloalkyl;
(al) unsubstituted aromatic carbocyclylalkyl or aromatic carbocyclylalkyl substituted with one or more substituents selected from the substituent group α, azide, alkyl and haloalkyl;
(am) unsubstituted non-aromatic carbooyclylalkyl or non-aromatic carbocyclylalkyl substituted with one or more substituents selected from the substituent group α, azide, alkyl and haloalkyl;
(an) unsubstituted aromatic heterocyclylalkyl or aromatic heterocyclylalkyl substituted with one or more substituents selected from the substituent group α, azide, alkyl and haloalkyl;
(ao) unsubstituted non-aromatic heterocyclylalkyl or non-aromatic heteroryclylalkyl substituted with one or more substituents selected from the substituent group α, azide, alkyl and haloalkyl;
(ap) unsubstituted aromatic carbocyclyoxy or aromatic carbocyclyloxy substituted with one or more substituents selected from the substituent group α, azide, alkyl and haloalkyl;
(aq) unsubstituted non-aromatic carbocyclyloxy or non-aromatic carbocyclyloxy substituted with one or more substituents selected from the substituent group α, azide, alkyl and haloalkyl;
(ar) unsubstituted aromatic heterocyclyloxy or aromatic heterocylyloxy substituted with one or more substituents selected from the substitueut group α, azide, alkyl and haloalkyl;
(as) unsubstituted non-aromatic heterocyclyloxy or non-aromatic heterocyclyloxy substituted with one or more substituents selected from the substituent group α, azide, alkyl and haloalkyl;
(at) unsubstituted aromatic carbocyclylalkyloxy or aromatic carbocyclylalkyloxy substituted with one or more substituents selected from the substituent group α, azide, alkyl and haloalkyl;
(au) unsubstituted non-aromatic carbocyclalkyloxy or non-aromatic carbocyclylalkyloxy substituted with one or more substituents selected from the substituent group α, azide, alkyl and haloalkyl;
(av) unsubstituted aromatic hetorocyclylalkyloxy or aromatic heterocyclylalkyloxy substituted with one or more substituents selected from the substituent group α, azide, alkyl and haloalkyl;
(aw) unsubstituted non-aromatic heteorcyclylalkyloxy ar non-aromatic heterocyclylalkyloxy substituted with one or more substituents selected from the substituent group α, azide, alkyl and haloalkyl;
(ax) unsubstituted aromatic carbocyclylalkyloxycarbonyl or aromatic carbocyclylalkyloxycarbonyl substituted with one or more substituents selected from the substituent group α, azide, alkyl and haloalkyl;
(ay) unsubstituted non-aromatic carbocyclylalkyloxycarbonyl or non-aromatic carbocyclylalkyloxycarbonyl substituted with one or more substituents selected from the substituent group α, azide, alkyl and haloalkyl;
(az) unsubstituted aromatic heterocyclylalkyloxycarbonyl or aromatic heterocyclylalkyloxycarbonyl substituted with one or more substituents selected from the substituent group α, azide, alkyl and haloalkyl;
(ba) unsubstituted non-aromatic heterocyclylalkyloxycarbonyl or non-aromatic heterocyclylalkyloxycarbonyl substituted with one or more substituents selected from the substituent group α, azide, alkyl and haloalkyl;
(bb) unsubstituted aromatic carbocyclylsulfanyl or aromatic carbocyclylsulfanyl substituted with one or more substituents selected from the substituent group α, azide, alkyl and haloalkyl;
(bc) unsubstituted non-aromatic carbocyclylsulfanyl or non-aromatic carbocyclylsulfanyl substituted with one or more substituents selected from the substituent group α, azide, alkyl and haloalkyl;
(bd) unsubstituted aromatic heterocyclylsulfanyl or aromatic heterocyclylsulfanyl substituted with one or more substituents selected from the substituent group α, azide, alkyl and haloalkyl;
(bc) unsubstituted non-aromatic heterocyclylsulfanyl or non-aromatic heterocyclylsulfanyl substituted with one or more substituents selected from the substituent group α, azide, alkyl and haloalkyl;
(bf) unsubstituted aromatic carbocyclylamino or aromatic carbocyclylamino substituted with one or more substituents selected from the substituent group α, azide, alkyl and haloalkyl;
(bg) unsubstituted non-aromatic carbocyclylamino or non-aromatic carbocyclylamino substituted with one or more substituents selected from the substituent group α, azide, alkyl and haloalkyl;
(bh) unsubstituted aromatic heterocyclylamino or aromatic heterocyclylamino substituted with one or more substituents selected from the substituted group α, azide, alkyl and haloalkyl;
(bi) unsubstituted nonaromatic heterocyclylamino or non-aromatic heterocyclylamino substituted with one or more substituents selected from the substituent group α, azide, alkyl and haloalkyl;
(bj) unsubstituted aromatic carbocyclylalkylamino or aromatic carbocyclylalkylamino substituted with one or more substituents selected from the substituent group α, azide, alkyl and haloalkyl;
(bk) unsubstituted non-aromatic carbocyclylalkylamino or non-aromatic carbocyclylalkylamino substituted with one or mere substituents selected from the substituent group α, azide, alkyl and haloalkyl;
(bl) unsubstituted aromatic heterocyclylalkylamino or aromatic heterocyclylalkylamine substituted with one or more substituents selected from the substituent group α, azide, alkyl and haloalkyl;
(bm) unsubstituted nonaromatic heterocyclylalkylamino or non-aromatic heterocyclylalkylamino substituted with one or more substituents selected from the substituent group α, azide, alkyl and haloalkyl;
(bn) unsubstituted aromatic carbocyclylsulfamoyl or aromatic carbocyclylsulfamoyl substituted with one or more substituents selected from the substituent group α, azide, alkyl and haloalkyl;
(bo) unsubstituted non-aromatic carbocyclylsulfamoyl or non-aromatic carbocyclylsulfamoyl substituted with one or more substituents selected from the substituent group α, oxide, alkyl and haloalkyl;
(bp) unsubstituted aromatic heterocyclylsulfamoyl or aromatic heterocyclylsulfamoyl substituted with one or more substituents selected from the substituent group α, azide, alkyl and haloalkyl;
(bq) unsubtituted non-aromatic heterocyclylsulfamoyl or non-aromatic heterocyclylsulfamoyl substituted with one or more substituents selected from the substituent group α, azide, alkyl and haloalkyl;
(br) unsubstituted aromatic carbocyclylsulfonyl or aromatic carbocylylsulfonyl substituted with one or more substituents selected from the substituent group α, azide, alkyl and haloalkyl;
(bs) unsubstituted non-aromatic carbocyclylsulfonyl or non-aromatic carbocyclylsulfonyl substituted with one or more substituents selected from the substituent group α, azide, alkyl and haloalkyl;
(bt) unsubstituted aromatic heterocyclylsulfonyl or aromatic heterocyclylsulfonyl substituted with one or more substituents selected from the substituent group α, azide, alkyl and haloalkyl;
(bu) unsubstituted non-aromatic heterocyclylsulfonyl or non-aromatic heterocyclylsulfonyl substituted with one or more substituents selected from the substituent group α, azide, alkyl and haloalkyl;
(bv) unsubstituted aromatic carbocyclycarbamoyl or aromatic carbocyclylcarbamoyl substituted with one or more substituents selected from the substituent group α, azide, alkyl and haloalkyl;
(bw) unsubstituted non-aromatic carbocyclylcarbamoyl or non-aromatic carbocyclylcarbamoyl substituted with one or more substituents selected from the substituent group α, azide, alkyl and haloalkyl;
(bx) unsubstituted aromatic heterocyclylcarbamoyl or aromatic heterocyclylcarbamoyl substituted with one or more substituents selected from the substituent group α, azide, alkyl and haloalkyl;
(by) unsubstituted non-aromatic heterocyclylcarbamoyl or non-aromatic heterocyclylcarbamoyl substituted with one or more substituents selected from the substituent group α, azide, alkyl and haloalkyl;
(bz) unsubstituted aromatic carbocylylalkylcarbamoyl or aromatic carbocyclylalkylcarbamoyl substituted with one or more substituents selected from the substituent group α, azide, alkyl and haloalkyl;
(ca) unsubstituted non-aromatic carbocyclylalkylcarbamoyl or non-aromatic carbocyclylalkylcarbamoyl substituted with one or more substituents selected from the substituent group α, azide, alkyl and haloalkyl;
(cb) unsubstituted aromatic heterocyclylalkylcarbamoyl or aromatic heterocyclylalkylcarbamoyl substituted with one or more substituents selected from the substituent group α, azide, alkyl and haloalkyl;
(cc) unsubstituted non-aromatic heterocyclylalkylcarbamoyl or non-aromatic heterocyclylalkylcarbamoyl substituted with one or more substituents selected from the substituent group α, azide, alkyl and haloalkyl;
(cd) unsubstituted aromatic carbocyclyloxycarbonyl or aromatic carbocyclyloxycarbonyl substituted with one or more substituents selected from the substituent group α, azide, alkyl and haloalkyl;
(ce) unsubstituted non-aromatic carbocyclyloxycarbonyl or non-aromatic carbocyclyloxycarbonyl substituted with one or more substituents selected from the substituent group α, azide, alkyl and haloalkyl;
(cf) unsubstituted aromatic heterocycyloxycarbonyl or aromatic heterocycyloxycarbonyl substituted with one or more substituents selected from the substituent group α, azide, alkyl and haloalkyl;
(cg) unsubstituted non-aromatic heterocyclyloxycarbonyl or non-aromatic heterocyclyloxycarbonyl substituted with one or more substituents selected from the substituent group α, azide, alkyl and haloalkyl;
(ch) unsubtituted alkylenedioxy or alkylenedioxy substituted with halogen;
(ci) oxo; and
(cj) azide.

Each cyclic group in “substituted or unsubstituted aromatic carbocycle”, “substituted or unsubstituted non-aromatic carbocycle”, “substituted or unsubstituted benzene”, “substituted or unsubstituted aromatic, heterocycle”, “substituted or unsubstituted non-aromatic heterocycle”, “substituted or unsubstituted pyridine”, “substituted or unsubstituted pyrazine”, “substituted or unsubstituted oxazole”, “substituted or unsubstituted pyrimidine”, or “substituted or unsubstituted pyrtdazine” may be substituted with one or more substituents selected from the above substituents.

Examples of substituents of “substituted or unsubstituted aromatic carbocycle”, “substituted or unsubstituted nonaromatic carbocycle”, “substituted or unsubstituted benzene”, “substituted or unsubstituted aromatic heterocycle”, “substituted or unsubstituted nonaromatic heterocycle”, “substituted or unsubstituted pyridine”, “substituted or unsubstituted pyrazine”, “substituted or unsubstituted oxazole”, “substituted or unsubstituted pyrimidine” or “substituted or unsubstituted pyridazine” in ring A and ring B are one or more selected from

halogen;
cyano;
hydroxy;
nitro;
carboxy;
alkyl substituted with one or more substituents selected from the substituent group α,
unsubstituted alkyl;
alkenyl substituted with one or more substituents selected from the substituent group α,
unsubstituted alkenyl;
alkynyl substituted with one or more substituents selected from tbe substituent group α,
unsubstituted alkynyl;
alkyloxy substituted with one or more substituents selected from the substituent group α,
unsubstituted alkyloxy;
alkenyloxy substituted with one or more substituents selected from the substituent group α,
unsubstituted alkenyloxy;
alkynyloxy substituted with one or more substituents selected from the substituent group α,
unsubstituted alkynyloxy;
alkylsulfanyl substituted with one or more substituents selected from the substituent group α,
unsubstituted alkylsulfonyl;
alkenylsulfanyl substituted with one or more substituents selected from the substituent group α,
unsubstituted alkenylsulfanyl;
alkynylsulfanyl substituted with one or more substituents selected from the substituent group α,
unsubstituted alkynylsulfanyl;
amino substituted with one or more substituents selected from the substituent gruop α,
unsubstituted amino;
monoalkylamino substituted with one or more substituents selected from the substituent group α,
unsubstituted monoalkylamino:
dialkylamino substituted with one or more substituents selected from the substituent group α,
unsubstituted dialkylamino;
cycloalkylamino substituted with one or more substituents selected from the substituent group α,
unsubstituted cycloalkylamino;
carbamoyl substituted with one or more substituents selected from the substituent group α,
unsubstituted carbamoyl;
monoalkylcarbamoyl substituted with one or more substituents selected from the substituent group α,
unsubstituted monoalkylcarbamoyl;
dialkylcarbamoyl substituted with one or more substituents selected from the substituent group α,
unsubstituted dialkylcarbamoyl;
alkyloxycarbonyl substituted with one or more substituents selected from the substituent group α,
unsubstituted alkyloxycarbonyl;
aromatic carbocyclyl substituted with one or more substituents selected from the substituent group α, unsubstituted alkyl, and alkyl substituted with one or more substituents selected from the substituent group α;
unsubstituted aromatic carbocyclyl;
non aromatic carbocyclyl substituted with one or more substituents selected from the substituent group α, unsubstituted alkyl, and alkyl substituted with one or more substituents selected from the substituent group α;
unsubstituted non-aromatic carbocyclyl;
aromatic heterocyclyl substituted with one or more substituents selected from the substituent group α, unsubstituted alkyl, and alkyl substituted with one or more substituents selected from the substituent group α;
unsubstituted aromatic heterocyclyl;
nonaromatic heterocyclyl substituted with one or more substituents selected from the substituent group α, unsubstituted alkyl, and alkyl substituted with one or more substituents selected from the substituent group α; and
unsubstituted non-aromatic heterocyclyl.

In one embodiment, substituents are one or more selected from halogen, cyano, hydroxy, alkyl, haloalkyl, cycloalkylalkyl, alkyloxy, haloalkyloxy, alkyloxyalkyloxy, cyanoalkyloxy, alkenyl, haloalkenyl, alkynyl, haloalkynyl, alkenyloxy, alkynyloxy, alkylsulfanyl, cyanoalkylsulfanyl, amino, monoalkylamino, dialkylamino, cycloalkylamino and cycloalkyl.

In another embodiment, substituents are one or more selected from halogen, cyano, alkyl, haloalkyl, alkyloxy, haloalkyloxy, cycloalkylalkyloxy, alkynyloxy.

In another embodiment, substituents of ring A are one or more selected from halogen.

In another embodiment, substituents of ring B are one or more selected from halogen, cyano, alkyl, haloalkyl, alkyloxy and haloalkyloxy.

In one embodiment, the substituents of “substituted or unsubstituted cycloalkyl” are one or more selected from the substituent group α, unsubstituted alkyl and alkyl substituted with one or more substituents selected from the substituent group α.

In another embodiment, “substituted or unsubstituted cycloalkyl” is unsubstituted cycloalkyl.

Specific embodiments of the present invention are illustrated below. The embodiments are the compound of the following formulas (IA) to (IO):

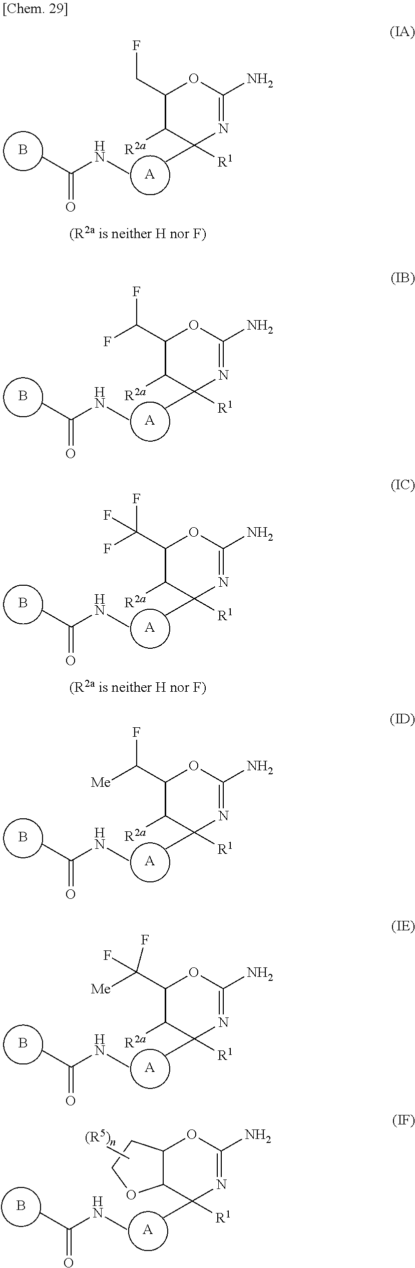

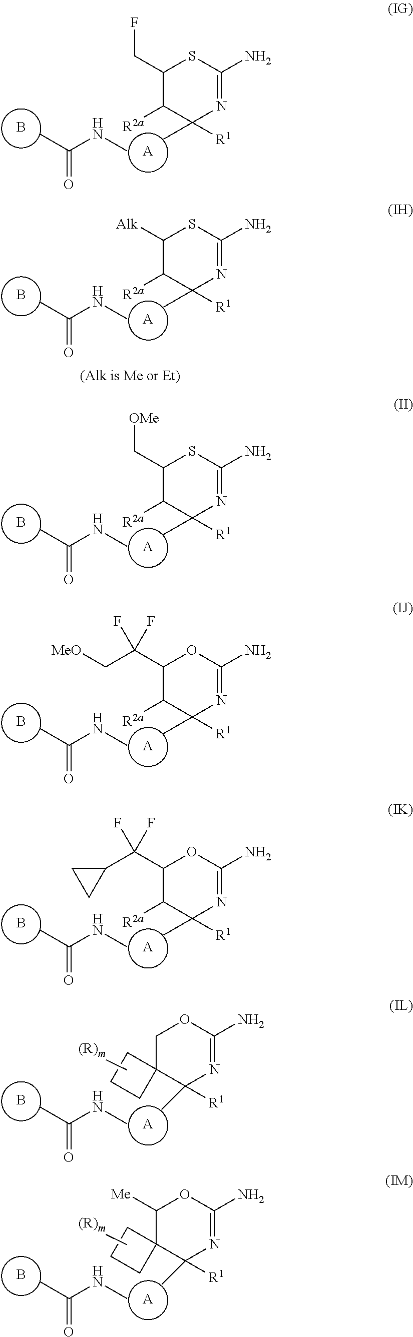

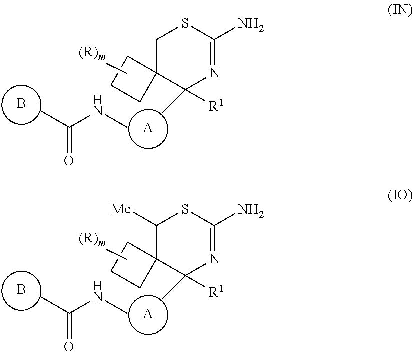

wherein each symbol is the same as defined above,
or a pharmaceutically acceptable salt thereof.

In one embodiment of the compound of formula (IA), (IB) (IC), (IJ), or (IK), R^{2a} is alkyl or haloalkyloxy.

In one embodiment of the compound of formula (ID), (IE), (IJ) or (IK), R^{2a} in halogen.

In one embodiment of the compound of formula (IL), (IM), (IN) or (IO), R is halogen and m is an integer of 2.

In one embodiment of the compound of formula (IF), R³ is halogen and n is an integer of 1 or 2.

In one embodiment of the compound of formula (IG), (IH) or (II), R^{2a} in halogen.

In one embodiment of the compound of formula (IH), R^{2a} is alkyloxy.

Specific embodiments of each symbol of the formula (I) are illustrated below. All combination of those embodiments are examples of the compounds of formula (I).

In the formula (I),
X is —O—, R^{3a} is haloalkyl, R^{3b} is alkyl, R^{2a} is H, R^{2b} is H, and R¹ is alkyl or haloalkyl (hereinafter referred to as X1),
X is —O—, R^{3a} is CF₃, R^{3b} is alkyl, R^{2a} is H, R^{2b} is H, and R¹ is alkyl or haloalkyl (hereinafter referred to as X2),
X is —O—, R^{3a} is CHF₂, R^{3b} is H, R^{2a} is halogen, R^{2b} is H, and R¹ is alkyl or haloalkyl (hereinafter referred to as X3),
X is —O—, R^{3a} CHFCH₃, R^{3b} is H, R^{2a} is halogen, R^{2b} is H, and R¹ is alkyl or haloalkyl (hereinafter referred to as X4).
X is —O—, R^{3a} is CF₂CH₃, R^{3b} is H, R^{2a} is halogen, R^{2b} is H, and R¹ is alkyl or haloalkyl (hereinafter referred to as X5),
X is —O—, R^{3a} is haloalkyl substituted with alkyloxy or cycloalkyl, R^{3b} is H, R^{2a} is halogen, R^{2b} is H, and R¹ is alkyl or haloalkyl (hereinafter referred to as X6),
X is —O—, R^{3a} is haloalkyl, R^{3b} is alkyl, R^{2a} is halogen, R^{2b} is H, and R¹ is alkyl or haloalkyl thereinafter referred to as X7),
X is —O—, R^{3a} is haloalkyl, R^{3b} is H, is alkyl, R^{2b} is H, and R¹ is alkyl or haloalkyl (hereinafter referred to as X8),
X is —O—, R^{3a} is CHF₂, R^{3b} is H, R^{2a} is CH₃, R^{2b} is H, and R¹ is alkyl or haloalkyl 1 (hereinafter referred to as X8),
X is —O—, R^{3a} is CF₃, R^{3b} is H, R^{2a} is CH₃, R^{2b} is H, and R¹ is alkyl or haloalkyl (hereinafter referred to as X10),
X is —O—, R^{3a} is haloalkyl, R^{3b} is alkyl, R^{2a} alkyl, R^{2b} is H, and R¹ is alkyl or haloalkyl (hereinafter referred to as X11),
X is —O—, R^{3a} is haloalkyl, R^{2a} is H, R^{2a} is alkyloxy, R^{2b} is H, and R¹ is alkyl or haloalkyl (hereinafter referred to as X12),
X is —O—, R^{3a} is CH₂F, R^{3b} is H, R^{2a} in OCH₃, R^{2b} is H, and R¹ is alkyl or haloalkyl (hereinafter referred to as X13),
X is —O—, R^{3a} is CF₃, R^{3b} is H, R^{2a} is OCH₃, R^{2b} is H, and R¹ is alkyl or haloalkyl thereinafter referred to a X14),
X is —O—, R^{3a} is haloalkyl, R^{3b} is alkyl, R^{2a} is alkyloxy, R^{2b} is H, and R¹ is alkyl or haloalkyl (hereinafter referred to as X15),
X is —O—, R^{3a} is haloalkyl, R^{3b} is H, R^{2a} is haloalkyloxy, R^{2b} is H, and R¹ is alkyl or haloalkyl (hereinafter referred to as X16),
X is —O—, R^{3a} is CH₂F, R^{3b} is H, R^{2a} is OCH₃CF₃, R^{3b} is H, and R¹ is alkyl or haloalkyl (hereinafter referred to as X17),
X is —O—, R^{3a} is haloalkyl, R^{3b} is alkyl, R^{2a} is haloalkyloxy, R^{2b} is H, and R¹ is alkyl or haloalkyl (hereinafter referred to as X18).
X is —S—, R^{3a} is alkyl, R^{3b} is H, R^{2a} is halogen, R^{2b} is H, and R¹ is alkyl or haloalkyl (hereinafter referred to as X19),
X is —S—, R^{3a} is is CH₃ or CH₂CH₃, R^{3b} is H, R^{2a} is halogen, R^{2b} is H, and R¹ is alkyl or haloalkyl (hereinafter referred to as X20),
X is —S—, R^{3a} is alkyl, R^{3b} is alkyl, R^{2a} is halogen, R^{2b} is H, and R¹ is alkyl or haloalkyl (hereinafter referred to as X21),
X is —S—, R^{3a} is alkyl, R^{3b} is H, R^{2a} is alkyloxy, R^{2b} is H, and R¹ is alkyl or haloalkyl (hereinafter referred to as X22),
X is —S—, R^{3a} is alkyl, R^{3b} is alkyl, R^{2a} is alkyloxy, R^{2b} is H, and R¹ is alkyl or haloalkyl (hereinafter referred to as X23),
X is —S—, R^{3a} is alkyl, R^{3b} is H, R^{2a} is haloalkyloxy, R^{2b} is H, and R¹ is alkyl or haloalkyl (hereinafter referred to as X24),
X is —S—, R^{3a} is alkyl, R^{3b} is alkyl, R^{2a} is haloalkyloxy, R^{2b} is H, and R¹ is alkyl or haloalkyl (hereinafter referred to as X25),
X is —S—, R^{3a} is haloalkyl, R^{3b} is H, R^{2a} is halogon, R^{2b} is H, and R¹ is alkyl or haloalkyl (hereinafter referred to as X26),
X is —S—, R^{3a} is CH₂F, R^{3b} is H, R^{2a} is halogen, R^{2b} is H, and R¹ is alkyl or haloalkyl (hereinafter referred to as X27),
X is —S—, R^{3a} is haloalkyl, R^{3b} is alkyl, R^{2a} is halogen, R^{2b} is H, and R¹ is alkyl or haloalkyl (hereinafter referred to at X28),
X is —S—, R^{3a} is haloalkyl, R^{3b} is H, R^{2a} is alkyloxy, R^{2b} is H, and R¹ is alkyl or haloalkyl (hereinafter referred to as X29),
X is —S—, R^{3a} is haloalkyl, R^{3b} is alkyl, R^{2a} is alkyloxy, R^{2b} is H, and R¹ is alkyl or haloalkyl (hereinafter referred to as X30),
X is —S—, R^{3a} is haloalkyl, R^{3b} is H, R^{2a} is haloalkyloxy, R^{2b} is H, and R¹ is alkyl or haloalkyl (hereinafter referred to as X31),
X is —S—, R^{3a} is haloalkyl, R^{3b} is alkyl, R^{2a} is haloalkyloxy, R^{2b} is H, and R¹ is alkyl or haloalkyl (hereinafter referred to as X32),
X is —S—, R^{3a} is haloalkyl, R^{3b} is H, R^{2a} is alkyl, R^{2b} is H, and R¹ is alkyl or haloalkyl (hereinafter referred to as X33),
X is —S—, R^{3a} is haloalkyl, R^{3b} is alkyl, R^{2a} is alkyl, R^{2b} is H, and R¹ is alkyl or haloalkyl (hereinafter referred to as X34),
X is —S—, R^{3a} is alkyloxyalkyl, R^{3b} is H, R^{2a} is halogen, R^{2b} is H, and R¹ is alkyl or haloalkyl (hereinafter referred to as X35),
X is —O—,

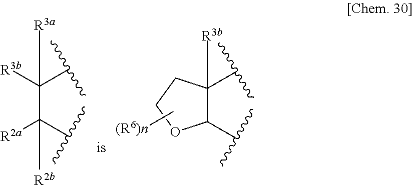

R^{3b} in H, n is 0, and R¹ is alkyl or haloalkyl (hereinafter referred to as X36),
X is —O—,

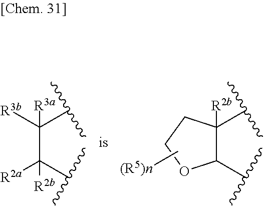

R^{3b} is alkyl, n is 0, and R¹ is alkyl or haloalkyl (hereinafter referred to as X37),
X is —O—,

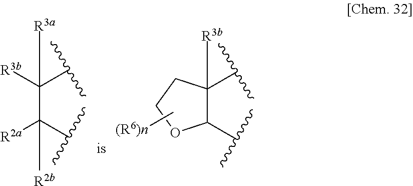

R^{3b} is H, n is 1, R⁵ is halogen, and R¹ is alkyl or haloalkyl (hereinafter referred to as X38),
X is —O—,

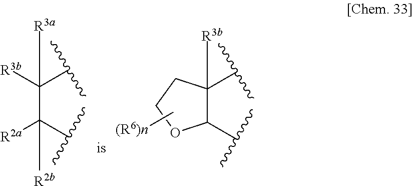

R^{3b} is alkyl, n is 1, R⁵ is halogen, and R¹ is alkyl or haloalkyl (hereinafter referred to as X39),
X is —O—,

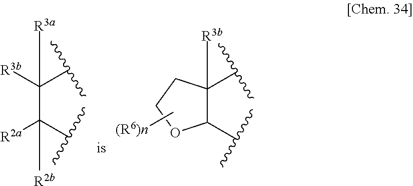

R^{3b} is H, n is 2, R⁵ is halogen, and R¹ is alkyl or haloalkyl (hereinafter referred to as X40),
X is —O—,

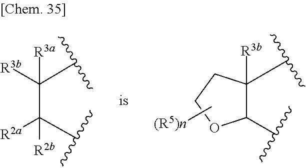

R^{3b} is alkyl, n is 2, R⁵ is halogen, and R¹ is alkyl or haloalkyl (hereinafter referred to as X41),
X is —S—,

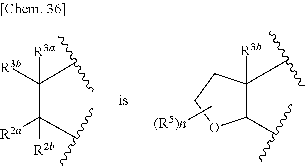

R^{3b} is H, n is 0, and R¹ is alkyl or haloalkyl (hereinafter referred to as X42),
X is —S—,

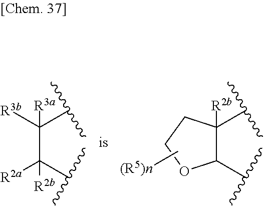

R^{3b} is alkyl, n is 0, and R¹ is alkyl or haloalkyl (hereinafter referred to as X43),
X is —S—,

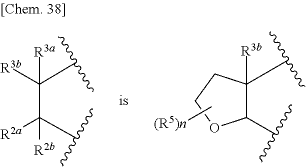

R^{3b} is H, n is 1, R⁵ is halogen, and R¹ is alkyl or haloalkyl (hereinafter referred to as X44),
X is —S—,

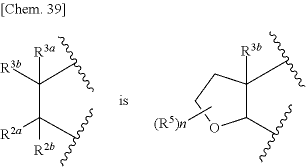

R^{3b} is alkyl, n is 1, R⁵ is halogen, and R¹ is alkyl or haloalkyl (hereinafter referred to as X45),
X is —S—,

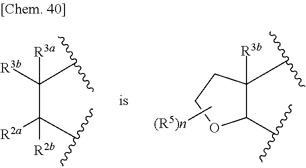

R^{3b} is H, n is 2, R⁵ is halogen, and R¹ is alkyl or haloalkyl (hereinafter referred to as X46),
X is —S—,

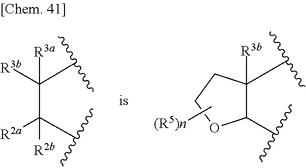

R^{3b} is alkyl, n is 2, R⁵ is halogen, and R¹ is alkyl or haloalkyl (hereinafter referred to as X47),
X is —S— or —O—, R^{3a} is H or alkyl, R^{3b} is H, R^{2a} and R^{2b} together with the carbon atom to which they are attached may form cycloalkane substituted with 1 or 2 halogens, and R¹ is alkyl or haloalkyl (hereinafter referred to as X48),
ring A is a substituted or unsubstituted benzene and ring B is a substituted or unsubstituted pyridine(hereinafter referred to as AB1).
ring A is a substituted ur unsubstituted benzene and ring B in a substituted or unsubstituted pyradine (hereinafter referred to as AB2),
ring A is a substituted or unsubstituted benzene and ring B is a substituted or unsubstituted oxazole (hereinafter referred to as AB3),
ring A is a substituted or unsubstituted benzene and ring B is a substituted or unsubstituted pyrimidine or a substituted or unsubstituted pyridazine (hereinafter referred to as AB4),
ring A in a substituted or unsubstituted pyridine, and ring B is a substituted or unsubtituted pyridine (hereinafter referred to as AB5),
ring A is a substituted or unsubstituted pyridine, and ring B is a substituted or unsubstituted pyrazine (hereinafter referred to as AB6),
ring A is a substituted or unsubstituted pyridine, and ring B is a substituted or unsubstituted oxazole (hereinafter referred to as AB7),
ring A is a benzene substituted with halogen such as F or Cl, and ring B is a pyridine substituted with one or two substituents selected from halogen, cyano, aIkyl, alkyloxy and haloalkyloxy (hereinafter referred to as AB8),
ring A is a benzene substituted with halogen such as F or Cl, and ring B is a pyrazine substituted with one or two substituents selected from haloalkyl, alkyloxy, haloalkyloxy, alkynyloxy and cycloalkylalkyloxy (hereinafter referred to as AB9),
ring A is a benzene substituted with halogen such as F or Cl, and ring B is a oxazole substituted with one or two substituents selected from alkyl and haloalkyl (hereinafter referred to as AB10),
ring A is a benzene substituted with halogen such as F or Cl, and ring B is a pyrimidine substituted with one or two substituents selected from haloalkyloxy or a or a pyridazine substituted with one or two substituents selected from alkyloxy (hereinafter referred to as AB11),
ring A is a pyridine substituted with halogen and ring B is a pyridine substituted with one or two substituents selected from halogen and cyano (hereinafter referred to as AB12).
ring A is a pyridine substituted with halogen and ring B is a pyraxine substituted with one or two substituents selected from alkyloxy and haloalkyloxy (hereinafter referred to as AB13).

Examples of combination of “X, R^{3a}, R^{3b}, R^{2a}, R^{2b} and R¹”, and “ring A and ring B”, (X, AB) of the compounds of formula (1) are as follows:

(X1,AB1),(X1,AB2),(X1,AB3),(X1,AB4),(X1,AB5),(X1,AB6),(X1,AB7),(X1,AB8),(X1,AB9),(X1,AB10),(X1,AB11),(X1,AB12),(X1,AB13),(X2,AB1),(X2,AB2),(X2,AB3),(X2,AB4),(X2,AB5),(X2,AB6),(X2,AB7),(X2,AB8),(X2,AB9),(X2,AB10),(X2,AB11),(X2,AB12),(X2,AB13),(X3,AB1),(X3,AB2),(X3,AB3),(X3,AB4),(X3,AB5),(X3,AB6),(X3,AB7),(X3,AB8),(X3,AB9),(X3,AB10),(X3,AB11),(X3,AB12),(X3,AB13),(X4,AB1),(X4,AB2),(X4,AB3),(X4,AB4),(X4,AB5),(X4,AB6),(X4,AB7),(X4,AB8),(X4,AB9),(X4,AB10),(X4,AB11),(X4,AB12),(X4,AB13),(X5,AB1),(X5,AB2),(X5,AB3),(X5,AB4),(X5,AB5),(X5,AB6),(X5,AB7),(X5,AB8),(X5,AB9),(X5,AB10),(X5,AB11),(X5,AB12),(X5,AB13),(X6,AB1),(X6,AB2),(X6,AB3),(X6,AB4),(X6,AB5),(X6,AB6),(X6,AB7),(X6,AB8),(X6,AB9),(X6,AB10),(X6,AB11),(X6,AB12),(X6,AB13),(X7,AB1),(X7,AB2),(X7,AB3),(X7,AB4),(X7,AB5),(X7,AB6),(X7,AB7),(X7,AB8),(X7,AB9),(X7,AB10),(X7,AB11),(X7,AB12),(X7,AB13),(X8,AB1),(X8,AB2),(X8,AB3),(X8,AB4),(X8,AB5),(X8,AB6),(X8,AB7),(X8,AB8),(X8,AB9),(X8,AB10),(X8,AB11),(X8,AB12),(X8,AB13),(X9,AB1),(X9,AB2),(X9,AB3),(X9,AB4),(X9,AB5),(X9,AB6),(X9,AB7),(X9,AB8),(X9,AB9),(X9,AB10),(X9,AB11),(X9,AB12),(X9,AB13),(X10,AB1),(X10,AB2),(X10,AB3),(X10,AB4),(X10,AB5),(X10,AB6),(X10,AB7),(X10,AB8),(X10,AB9),(X10,AB10),(X10,AB11),(X10,AB12),(X10,AB13),(X11,AB1),(X11,AB2),(X11,AB3),(X11,AB4),(X11,AB5),(X11,AB6),(X11,AB7),(X11,AB8),(X11,AB9),(X11,AB10),(X11,AB11),(X11,AB12),(X11,AB13),(X12,AB1),(X12,AB2),(X12,AB3),(X12,AB4),(X12,AB5),(X12,AB6),(X12,AB7),(X12,AB8),(X12,AB9),(X12,AB10),(X12,AB11),(X12,AB12),(X12,AB13),(X13,AB1),(X13,AB2),(X13,AB3),(X13,AB4),(X13,AB5),(X13,AB6),(X13,AB7),(X13,AB8),(X13,AB9),(X13,AB10),(X13,AB11),(X13,AB12),(X13,AB13),(X14,AB1),(X14,AB2),(X14,AB3),(X14,AB4),(X14,AB5),(X14,AB6),(X14,AB7),(X14,AB8),(X14,AB9),(X14,AB10),(X14,AB11),(X14,AB12),(X14AB13),(X15,AB1),(X15,AB2),(X15,AB3),(X15,AB4),(X15,AB5),(X15,AB6),(X15,AB7),(X15,AB8),(X15,AB9),(X15,AB10),(X15,AB11),(X15,AB12),(X15,AB13),(X16,AB1),(X16,AB2),(X16,AB3),(X16,AB4),(X16,AB5),(X16,AB6),(X16,AB7),(X16,AB8),(X16,AB9),(X16,AB10),(X16,AB11),(X16,AB12),(X16,AB13),(X17,AB1),(X17,AB2),(X17,AB3),(X17,AB4),(X17,AB5),(X17,AB6),(X17,AB7),(X17,AB8),(X17,AB9),(X17,AB10),(X17,AB11),(X17,AB12),(X17,AB13),(X18,AB1),(X18,AB2),(X18,AB3),(X18,AB4),(X18,AB5),(X18,AB6),(X18,AB7),(X18,AB8),(X18,AB9),(X18,AB10),(X18,AB11),(X18,AB12),(X18,AB13),(X19,AB1),(X19,AB2),(X19,AB3),(X19,AB4),(X19,AB5),(X19,AB6),(X19,AB7),(X19,AB8),(X19,AB9),(X19,AB10),(X19,AB11),(X19,AB12),(X19,AB13),(X20,AB1),(X20,AB2),(X20,AB3),(X20,AB4),(X20,AB5),(X20,AB6),(X20,AB7),(X20,AB8),(X20,AB9),(X20,AB10),(X20,AB11),(X20,AB12),(X20,AB13),(X21,AB1),(X21,AB2),(X21,AB3),(X21,AB4),(X21,AB5),(X21,AB6),(X21,AB7),(X21,AB8),(X21,AB9),(X21,AB10),(X21,AB11),(X21,AB12),(X21,AB13),(X22,AB1),(X22,AB2),(X22,AB3),(X22,AB4),(X22,AB5),(X22,AB6),(X22,AB7),(X22,AB8),(X22,AB9),(X22,AB10),(X22,AB11),(X22,AB12),(X22,AB13),(X23,AB1),(X23,AB2),(X23,AB3),(X23,AB4),(X23,AB5),(X23,AB6),(X23,AB7),(X23,AB8),(X23,AB9),(X23,AB10),(X23,AB11),(X23,AB12),(X23,AB13),(X24,AB1),(X24,AB2),(X24,AB3),(X24,AB4),(X24,AB5),(X24,AB6),(X24,AB7),(X24,AB8),(X24,AB9),(X24,AB10),(X24,AB11),(X24,AB12),(X24,AB13),(X25,AB1),(X25,AB2),(X25,AB3),(X25,AB4),(X25,AB5),(X25,AB6),(X25,AB7),(X25,AB8),(X25,AB9),(X25,AB10),(X25,AB11),(X25,AB12),(X25,AB13),(X26,AB1),(X26,AB2),(X26,AB3),(X26,AB4),(X26,AB5),(X26,AB6),(X26,AB7),(X26,AB8),(X26,AB9),(X26,AB10),(X26,AB11),(X26,AB12),(X26,AB13),(X27,AB1),(X27,AB2),(X27,AB3),(X27,AB4),(X27,AB5),(X27,AB6),(X27,AB7),(X27,AB8),(X27,AB9),(X27,AB10),(X27,AB11),(X27,AB12),(X27,AB13),(X28,AB1),(X28,AB2),(X28,AB3),(X28,AB4),(X28,AB5),(X28,AB6),(X28,AB7),(X28,AB8),(X28,AB9),(X28,AB10),(X28,AB11),(X28,AB12),(X28,AB13),(X29,AB1),(X29,AB2),(X29,AB3),(X29,AB4),(X29,AB5),(X29,AB6),(X29,AB7),(X29,AB8),(X29,AB9),(X29,AB10),(X29,AB11),(X29,AB12),(X29,AB13),(X30,AB1),(X30,AB2),(X30,AB3),(X30,AB4),(X30,AB5),(X30,AB6),(X30,AB7),(X30,AB8),(X30,AB9),(X30,AB10),(X30,AB11),(X30,AB12),(X30,AB13),(X31,AB1),(X31,AB2),(X31,AB3),(X31,AB4),(X31,AB5),(X31,AB6),(X31,AB7),(X31,AB8),(X31,AB9),(X31,AB10),(X31,AB11),(X31,AB12),(X31,AB13),(X32,AB1),(X32,AB2),(X32,AB3),(X32,AB4),(X32,AB5),(X32,AB6),(X32,AB7),(X32,AB8),(X32,AB9),(X32,AB10),(X32,AB11),(X32,AB12),(X32,AB13),(X33,AB1),(X33,AB2),(X33,AB3),(X33,AB4),(X33,AB5),(X33,AB6),(X33,AB7),(X33,AB8),(X33,AB9),(X33,AB10),(X33,AB11),(X33,AB12),(X33,AB13),(X34,AB1),(X34,AB2),(X34,AB3),(X34,AB4),(X34,AB5),(X34,AB6),(X34,AB7),(X34,AB8),(X34,AB9),(X34,AB10),(X34,AB11),(X34,AB12),(X34,AB13),(X35,AB1),(X35,AB2),(X35,AB3),(X35,AB4),(X35,AB5),(X35,AB6),(X35,AB7),(X35,AB8),(X35,AB9),(X35,AB10),(X35,AB11),(X35,AB12),(X35,AB13),(X36,AB1),(X36,AB2),(X36,AB3),(X36,AB4),(X36,AB5),(X36,AB6),(X36,AB7),(X36,AB8),(X36,AB9),(X36,AB10),(X36,AB11),(X36,AB12),(X36,AB13),(X37,AB1),(X37,AB2),(X37,AB3),(X37,AB4),(X37,AB5),(X37,AB6),(X37,AB7),(X37,AB8),(X37,AB9),(X37,AB10),(X37,AB11),(X37,AB12),(X37,AB13),(X38,AB1),(X38,AB2),(X38,AB3),(X38,AB4),(X38,AB5),(X38,AB6),(X38,AB7),(X38,AB8),(X38,AB9),(X38,AB10),(X38,AB11),(X38,AB12),(X38,AB13),(X39,AB1),(X39,AB2),(X39,AB3),(X39,AB4),(X39,AB5),(X39,AB6),(X39,AB7),(X39,AB8),(X39,AB9),(X39,AB10),(X39,AB11),(X39,AB12),(X39,AB13),(X40,AB1),(X40,AB2),(X40,AB3),(X40,AB4),(X40,AB5),(X40,AB6),(X40,AB7),(X40,AB8),(X40,AB9),(X40,AB10),(X40,AB11),(X40,AB12),(X40,AB13),(X41,AB1),(X41,AB2),(X41,AB3),(X41,AB4),(X41,AB5),(X41,AB6),(X41,AB7),(X41,AB8),(X41,AB9),(X41,AB10),(X41,AB11),(X41,AB12),(X41,AB13),(X42,AB1),(X42,AB2),(X42,AB3),(X42,AB4),(X42,AB5),(X42,AB6),(X42,AB7),(X42,AB8),(X42,AB9),(X42,AB10),(X42,AB11),(X42,AB12),(X42,AB13),(X43,AB1),(43,AB2),(X43,AB3),(X43,AB4),(X43,AB5),(X43,AB6),(X43,AB7),(X43,AB8),(X43,AB9),(X43,AB10),(X43,AB11),(X43,AB12),(X43,AB13),(X44,AB1),(X44,AB2),(X44,AB3),(X44,AB4),(X44,AB5),(X44,AB6),(X44,AB7),(X44,AB8),(X44,AB9),(X44,AB10),(X44,AB11),(X44,AB12),(X44,AB13),(X45,AB1),(X45,AB2),(X45,AB3),(X45,AB4),(X45,AB5),(X45,AB6),(X45,AB7),(X45,AB8),(X45,AB9),(X45,AB10),(X45,AB11),(X45,AB12),(X45,AB13),(X46,AB1),(X46,AB2),(X46,AB3),(X46,AB4),(X46,AB5),(X46,AB6),(X46,AB7),(X46,AB8),(X46,AB9),(X46,AB10),(X46,AB11),(X46,AB12),(X46,AB13),(X47,AB1),(X47,AB2),(X47,AB3),(X47,AB4),(X47,AB5),(X47,AB6),(X47,AB7),(X47,AB8),(X47,AB9),(X47,AB10),(X47,AB11),(X47,AB12),(X47,AB13),(X48,AB1),(X48,AB2),(X48,AB3),(X48,AB4),(X48,AB5),(X48,AB6),(X48,AB7),(X48,AB8),(X48,AB9),(X48,AB10),(X48,AB11),(X48,AB12) or (X48,AB13).

The compound of formula (I) is not limited to a specific isomer, and includes all possible isomers such as keto-enol isomers, imine-enamine isomers, diastereoisomers, optical isomers and rotation isomers, racemate and the mixture thereof. For example, the compound of formula (I) includes the following tautomers.

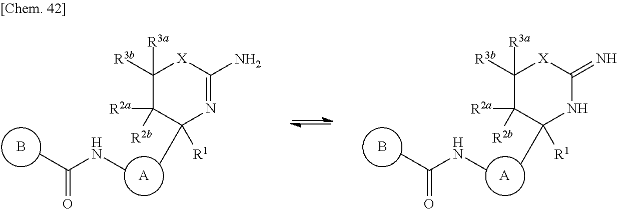

The compound of formula (I) has an asymmetric carbon atom and the compound includes the following optical isomers.

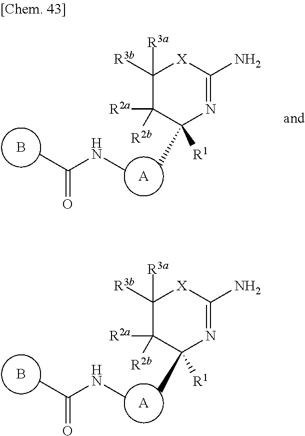

In one embodiment the compound of the present invention is as follows:

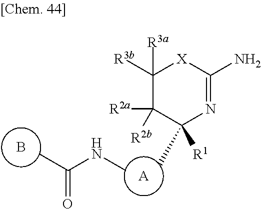

Optically active compounds of formula (I) can be produced by emptying an optically active starting material, by obtaining an optically active immediate by assymmetry synthesis at a suitable stage, or by performing optical resolution at an intermediate or an objective compound, each of which is a racemate, at a suitable stage. Examples of a method for optical resolution is separation of an optical isomer using an optically acitve column: kinetic optical resolution utilizing an enzymatic reaction; crystallization resolution of a diastereomer by salt formation using a chiral acid or a chiral base; and preferential crystallization method.

One or more hydrogen, carbon and/or other atoms of a compound of formula (I) can be replaced with an isotope of hydrogen, carbon and/or other atoms, respectively. Examples of isotopes include isotopes of hydrogen, carbon, nitrogen, oxygen, phosphorous, sulfur, fluorine, iodine and chlorine, such as ²H, ³H, ¹¹C, ¹³C, ¹⁴C, ¹⁶N, ¹⁸O, ¹⁷O, ³¹P, ³²P, ²⁰S, ¹⁸F, ¹²³I and ³⁶Cl, respectively. The compounds of formula (I) also includes the compound replaced with such isotopes. The compound replaced with such isotopes is useful also as a medicament, and includes all the radiolabeled compounds of the compound of formula (I). The invention includes “radiolabelling method” for manufacturing the “radiolabeled compound” and the method is useful as a tool of metabolic pharmacokinetic research, the research in binding assay and/or diagnosis.

A radiolabeled compound of the compound of formula (I) can be prepared by methods known in the art. For example, tritiated compounds of formula (I) can be prepared by introducing tritium into the particular compound of formula (I) such as by catalytic dehalogenation with tritium. This method may include reacting a suitably halogenated precursor of a compound of formula (I) with tritium gas in the presence of a suitable catalyst such as Pd/C, in the presence or absence of a base. Other suitable methods for preparing tritiated compounds can be found in Isotopes in the Physical and Biomedical Sciences, Vol. 1, Lableled Compounds (Part A), Chapter 6 (1987). A ¹⁴C-labeled compound can be prepared by employing starting materials having ¹⁴C carbon.

As pharmaceutically acceptable salt of the compound of formula (I), examples include salts with alkaline metals (e.g., lithium, sodium and potassium), alkaline earth metals (e.g. calcium and barium), magnesium, transition metal (e.g., zinc and iron), ammonia, organic bases (e.g. trimethylamine, triethylamine, dicyclohexylamine, ethanolamine, diethanolamine, triethanolamine, meglumaine, diethanolamine, ethylenediamine, pyridine, picoline, quinoline), and amino acids, and salts with inorganic acids (e.g. hydrochloric acid, sulfuric acid, nitric acid, carbonic acid, hydrobromic acid, phosphoric acid and hydroiodic acid) and organic acids (e.g. formic acid, acetic acid, propionic acid, trifluoroacetic acid, citric acid, lactic acid, tartaric acid, oxalic acid, maleic acid, furmaric acid, mandelic acid, glutaric acid, malic acid, benzoic acid, phthalic acid, ascorbic acid, benzenesulfonic acid, p-toluenesulfonic acid, methanesulfonic acid and ethanesulfonic acid). Specific Examples are salts with hydrochloric acid, sulfuric acid, phosphoric acid, tartaric acid, or methanesulfonic acid. These salts can be formed by the usual method.

The compounds of the present invention represented by formula (I) or pharmaceutically acceptable salts thereof may form solvates (e.g. hydrates etc.) and/or crystal polymorphs. The present invention encompasses those various solvates and crystal polymorphs. “Solvates” may be those wherein any number of solvent molecules (e.g., water molecules etc.) are coordinated with the compounds represented by formula (I). When the compounds represented by formula (I) or pharmaceutically acceptable salts thereof are allowed to stand in the atmosphere, the compounds may absorb water, resulting in attachment of adsorbed water or formation of hydrates. Recrystallization of the compounds represented by formula (I) or pharmaceutically acceptable salts thereof may produce crystal polymorphs.

The compounds of the present invention represented by formula (I) or pharmaceutically acceptable salts thereof may form prodrugs. The present invention also encompasses such various prodrugs. Prodrugs are derivatives of the compounds of the present invention that have chemically or metabolically degradable groups and are compounds that are converted to the pharmaceutically active compounds of the present invention through solvolysis or under physiological conditions in vivo. Prodrugs include compounds that are converted to the compounds represented by formula (I) through enzymatic oxidation, reduction, hydrolysis and the like under physiological conditions in vivo and compounds that are converted to the compounds represented by formula (I) through hydrolysis by gastric acid and the like. Methods for selecting and preparing suitable prodrug derivatives are described, for example, in the Design of Prodrugs, Elsevier, Amsterdam 1985. Prodrugs themselves may be active compounds.

When the compounds of formula (1) or pharmaceutically acceptable salts thereof have a hydroxy group, prodrugs include acyloxy derivatives and sulfonyloxy derivatives which can be prepared by reacting a compound having a hydroxy group with a suitable acid halide, suitable acid anhydride, suitable sulfonyl chloride, suitable sulfonylanhydride and mixed anhydride or with a condensing agent. Examples are CH₃COO—, C₂H₅COO—, t-BuCOO—, C₁₆H₃₁COO—, PhCOO—, (m-NaOOCPh)COO—, NaOOCCH₂CH₂COO—, CH₃CH(NH₂) COO—, CH₂N(CH₃)₂COO—, CH₃SO₃—, CH₃CH₂SO₃—, CFaSO₃—, CH₂FSO₃—, CF₂CH₂SO₃—, p-CH₃—O—PhSO₃—, PhSO₃— and p-CH₃PhSO ₃—.

The compounds of formula (I) may be prepared by the methods described below, together with synthetic methods known to a person skilled in the art.

The starting materials are commercially available or may be prepared in accordance with known methods.

During any of the following synthesis, it may be necessary or preferable to protect sensitive or reactive groups on any of molecules. In such case, these protection can be achieved by means of conventional protective groups such as those described in Greene's Protective Group in Organic Synthesis, John Wily & Sons, 2007.

It will be understood by a person killed in the art that the compounds described below will be generated a mixture of diastereomers and/or enantiomers, which may be separated at relevant stages of the following procedures using conventional techniques such as crystallisation, silica gel chromatography, chiral or achiral high performance liquid chromatography (HPLC), and chiral supercritical fluid (SFC) chromatography to provide the single cnantiomers of the invention.

During all Ihe following steps, the order of ihe steps to be performed may be appropriately changed. In each step, an intermediate may be isolated and then used in the next step. All of reaction time, reaction temperature, solvents, reagents, protecting groups, etc. are mere exemplification and not limited as long as they do not cause an adverse effect on a reaction.

General procedure A:

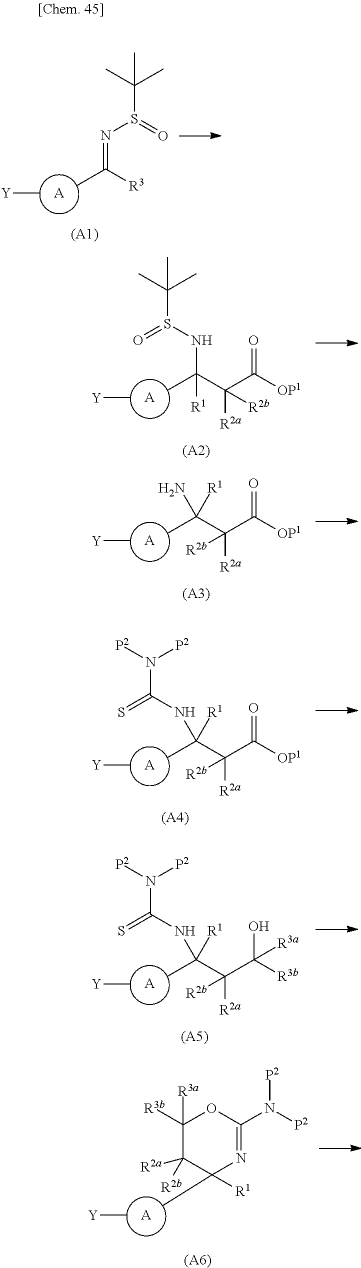

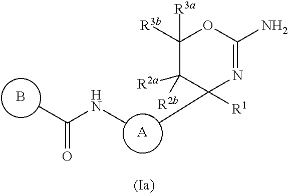

wherein P¹ is alkyl, each of P² is hydrogen or protective groups such an alkyl, benzoyl, benzyl, 4-methoxybenzyl or 2,4-dimethoxybenzylre, Y is halogen (e.g., Br, I), nitro, or trifluoroacetylamine (—NHCOCF₃), and other symbols are the same as defined above.

General Procedare A is a method for preparing compounds of formula (Ia) from compounds of formula (A1) through multiple steps of Step 1 to Step 7. Those skilled in the art will be appreciate that protective groups P¹ and P² can be chosen depending on the reaction conditions used in later steps. The starting material of formula (A1) can be prepared in a manner similar to the conditions described in Chem. Rev. 2010, 110, 3600-3740.

### Step 1:

Compounds of formula (A2) can be prepared by Mannich reaction of sulfinyl imine (A1) with enolates derived from the corresponding esters. This type of reactions can be conducted using the conditions described in Chem. Rev. 2010, 110, 3600-3740, Preferably, the enolates can be prepared from the corresponding esters, lithium diisopropylamide (LDA), and TiCl(Oi-Pr)₃, which can be then reacted with (A1) to give compounds of formula (A2). The solvent used in this step is not particularly limited in so far as it does not interfere with the reaction. Examples of the solvent include tetrahydrofuran, 1,4-dioxanne, 1,2-dimethoxyethane, diethyl ether, toluene, and benzene. The reaction temperature is preferably −78° C. to −30° C. The reaction time is not particularly limited and is usually 5 minutes to 24 hours, preferably 30 minutes to 21 hours.

### Step 2:

Compounds of formula (A3) can be prepared by deprotection of (A2). This deprotection reaction is known to a person skilled in the art and can be performed under the conditions described in Chem. Rev. 2010, 110, 3600-3740. The reaction can be conducted under acidic conditions using e.g. hydrochloric acid at room temperature to 60° C. Examples of the solvent include methanol, 1,4-dioxane, and ethyl acetate. The reaction time is not particularly limited and is usually 1 hour to 24 hours, preferably 1 hour to 6 hours.

### Step 3:

Compounds of formula (A4) can be prepared by reaction of (A3) with reagents such as benzoyl isothiocyanate and benzyl isothiocyanate. Those skilled in the art will appreciate that the isothiocyanate generated from (A3) and reagents such as thiophosgene and thiocarbonyl diimidazole can be reacted with primary or secondary amines to afford compounds of formula (A4). The solvent used in this step is not particularly limited in so far as it does not interfere with the reaction. Examples of the solvent include dichloromethane, tetrahydrofuran, 1,4-dioxane, 1,2-dimethoxyethane, and toluene. The reaction time is not particularly limited and is usually 1 hour to 24 hours, preferably 3 hours to 6 hours. The reaction temperature is usually 0° C. to 60° C., preferably 0° C. to room temperature. Reagents for the thiourea formation in this step are not particularly limited if these can be deprotected in Step 6, and a preferable reagent is benzoyl isothiocyanate.

### Step 4:

Compounds of formula (A5) can be prepared by reaction of (A4) with Grignard reagents such as methyl magnesium bromide and ethyl magnesium bromide and alkyl lithium reagents such as methyllithium, butyllithium, and phenyllithium. Stepwise addition of there nucleophiles can allow for compounds of formula (A5) with various substituents of R^{3a} and R^{3b}. The solvent used is not particularly limited in so far as it does not interfere with the reaction. Preferable examples of the solvent include tetrahydrofuran, 1,4-dioane, 1,2-dimethoxyethane, diethyl ether, toluene, and benaene. The reaction temperature is not particularly limited and is usually 5 minutes to 24 hours, preferably 5 minutes to 6 hours. The reaction temperature is usually −100° C. to room temperature, preferably −78° C. to 0° C.

### Step 5:

Compounds of formula (A6) can be prepared by cyclical ion reaction of (A5) using reagents such as m-CPRA, hydrogen peroxide, and carbobiimide reagents (e.g. 1-ethyl-3-(3-dimethylaminopropylcarbodiimide). Alternatively, compounds of formula (A6) can be obtained by reacting (A5) with alkylating reagents followed by cyclization reaction under basic conditions. In the former case, suitable reagents include m-CPBA, and the reaction temperature is usually 0° C. to room temperature and preferably room temperature. Preferable solvents include dichloromethane and chloroform. In the latter case, suitable alkylating reagents include methyl indide, and suitable bases include sodium hydride, sodium bicarbonate, and potassium carbonate.

### Step 6:

Compounds of formula (1a) can be prepared by the following reaction sequence: 1) Y=H; deprotection of P² in formula (A6), nitration, protection, reduction, followed by amide coupling reaction with amines to afford compounds of formula (1a), 2) Y=Br or I; Buchwald-Hartwig reaction of formula (A6) with amides followed by deprotection of P² to afford compounds of formula (1a), 3) Y=trifluoroacetylamino: deprotection of the trifluoroacetylamino, amide coupling reaction, followed by deprotertion of P² to afford compounds of formula (1a). Examples of reaction conditions for 1)˜3) are described below:

### 1) Y=H:

Compounds of formula (A6) can be dopwiccted under the conditions described in Greene's Protective Groups in Organic Synthesis. When P² is benzoyl, the deprotection can be conducted with bases such as hydrazine hydrate or potassium carbonate using the solvent such as methanol and ethanol at room temperature to 80° C.

Nitration of tho deprotected compounds can be conducted by methods known to a person skilled in the art. For example, the nitrated compounds can be obtained by use of nitric acid or nitrate in aolveilta such as sulfuric acid or mixed solvent of sulfuric and trifluoroacetic acid. The reaction temperature is usually −20° C. to 0° C. The reaction time is usually 1 minute to 1 hour.

The amidlne group in the deprotected compounds can be protected by Boc under the conditions described in Greene's Protective Groups in Organic Synthesis. For example, the Hoc protection can be conducted using Boc₂O and a catalytic amount of N,N-dimethyl-4-aminopyridine in solvents such as dichloromethane and tetrahydrofuran at room temperature to 50° C.

Reduction of the nitrated compounds can be conducted by methods known to a person skilled in the art to afford the corresponding anilines: the following conditions can be used: 1) a method using iron powder in the presence of hydrochloric acid or ammonium chloride; 2) a method using palladium on carbon under hydrogen atmosphere. Examples of the solvent include solvents such as water. methanol, ethanol. ethyl acetate, tetrahydrofuran, and mixtures of those solvents.

Amide coupling reaction of the aniline with amines can be conducted by a method known to a person skilled in the art, and suitable coupling conditions can be found in Chem. Rev. 2011, 111, 6557-6602, which includes, a) reactions using condensation reagents: b) reactions using acid chlorides or fluorides.

Reaction a) can be conducted by use of condensation reagents such as dicyclohexycarbodiimide (DCC), diisopropylcarbodiimidr (DCC), 1-ethyl-3-(3-dimethylaminopropyl) carbodiimide hydrochloride (EDC hydrochloride), O-(7-aza-1H-benzotriazol-1-yl)-N,N,N′,N′-tetramethyluronium hexafluorophosphate (HATU), and 1H-Benazotriazol-1-yloxy-tri(pyrrolidino) phosphonium hexafluorophospate (PyBOP). When using uronium or phosphonium salts such as HATU and PyBOP, the reaction can be performed in the presence of bases such as triethylamine and diisopropylethylamine. The reaction may be accelerated by use of catalysts such as 1-hydroxy-benzoltriazole (HOBt) and 1-hydroxy-7-aza-bensotriazole (HOAt). The solvent used in the reaction is not particularly limited in so far as it does not interfere with the reaction. Examples of the solvent include dichloromomethane, N,N-dimethylformamide, N-methylpyrrolidone, and tetrahydrofuran. The reaction temperature is usually 0° C. to 50° C. and is preferably room temperature.

Reaction b) can be performed by use of commercially available acid chlorides or those synthesized by known methods to a person skilled in the art in solvents such as dichloromethane, tetrahydrofuran, and ethyl acetate in the presence of bases such as triethylamine, diisopropylethylamine, pyridine, and N,N-dimethyl-aminopyridine. The reaction temperature is usually 0° C. to 60° C. and is preferably 0° C. in room temperature. The reaction time is not particularly limited and is usually 5 minutes in 21 hours, preferably 20 minutes in 6 hours.

### 2) Y=Br or I:

Buchwald-Hartwig reaction of compounds of formula (A6) with amide derivatives can be conducted by a methods described in Metal-Catalyzed Cross-Coupling Reactions, 2nd ed. For example, this reaction can be performed by use of transition metal catalysis such as tris(dibenzylideneacetone) dipalladium and palladium acetate and ligands such as 2,2′bis(diphenylphosphino)-1,1′-binaphthyl (BINAP), 4,5-Bis(diphenylphosphino)-9,9-dimethylxanthene (Xantphos), and 2-Dicyclohexylphosphino-2′,4′,6′-triisopropylbiphenyl (X-Phos) in the presence of bases such as sodium tert-butoxide, cesium carboiute, and potassium phosphate. The reaction temperature is usually 40° C. to 150° C. and is preferably 60° C. to 100° C. This reaction may be accelerated by microwave irradiation. Examples of the solvent include toluene, benzene, xylene, tetrahydrofuran, 1,4-dioxane, and 1,2-dimethoxyethane.

After the Buchwald-Hartwig reaction, the deprotection of P² in the resulting compound can be performed under the conditions described above.

3) Y=trifluoroacetylamino:

Deprotection of the trifluoroacetylamino group in compounds of formula (A6) can be conducted by a methods known to a person skilled in the art. Suitable conditions can be found in Greene's Protective Groups in Organic Synthesis. For example, use of potassium carbonate in methanol at room temperature may be a usual method, but not limited to. The following amide coupling reaction and deprotection of P² can be conducted under the same conditions described above.

General Procedure B:

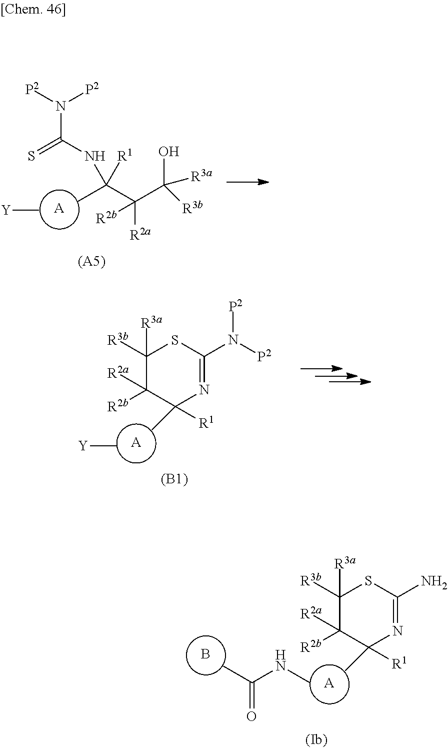

wherein the symbols are the same as defined in General Procedure A.

General Procedure B is a method for preparing compounds of formula (Ib) from compounds of formula (A5) through multiple steps. Using compounds of formula (B1), compounds of formula (1L) can be prepared according to the methods described in General Procedure A.

### Step 1:

Compounds of formula (B1) can be prepared by cyclixation reaction of compounds of formula (A5) by converting the hydroxy group into leaving groups such as Cl, Br, and triflate. The reaction conditions are known to those skilled in the art. For example, chlorination followed by cyclization may be achieved using reagents such as 1-chloro-N,N,2-trimethylpropenylamine. Alternatively, triflicanhydride may be used in tbe presence of bases such as N,N-dimethyl-4-aminopyridine and pyridine. Examples of the solvent include dichloromethane and tetrahydrofuran. The reaction temperature is usually 0° C. to room temperature and preferably 0° C. The reaction time is not particularly limited and is usually 0.5 to 3 hours.

### General Procedure C:

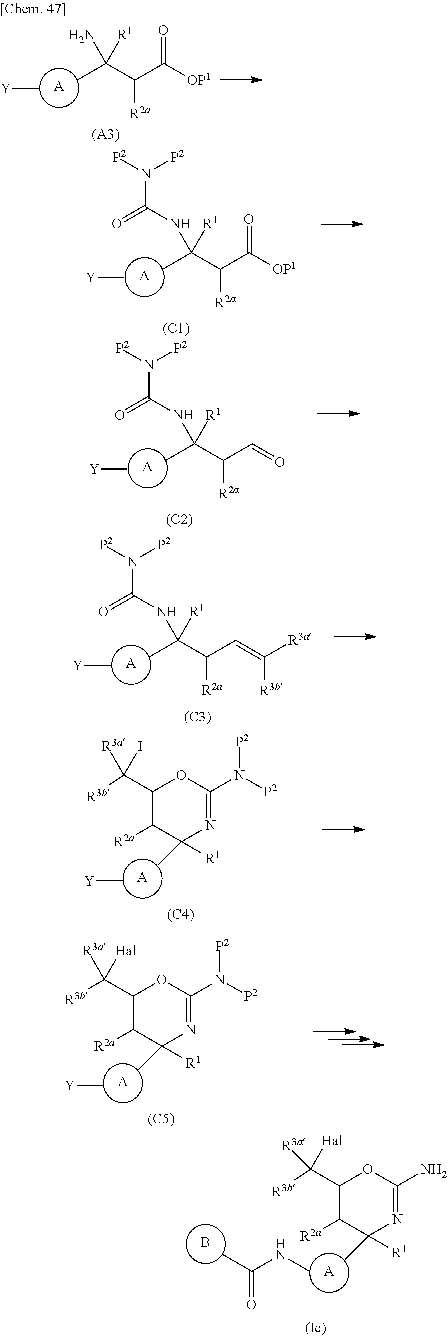

wherein Hal is halogen, R^{3a′} and R^{3b′} are each independently hydrogen or alkyl, and other symbols are the same as defined in General Procedure A.

General Procedure C is a method for preparing compuunds of formula (Ic) from compounds of formula (A3) through multiple steps. Using compounds of formula (C6), compounds of formula (Ic) can be prepared according to the methods described in General procedure A.

### Step 1:

Compounds of formula (C1) can be prepared by urea formation of compouuds of formula (A3). This type of reaction is known to those skilled in the art and is usually performed by treatment of compounds of formula (A3) with reagents such as triphosgene, 4-nitrophenyl chloroformate, and carbonyl diimidazole followed by addition of amines such as bis(2,1-dimethoxybenyl)amine. Preferable combination of these reagents may be 4-nitrophenyl chloroformate and bis(2,4-dimethoxybenzyl)amine. In such case, the reaction can be performed in the presence of bases such as solium bicarbonate in solvents such as water, tetrahydrofuran, ethyl acetate, and mixture of these solvents. The reaction temperature is usually 0° C. to room temperature. The reaction time is not particularly limited and is usually 1 to 12 hours.

### Step 2:

Compounds of formula (C2) can be prepared by reduction of compounds of formula (C1). This reaction is known to those skilled in the art and is usually preformed using diisobutylaluminium hydride (DIBAL-H). Examples of the solvents include dichloromethane, tetrahydrofuran, and toluene. The reaction temperature is usually below −60°C. and preferably below −70° C. The reaction time is not particularly limited and is usually 1 to 12 hours.

### Step 3

Compounds of formula (C3) can be prepared by Wittig reaction of compounds of formula (C2) with the corresponding phosphonium ylides. Alternatively, Peterson olefination, Horner-Wadsworth-Emmons reaction, Julia coupling, and Knoevenagel condensation may be considered. These reactions are known to those skilled in the art. For example, Wittig reaction can be generally conducted by treatment of the corresponding alkyl halide with triphenylphosphine followed by bases such as n-butyl lithium, which can be then added to compounds of formula (C3) in solvents such as tetrahydrofuran. The reaction time is not particularly limited and is usually 1 to 12 hours.

### Step 4:

Compounds of formula (C1) can be prepared hy cyclization of compounds of formula (C3) using iodine. Examples of the solvent include acetonilrile, tetrahydrofuran, and dichloromethane. The reaction temperature is usually 0° C. to 5° C. and preferably room temperature. The reaction time is not particularly limited and is usually 1 to 12 hours.

### Step 5:

Compounds of formula (C5) can be prepared by 1) halogenation of commpounds of formula (C4): 2) hydroxylation of compounds of formula (C4) followed by deoxohalogenation of the corresponding alcohol.

As for 1), halogenation, e.g., fluorination, of compounds of formula (C4) can be performed with reagents such as tetrabutylammonium fluoride (TBAF). Examples of the solvent include acetonitrile and tetrahydrofuran. The reaction temperature is usually 0° C. to 50° C. and preferably room temperature. The reaction time is not particularly limited and is usually 1 to 12 hours.

As for 2), hydroxylation of compounds of formula (C4) can be conducted with reagents such as potassium superoxide (KO₂), silver trifluoroacetate, and silver irifluoroborate. Preferable examples of the solvent include dimethyl sulfoxide (DMSO) for KO₂, nitromethane-water for silver trifluoroacetate, and DMSO-water for silver trifluoroborate. The reaction temperature is not particularly limited and is preferably room temperature for KO₂, 60° C. to 80°C. for silver trifluoroacetate, and 60° C. to 80° C. for silver trifluoroborate. The following deoxohalogenation, e.g., deoxofluorination, can be conducted with reagents such as N,N-diethylaminosulfur trifluoride (DAST), and bis(2-methoxyethyl)aminosulfur trifluoride (Deoxofluor: Trademark). Examples of the solvent include dichloromethane, acetonitrile, and tetrahydrofuran. The reaction temperature is usually −78° C. to room temperature and is preferably −78° C. to 0° C. Alternative conditions can be found in Synthesis 2002, 2561-2578.

### General Pprocedure D:

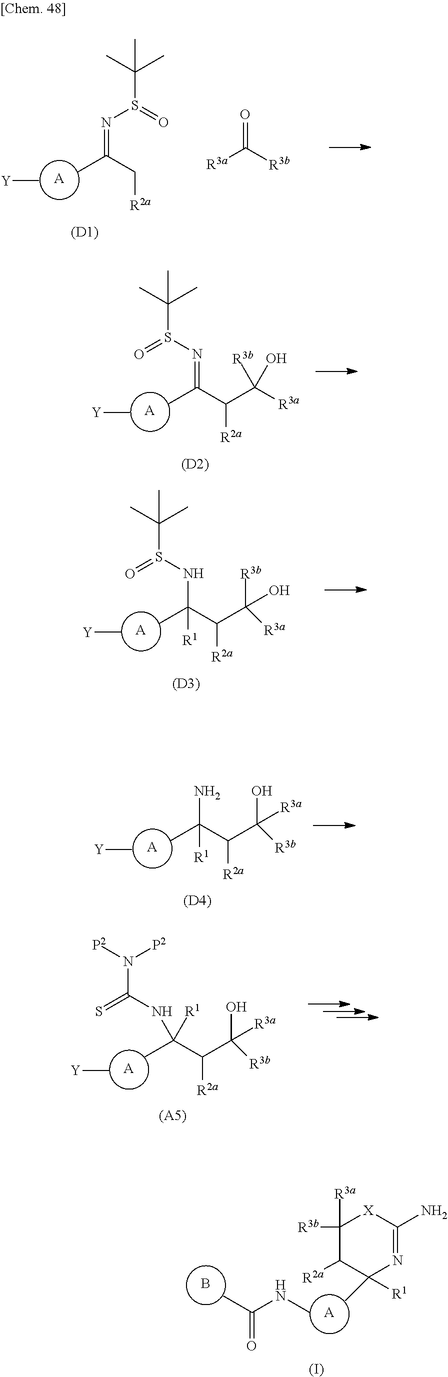

wherein the symbols are ihe same as defined in General Procedure A.

General Procedure D is a method for preparing compounds of formula (I) from compounds of formula (D1) through multiple steps. Using compounds of formula (A5), compounds of formula (I) can be prepared according to the methods described in General procedure A and B. The starting material of formula (D1) can be prepared in a manner similar to the conditions described in Chem. Rev. 2010, 110, 3600-3740.

### Step 1:

Compounds of formula (D2) can be prepared by addition of compound of formula (D1) to ketones of formula (R^{3a}COR^{3b}). This reaction can be performed under conditions similar to those described in Chem. Rev. 2010, 110, 3600-3740. For example, the ketimines derived from formula (D1) can be prepared using lithium diisopropylamide followed by addition of ketones (R^{3a}COR^{3b}) to afford (D2). Examples of the solvent include tetrahydrofuran and toluane. The reaction temperature is usually below −60− C. and preferably below −70° C. The reaction time is not particularly limited and is usually 1 to 12 hours.

### Step 2:

Compounds of formula (D3) can be prepared by reaction of (D2) with Grignard reagents such as methyl magnesium bromide and ethyl magnesium bromide and alkyl lithium reagents such as methyllithium, butytlithium, and phenyllithium. The solvent is not particularly limited in so for as it does not interfere with the reaction. Preferable examples of the solvent include tetrahydrofuran, 1,4-dioxane, 1,2-dimethoxyethane, diethyl ether, toluene, and benzene. The reaction temperature is not particularly limited and is usually 5 minutes to 24 hours, preferably 5 minutes to 6 hours. The reaction temperature is usually −78° C. to room temperature and is preferably −78° C. to −40° C.

### Step 3:

Compounds of formula (D4) can be prepared according to the method described in Step 2 of General Procedure A.

### Step 4:

Compounds of formula (A5) can be prepared according to the method described in Step 3 of General Procedure A.

General procedure E:

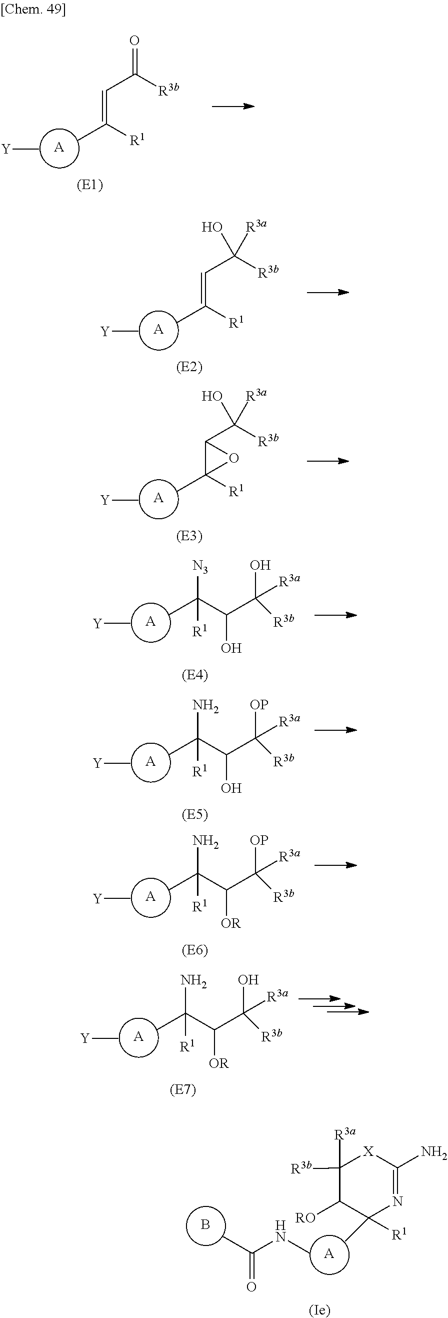

wherein P is a protective group for the hydroxy group such as benzyl and tert-butyldimethylsilyl, R is alkyl or haloalkyl, and the other symbols are the same as defined in General Procedure A.

General Procedure E is a method for preparing compounds of formula (I) from compounds of formula (E1) through multiple steps. Using compounds of formula (E8), compounds of formula (1) can be prepared according to the methods described in General procedure A and B.

Step 1:

Compounds of formula (E2) can be prepared by addition reaction of such as Me₃SiCF₃, Me₃SiCHF₂, and Me₃SiCH₂F in the presence of a catalytic amount of bases such as TBAF, cesium fluoride, and potassium fluoride. Examples of the solvent include tetrahydrofuran, N,N-dimethylfurmamide (DMF), acetonitrile, and toluene. The reaction temperature is usually −20° C. to room temperature and is preferably room temperature. Alternatively, this reaction can be performed by use of alkyl or haloalkyl cerium reagents prepared by cerium (III) chloride and alkyl lithium or Grignard reagents to afford compounds of formula (E2). Use of alkyl or haloalkyl lithium or Grignard reagents, without cerium (III) chloride may provide (E2) according to a method known to a person skilled in the art.

Step 2:

Compounds of formula can be prepared by epoxidation of compounds of formula (E2). Epoxidation is known to a person skilled in the art and is performed by use of oxidants such as m-CPBA and tert-butyl hydroperoxide in solvents such as dichloromethane and chloroform. The reaction time is not particularly limited and is usually 0.5 to 3 hours. The reaction temperature is usually −50° C. to room temperature. Asymmetric epoxidation such as Sharpless asymmetric epoxidation can be also applied to this step using methods known to those skilled in the art, which may be helpful to synthesize chiral compounds without chirol separation. Suitable conditions can be found in Comprehensive Organic Synthesis 1991, 7, 389.

### Step 3:

Compounds of formula (E4) can be prepared by ring opening reaction of compounds of formula (E3) using sodium azide in the presence of Lewis acids such as Ti(OEt)₄. Examples of the solvent include solvents such as tetrahydrofuran, toluene, and ethyl ether. The reaction time is not particularly limited and is usually 1 to 24 hours. The reaction temperature is usually room temperature.

### Step 4:

Protection of compounds of formula (E4) can be conducted by benzyl bromide or tert-butyldimetylsilyl chloride to afford compounds of formula (E5). When protecting by benzyl. The protection may be conducted using benzyl bromide in the presence of dibutyltin oxide. Examples of the solvent include toluene, methanol, DMF, and these mixed solvents. The reaction temperature is usually 60° C. to 100° C. When protecting by tert-butyldimethylsilyl, suitable conditions can be found in Greene's Protective Groups in Organic Synthesis. For example, the protection may be conducted using tert-butyldimetylsilyl chloride in the presence of imidazole as a base. Examples of the solvent include tetrahydrofuran, dichloro methane, and DMF. The reaction temperature is usually 0° C. to room temperature.

### Step 5:

Compounds of formula (E6) can be prepared by alkylation of compounds of formula (E5). This reaction is known to a person skilled in tbe art and is usually performed using alkylating reagents such as alkyl iodide, alkyl bromide, and alkyl triflate in the presence of bases such its sodium hydride, potassium carbonate, and sodium carbonate. Examples of the solvent include tetrahydrofuran, DMF, toluene, acetone, and acetonitrile. The reaction temperature is usually 0° C. to room temperature.

### Step 6:

Compounds of formula (E7) can be deproteeicd under conditions similar to those described in Greene's Protective Groups in Organic Synthesis. For example, when P is benzyl, the deprotection can be informed by hydrogenation in the presence of a catalytic amount of palladium carbon or palladium hydroxide. When P is tert-butyldimethylsilyl, the deprotection can be performed by TBAF in solvents such as tetrahydrofuran at 0° C. to room temperature.

### General Procedure F:

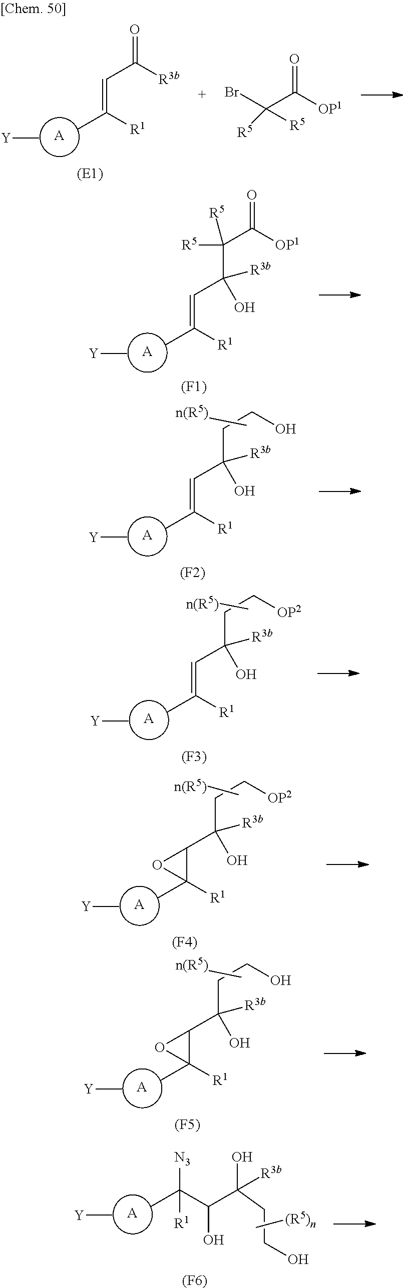

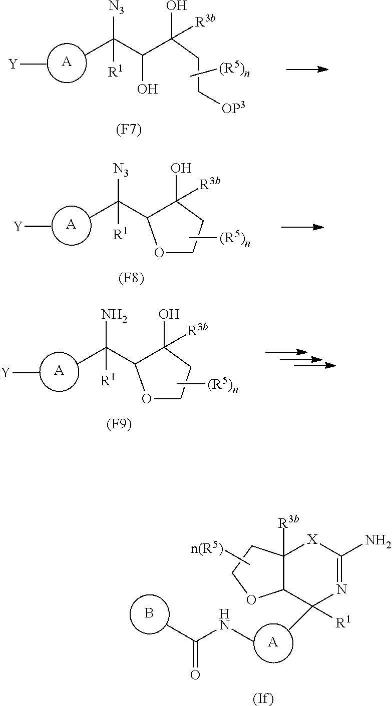

wherein P¹ is alkyl; P² is a protective group for the hydroxy group such as tert-butyldimethylsilyl; P³ is methanesulfonyl or toluenesulfonyl, and the other symbols are the same as defined to General Procedure A.

General Procedure F is a method for preparing compounds of formula (If) from compounds of formula (E1) through multiple steps. Using compounds of formula (F9), compounds of formula (I) can be prepared according to the methods described in General procedure A and B.

### Step 1:

Compounds of formula (F1) can be prepared by Reformatsky reaction of compounds of formula (E1) with α-haloesters. This reaction is known to a person skilled in the art and is usually performed under conditions described in Tetrahedron 2004, 42, 9325-9374. For example, a mixture of compounds of formula (F1) and α-haloesters in solvents such as tetrahydrofuran, acetonitrile, and toluene is reacted in the presence of zinc powder at room temperature to 100° C. The reaction time is not particularly limited and is usually 1 hour to 12 hours.

### Step 2:

Compounds of formula (F2) can be prepared by the method described in Step 2 of General Procedure C.

### Step 3:

Compounds of formula (F3) can be prepared by protection of the alcohol of formula (F2). The protective group jan be selected depending on reaction conditions used in the next step. Suitable protective groups can be found in Greene's Protective Groups in Organic Synthesis. For example, when tert-butyldimethylsilyl group is selected, the protection can be performed using tert-butyldimethylsilyl chloride in the presence of bases such as imidazole and sodium hydride in solvents such as DMF, tetrahydrofuran, and acetonitrile at 0° C. to room temperature. The reaction time is not particularly limited and is usually 0.5 to 6 hours. If the yield is low, use of tert-butyldimethylsilyl triflate instead of the corresponding chloride may be a proper choice.

### Step 4:

Compounds of formula (F4) can be prepared according to the conditions described in Step 2 of General Procedure E.

### Step 5:

Compounds of formula (F5) can be prepared by deprotection of compounds of formula (F4). Depending on the protective group in formula (F4), a deprotection condition can be selected according to Greene's Protective Groups in Organic Synthesis. When P2 is tert-butyldimethylsilyl, the deprotection can be conducted using TBAF in solvents such as tetrahydrofuran, DMF, and acetronitrile at 0° C. to room temperature. The reaction time is not particularly limited and is usually 0.5 to 6 hours.

### Step 6:

Compounds of formula (F6) can be prepared according to the conditions described in Step 3 of General Procedure E.

### Step 7:

The terminal alcohol of compounds of formula (F6) can be converted into the corresponding leaving group such as methanesulfonate or toluenesulfonate in this step. This reaction is known to a person skilled in the art and is usually conducted according to the method described in Greene's Protective Groups in Organic Synthesis. For example, protection of toluenesulfonyl can be performed using toluenesulfonyl chloride in the presence of bases such as N,N-dimethylamino-4-pyridine, pyridine, and triethylamine in solvents such as dichloromethane, tetrahydrofuran, and acetonitrile at 0° C. to room temperature. The reaction time is not particularly limited and is usually 0.5 to 6 hours.

### Step 8

Compounds of formula (F8) can be prepared by cyclization of compounds of formula (F7). This reaction can be achieved by use of bases such as potassium carbonate and sodium carbonate in solvents such as methanol, ethanol, and acetone at room temperature. The reaction time is not particularly limited and is usually 1 to 6 hours.

### Step 9:

Compounds of formula (F9) can be prepared according to the conditions described in Step 4 of General Procedure E.

The compounds of the present invention have BACE1 inhibitory activity and are effective in treatment and/or prevention, symptom improvement, and prevention of the progression of disease induced by the production, secretion or deposition of-amyloid β protein, such as Alzheimer's disease. Alzheimer dementia, senile dementia of Alzheimer type, mild cognitive impairment (MCI), prodromal Alzheimer's disease (e.g., MCI due to Alzheimer's disease), Down's syndrome, memory impairment, prion disease (Creutzfeldt-Jakob disease), Dutch type of hereditary cerebral hemorrhage with amyloidosis, cerebral amyloid angiopathy, other type of degenerative dementia, mixed dementia such as coexist Alzheimer's disease with vascular type dementia, dementia with Parkinson's Disease, dementia with progressive supranuclear palsy, dementia with Cortiro-based degeneration. Alzheimer's disease with diffuse Lewy body disease, age-related macular degeneration, Parkinson's Disease, amyloid angiopathy or the like.

Furthermore, the compounds of the present invention are effective in preventing the progression in a patient asymptomatic at risk for Alzheimer dementia (preclinical Alzheimer's disease).

“A patient asymptomatic at risk for Alzheimer dementia” includes a subject who is cognitively and functionally normal but has potential very early signs of Alzheimer's disease or typical age related changes (e.g., mild while matter hyper intensity on MRI), and/or have evidence of amyloid deposition its demonstrated by low cerebrospinal fluid AB₁₋₄₂ levels. For example, “a patient asymptomatic at risk for Alzheimer dementia” includes a subject whose score of the Clinical Dementia Rating (CDR) or Clinical Dementia Rating-Japanese version (CDR-J) is 0, and/or whose stage of the Functional Assessment Staging (FAST) is stage 1 or stage 2.

The compound of the present invention has not only BACE1 inhibitory activity but the beneficialness as a medicament. The compound has any or all of the following superior properties.
a) The compound has weak inhibitory activity for CYP enzymes such as CYP1A2, CYP2C9, CYP2C19, CYP2C6, CYP3A4.
b) The compound show excellent pharmacokinetics profiles such as high bioavailability or low clearance.
c) The coumpound has a high metabolic stability.
d) The compound does not show irreversible inhibitions to CYP enzymes such as CYP3A4 in the range of the concentrations of the measurement conditions described in this description.
e) The compound does not show a mutagenesis.
f) The compound is at a low risk for cardiovascular systems.
g) The compound shows a high solubility.
h) The compound shows a high brain distribution.
i) The compound has a high oral absorption.
j) The compound has a long half-life period.
k) The compound has a high protein unbinding ratio.
l) The compound is negative in the Ames test.
m) The compound has a high BACE1 selectivity over BACE2.

Since the compound of the present invention has high inhibitory activity on BACE1 and/or high selectivity on other ensymes, for example. BACE2, it can be a medicament with reduced side effect. Further, since the compound has high effect of reducing amyloid β production in a cell system, particularly, has high effect of reducing amyloid β production in brain, it can be an excellent medicament. In addition, by converting the compound into an optically active compound having suitable stereochemistry, the compound can be a medicament having a wider safety margin on the side effect.

When a pharmaceutical composition of the present invention is administered, it can be administered orally or parenterally. The composition for oral administration can be administered in usual dosage forms such as oral solid formulations (e.g., tablets, powders, granules, capsules, pills, films or tbe like), oral liquid formulations (e.g., suspension, emulsion, elixir, syrup, lemonade, spirit, aromatic water, extract, decoction, tincture or the like) and the like may prepared according to the usual method and administered. The tablets can be sugar-coated tablets, film-coated tablets, enteric-coating tablets, sustained-release tablets, troche tablets, sublingual tablets, buccal tablets, chewable tablets or orally disintegrated tablets. Powders and granules can be dry syrups. Capsules can be soft capsules, micro capsules or sustained-release capsules.

The composition for parenteral administration can be administered suitably in usual parenteral dosage forms such as dermal, subcutaneous, intravenous, intraarterial, intramuscular, intraperitoneal, transmucosal, inhalation, transnasal, ophthalmic, inner ear or vaginal administration and the like. In case of parenteral administration, any forms, which are usually used, such as injections, drips, external preparations (e.g.m ophthalmic drops, nasal drops, ear drops, aerosols, inhalations, lotion, infusion, liniment, mouthwash, enema, ointment, plaster, jelly, cream, patch, cataplasm, external powder, suppository or the like) and the like can be preferably administered. Injections can be emulsions whose type is O/W, W/O, O/W/O, W/O/W or the like.

The compounds of the present invention can be preferably administered in an oral dosage form because of their high oral absorbability.

A pharmaceutical composition can be formulated by mixing various additive agents for medicaments, if needed, such as excipients, binders, disintegrating agents, and lubricants which are suitable for the formulations with an effective amount of the compound of the present invention. Furthermore, the pharmaceutical composition can be for pediatric patients, geriatric patients, serious cases or operations by appropriately changing the effective amount of the compound of the present invention, formulation and/or various pharmaceutical additives. The pediatric pharmaceutical compositions are preferable administered to patients under 12 or 15 years old. In addition, the pediatric pharmaceutical compositions can be administered to patients who am under 27 days old after the birth, 28 days to 23 months old after the birth, 2 to 11 years old, 12 to 16 years old, or 18 years old. The geriatric pharmaceutical compositions are preferably administered to patients who are 65 years old or over.

The dosage of a pharmaceutical composition of the present invention should be determined in consideration of the patient's age and body weight. The type and degree of diseases, the administration route and the like. The usual oral dosage for adults is in the range of 0.05 to 100 mg/kg/day and preferable is 0.1 to 10 mg/kg/day. For parenteral administration, the dosage highly varies with administration routes and the usual dosage is in the range of 0.005 to 10 mg/kg/day and preferably 0.01 to 1 mg/kg/day. The dosage may be administered once or several times per day.

The compound of the present invention can be used in combination with other drugs for treating Alzheimer's disease. Alzheimer dementia or the like such as acetylcholinesterase inhibitor (hereinafter referred to as a conconmitant medicament) for the purpose of enforcement of the activity of the compound or reduction of the amount of medication of the compound or the like. In this case, timing of administration of the compound of the present invention and the concomitant medicament is not limited and these may be administered to the subject simultaneously or at regular intervals. Furthermore, the compound of the present invention and concomitant medicament may be administered as two different compositions containing each active ingredient or as a single composition containing both active ingredient.

The dose of the concomitant medicament can be suitably selected on the basis of the dose used on clinical. Moreover, the mix ratio of the compound of the present invention and a concomitant medicament can be suitably selected in consideration of the subject of administration, administration route, target diseases, symptoms, combinations, etc. For example, when the subject of administration is human, the concomitant medicament can be used in the range of 0.01 to 100 parts by weight relative to 1 part by weight of the compounds of the present invention.

Examples of a concomitant medicament are Donepezil hydrochloride, Tacrine, Galanthamine, Rivastigmine, Zanapezil, Memantine and Vinpocetine.

Following examples and test examples illustrate the prsent invention in more detail, but the present invention is not limited by these examples.

In examples, the meaning of each abbreviation is as follows:
Ac acetyl
Et ethyl
Boc tert-butoxycarbonyl
Bn benzyl
Bz benzoyl
Me methyl
Ph phenyl
t-Bu tert-butyl
TBS tert-butyldimethylsilyl
TMS trimethylsilyl
AlBN azobisisobutyronitrile
DAST N,N-diethylaminosulfur trifluoride
DIBAL diisobutylaluminium hydride
DIPEA N,N-diisopropylethylamine
DMF N,N-dimethylformamide
DMAP 4-diimethylaminopyridine
DMSO dimethylsulfoxide
EDC 1-ethyl-3-(3-dimethylaminopropyl)carbodiimide
HATU 1-[bis(dimethylamino)methylene]-1H-1,2,3-triazolo[4,5-b]pyridinium 3-oxid hexafluorophosphate
LHMDS lithium hexamethyldisilaside
m-CPBA meta-chloroperbenzoic acid
TFA trifluoroacetic acid
THF tetrahydrofuran
TBAF tetrabutylammonium fluoride
WSCD 1-ethyl-3-(3-dimethylaminopropyl) carbodiimide hydrochloride

¹H NMR spectra were recorded on Bruker Advance 400 MHz spectrometer with chemical shift reported relative to tetramethylsilane or the residual solvent peak (CDCl₃=7.20 ppm, DMSO-d₆=2.50 ppm).

Analytical LC/MS (ESI positive or negative, retention time (RT)) data were recorded on Shimadsu UFLC or Waters UPLC system under the following conditions:
Column: Shim-pack XR-ODS (2.2 μm, i.d. 50×3.0 mm) (Shimadzu)
Flow rate: 1.6 mL/min
Column oven: 50° C.
UV detection wavelength: 254 nm
Mobile phase: [A] 0.1% formic acid-containing aqueous solution: [B] 0.1% formic acid-containing acetonitrile solution
Gradient: linear gradient from 10% to 100% solvent [B] for 3 minutes and 100% solvent [B] for 1 minute

### EXAMPLE 1

### Synthesis of Compound I-5

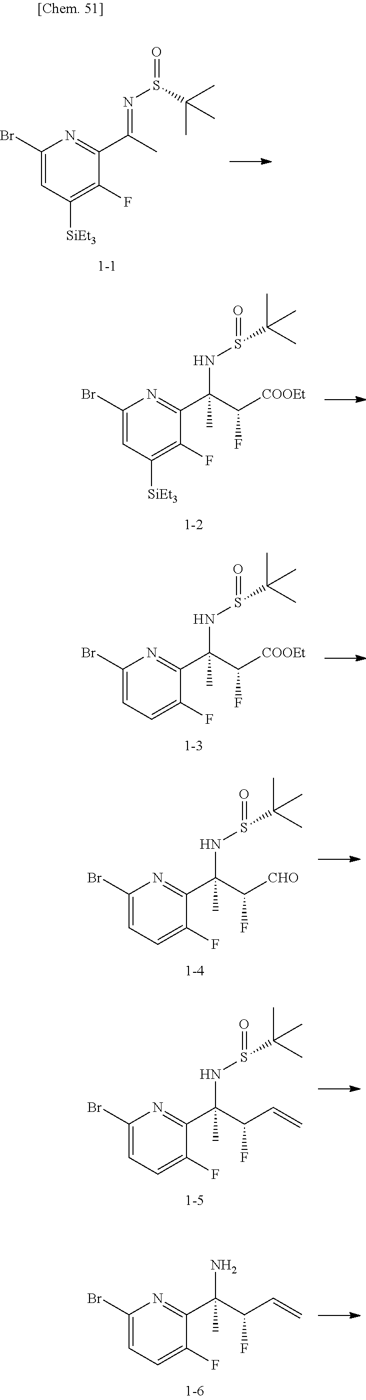

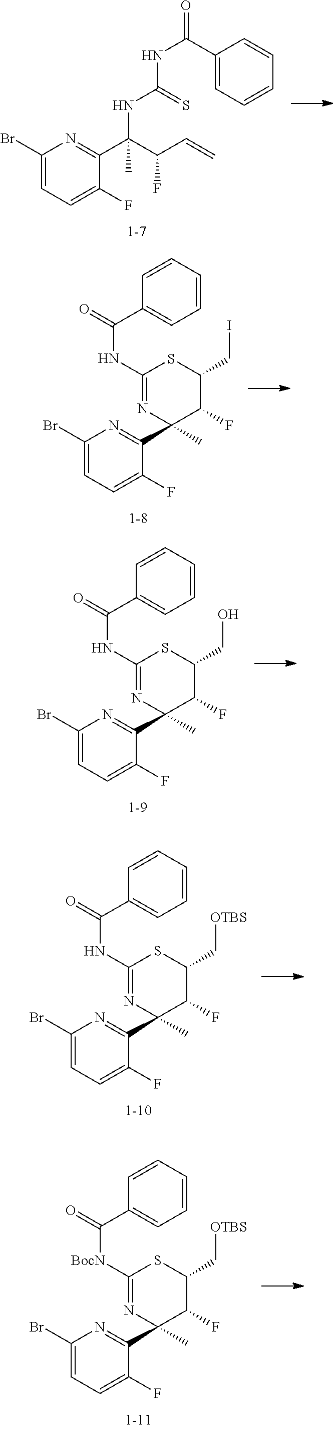 [SMILES: C=C[C@H](F)[C@](C)(NC(=S)NC(=O)c1ccccc1)c1nc(Br)ccc1F ; C[C@]1(c2nc(Br)ccc2F)N=C(NC(=O)c2ccccc2)S[C@H](CI)[C@@H]1F ; C[C@]1(c2nc(Br)ccc2F)N=C(NC(=O)c2ccccc2)S[C@H](CO)[C@@H]1F ; CC(C)(C)[Si](C)(C)OC[C@H]1SC(NC(=O)c2ccccc2)=N[C@](C)(c2nc(Br)ccc2F)[C@H]1F ; CC(C)(C)OC(=O)N(C(=O)c1ccccc1)C1=N[C@](C)(c2nc(Br)ccc2F)[C@@H](F)[C@@H](CO[Si](C)(C)C(C)(C)C)S1]

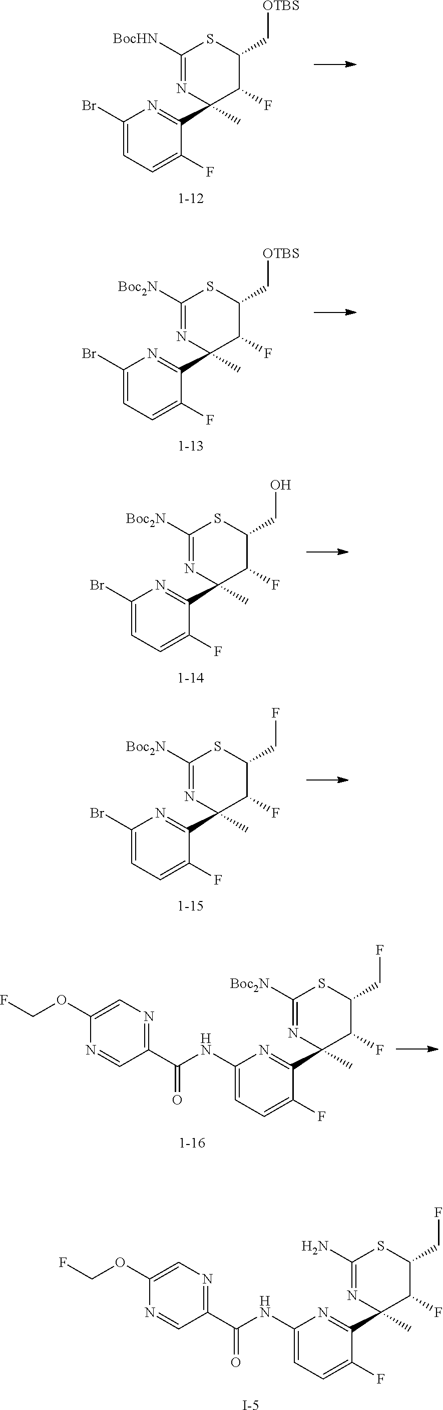

### Step 1

A Stirred suspension or zinc (1.40 g, 21.4 mmol) in THF (80 ml) was heated to reflux. To the suspension were added a solution of compound 1-1 (8.46 g, 19.4 mmol) in THF (20 ml) and a solution of ethyl 2-bromo-2-fluoroacetate (3.95 g, 21.4 mmol) in THF 10 ml). After stirring for 3 h at the same temperature, the reaction mixture was treated with saturated aqueous NH₄Cl and the aqueous layer was extracted with AcOEt. The combined organic layers were washed with brine, dried over Na₂SO₄, filtered and concentrated. The crude product was added to a silica gel column and eluted with Hexane/EtOAc 25%. Collected fractions were evaporated to afford compound 1-2 (5.76 g, 10.6 mmol, 55%) as a brown oil.

¹H-NMR (400 MHz, CDCl₃) δ: 0.84-0.89 (m, 6H), 0.94-0.98 (m, 9H), 1.24 (s, 9H), 1.29 (t, J=7.2 Hz, 3H), 1.96 (s, 3H), 4.26 (m, 2H), 5.16 (s, 1H), 5.34 (d, J=46.4 Hz, 1H), 7.37 (d, J=2.6 Hz, 1H).

### Step 2

KF (1.24 g, 21.3 mmol) was added to a solution of compound 1-2 (5.76 g, 10.6 mmol) and AcOH (1.22 ml, 21.3 mmol) in THF (30 ml). DMF (30 ml) was added, and the mixture was stirred at room temperature. After stirring for 2.5 h at the same temperature, the reaction mixture was treated with saturated aqueous NaHCO₃, and the aqueous layer was extracted with AcOEt. The combined organic layers were washed with brine, dried over Na₂SO₄, filtered and concentrated. The crude product was added to a silica gel column and eluted with Hexane/EtOAc 30% to 50%. Collected fractions were evaporated to afford compound 1-3 (1.01 g, 9.38 mmol, 88%) as a brown oil.

¹H-NMR (400 MHz, CDCl₃) δ: 1.25 (s, 9H0, 1.30 (t, J=7.2 Hz, 3H), 1.97 (s, 3H), 4.27 (m, 2H), 5.14 (s, 1H), 5.35 (d, J=46.4 Hz, 1H), 7.29 (dd, J=10.8, 8.4 Hz, 1H), 7.45 (dd, J=8.4. 2.9 Hz, 1H).

### Step 3

To a solution of compound 1-3 (3.94 g, 9.22 mmol) in CH₂Cl₂ (40 ml) was added 1.02 mol/L DIBAL (27.1 ml, 27.7 mmol) at −76° C. After stirring for 15 min nt the same temperature, the mixture was treated with saturated aqueous Rochelle's salt and stirred for 2.5 h. The aqueous layer was extracted with AcOEt, and the combined organic layers were washed with brine, dried over Na₂SO₄ and filtered. The nitrate was concentrated under vacuum to give compound 1-4 as a yellow amorphous that was used for the next step without purification.

To a solution of methyltriphenylphosphonium bromide (8.23 g, 23.0 mmol) in toluene (85 ml) was added 1.00 mol/L t-BuOK solution in THF (21.2 ml, 21.2 mmol) at room temperature. After stirring for 1 h at the same temperature, a solution of compound 1-4 in toluene (30 ml) was added at 0° C. After stirring for 90 min at room temperature, the reaction mixture was treated with saturated aqueous NH₄Cl, and the aqueous layer was extracted with AeOEt. The combined organic laser was washed with brine, dried over Na₂SO₄, filtered and concentrated. The crude product was added to a silica gel column and eluted with Hexane/EtOAc 10% to 50%. Collected fractions were evaporated to afford compound 1-5 (1.57 g, 4.12 mmol, 45%) as a white amorphous.

¹H-NMR (400 MHz, CDCl₃) δ=1.24 (s, 9H), 1.85 (t, J=1.8 Hz, 3H), 5.11 (s, 1H), 5.17-5.32 (m, 2H), 5.34 (d, J=1.1 Hz, 1H), 5.91-6.04 (m, 1H), 7.29 (dd, J=10.7, 8.5 Hz, 1H), 7.43 (dd, J=3.0, 8.5 Hz, 1H).

### Step 4

To a solution of compound 1-5 (1.57 g, 4.12 mmol) in MeOH (16 ml) was added 4 mol/L HCl in Dioxane (1.51 ml, 0.18 mmol) at room temperature. After stirring for 30 min at the same temperature, the reaction mixture was treated with aqueous NaHCO₃ and the aqueous layer was extracted with AcOEt. The combined organic layers were washed with H₂O and brine, dried nver Na₂SO₄, filtered and concentrated to give compound 1-6, which was used for the next step without purification.

To a solution of 1-6 in CH₂Cl₂ (11 ml) was added benzoyl isothiocyanate (0.848 ml, 2.58 mmol) at 0° C. After stirring for 30 min at room temperature, the reaction mixture was concentrated, and the resulting residue was added to a silica gel column and eluted with Hexane/EtOAc 0% to 25%. Collected tractions were evaporated to afford compound 1-7 (1.81 g, 4.12 mmol, quant) as a yellow oil.

¹H-NMR (400 MHz, CDCl₃) δ: 2.12 (d, J=0.9 Hz, 3H), 5.43-5.61 (m, 1H), 5.90-6.03 (m, 1H), 7.19 (dd, J=10.6, 8.5 Hz, 1H), 7.40 (dd, J=8.5, 3.0 Hz, 1H), 7.52 (t, J=7.7 Hz, 2H), 7.63 (t, J=7.7 Hz, 1H), 7.85 (d, J=7.7 Hz, 2H), 8.83 (s, 1H), 11.53 (s, 1H).

### Step 5

To a solution of iodine (2.09 g, 8.24 mmol) in MeCN (40 ml) was added compound 1-7 (1.81 g, 4.12 mmol) in MeCN (14 ml) at 0° C. After stirring for 20 min at the same temperature, the reaction mixture was treated with aqueous NaHCO₃ and Na₂S₂O₃. The aqueous layer was extracted with AcOEt. The combined organic layer was washed with brine, dried over Na₂SO₄, filtered and concentrated. The crude product was added to a silica gel column and eluted with hexane/EtOAc 0% to 35%. Collected fractions were evaporated to afford compound 1-8 (2.18 g, 3.85 mmol, 94%) as a yellow amorphous.

¹H-NMR (400 MHz, CDCl₃) δ: 1.90 (s, 3H), 3.29 (dd, J=10.4, 5.0 Hz, 1H), 3.58 (t, J=10.4 Hz, 1H), 3.94-3.79 (m, 1H), 5.75 (d, J=47.3 Hz, 1H), 7.52-7.32 (m, 6H), 8.16 (d, J=6.9 Hz, 2H).

### Step 6

To a solution of compound 1-8 (1.52 g, 2.68 mmol) in DMSO (1 ml) and H₂O (0.1 ml) was added AgBF₄ (1.05 g, 5.37 mmol) at room temperature. After stirring for 2 h at the same temperature, the reaction mixture was treated with aqueous NaHCO₃. The aqueous layer was extracted with AcOEt, and the organic layer was dried over Na₂SO₄, filtered and concentrated. The crude product was added to a silica gel column and eluted with hexane/EtOAc 0% to 50%. Collected fractions were evaporated to afford compound 1-9 (684 mg, 1.50 mmol, 56%) as a white amorphous

¹H-NMR (400 MHz, CDCl₃) δ: 1.88 (s, 3H), 3.74-3.91 (m, 2H), 4.14 (dd, J=7.3, 2.8 Hz, 1H), 5.67 (d, J=47.2 Hz, 1H), 7.31-7.51 (m, 6H), 8.18 (d, J=7.3 Hz, 2H).

### Step 7

To a solution of compound 1-9 (325 mg, 0.712 mmol) in DMF (4 ml) were added imidazole (194 mg, 2.85 mmol) and TBSCl (215 mg, 1.42 mmol) at 0° C. After stirring for 20 min at room temperature, the reaction mixture was treated with H₂O. The aqueous layer was extracted with AcOEt, and the organic layers were dried over Na₂SO₄, filtered and concentrated. The crude product was added to a silica gel column and eluted with hexane/EtOAc 0% to 30%. Collected fractions were evaporated to afford compound 1-10 (384 mg, 0.673 mmol, 95%) as a white amorphous.

¹H-NMR (400 MHz, CDCl₃) δ: 0.11 (s, 3H), 0.12 (s, 3H), 0.92 (s, 9H), 1.87 (s, 3H), 3.64-3.69 (m, 1H), 3.72-3.79 (m, 1H), 4.03-4.09 (m, 1H), 5.60 (dd, J=46.9, 1.3 Hz, 1H), 7.50-7.30 (m, 5H), 8.20 (d, J=7.3 Hz, 2H).

### Step 8

To a solution of compound 1-16 (384 mg, 0.673 mmol) in THF (4 ml) were added Boc₂O (0.234 ml, 1.01 mmol) and DMAP (32.9 mg, 0.269 mmol) at room temperature. After stirring for 20 min at the same temperature, the mixture was concentrated under vacuum. The crude product was added to a silica gel column and eluted with Hexane/EtOAc 0% to 30%. Collected fractions were evaporated to afford compound 1-11 (451 mg, 0.672 mmol, quant) as a while amorphous.

¹H-NMR (400 MHz, CDCl₃) δ: 0.08 (s, 3H), 0.09 (s, 3H), 0.90 (s, 9H), 1.39 (s, 9H), 1.62 (d, J=2.4 Hz, 3H), 3.71-3.76 (m, 1H), 3.89 (dt, J=30.3, 7.7 Hz, 1H), 4.06 (dd, J=9.8, 7.7 Hz, 1H), 5.33 (dd, J=47.4, 1.6 Hz, 1H), 7.19 (t, J=9.2 Hz, 1H), 7.33-7.40 (m, 3H), 7.48 (t, J=7.3 Hz, 1H), 7.76 (d, J=7.3 Hz, 2H).

### Step 9

To a solution of compound 1-11 (451 mg, 0.672 mmol) in THF (2 ml). MeOH (2 ml) and H₂O (2 ml) was added K₂CO₃(279 mg, 2.02 mmol) at room temperature. After stirring for 2 h at 50° C., the reaction mixture was treated with H₂O. The aqueous layer was extracted with AcOEt, and tho organic layer was dried over Na₂SO₄, filtered and concentrated. The crude product was added to a silica gel column and eluted with hexane/KtOAc 0% to 30%. Collected fractions were evaporated to afford compound 1-12 (346 mg, 0.611 mmol, 91%) as a white amorphous.

¹H-NMR (400 MHz, CDCl₃) δ: 0.07 (s, 3H), 0.08 (s, 3H), 0.89 (s, 9H), 1.50 (s, 9H), 1.79 (s, 3H), 3.49-3.70 (m, 2H), 3.99 (t, J=9.0 Hz, 1H), 5.53 (d, J=47.2 Hz, 1H), 7.30 (t, J=9.4 Hz, 1H), 7.45-7.43 (m, 1H).

### Step 10

To a solution of compound 1-12 (316 mg, 0.611 mmol) in THF (4 ml) were added Boc₂O (0.213 ml, 0.916 mmol) and DMAP (29.8 mg, 0.241 mmol) at room temperature. After stirring for 30 min at the same temperature, the mixture was concentrated under vacuum. The crude product was added to a silica gel column and eluted with Hexane/EtOAc 0% to 30%. Collected fractions were evaporated to afford compound 1-13 (370 mg, 0.555 mmol, 91%) as a white solid.

¹H-NMR (400 MHz, CDCl₃) δ: 0.11 (s, 3H), 0.11 (s, 3H), 0.92 (s, 9H), 1.42 (s, 18H), 1.82 (d, J=1.9 Hz, 3H), 3.73-3.79 (m, 1H), 3.97-4.15 (m, 2H), 5.45 (d, J=47.7 Hz, 1H), 7.25-7.31 (m, 1H), 7.39 (dd, J=8.4, 3.0 Hz, 1H).

### Step 11

To a solution of compound 1-13 (100 mg, 0.600 mmol) in THF (8 ml) were added AcOH (0.0510 ml, 0.900 mmol) and TBAF (1.00 mol/L solution in THF, 1.80 ml, 1.80 mmol) at room temperature. After stirring for 1 h at the same temperature, the reaction mixture was treated with H₂O. The aqueous layer was extracted with AcOEt, and the organic layer was dried over Na₂SO₄, filtered and concentrated. The crude product was added to a silica gel column and eluted with hexane/EtOAc 10% to 50%. Collected fractions were evaporated to afford compound 1-14 (323 mg, 0.585 mmol, 98%) as a white amorphous.

¹H-NMR (400 MHz, CDCl₃) δ: 1.43 (s, 9H), 1.84 (s, 3H), 3.84-3.95 (m, 1H), 4.04-4.19 (m, 2H), 5.51 (d, J=47.1 Hz, 1H), 7.27-7.42 (m, 2H).

### Step 12

To a solution of compound 1-14 (323 mg, 0.585 mmol) in CH₂Cl₂ (10 ml) was added DAST (0.257 ml, 1.75 mmol) at −78° C. After stirring for 40 min at room temperature, the reaction mixture was treated with aqueous NaHCO₃. The aqueous layer wan extracted with AvOEt, and the organic layer was dried over Na₂SO₄, filtered and concentrated. The crude product was added to a silica gel column and eluted with hexane/EtOAc 0% to 30%. Collected fractions were evaporated to afford compound 5-15 (310 mg, 0.559 mmol, 96%) as a white amorphous.

¹H-NMR (100 MHz, CDCl₃) δ: 1.42 (s, 18H), 1.85 (d, J=2.1 Hz, 3H), 4.28-4.61 (m, 2H), 4.90 (dt, J=46.6, 8.4 Hz, 1H), 5.50 (dd, J=47.1, 1.8 Hz, 1H), 7.30 (t, J=9.0 Hz), 7.42 (dd, J=9.0, 3.1 Hz, 1H).

### Step 13

A degassed mixture of Pd₂(dba)₃ (11.6 mg, 0.0130 mmol) and xantphos (21.9 mg, 0.0380 mmol) in dioxane (1 ml) was stirred for 1 h at room temperature. To this mixture were added dioxane (3 ml), compound 1-15 (70.0 mg, 0.126 mmol), 5-(fluoromethoxy)pyrazine-2-carboxamide (25.9 mg, 0.152 mmol) and cesium carbonate (49.4 mg, 0.152 mmol). After stirring for 6 h at 90° C., to the reaction mixture were further added 5-(fluoromethoxylpyrazine-2carboxamide (25.9 mg, 0.152 mmol) and centum carbonate (49.4 mg, 0.152 mmol). After stirring for additional 11 h, the reaction mixture was treated with aqueous citric acid and filtered. The aqueous layer was extracted with AeOEt, and the organic layer was dried over Na₂SO₄, filtered and concentrated. The crude product was added to a silica gel column and eluted with hexane/EtOAc 0% to 35%. Collected fractions were evaporated to afford compound 1-16 (55.0 mg, 0.0850 mmol, 68%) as a yellow oil.

¹H-NMR (400 MHz, CDCl₃) δ: 1.48 (s, 18H), 1.89 (s, 3H), 3.99-4.11 (m, 1H), 4.37-4.53 (m, 1H), 4.85 (dt, J=47.0, 8.8 Hz, 1H), 5.49 (d, J=48.4 Hz, 1H), 6.16 (d, J=50.9 Hz, 2H), 7.52 (t, J=0.3 Hz, 1H), 8.31 (s, 1H), 8.39 (dd, J=9.3-3.0 Hz, 1H), 8.09 (s, 1H), 10.01 (s, 1H).

### Step 14

A solution of compound 1-16 (55.0 mg, 0.0850 mmol) in formic acid (0.982 ml) was stirred for 22 h at room temperature. The reaction mixture was treated with aqueous K₂CO₃. The aqueous layer was extracted with AeOEt, and the organic layer was dried over Na₂SO₄, filtered and concentrated to afford compound 1-5 (24.0 mg, 0.0510 mmol, 63%) as a tan solid.

### EXAMPLE 2

### Synthesis of Compound I-7

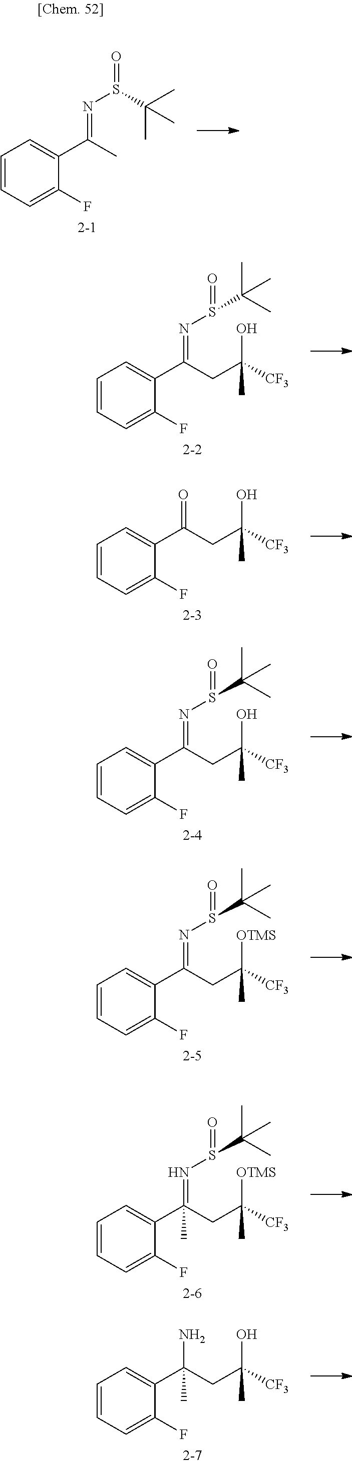

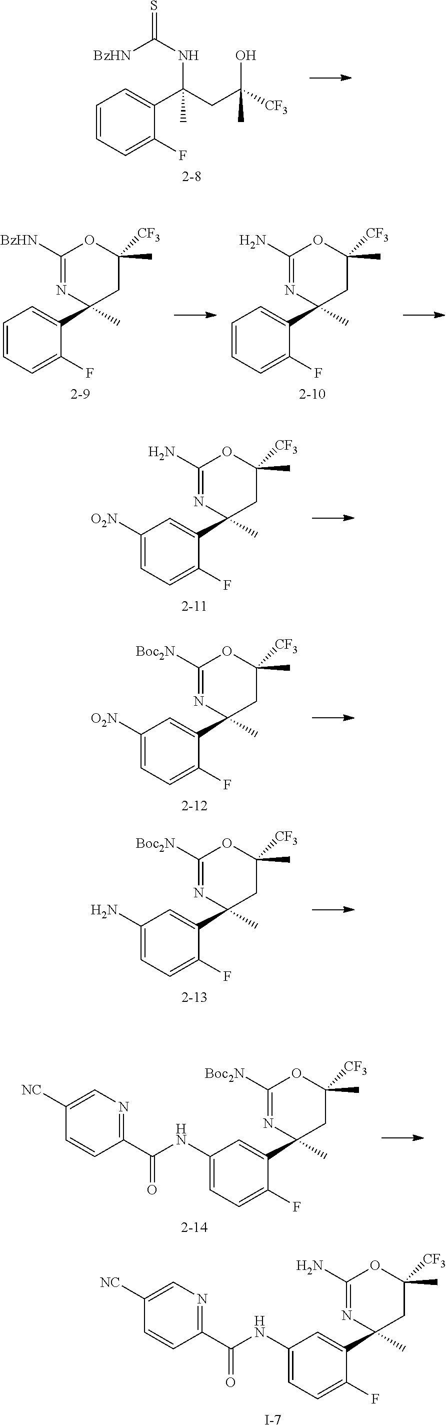

### Step 1

To a solution of compound 2-1 (2.00 g, 8.29 mmol) in THF (20 ml) was added a solution of LHMDS (1.00 mmol/L in THF, 16.6 mL, 16.6 mmol) at −78° C. After stirring for 40 min, a solution of 1,1,1-trifluoropropan-2-one (1.48 ml, 16.6 mmol) in THF (5 ml) was added and the mixture was stirred for 20 min at the same temperature. The reaction mixture was treated with aqueous NH₄Cl. The aqueous layer was extracted with AcOEt, and the organic layer was dried over Na₂SO₄, filtered and concentrated. The crude product was added to a silica gel column and eluted with hexane/EtOAc 0% to 20%. Collected fractions were evaporated to afford compound 2-2 (1.79 g, 5.07 mmol, 61%) as a yellow solid.

¹H-NMR (400 MHz, CDCl₃) δ: 1.04 (s, 3H), 1.39 (s, 9H), 3.45 (d, J=12.7 Hz, 1H), 3.80 (d, J=12.7 Hz, 1H), 6.31 (s, 1H), 7.15 (dd, J=11.4, 8.3 Hz, 1H), 7.24 (t, J=8.3 Hz, 1H), 7.55-7.44 (m, 2H).

### Step 2

To a solution of compound 2-2 (896 mg, 2.54 mmol) in MeOH (12 ml) was added 4 mol/L HCl in H₂O (6.34 ml, 12.7 mmol) at room temperature. After stirring for 1 h at the same temperature, the reaction mixture was treated with aqueous NaHCO₃, and the aqueous layer was extracted with AcOEt. The combined organic layer was washed with H₂O and brine, dried over Na₂SO₄ filtered and concentrated. The crude product was added to a silica gel column and eluted with hexane/EtOAc 20%. Collected fractions were evaporated to afford compound 2-3 (603 mg, 2.41 mmol, 95%) as a white solid.

¹H-NMR (400 MHz, CDCl₃) δ: 1.50 (s, 3H), 3.17 (d, J=17.8 Hz, 1H), 3.53 (d, J=17.8 Hz, 1H), 5.06 (s, 1H), 7.18 (t, J=9.8 Hz, 1H), 7.26-7.31 (m, 1H), 7.63-7.56 (m, 1H), 7.86 (t, J=7.5 Hz, 1H).

### Step 3

To a solution of compound 2-3 (500 mg, 2.00 mmol) in toluene (5 ml) were added titanium ethoxide (0.836 ml, 1.00 mmol) and (S)-2-methylpropane-2-sulfinamide (363 mg, 3.00 mmol). After stirring for 15 min at 80° C., the reaction mixture were added MeCN (10 ml) and H₂O (0.25 mL) at room temperature, and the insoluble material was removed by filtration. The filtrate was evaporated under reduced pressure. The crude product was added to a silica gel column and eluted with hexane/EtOAc 30%. Collected fractions were evaporated to afford compound 2-4 (588 mg, 1.66 mmol, 83%) as a while solid.

¹N-NMR (100 MHz, CDCl₃) δ: 1.33 (s, 3H), 1.37 (s, 3H), 3.35 (d, J=13.0 Hz, 1H), 3.95 (d, J=13.0 Hz, 1H), 5.89 (s, 1H), 7.07-7.25 (m, 2H), 7.54-7.40 (m, 2H).

### Step 4

To a solution of compound 2-4 (550 mg, 1.56 mmol) in DMF (5 ml) were added imidazole (318 mg, 4.67 mmol), TMSCl (338 mg, 3.11 mmol) and DMAP (95.0 mg, 0.778 mmol) at room temperature. After stirring for 40 min at the same temperature, the reaction mixture was treated with H₂O. The aqueous layer was extracted with AcOEt, and the organic layer was dried over Na₂SO₄, filtered and concentrated. The crude product was added to a silica gel column and eluted with hexane/EtOAc 0% to 26%. Collected fractions were evaporated to afford compound 2-5 (406 mg, 0.954 mmol, 61%) as a yellow oil.

¹H-NMR (400 MHz, CDCl₃) δ: −0.14 (s, 9H), 1.30 (s, 9H), 1.42 (s, 3H), 3.61 (d, J=13.2 Hz, 1H), 3.99 (d, J=13.2 Hz, 1H), 7.07 (dd, J=8.4, 10.7 Hz, 1H), 7.17 (t, J=7.4 H, 1H), 7.43-7.35 (m, 1H), 7.46-7.53 (m, 1H).

### Step 5

To a solution of compound 2-5 (200 mg, 0.470 mmol) in THF (3 ml) was added a solution of MeLi (1.13 mmol/L in ethyl ether, 1.25 mL, 1.41 mmol) at −78° C. After stirring for 30 mm at 0° C., the reaction mixture was treated with aqueous NH₄Cl. The aqueous layer was extracted with AcOEt, and the organic layer was dried over Na₂SO₄, filtered and concentrated. The crude product was added to a silica gel column and eluted with hexane/EtOAc 0% to 30%. Collected fractions were evaporated to afford compound 2-6 (18.0 mg, 0.0408 mmol, 8.7%) as a colorless oil.

¹H-NMR (400 MHz, CDCl₃) δ: 0.01 (s, 9H), 1.06 (s, 3H), 1.28 (s, 9H), 1.69 (s, 3H), 2.30 (d, J=15.1 Hz, 1H), 2.53 (d, J=15.1 Hz, 1H), 5.54 (s, 1H), 6.96-7.03 (m, 1H), 7.24-7.17 (m, 2H), 8.14 (t, J=8.0 Hz, 1H).

### Step 6

To a solution of compound 2-6 (86.0 mg, 0.195 mmol) in MeOH (1 ml) was added 4 mol/L HCl in Dioxane (0.298 ml, 1.17 mmol) at room temperature. After stirring for 17 h at the same temperature, the reaction mixture was treated with aqueous NaHCO₃, and the aqueous layer was extracted with CHCl₃. The combined organic layer was dried over NO₂SO₄, filtered and concentrated to give compound 2-7, which was used for the next step without purification.

To a solution of 2-7 in CH₂Cl₂ (1 ml) was added benzoyl isothiocyanate (0.0400 ml, 0.292 mmol) at 0° C. After stirring for 2.5 h at room temperature, the reaction mixture was concentrated. The resulting residue was added to a silica gel column and eluted with Hexane/EtOAc 0% to 25%. Collected fractions were evaporated to afford 2-8 (72.0 mg, 0.168 mmol, 86%) as a yellow oil.

¹H-NMR (100 MHz, CDCl₃) δ: 1.31 (s, 3H), 2.25 (s, 3H), 2.66 (d, J=15.2 Hz, 1H), 2.98 (d, J=15.2 Hz, 1H), 7.02-7.09 (m, 1H), 7.16 (t, J=7.1 Hz, 1H), 7.27-7.33 (m, 1H), 7.45 (t, J=7.7 Hz, 1H), 7.52 (t, J=7.7 Hz, 2H), 7.68 (t, J=7.7 Hz, 1H), 7.86 (d, J=7.7 Hz, 2H), 8.83 (s, 1H), 11.65 (s, 1H).

### Step 7

To a solution of compound 2-8 (72.0 mg, 0.168 mmol) in MeCN (1 ml) was added WSCD-HCl (64.4 mg, 0.336 mmol) at room temperature. After stirring for 20 h at the same temperature, the reaction mixture was concentrated. The remitting residue was added to a silica gel column and eluted with Hexane/EtOAc 0% to 20%. Collected fractions were evaporated to afford compound 2-9 (55.0 mg, 0.139 mmol, 83%) as a colorless oil.

¹H-NMR (400 MHz, CDCl₃) δ: 1.11 (s, 3H), 1.82 (s, 3H), 2.43 (d, J=14.3 Hz, 1H), 3.06 (d, J=14.3 Hz, 1H), 7.10-7.21 (m, 2H), 7.32-7.47 (m, 4H), 7.52 (t, J=7.3 Hz, 1H), 8.28 (d, J=7.3 Hz, 2H), 11.94 (s, 1H).

### Step 8

To a solution or compound 2-9 (55.0 mg, 0.139 mmol) in MeOH (1 ml) was added K₂CO₃ (57.8 mg, 0.418 mmol) at room temperature. After stirring for 4 h at 50° C., the reaction mixture was treated with H₂O, and the aqueous layer was extracted Willi AcOEt. The combined organic layers were dried over Na₂SO₄, filtered and concentrated to give compound 2-10, which was used for the next step without purification.

To a solution of 2-10 in TFA (1 ml) was added auifuric and (0.245 ml, 4.60 mmol) at −20° C. After stirring for 5 min at 0° C., the reaction mixture was added to HNO₃ (0.001935 ml, 0.209 mmol) at −20° C. After stirring tor 15 min at 0° C. The reaction mixture was treated with aqueous K₂CO₃. The aqueous layer was extracted with AcOEt and the organic layer was dried over Na₂SO₄. The filtrate was concentrated under vacuum to give compound 2-11 as a yellow oil that was used for the next step without purification.

To a solution of 2-11 in THF (1 ml) were added Boc₂O (0.0970 ml, 0.419 mmol) and DMAP (0.82 mg, 0.0560 mmol) at room temperature. After stirring for 1 h at the name temperature, the mixture was concentrated under vacuum. The crude product was added to a silica gel column and eluted with hexane/EtOAc 0% to 20%. Collected fractions were evaporated to afford compound 2-12 (70.0 mg, 0.131 mmol, 94%) as a white solid.

¹H-NMR (400 MHz, CDCl₃) δ: 1.09 (s, 3H), 1.53 (s, 18H), 1.66 (s, 3H), 2.19 (d, J=14.8 Hz,1H), 2.90 (d, J=14.8 Hz, 1H), 7.28-7.24 (m, 1H), 8.19-8.24 (m, 1H), 8.59 (dd, J=6.9. 2.9 Hz, 1H).

### Step 9

A suspension of 2-12 (70.0 mg, 0.131 mmol) and 10% Pd/C (7.05 mg) in MeOH (3 ml) was stirred under a hydrogen atmosphere at room temperature. After stirring for 1.5 h at the same temperature, the mixture was filtrated through a pad of Celite (Registered trademark). The filtrate wae concentrated under vacuum to give compound 2-13 (57.0 mg, 0.113 mmol, 86%) as a white solid that was used for the next step without purification.

¹H-NMR (400 MHz, CDCl₃) δ: 1.14 (s, 3H), 1.52 (s, 18H), 1.01 (s, 3H), 2.04 (d, J=14.2 Hz, 1H), 2.93 (d, J=14.2 Hz, 1H), 3.51 (s, 2H), 6.55-6.50 (m, 1H), 6.89-6.81 (m, 2H).

### Step 10

To a solution of 2-13 (57.0 mg, 0.113 mmol) in DMF (1 ml) were added 5-cyanopicolinic acid hydrate (18.7 mg, 0.113 mmol), HATU (51.4 mg, 0.135 mmol) and DIPEA (0.0390 ml, 0.226 mmol) at room temperature. After stirring for 1 h at the same temperature, the reaction mixture was treated with H₂O. The aqueous layer was extracted with AcOEt, and the organic layer was dried over Na₂SO₄, filtered and concentrated. The crude product was added to a silica gel column and eluted with hexane/EtOAc 0% to 30%. Collected fractions were evaporated to afford compound 2-14 (70.0 mg, 0.110 mmol, 98%) as a white amorphous.

¹H-NMR (400 MHz, CDCl₃) δ: 1.11 (s, 3H), 1.54 (s, 18H), 1.67 (s, 3H), 2.12 (d, J=14.3 Hz, 1H), 2.94 (d, J=14.3 Hz, 1H), 7.13 (dd, J=11.5, 9.0 Hz, 1H), 7.53 (dd, J=7.0, 2.6 Hz, 1H), 8.21 (dd, J=8.2, 1.8 Hz, 1H), 8.32-8.27 (m, 1H), 8.43 (d, J=8.2 Hz, 1H), 8.80 (s, 1H), 9.91 (s, 1H).

### Step 11

A solution of compound 2-14 (70.0 mg, 0.110 mmol) in formic acid (0.972 ml) was stirred for 19 h at room temperature. The reaction mixture was treated with aqueous K₃CO₃. The aqueous layer was extracted with AeOEt, and the organic layer was dried over Na₂SO₄, filtered and concentrated to afford compound 1-7 (40.0 mg, 0.0920 mmol, 83%) as a white solid.

### EXAMPLE 3

### Synthesis of Compound I-8

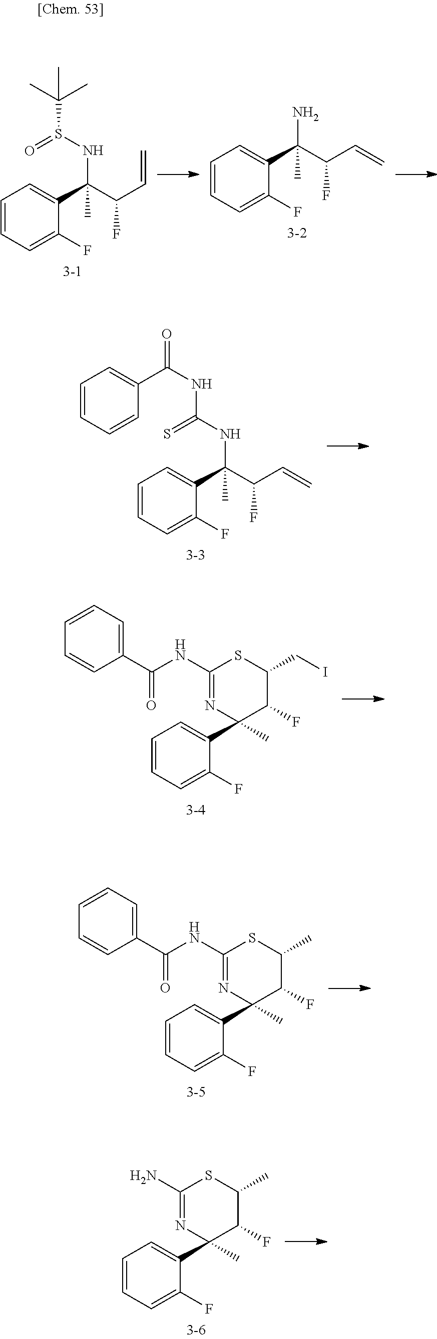 [SMILES: C=C[C@H](F)[C@](C)(N[S@](=O)C(C)(C)C)c1ccccc1F ; C=C[C@H](F)[C@](C)(N)c1ccccc1F ; C=C[C@H](F)[C@](C)(NC(=S)NC(=O)c1ccccc1)c1ccccc1F ; C[C@]1(c2ccccc2F)N=C(NC(=O)c2ccccc2)S[C@H](CI)[C@@H]1F ; C[C@H]1SC(NC(=O)c2ccccc2)=N[C@](C)(c2ccccc2F)[C@H]1F ; C[C@H]1SC(N)=N[C@](C)(c2ccccc2F)[C@H]1F]

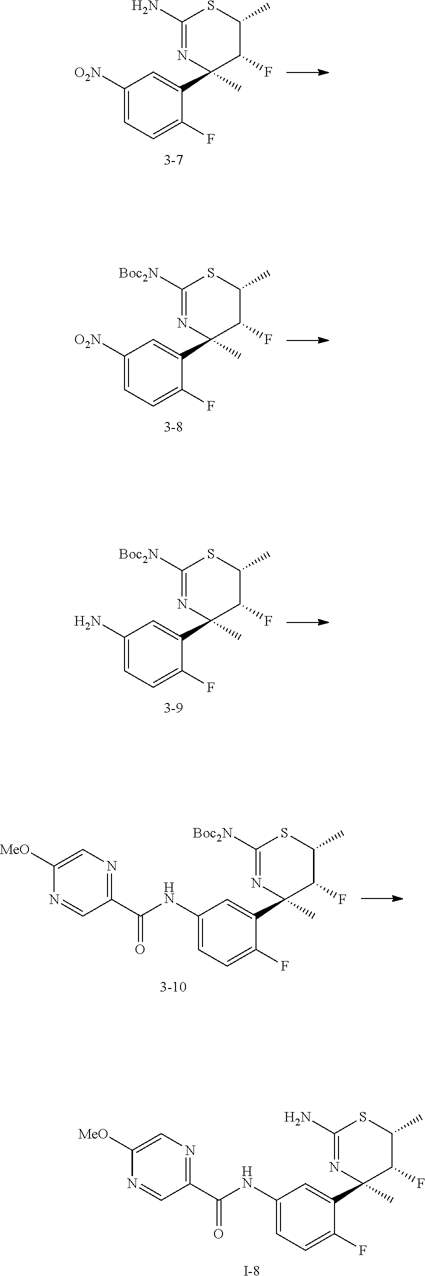

### Step 1

To a solution of compound 3-1 (373 mg, 1.24 mmol) in MeOH (4 ml) was added 4 mol/L HCl in dioxane (0.464 ml, 1.86 mmol) at room temperature. After stirring for 30 min at the same temperature, the reaction mixture was treated with aqueous NalHCO₃, and the aqueous layer was extracted with AcOEt. The combined organic layers were washed with H₂O and brine, dried over Na₂SO₄ and filtered. The filtrate was concentrated under vacuum to give compound 3-2 as a brown oil that was used for the next step without purification.

To a solution of compound 3-2 in CH₂Cl₃ (3 ml) was added benzoyl isothiocyanate (0.255 ml, 1.86 mmol) at 0° C. After stirring for 30 min at room temperature the reaction mixture was concentrated. The resulting residue was added to a silica gel column and eluted with Hexane/EtOAc 0% to 25%K. Collected fractions were evaporated to afford compound 3-3 (397 mg, 1.10 mmol, 89%) as a white amorphous.

¹H-NMR 400 MHz, CDCl₃) δ: 2.13 (s, 3H), 5.48 (d, J=10.6 Hz, 1H), 5.60-5.76 (m, 2H), 5.82-5.96 (m, 1H), 7.00-7.07 (m, 1H), 7.16 (t, J=7.7 Hz, 1H), 7.27-7.33 (m, 1H), 7.44 (t, J=7.7 Hz, 1H), 7.51 (t, J×7.7 Hz, 2H), 7.63 (t, J=7.7 Hz, 1H), 7.85 (d, J=7.7 Hz, 2H), 8.82 (s, 1H), 11.50 (s, 1H).

### Step 2

To a solution of iodine (559 mg, 2.20 mmol) in MeCN (30 ml) was added compound 3-3 (397 mg, 1.10 mmol) in MeCN (10 ml) at 0° C. After stirring for 20 min at the same temperature, the reaction mixture was treated with aqueous NaHCO₂ and Na₂S₂O₃ The aqueous layer was extracted with AcOEt. The combined organic layers were washed with brine, dried over NaₛSO₄, filtered and concentrated. The crude product was added to a silica gel column and eluted with hexane/EtOAc 0% to 20%. Collected fractions were evaporated to afford compound 3.4 (489 mg, 1.01 mmol, 91%) as a white amorphous.

¹H-NMR (400 MHz, CDCl₃) δ: 1.90 (s, 3H), 3.14-3.28 (m, 2H), 3.50 (t, J=9.3 Hz, 1H), 5.70 (d, J=47.3 Hz, 1H), 7.12-7.23 (m, 2H), 7.31-7.48 (m, 4H), 7.53 (t, J=7.3 Hz, 1H), 8.21 (d, J=7.3 Hz, 2H).

### Step 3

To a solution of compound 3-4 (489 mg, 1.01 mmol) in toluene (5 ml) were added Bu₃SnH (0.320 ml, 1.21 mmol) and AIBN (8.26 mg, 0.0500 mmol) at room temperature. After stirring for 100 min at 80° C. the reaction mixture was concentrated. The resulting residue was added to an amino silica gel column and eluted with Hexane/EtOAc 0% to 20%. Collected fractions were evaporated to afford compound 3-5 (330 mg, 0.932 mmol, 93%) as a white amorphous.

¹H-NMR (400 MHz, CDCl₃) δ: 1.40 (d, J=6.7 Hz, 3H), 1.88 (s, 3H), 3.13 (dq, J=31.4, 6.7 Hz, 1H), 5.25 (d, J=17.2 Hz, 1H), 7.11 (dd, J=12.3, 8.0 Hz, 1H), 7.20 (t, J=7.5 Hz, 1H), 7.33-7.46 (m, 4H), 7.51 (t, J=7.5 Hz, 1H), 8.22 (d, J=7.5 Hz, 2H), 12.13 (br s, 1H).

### Step 4

To a solution of compound 3-5 (336 mg, 0.932 mmol) in EtOH (3 ml) was added hydrazine hydrate (0.226 ml, 4.66 mmol) at room temperature. After stirring for 14 h at the same temperature, the reaction mixture was concentrated. The resulting residue was added to an amino silica gel column and eluted with Hexane/EtOAc 10% to 541%. Collected fractions were evaporated to afford compound 3-6 (195 mg, 0.761 mmol, 82%) as a white solid.

¹H-NMR (400 MHz, CDCl₃) δ: 1.32 (d, J=6.8 Hz, 3H), 3.08-2.94 (m, 1H), 5.09 (d, J=47.4 Hz, 1H), 6.09-7.07 (m, 1H), 7.12 (t, J=7.4 Hz, 1H), 7.23-7.31 (m, 2H).

### Step 5

To a solution of compound 3-6 in TFA (2 ml) was added sulfuric acid (0.507 ml, 9.51 mmol) at −20° C. After stirring for 5 min at 0° C. the reaction mixture was added to HNO₃ (0.0510 ml, 1.14 mmol) at −20° C. After stirring for 20 min at 0° C., the reaction mixture was treated with aqueous K₂CO₃. The aqueuos layer was extracted with AeOEt and the organic layer was dried over Na₂SO₄. The filtrate was concentrated under vacuum to give compound 3-7 as a yellow oil that was used for the next step without purification.

To a solution of compound 3-7 in THF (2 ml) were added Boc₂O (0.529 ml, 2.28 mmol) and DMAP (37.1 mg, 0.304 mmol) at room temperature. After stirring for 50 min at the same temperature, the mixture was concentrated under vacuum. The crude product was added to a silica gel column and eluted with hexane/EtOAc 0% to 40%. Collected fractions were evaporated to afford compound 3-8 (381 mg, 0.760 mmol, quant) as a white amorphous.

¹H-NMR (400 MHz, CDCl₃) δ=1.37 (d, J=6.8 Hz, 3H), 1.55 (s, 18H), 1.82 (s, 3H), 3.09 (dq, J=29.6, 6.8 Hz, 1H), 5.12 (d, J=46.7 Hz, 1H), 7.23 (dd, J=10.9, 9.1 Hz, 1H), 8.23-8.18 (m, 1H), 8.52 (dd, J=6.6, 2.8 Hz, 1H).

### Step 6

To a rotation of compound 3-8 (381 mg, 0.760 mmol) in EtOH (4 ml), THF (2 ml) and H₂O (2 ml) were added NH₄Cl (488 mg, 9.12 mmol) and iron powder (339 mg, 6.08 mmol) at room temperature. After stirring for 90 min at 60° C., the mixture was treated with H₂O and filtrated through a pad of Celite (Registered trademark). The aqueous layer was extracted with AcOEt, and the organic layer was dried over Na₂SO₄, filtered and concentrated. The crude product was added to a silica gel column and eluted with hexane/EtOAc 0% to 40%. Collected fractions were evaporated to afford compound 3-9 (247 mg, 0.524 mmol, 69%) as a while amorphous.

¹H-NMR (400 MHz, CdCl₃) δ: 1.33 (d, J=7.0 Hz, 3H), 1.54 (s, 18H), 1.80 (t, J=2.0 Hz, 3H), 3.19 (dq, J=29.6, 7.0 Hz, 1H), 3.57 (s, 2H), 5.05 (d, J=48.2 Hz, 1H), 6.55-6.51 (m, 1H), 6.88-6.80 (m, 2H).

### Step 7

To a solution of compound 3-9 (60.0 mg, 0.127 mmol) in DMF (1 ml) were added 5-methoxypyrazine-2-carboxylic acid (20.6 mg, 0.134 mmol), HATU (58.1 mg, 0.153 mmol) and DIPEA (0.0440 ml, 0.254 mmol) at room temperature. After stirring for 20 min at the same temperature, the reaction mixture was treated with H₂O. The aqueous layer was extracted with AeOEt, and the organic layer was dried over Na₂SO₄, filtered and concentrated. The crude product was added to a silica gel column and eluted with hexane/EtOAc 0% to 30%. Collected fractions were evaporated to afford compound 3-10 (70.0 mg, 0.115 mmol, 91%) as a white solid.

¹H-NMR (400 MHz, CDCl₃) δ: 1.35 (d, J=7.0 Hz, 3H), 1.58 (s, 18H), 1.86 (s, 3H), 3.17 (dq, J=30.5, 7.0 Hz, 1H), 4.06 (s, 3H), 5.09 (d, J=46.8 Hz, 1H), 7.10 (dd, J=9.3, 11.7 Hz, 1H), 7.51 (d, J=6.7 Hz, 1H), 8.06 (s, 1H), 8.44-8.39 (m, 1H), 9.01 (s, 1H), 9.69 (s, 1H).

### Step 8

A solution of compound 3-10 (70.0 mg, 0.115 mmol) in formic acid (0.972 ml) was stirred for 19 h at room temperature. The reaction mixture was treated with aqueous K₂CO₃. The aqueous layer was extracted with AcOEt, and Ihe organic layer was dried over Na₂SO₄, filtered and concentrated to afford compound 1-8 (35.0 mg, 0.0860 mmol, 75%) as a white solid.

### EXAMPLE 4

### Synthesis of Compound I-13

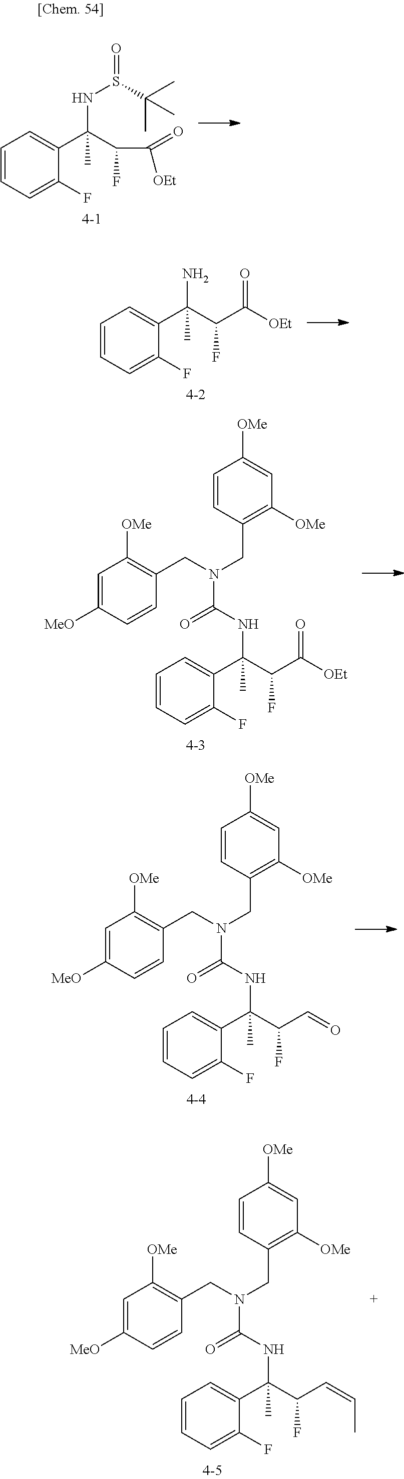 [SMILES: CCOC(=O)[C@H](F)[C@](C)(N[S@](=O)C(C)(C)C)c1ccccc1F ; CCOC(=O)[C@H](F)[C@](C)(N)c1ccccc1F ; CCOC(=O)[C@H](F)[C@](C)(NC(=O)N(Cc1ccc(OC)cc1OC)Cc1ccc(OC)cc1OC)c1ccccc1F ; COc1ccc(CN(Cc2ccc(OC)cc2OC)C(=O)N[C@](C)(c2ccccc2F)[C@@H](F)C=O)c(OC)c1 ; C/C=C\[C@H](F)[C@](C)(NC(=O)N(Cc1ccc(OC)cc1OC)Cc1ccc(OC)cc1OC)c1ccccc1F]

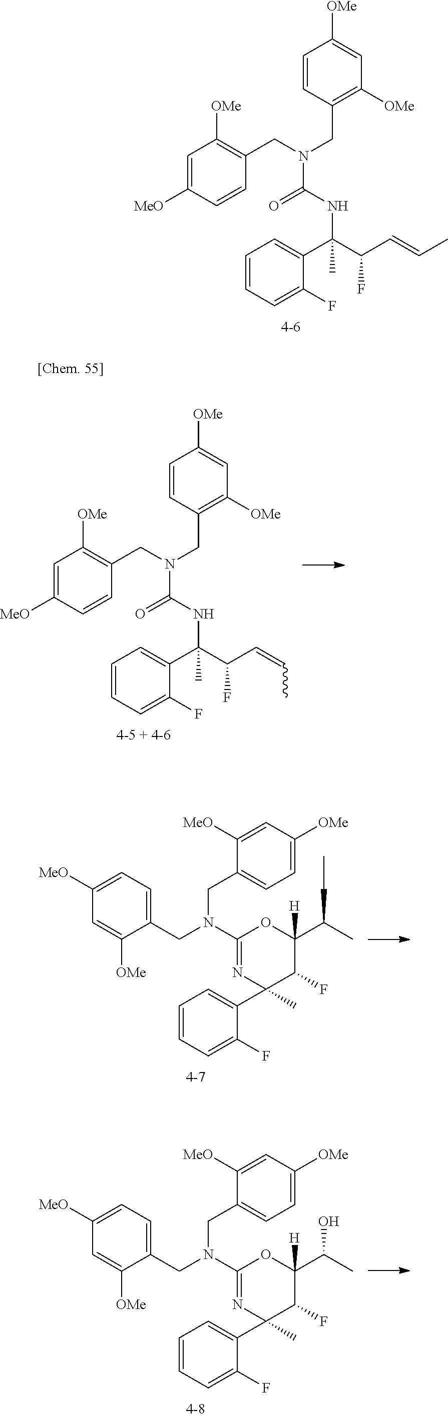

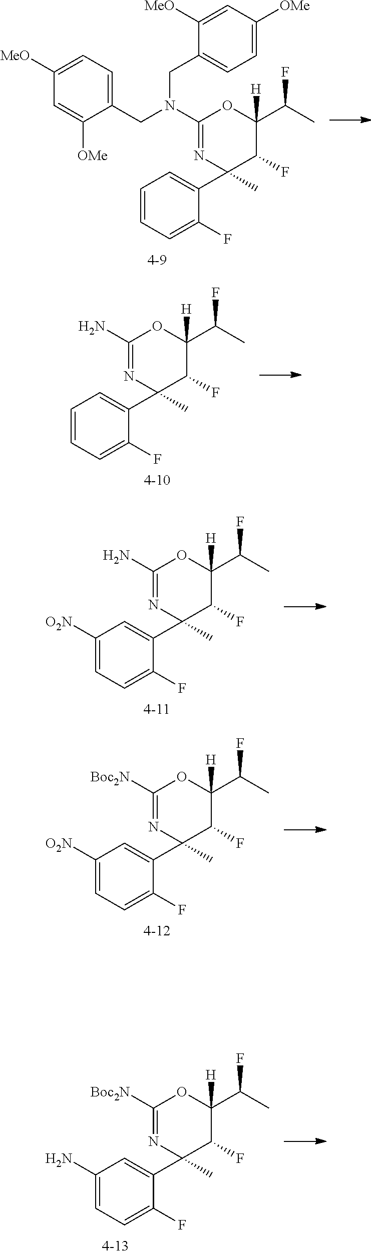

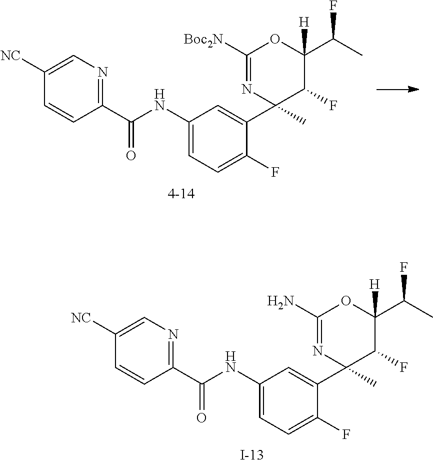

### Step 1

To a solution of compound 4-1 (15.0 g, 43.2 mmol) in methanol (150 ml) was added HCl-dioxane (4M, 15.1 ml, 60.4 mmol). After stirring for 1 h at room temperature, the reaction mixture was quenched with saturated aqueous NaHCO₃ solution. The aqueous phase was extracted with AcOEt. The organic layer was dried over Na₂SO₄ and concentrated to afford compound 4-2 (10.5 g, quant), which was used in the next reaction without further purification.

LC/MS(Shimadzu): RT 0.83, MS calcd for 244.10 (M+H⁺), found 244.30.

### Step 2

To a solution of compound 4-2 (10.5 g, 43.2 mmol) and NaHCO₅ (12.7 g, 151 mmol) in AcOEt (100 ml) and H₂O (50 ml) was added 4-nitrophenyl carbonochloridate (8.71 g, 43.2 mmol) at 0° C. After being stirred for 1 h at 0° C. bis(2,4-dimethoxybenxyl)amine (13.7 g, 43.2 mmol) was added. After an additional stirring for 1 h at 0° C., the reaction mixture was quenched with H₂O, and the aqueous phase was extracted with AcOEt. The organic phase was washed with aqueous K₂CO₃ and H₂O in twice to remove 4-nitrphenol. The organic layer was dried over Na₂SO₄ and concentrated. The residue was purified by silica gel chromatography to afford compound 4-3 (25.3 g, 43.1 mmol, 100%, including small amount of 4-nitrophenol).

¹H-NMR (CDCl₃) δ: 0.97 (t, J=7.2 Hz, 3H), 1.95 (s, 3H), 3.80 (s, 6H), 3.81 (s, 6H), 3.87 (dq, J=10.6, 7.2 Hz, 1H), 4.01 (dq, J=10.6, 7.2 Hz, 1H), 4.33 (d, J=16.2 Hz, 2H), 4.42 (d, J=16.2 Hz, 2H), 5.58 (d, J=47.8 Hz, 1H), 6.07 (s, 1H), 6.42-6.49 (m, 4H), 7.00 (m, 1H), 7.08 (m, 1H), 7.18 (d, J=8.8 Hz, 2H), 7.24 (m, 1H), 7.36 (m, 1H).

### Step 3

To a solution of compound 4-3 (25.3 g, 43.2 mmol, including small amount of 4-nitrophenol) in CH₂Cl₂ (125 ml) was added DIBAL (1.02 mol/L in toluene, 127 ml, 130 mmol at −65° C. After being stirred for 1 h at −65° C., the reaction mixture was quenched with AcOEt and Rochelle's salt (98 g, 346 mmol) in H₂O. After an additional stirring for 2 h at room temperature, the aqueous phase was extracted with AcOEt. The organic layer wan dried over Na₂SO₄ and concentrated. The residue was purified by silica gel chromatography to afford compound 4-4 (19.5 g, 35.9 mmol, 83% in 3 steps).

¹H-NMR (CDCl₃) δ: 1.67 (s, 3H), 3.80 (s, 6H), 3.81 (s, 6H), 4.36 (d, J=15.9 Hz, 2H), 4.43 (d, J=15.9 Hz, 2H), 5.76 (d, J=46.9 Hz, 1H), 5.77 (s, 1H), 6.43-6.51 (m, 4H), 7.00-7.20 (m, 4H), 7.22-7.35 (m, 2H), 9.51 (d, J=10.0 Hz, 1H).

### Step 4

To a solution of ethyltriphenylphosphonium bromide (28.2 g, 76.0 mmol) in THF (129 ml) was added KHMDS (0.5 mol/L in toluene, 143 ml, 71.3 mmol) at 0° C. After dropwise of KHMDS, compound 4-1 (12.9 g, 23.8 mmol) in THF (80 ml) was added rapidly to the reaction mixture. After being stirred for 30 min at 0° C. the temperature was warmed to 50° C. After an additional stirring for 1 h, the reaction mixture was cooled to 0° C. and quenched with H₂O. The aqueous phase was extracted with AeOEt, and the organic layer was dried over Na₂SO₄ and concentrated. The residue was purified by silica gel chromatography to afford compound 4-5 and 4-6 as a mixture (12.4 g, 22 4 mmol, 94%, compound 4-5:compound 4-6=1.5:1).

### Compound 4-5:

¹H-NMR (CDCl₃) δ: 1.54 (m, 3H), 1.87 (s, 3H), 3.78 (s, 6H), 3.81 (s, 6H), 4.31-4.47 (m, 4H), 5.31 (m, 1H), 5.58-5.83 (m, 3H), 6.42-6.48 (m, 4H), 6.97 (m, 4H), 7.07 (m, 1H), 7.10-7.17 (m, 2H), 7.19 (m, 1H), 7.37 (m, 1H).

### Compound 4-6:

¹H-NMR (CDCl₃) δ: 1.57 (m, 3H), 1.82 (s, 3H), 3.78 (s, 6H), 3.81 (s, 6H), 4.37 (m, 4H), 5.30 (m, 1H), 5.45 (dd, J=47.3, 6.8 Hz, 1H), 5.60 (s, 1H), 5.71 (m, 1H), 6.40-6.49 (m, 4H), 6.98 (dd, J=12.5, 8.2 Hz, 1H), 7.08 (m, 1H), 7.13 (d, J=8.5 Hz, 2H), 7.21 (m, 1H), 7.38 (dd, J=8.2, 8.0 Hz, 1H).

### Step 5

To a solution of iodine (11.4 g, 44.7 mmol) in acetonitrile (500 ml) was added compounds 4-5 and 4-6 (12.4 g, 22.4 mmol) in acetonitrile (125 ml) at 0° C. After being stirred tor 1.5 h at 0° C., the reaction mixture was quenched with aqueous Na₂S₂O₃ and saturated aqueous NaHCO₃. After removal of acetontirile in vacuo, the aqueoua phase was extracted with AcOEt. The organic layer was dried over Na₂SO₄ and concentrated. The residue was purified by silica gel chromatography to afford compound 4-7 (8.81 g, 13.0 mmol, 58%) and recovered compound 4-6 (13.17 g, 5.72 mmol, 26%).

¹H-NMR (CDCl₃) δ: 1.63 (s, 3H), 1.84 (d, J=7.0 Hz, 3H), 3.63 (dd, J=28.6, 9.2 Hz, 1H), 3.75 (s, 6H), 3.82 (s, 6H), 4.12 (m, 1H), 4.49 (d, J=15.9 Hz, 2H), 4.65 (d, J=15.9 Hz, 2H), 5.37 (d, J=48.8 Hz, 1H), 6.41-6.50 (m, 4H), 7.00 (dd, J=11.9, 8.3 Hz, 1H), 7.06 (dd, J=7.4, 7.4 Hz, 1H), 7.16-7.28 (m, 3H), 7.42 (dd, J=8.3, 7.4 Hz, 1H).

### Step 6

To a solution of 18-crown-6 (5.45 g, 20.6 mmol) and KO₂ (1.47 g, 20.6 mmol) in DMSO (40 ml) was added compound 4-7 (3.51 g, 5.16 mmol) in DMSO (25 ml) at room temperature. After being stirred for 25 mm, the reaction mixture was quenched with saturated aqueous Na₂S₂O₃. The aqueous phase was extracted with AcOEt, and the organic layer was dried over Na₂SO₄. This reaction was repeated 3 times (300 mg, 2.0 g and 3.0 g of 4-7 were used in each step). The combined organic layers obtained in each step were concentrated, and the residue was purified by silica gel chromatography to afford compound 4-8 (2.05 g, 3.59 mmol, 28%. 7.6:1 diastereo mixture at C7 position).

¹H-NMR (CDCl₃) δ: 1.07 (d, J=6.3 Hz, 3H), 1.66 (s, 3H), 3.35 (dd, J=30.4, 7.8 Hz, 1H), 3.75 (s, 6H), 3.81 (s, 6H), 3.85 (m, 1H), 4.45 (d, J=15.9 Hz, 2H), 4.61 (d, J=15.9 Hz, 2H), 5.38 (d, J=48.6 Hz, 1H), 6.41-6.50 (m, 4H), 6.98 (dd, J=12.0, 8.2 Hz, 1H), 7.05 (dd, J=7.7, 7.3 Hz, 1H), 7.17-7.28 (m, 3H), 7.42 (dd, J=8.2, 7.7 Hz, 1H).

### Step 7

To a solution of compound 4-8 (1.42 g, 2.49 mmol) and nonafluorobutanesulfonyl fluoride (1.61 ml, 8.96 mmol) in toluene (28 ml) was added DBU (1.34 ml, 8.96 mmol) at room temperature. After being stirred for 12 h at room temperature, the reaction mixture was quenched with saturated aqueous NH₄Cl, water and 2 mol/L aqueous HCl. The aqueous phase was extracted with AcOEt and the organic phase was washed with 2 mol/L aqueous NaOH. The organic layer was dried over Na₂SO₄ and concentrated. The residue was purified by silica gel chromatography to afford a mixture of compound 4-9 and the corresponding exo olefin (1.0 g, compound 4-9-exo olefin=1.1, inseparable mixture).

LC/MS(Shimadzo): RT 1.98, MS calcd for 573.26 (M+H⁺), found 573.25.

### Step 8

To a mixture of compound 4-9 and exo olefin (1.0 g, compound 4-9:exo olefin=1:1, inseparable mixture) and anisole (1.34 ml, 12.2 mmol) was added TFA (6.73 ml, 87.0 mmol) at room temperature. After being stirred for 13.5 h at 80° C., the reaction mixture was cooled to 0° C. and quenched with aqueous K₂CO₃. The aqueous phase was extracted with AcOEt. The organic layer was dried over Na₂SO₄ and concentrated. The residue was purified by silica gel chromatography to afford compound 4-10 (251.8 mg, 925 μmol, 37%, 2 steps).

¹H-NMR (CDC₃) δ: 1.40 (dd, J=24.1, 6.4 Hz, 3H), 1.77 (s, 3H), 3.89 (ddd, J=30.2, 12.9, 7.8 Hz, 1H), 4.88 (ddq, J=49.0, 7.8, 6.4 Hz, 1H), 5.25 (d, J=47.2 Hz, 1H), 7.10 (dd, J=12.3, 8.2 Hz, 1H), 7.24 (m, 1H), 7.32-7.45 (m, 2H).

### Step 9

To a solution of compound 4-10 (251.8 mg, 925 μmol) in TFA (2.4 ml) was added H₂SO₄ (0.6 ml) at −20° C. After being stirred for 5 min at 0° C., tbe reaction mixture was cooled to −20° C. and HNO₃ (62 μl, 1.39 mmol) was added. After an additional stirring for 30 min at 0° C., the reaction mixture was quenched with aqueous K₂CO₃ The aqueous phase was extracted with AcOEt. The organic layer was dried over Na₂SO₄ and concentrated. The residue was purified by silica gel chromatography to afford compound 4-11 (240.1 mg, 757 μmol, 82%),

¹H-NMR (CDCl₃) δ: 1.39 (dd, J=24.2, 6.4 Hz, 3H), 1.65 (s, 3H), 3.67 (ddd, J=30.9, 13.2, 7.1 Hz, 1H), 4.42 (brs, 2H), 4.86 (ddq, J=18.8, 7.4, 6.4 Hz, 1H), 5.15 (d, J=47.7 Hz, 1H), 7.23 (m, 1H), 8.22 (m, 1H), 8.45 (m, 1H).

### Step 10

To a solution of compound 4-11 (240.1 mg, 757 μmol) and DMAP (18.5 mg, 151 μmol) in CH₂Cl₂ (2.4 ml) was added Boc₂-O (439 μl, 1.89 mmol) at room temperature. After being stirred for 35 min at room temperature, the reaction mixture was concentrated. The residue was purified by silica gel chromatography to afford compound 4-12. The residue was triturated from AcOEt/hexane to give compound 4-12 (241.6 mg, 473 μmol, 62%). The stereochemistry at C7 position of compound 4-12 was determined by X-ray crystal togrnphic analysis.

¹H-NMR (CDCl₃) δ: 1.40 (dd, J=24.1, 6.5 Hz, 3H), 1.52 (s, 18H), 1.72 (s, 3H), 3.80 (ddd, J=29.9, 12.9, 7.2 Hz, 1H), 4.91 (ddq, J=48.1, 7.2, 6.5 Hz, 1H), 5.19 (d, J=47.2 Hz, 1H), 7.28 (m, 1H), 8.26 (m, 1H), 8.57 (m, 1H).

### Step 11

To a solution of compound 4-12 (211.2 mg, 466 μmol) in THF (3 ml) and MeOH (1.5 ml) was added Pd/C (24.8 mg) at room temperature. After being stirred for 2 h under H₂ atmosphere at room temperature, the reaction mixture was filtered through a pad of Celite (Registered trademark) and washed with AeOEt. The filtrate was concentrated, and the residue was purified by silica gel chromatography to afford compound 4-13 (214.2 mg, 439 μmol, 94%).

¹H-NMR (CDCl₃) δ: 1.39 (dd, J=24.1, 6.4 Hz, 3H), 1.52 (s, 18H), 1.68 (s, 3H), 3.58 (brs, 2H), 3.91 (ddd, J=30.0, 13.4, 7.4 Hz, 1H), 4.88 (ddq, J=48.8, 7.4, 6.4 Hz, 1H), 5.16 (d, J=47.7 Hz, 1H), 6.56 (m, 1H), 6.82-6.91 (m, 2H).

### Step 12

To a solution of compound 4-13 (70.0 mg, 144 μmol), 5-cyanopicolinic acid hydrate (28.6 mg, 172 μmol) and diisopropylethylamine 150 μl, 287 μmol) in DMF (1 ml) was added HATU (65.5 mg, 172 μmol) at room temperature. After being stirred for 1.5 h at room temperature, the reaction mixture was quenched with saturated aqueous NH₄Cl. The aqueous phase was extracted with AcOEt, and the organic phase was washed with H₂O. The organic layer was dried over Na₂SO₄ and concentrated. The residue was purified by silica gel chromatography to afford compound 4-14 (90.0 mg, quant.).

¹H-NMR (CDCl₃) δ: 1.40 (dd, J=24.0, 6.4 Hz, 3H), 1.56 (s, 18H), 1.75 (s, 3H), 3.90 (ddd, J=30.6, 13.2, 7.4 Hz, 1H), 4.89 (ddq, J=48.8, 7.4, 6.4 Hz, 1H), 5.18 (d, J=17.3 Hz, 1H), 7.15 (m, 1H), 7.60 (m, 1H), 8.21 (d, J=8.3 Hz, 1H), 8.10 (m, 1H), 8.43 (d, J=8.3 Hz, 1H), 8.80 (s, 1H), 9.99 (s, 1H).

### Step 13

Compound 4-14 (90.0 mg, 144 μmol) was solved in formic acid (1 ml) and aqueous for 16 h at room temperature. The reaction mixture was quenched with aqueous K₂CO₂, and the aqueous phase was extracted with AcOEt. The organic layer was dried over Na₂SO₄ and concentrated. The residue was triturated from AcOEt/hexane to give compound I-13 (48.8 mg, 117 μmol, 81% in 2 steps).

### EXAMPLE 5

### Synthesis of Compound I-16

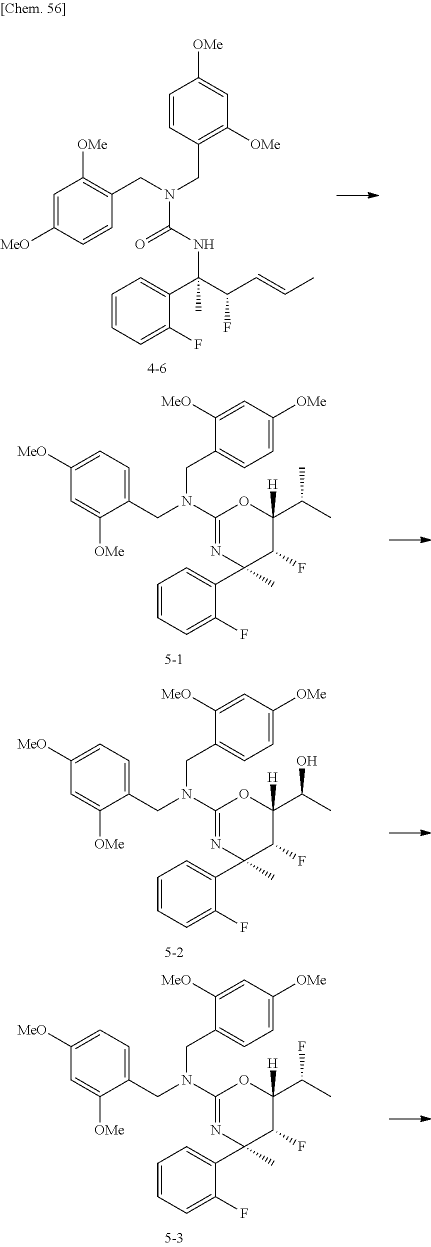

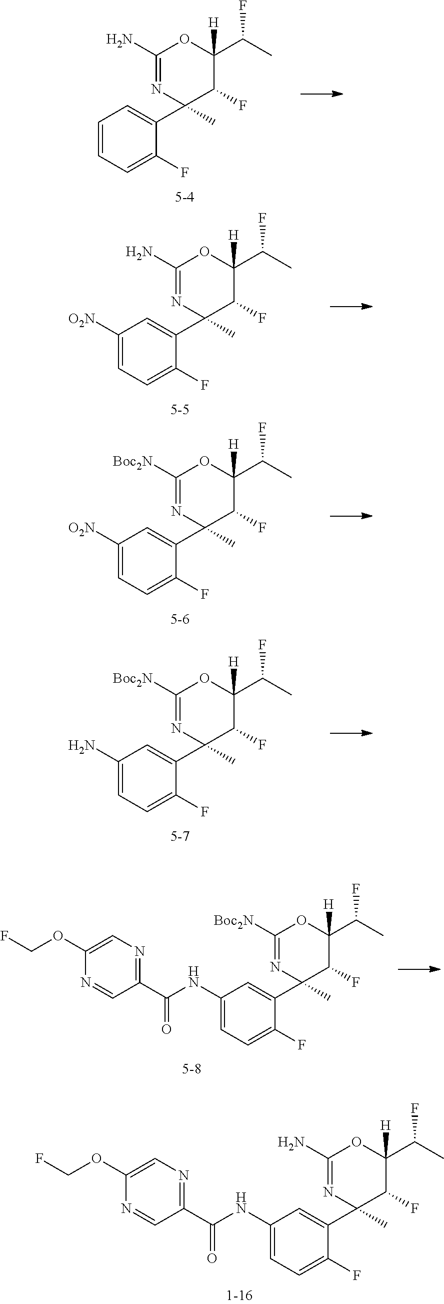

### Step 1

To a solution of iodine (1.05 g, 4.15 mmol) in acetonitrile (45 ml) was added compound 4-6 (1.15 g, 2.07 mmol) in acetonitrile (15 ml) at 0° C. After being stirred for 3 d at 0° C., the reaction mixture was quenched with saturated aqueous NaHCO₃ followed by addition of aqueous Na₂S₂O₃. The aqueous phase was extracted with AcOEt. The organic layer was dried over Na₂SO₄ and concentrated. The residue was purified by silica gel chromatography to afford compound 5-1 (1.20 g, 1.76 mmol, 85%).

¹H-NMR (CDCl₃) δ: 1.65 (s, 3H), 1.74 (d, J=6.8 Hz, 3H), 3.61 (dd, J=27.5, 10.0 Hz, 1H), 3.74 (s, 6H), 3.81 (s, 6H), 4.10 (m, 1H), 4.45 (d, J=16.1 Hz, 2H), 4.61 (d, J=16.1 Hz, 2H), 5.66 (d, J=48.3 Hz, 1H), 6.41-6.49 (m, 4H), 7.03 (m, 1H), 7.06 (m, 1H), 7.19 (d, J=8.2 Hz, 2H), 7.22 (m, 1H), 7.41 (m, 1H).

### Step 2

To a solution of 18-crown-6 (1.62 g, 6.11 mmol) and KO₂ (435 mg, 6.11 mmol) in DMSO (25 ml) was added compound 5-1 (1.04 g, 1.53 mmul) in DMSO (15 ml) at room temperature. After being stirred for 30 min, the reaction mixture was quenched with saturated aqueous Na₂S₂O₃ at 0° C. The aqueous phase was extracted with AcOEt. The organic layer was dried over Na₂SO₄ and concentrated. The residue was purified by allien gel chromatography to afford compound 5-2 (291 mg, 510 μmol, 33%).

¹H-NMR (CDCl₃) δ: 1.07 (d, J=6.3 Hz, 3H), 1.62 (s, 3H), 2.25 (brs, 1H), 3.34 (dd, J=30.9, 8.0 Hz, 1H), 3.76 (s, 6H), 3.81 (s, 6H), 3.83 (m, 1H), 4.49 (d, J=15.9 Hz, 2H), 4.67 (d, J=15.9 Hz, 2H), 5.14 (d, J=48.8 Hz, 1H), 6.42-6.51 (m, 4H), 6.98 (m, 1H), 7.03 (m, 1H), 7.17-7.29 (m, 3H), 7.37 (m, 1H).

### Step 3

To a solution of compound 5-2 (290 mg, 508 μmol) in CH₂Cl₂ (6 ml) was added DAST (537 μl, 4.06 mmol) at −65° C. After being stirred for 22.5 h at room temperature, the reaction mixture was quenched with saturated aqueuus NaHCO₃ at 0° C. The aqueous phase was extracted with AcOEt. The organic layer was dried over Na₂SO₄ and concentrated. The residue was purified by silica gel chromatography to afford a mixture of compound 5-3 and the corresponding endo olefin (212 mg, compound 5-3:endo olefin=2.5:1, inseparable mixture). The yield of compound 5-3 was calculated by ¹H NMR ratio to be 53%.

¹H-NMR (CDCl₃) δ: 1.17 (dd, J=25.5, 6.1 Hz, 3H), 1.66 (s, 3H), 3.53 (m, 1H), 3.75 (s, 6H), 3.81 (s, 6H), 4.38-4.75 (m, 5H), 5.30 (d, J=48.3 Hz, 1H), 6.40-6.50 (m, 4H), 7.00 (m, 1H), 7.06 (m, 1H), 7.15-7.29 (m, 3H), 7.41 (m, 1H).

### Step 4

To a mixture of compound 5-3 ) and exo olefin (net weight of 5-3: 172 mg, 300 μmol) and anisole (316 μl, 2.89 mmol) was added TFA (1.59 ml, 20.7 mmol) at room temperature. After being stirred for 17 h at 80° C., the reaction mixture was cooled to 0° C. and quenched with aqueous K₂CO₃. The aqueous phase was extracted with AcOEt. The organic layer was dried over Na₂SO₄ and concentrated. The residue was purified by silica gel chromatography to afford compound 5-4 (103 mg, quant.),

¹H-NMR (CDCl₃) δ: 1.44 (dd, J=25.3, 6.0 Hz, 3H), 1.85 (s, 3H), 3.84 (m, 1H), 4.86 (m, 1H), 5.14 (d, J=46.4 Hz, 1H), 7.12 (m, 1H), 7.26 (m, 1H), 7.33-7.43 (m, 2H).

### Step 5

To a solution of compound 5-4 (103 mg, 300 μmol) in TFA (1.2 ml) was added H₂SO₄ (0.3 ml) at −20° C. After being stirred for 5 min at 0° C., the reaction mixture was cooled to −20° C. and HNO₂ (25 μl, 567 μmol) was added. After being stirred for 30 min at 0° C., the reaction mixture was quenched with aqueous K₂CO₃. The aqueous phase was extracted with AcOEt. The organic layer was dried over Na₂SO₄ and concentrated. The residue was purified by silica gel chromnatography to afford compound 5-5 (86.3 mg, 272 μmol, 91%, 2 steps),

¹H-NMR (CDCl₃) δ: 1.40 (dd, J=25.5, 0.1 Hz, 3H), 1.67 (s, 3H), 3.53 (m, 1H), 4.29 (brs, 2H), 4.81 (m, 1H), 5.30 (d, J=47.2 Hz, 1H), 7.23 (m, 1H), 8.21 (m, 1H), 8.44 (m, 1H).

### Step 6

To a solution of compound 5-5 (86.3 mg, 272 μmol) and DMAP (16.6 mg, 135 μmol) in CH₂Cl₂ (2 ml) was added Boc₂O (158 μl, 680 μmol) at room temperature. After being stirred for 50 min at room temperature, the reaction mixture was concentrated. The residue was purified by silica gel chromatography to afford compound 5-6 (129 mg, 248 μmol, 91%).

¹ H-NMR (CDCl₃) δ: 1.41 (dd, J=25.5, 6.1 Hz, 3H), 1.53 (s, 18H), 1.74 (s, 3H), 3.65 (ddd, J=27.4, 8.3, 4.8 Hz, 1H), 4.87 (ddq, J=46.7, 8.3, 6.1 Hz, 1H), 5.35 (d, J=46.9 Hz, 1H), 7.27 (m, 1H), 8.26 (m, 1H), 8.52 (m, 1H).

### Step 7

To a solution of compound 5-6 (129 mg, 248 μmol) in THF (2 ml) and MeOH (1 ml) was added Pd/C (26.4 mg) at room temperature. After being stirred for 7.5 h under H₂ atmosphere at room temperature, the reaction mixture was filtered through a pad of Celite (Registered trademark) and washed with AcOEt. The filtrate was concentrated, and the residue was purified by silica gel chromatography to afford compound 5-7 (104 mg, 214 μmol, 86%).

¹H-NMR (CDCl₃) δ: 1.40 (dd, J=25.4, 6.1 Hz, 3H), 1.53 (s, 18H), 1.70 (s, 3H), 3.56 (brs, 2H), 3.80 (m, 1H), 4.84 (m, 1H), 5.33 (d, J=47.3 Hz, 1H), 6.56 (m, 1H), 6.83 (m, 1H), 6.86 (m, 1H).

### Step 8

To a solution of compound 5-7 (44.2 mg, 90.7 μmol), 5-(fluoromethoxy)pyrazine-2-carboxylic acid (18.7 mg, 109 μmol) and diisopropylethylamine (32 μl, 181 μmol) in DMF (1 ml) was added HATU (41.4 mg, 109 μmol) at room temperature. After being stirred for 50 min at room temperature, the reaction mixture was quenched with saturated aqueous NH₄Cl. The aqueous phase was extracted with AcOEt. The organic layer was dried over Na₂SO₄ and concentrated. The residue was purified by silica gel chromatography to afford compound 5-8 (51.6 mg, 80.4 μmol, 89%).

¹H-NMR (CDCl₃) δ: 1.40 (dd, J=25.3, 6.1 Hz, 3H), 1.54 (s, 18H), 1.76 (s, 3H), 3.78 (m, 1H), 4.85 (m, 1H), 5.36 (d, J=47.2 Hz, 1H), 6.15 (dd, J=51.1, 14.8 Hz, 2H), 7.14 (m, 1H), 7.47 (m, 1H), 8.20 (s, 1H), 8.35 (m, 1H), 9.08 (s, 1H), 9.62 (s, 1H).

### Step 9

Compound 5-8 (51.6 mg, 80.4 μmol) was solved in formic acid (0.75 ml) and stirred for 18.5 h at room temperature. The reaction mixture was quenched with aqueous K₂CO₃ at 0° C., and the aqueous phase was extracted with AcOEt. The organic layer was dried over Na₂SO₄ and concentrated. The residue was triturated from AcOE/hexane to give compound I-16 (31.0 mg, 70.2 μmol, 87%).

### EXAMPLE 6

### Synthesis of Compound I-28

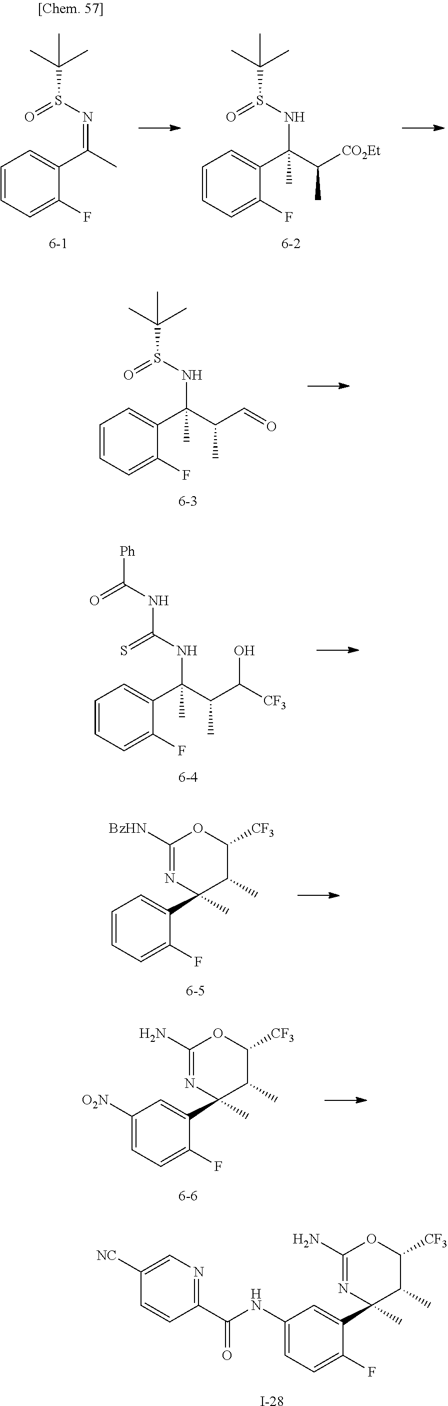

### Step 1

To a stirred solution of diisopropylamine (37.7 mL, 0.265 mol) to THF (260 mL) was added drop wise n-butyl lithium (2.65 mol/L in hexane, 100 mL, 0.265 mol) at −78° C. After being stirred for 25 min at 0° C., to the mixture was added dropwise ethyl propionate (30.4 mL, 0.265 mol) followed by chlorotriisopropoxytitanium (IV) (84.0 mL, 0.353 mol) in THF (70 mL) at −78° C. After being stirred for 30 min, compound 6-1 (21.3 g, 0.088 mol) in THF (70 mL) was added dropwise to the mixture at −78° C. The reaction mixture was stirred for 30 min at −78° C. The reaction was quenched with a saturated solution of ammonium chloride. The resulting mixture was filtered through Celite (Registered trademark), and the filtrate was extracted with ethyl acetate. The combined organic layer was washed with brine, dried over sodium sulfate, and filtered. The solvent was evaporated, and the crude product was purified by flash column chromatography (silica gel, gradient from 3:1 to 1:1 hexane:ethyl acetate) to give compound 6-2 (24.8 g, 82%) as a tan oil. This material was obtained as a mixture of diastereomers.

¹H NMR (400 MHz, CDCl₃) (major isomer) 1.14 (d, J=7.0 Hz, 3H), 1.28 (s, 9H), 1.95 (s, 3H), 3.14 (q, J=7.0 Hz, 1H), 3.98-4.04 (m, 1H), 4.89 (s, 1H), 7.01-7.43 (m, 5H).

### Step 2

To a stirred solution of diisopropylamine (37.7 ml, 0.265 mol) in THF (260 mL) was added dropwise n-butyl lithium (2.65 mol/L in hexane, 100 mL, 0.265 mol) at −78° C. After being stirred for 25 min at 0° C. ethyl to the mixture were added dropwise ethyl propionate (30.4 mL, 0.265 mol) followed by chlorotriisopropoxytitanium (IV) (84.0 mL, 0.353 mol) in THF (70 mL) at −78° C. After being stirred for 30 min, compound 6-1 (21.3 g, 0.088 mol) in THF (70 mL) was added dropwise to the mixture at −78° C. The reaction mixture was stirred for 30 min at −78° C. The reaction was quenched with a saturated solution of ammonium chloride. The resulting mixture was filtered through Celite (Registered trademark), and the filtrate was extracted with ethyl acetate. The combined organic layer was washed with brine, dried over sodium sulfate, and filtered. The solvent was evaporated, and the crude product was purified by flash column chromatography (silica gel, gradient from 3:1 to 1:1 hexane:ethyl acetate) to give compound 6-2 (24.8 g, 82%) as a tan oil. This material was obtained as a mixture of diastereomers.

¹H NMR (400 NHz, CDCl₃), 1.06 (d, J=7.5 Hz, 3H), 1.20 (s, 9H), 1.84 (s, 3H), 3.36 (q, J=7.5 Hz, 1H), 4.99 (s, 1H), 7.02-7.45 (m, 4H), 9.76 (s, 1H).

Under a nitrogen atmosphere, to a stirred solution of compound 6-3 (1.04 g, 3.47 mmol) in THF (20 mL) were added trimethyl(trifluoromethyl)silane (1.05 mL, 6.95 mmol) and TBAF (1 mol/L in THF, 0.243 mL, 0.243 mmol) at 0° C. After being stirred for 1 h at 0° C. the reaction was quenched with water. The mixture was extracted with ethyl acetate. The combined organic layers were washed with brine, dried over sodium sulfate, and filtered. The solvent was evaporated to give the crude product, which was used for the next reaction without further purification.

To a stirred solution of the crude product in methanol (15 mL) was added hydrogen chloride (4 mol/L in dioxane, 1.30 mL, 5.20 mmol) at 0° C. After being stirred for 20 h at room temperature, the reaction was quenched with a saturated solution of sodium hydrogen carbonate. The mixture was extracted with ethyl acetate. The combined organic layers were washed with brine, dried over sodium sulfate, and filtered. The solvent was evaporated to give the crude product, which was used for the next reaction without further purification.

To a stirred solution of Ihe crude product in dichloromethane (15 mL) was added benzoyl isothiocyannte (0.559 mL, 4.16 mmol) at 0° C. After being stirred for 2.5 h at room temperature, the mixture was evaporated. The crude product was purified by flash column chromatography (silica gel, 3:1 hexane:ethyl acetate) to give compound 6-4 (620 mg, 42%) as a yellow amorphous. This material was obtained as a mixture of diastereomers. MS: m/z=395.10 [M+H]⁺.

### Step 4

A suspension of compound 6-4 (620 mg, 1.45 mmol) and WSCD-HCl (555 mg, 2.89 mmol) in acetonitrile (12 mL) was stirred for 20 h at room temperature. Water was added to the reaction mixture, which was then extracted with ethyl acetate. The combined organic layers were washed with brine, dried over sodium sulfate, and filtered. The solvent was evaporated, and the crude product was purified by flash column chromatography (silica gel, 85:15 hexane:ethyl acetate) to give compound 6-5 (258 mg, 45%) as a colorless oil.

¹H NMR (400 MHz, CDCl₃) 1.30 (d, J=6.9 Hz, 3H), 1.79 (s, 3H), 2.96 (q, J=6.9 Hz, 1H), 4.35-4.39 (m, 1H), 7.13-7.55 (m, 7H), 8.27 (d, J=8.0 Hz, 2H), 11.6 (s, 1H).

### Step 5

A suspension of compound 6-5 (258 mg, 0.654 mmol) and potassium carbonate (271 mg, 1.96 mmol) in methanol (5 mL) was stirred for 3 days at room temperature. Water was added to the reaction mixture, which was then extracted with ethyl acetate. The combined organic layer was washed with brine, dried over sodium sulfate, and filtered. The solvent was evaporated to give the crude product, which was used for the next reaction without further purification.

To a stirred solution of the crude product in TFA (1.1 mL) were added sulfuric acid (0.28 mL, 5.25 mmol), followed by nitric acid (0.044 mL, 0.982 mmol) at −20° C. After being stirred at −20° C. to −10° C. for 75 min, the reaction was quenched with a saturated solution of potassium carbonate. The mixture was extracted with ethyl acetate. The combined organic layer was washed with brine, dried over sodium sulfate, and filtered. The solvent was evaporated, and the crude product was purified by flash column chromatography (silica gel, gradient from 2:1 to 1:1 hexane:ethyl acetate) to give compound 6-6 (148 mg, 68%) as a colorless amorphous.

¹H NMR (400 MHz, CDCl₃) 1.19 (d, J=7.0 Hz, 3H), 2.74 (q, J=7.0 Hz, 1H), 3.99-4.04 (m, 1H), 7.22 (t, J=9.9 Hz, 1H), 8.17-8.21 (m, 1H), 8.35 (dd, J=6.7, 2.8 Hz, 1H).

### Step 6

A suspension of compound 6-6 (85.7 mg, 0.256 mmol), iron powder (114 mg, 2.05 mmol), and ammonium chloride (164 mg, 3.07 mmol) in ethanol (0.8 mL)/THF (0.4 mL)/water (0.4 mL) was stirred at 60° C. for 2.5 h. The mixture was cooled to room temperature and filtered through a pad of Celite (Registered trademark). The filtrate was extracted with ethyl acetate. The combined organic layers were washed with brine, dried over sodium sulfate, and filtered. The solvent was evaporated to give the crude product, which was used for the next reaction without further purification.

To a stirred solution of the crude product and aqueous hydrochloric acid solution (2 mol/L, 0.081 mL, 0.161 mmol) in methanol (1 mL) were added 5-cyanopicolinic acid hydrate (29.5 mg, 0.177 mmol) and WSCD-HCl (37.1 mg, 0.161 mmol) at room temperature. After being stirred for 1 h at room temperature, the reaction was quenched with aqueous sodium hydroxide solution. The mixture was extracted with ethyl acetate. The combined organic layers were washed with brine, dried over sodium sulfate, and filtered. The solvent was evaporated, and the crude product was purified by flash column chromatography (amino silica gel, gradient from 1:1 to 0:1 hexane-ethyl acetate) to give compound I-28 (58.1 mg, 74%) as an off-white solid.

### EXAMPLE 7

### Synthesis of Compound I-33

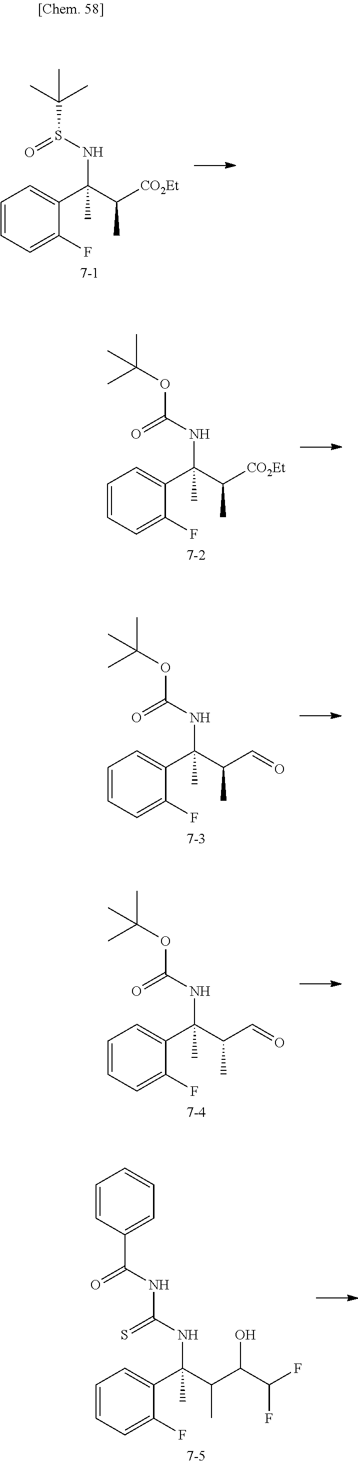 [SMILES: CCOC(=O)[C@@H](C)[C@](C)(N[S@](=O)C(C)(C)C)c1ccccc1F ; CCOC(=O)[C@@H](C)[C@](C)(NC(=O)OC(C)(C)C)c1ccccc1F ; C[C@H](C=O)[C@](C)(NC(=O)OC(C)(C)C)c1ccccc1F ; C[C@@H](C=O)[C@](C)(NC(=O)OC(C)(C)C)c1ccccc1F ; CC(C(O)C(F)F)[C@](C)(NC(=S)NC(=O)c1ccccc1)c1ccccc1F]

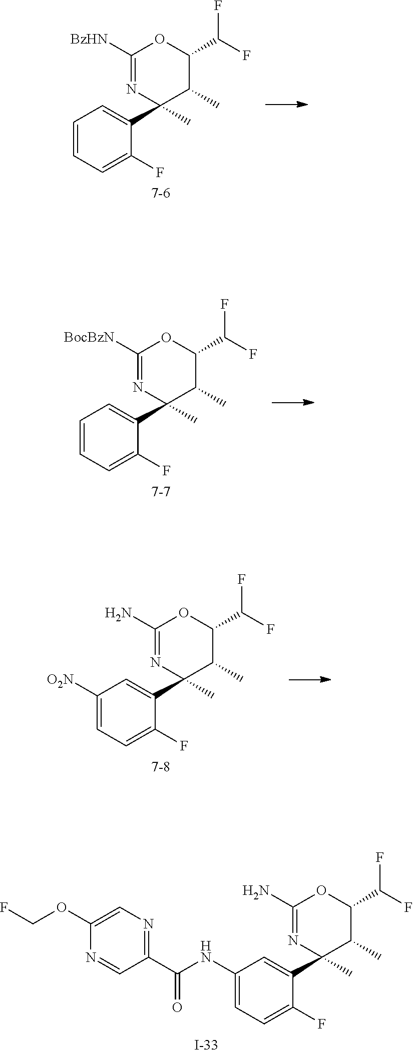

### Step 1

A notation of compound 7-1 (6.2 g, 18.1 mmol) (an intermediate for the synthensis of I-28) and hydrogen chloride solution (4 mol/L in dioxane, 6.77 mL, 27.1 mmol) in methanol (45 mL) was stirred at room temperature for 75 min. The reaction was quenched with a saturated solution of sodium hydrogen carbonate. The mixture was extracted with ethyl acetate. The combined organic layers were washed with brine, dried over sodium sulfate, and filtered. The solvent was evaporated to give the crude product, which was used for the next reaction without further purification.

A solution of the crude product and Boc₂O (5.79 mL, 27.1 mmol) in THF (40 mL) was stirred at room tempemture for 16 h and then heated to 50° C. for 24 h. The mixture was cooled to room temperature and evaporated. The crude product was purified by flash column chromatography (silica gel, 9:1 hexane:ethyl acetate) to give compound 7-2 (5.7 g, 93%) as a tan oil. This material was obtained as a mixture of diastereomers.

¹H NMR (400 MHz, CDCl₃) (major isomer) 0.96 (d, J=7.0 Hz, 3H), 1.23 (t, J=6.4 Hz), 1.40 (s, 9H), 1.91 (s, 3H), 3.01 (q, J=7.0 Hz, 1H), 4.10-4.16 (m, 3H), 5.95 (1H, brs), 6.97-7.31 (m, 5H).

### Step 2

To a stirred solution of compound 7-2 (5.12 g, 15.1 mmol) in THF (25 mL) and methanol (25 mL) was added slowly sodium borohydride (2.83 g, 75.0 mmol) at 0° C. After being stirred at 0° C. for 2 h, sodium borohydride (2.85 g, 75.0 mmol) was added slowly to the mixture at 0° C. The resulting mixture was stirred at room temperature for 16.5 h. The reaction was quenched with an aqueous solution of hydrogen chloride. The mixture was extracted with ethyl acetate. The combined organic layers were washed with brine, dried over sodium sulfate, and filtered. The solvent wan evaporated to give the crude product, which was used for the next reaction without, further purification.

Under a nitrogen atmosphere, to a stirred solution of the crude product in dichloromethane (50 mL) was added Dess-Martin periodinane (9.54 g, 22.5 mmol) at 0° C. After being stirred for 4.5 h at room temperature, the reaction was quenched with a saturated solution of sodium hydrogen carbonate and sodium thiosulfate. The mixture was extractor with dichloromethane. The combined organic layers were dried over sodium sulfate, and filtered. The solvent whs evaporated, and the crude product was purified by flash column chromatography (silica gel, gradient from 1:1 to 1:2 hexane-ethyl acetate) to give compound 7-3 (2.15 g, 49%) as a colorless gum. This material was obtained as a mixture of diastereomers.

¹H NMR (400 MHz, CDCl₃) (major isomer) 1.02 (d, J=6.9 Hz, 3H), 1.97 (s, 3H), 3.15-3.20 (m, 1H), 5.16 (s, 1H), 7.00-7.33 (5H, m), 9.71 (s, 1H).

### Step 3

A solution of compound 7-3 (2.15 g, 7.28 mmol) and Hunig's base (6.36 mL, 36.4 mmol) in THF (10 mL) was stirred at reflux for 25.5 h. The mixture was cooled to room temperature, and the solvent was evaporated. The crude product was purified by flush eolumn chromatography (silica gel, gradient from 87:13 to 85:15 hexane-ethyl acetate) to give compound 7-4 (1.81 g, 84%) as a colorless gum. This material was obtained as a mixture of diastereomers.

¹H NMR (400 MHz, CDCl₃) (major isomer) 1.12 (d, J=6.9 Hz, 3H), 1.76 (s, 3H), 3.09-3.14 (m, 1H), 5.17 (s, 1H), 7.02-7.33 (5H, m), 9.71 (s, 1H).

### Step 1

To a stirred solution of compound 7-4 (1.81 g, 6.13 mmol) in DMF (18 mL) were added difluoromethyl trimethylsilane (3.35 mL, 24.5 mmol) and cesium fluoride (279 mg, 1.84 mmol) at 0° C. After being stirred fur 4.5 days at room temperature, TBAF (1 mol/L in THF, 6.13 mL, 6.13 mmol) was added to the mixture at 0° C. The resulting mixture was stirred at 0° C. for 1 h, and water was added to the mixture. The mixture was extracted with ethyl acetate. The combined organic layers were washed with brine, dried over sodium sulfate, and filtered. The solvent was evaporated, and the residue was filtered through a pad of silica gel with aid of hexane and ethyl acetate (1:1). The filtrate was evaporated to give the crude product, which was used for the next reaction without further purification.

To a stilted solution of the crude product in dichloromethane (15 mL) was added TFA (1.73 mL, 22.5 mmol) at 0° C. After being stirred for 2.5 h at room temperature, the reaction was quenched with a saturated solution of sodium hydrogen carbonate. The mixture was extracted with dichloromethane. The combined organic layer was dried over sodium sulfate, nnd filtered. The solvent was evaporated to give the crude product, which was used for the next reaction without further purification.

To a stirred solution of the crude product in dichloromethane (15 mL) was added benzoyl isothiocyanate (0.724 mL, 5.39 mmol) at 0° C. After being stirred for 100 min at room temperature, the solvent was evaporated. The crude product was purified by flash column chromatography (silica gel, 3:1 hexane-ethyl acetate) to give compound 7-5 (749 mg, 41%) as an orange amorphous. This material was obtained as a mixture of diastereomers. MS: m/z=411.15 [M+H]⁺.

### Step 5

A suspension of compound 7-5 (749 mg, 1.83 mmol) and WSCD-HCl (700 mg, 3.65 mmol) in acetonitrile (7 mL) was stirred for 15 h at room temperature. Water was added to the reaction mixture, which was then extracted with ethyl acetate. The combined organic layers were washed with brine, dried over sodium sulfate, and filtered. The solvent was evaporated, and the crude product was purified by flash column chromatography (silica gel, gradient from 5:1 to 3:1 to 2:1 hexane:ethyl acetate) to give compound 7-6 (246 mg, 36%) as a yellow amorphous. This material was obtained as a mixture of diastereomers. MS: m/z=377.15 [M+H]⁺.

### Step 6

A solution of compound 7-6 (248 mg, 0.669 mmol), Boc₂O (0.214 mL, 0.988 mmol) and DMAP (16.1 mg, 0.132 mmol) in THF (3 mL) was stirred at room temperature for 3 h. The solvent was evaporated, and the crude product was purified by flash column chromatography (silica gel, gradient from 91:9 to 84:16 hexane:ethyl acetate) to give compound 7-7 (140 mg, 45%) as a colorless gum.

¹H NMR (400 MHz, CDCl₃) 1.19 (d, J=7.0 Hz, 3H), 1.41 (s, 9H), 1.54 (s, 3H), 2.67 (q, J=7.0 Hz, 1H), 4.00-4.05 (m, 1H), 5.82 (ddt, J=55.1, 6.5, 2.6 Hz, 1H), 7.06 (dd, J=12.5, 7.9 Hz, 1H), 7.14 (t, J=7.6 Hz), 7.46 (dd, J=15.4. 7.9 Hz, 2H), 7.56 (t, J=7.6 Hz, 1H), 7.77 (d, J=8.2 Hz, 1H).

### Step 7

A suspension of compound 7-7 (140 mg, 0.294 mmol) and potassium carbonate (122 mg, 0.881 mmol) in methanol (2 mL) was stirred for 2 h at room temperature. Water was added to the reunion mixture, which was then extracted with ethyl acetate. The combined organic layers were washed with brine, dried over sodium sulfate, and filtered. The solvent was evaporated to give the crude product, which was used for the next reaction without further purification. A solution of the crude product and TFA (0.226 mL, 2.93 mmol) in dichloromethane (2 mL) was stirred at room temperature for 6.5 h. The reaction was quenched with a saturated solution of potassium carbonate, which was then extracted with dichloromethane. The combined organic layers were dried over sodium sulfate, and filtered. The solvent was evaporated to give the crude product, which was used for the next reaction without further purification.

To a stirred solution of the crude product in TFA (1 mL) were added sulfuric acid (0.12 mL, 2.25 mmol), followed by nitric acid (0.020 mL, 0.441 mmol) at −25° C. After being stirred at −25° C. to −15° C. for 1.5 h, the reaction was quenched with a saturated solution of potassium carbonate. The mixture was extracted with ethyl acetate. The combined organic layers were washed with brine, dried over sodium sulfate, and filtered. The solvent was evaporated, and the crude product was purified by flash column chromatography (amino silica gel, gradient from 2:1 to 1:1 hexane:ethyl acetate) to give compound 7-8 (82.6 mg, 89%) as a colorless amorphous.

¹H NMR (400 MHz, CHCl₃) 1.14 (d, J=7.0 Hz, 3H), 1.54 (s, 3H), 2.64 (q, J=7.0 Hz, 1H), 3.74-3.79 (m, 1H), 5.76 (ddt, J=55.3, 6.3, 1.9 Hz, 1H), 7.21 (t, J=9.9 Hz, 1H), 8.16-8.20 (m, 1H), 8.35 (dd, J=6.8, 2.6 Hz, 1H).

### Step 8

A suspension of compound 7-8 (82.6 mg, 0.260 mmol), iron powder (116 mg, 2.08 mmol), and ammonium chloride (167 mg, 3.12 mmol) in ethanol (0.8 mL), THF (0.1 mL), and water (0.4 mL) was stirred at 60° C. for 100 min. The mixture was cooled to room temperature, filtered through a pad of Celite (Registered trademark). The filtrate was extracted with ethyl acetate. The combined organic layers were washed with brine, dried over sodium sulfate, and filtered. The solvent was evaporated to give the crude product (77.7 mg), which was used for the next reaction without further purification.

To a stirred solution of the crude product (18.8 mg) and aqueous hydrochloric acid solution (2 mol/L, 0.033 mL, 0.065 mmol) in methanol (1 mL) were added 5-fluoromethoxy)pyrazine-2-carboxylic acid (12.4 mg, 0.072 mmol) and WSCD-HCL (15.1 mg, 0.079 mmol) at room temperature. After being stirred for 1 h at room temperature, the reaction was quenched with aqueous sodium hydroxide solution. The mixture was extracted with ethyl acetate. The combined organic layers were washed with brine, dried over sodium sulfate, and filtered. The solvent was evaporated, and the crude product was purified by flash column chromatography (amino silica gel, gradient from 1:1 to 0:1 hexane:ethyl acetate) to give compound I-33 (25.0 mg, 87%) as an off-white solid.

### EXAMPLE 8

### Synthesis of Compound I-34

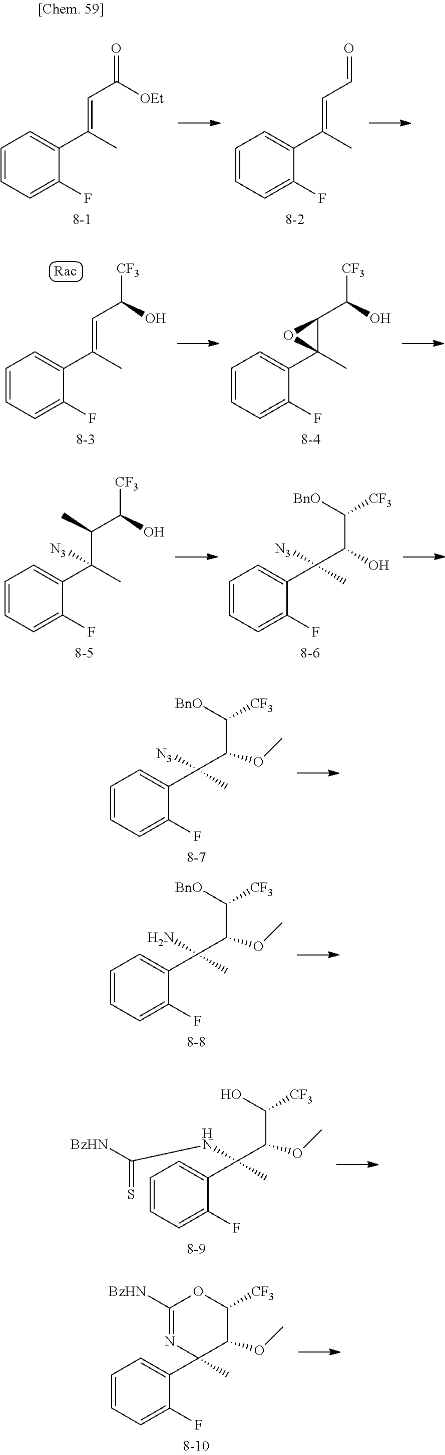

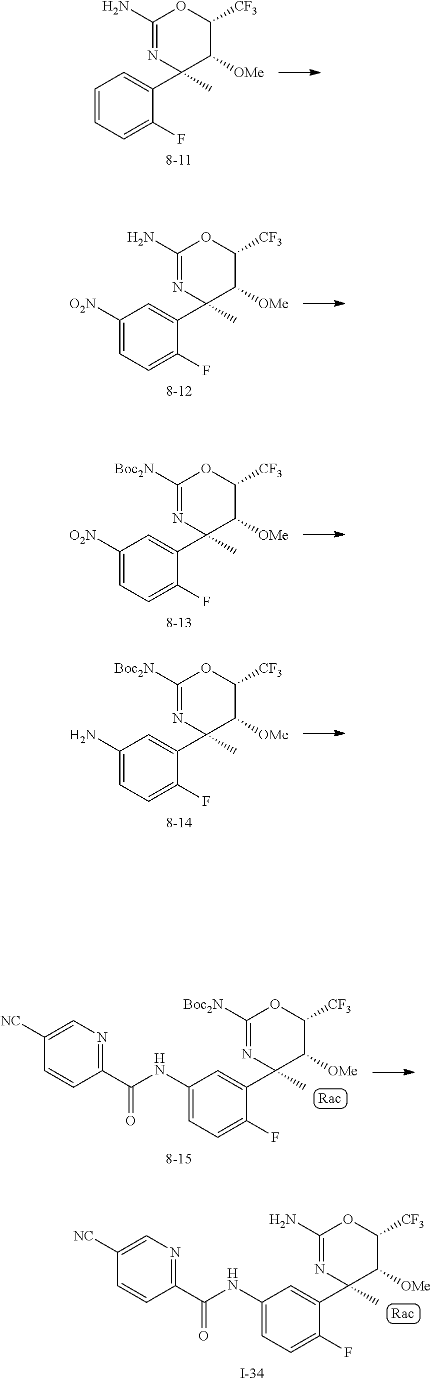

### Step 1

To a solution of compound 8-1 (4.59 g, 23.63 mmol) in THF (45 ml) was added 1.04 mol/L of DIBAL (56.8 ml, 59.1 mmol) in toluene at 0° C. After stirring for 2 h at the same temperature, the mixture was treated with saturated aqueous Rochelle's salt and stirred for 1.5 h. The aqueous layer was extracted with AcOEt, and the combined organic layer was washed with bride. The organic layer was dried over MgSO₄, filtered and concentrated. The crude compound was dissolved in CH₂Cl₂ (30 mL), and Dess-Martin periodinane (11.94 g, 28.2 mmol) was added at 0° C. After stirring for 2 h at room temperature, the mixture was treated with saturated aqueous NaHCO₃ and stirred fur 0.5 h. The aqueous layer was extracted with AcOEt, and the combined organic, layers were washed with brine, dried over MgSO₄ and filtered. The filtrate was concentrated in vacuo. The crude product was added to a silica gel column and etutad with Hexane/EtOAc 0% to 40%. Collected fractions were evaporated to afford compound 8-2 (3.67 g, 22.35 mmol, 95%) as a colorless oil.

¹H-NMR (400 MHz, CDCl₃) δ: 2.56 (s, 3H), 6.22 (d, J=8.0 Hz, 1H), 7.11 (dd, J=12.0, 8.0 Hz, 1H), 7.18 (t, J=8.0 Hz, 1H), 7.32 (m, 1H), 7.37 (m, 1H), 10.18 (d, J=8.0 Hz, 1H).

### Step 2

To a solution of compound 8-2 (2.01 g, 12.25 mmol) and (trifluoromethyl)trimethylsilane (2.61 g, 2.72 mmol) in THF (30 ml) was added 1.00 mol/L of TBAF in THF (0.123 ml, 0.123 mmol) at −10° C. After stirring for 1 h at 0° C. to the mixture was added 1.00 mol/L of TRAF (1.23 ml, 1.23 mmol) in THF, and the mixture was stirred for 1 h at room temperature. The reaction mixture was diluted with H₂O, and the aqueous layer was extracted with AcOEt. The combined organic layers were washed with H₂O and brine, dried over MgSO₄ and filtered. The filtrate was concentrated in vacuo. The crude product was added to a silica gel column and eluted with Hexane/EtOAc 0% to 30%. Collected fractions were evaporated to afford compound 8-3 (2.81 g, 12.00 mmol, 98%) as a yellow oil.

¹H-NMR (400 MHz, CDCl₃) δ: 2.19 (d, J=8.0 Hz, 1H), 2.20 (s, 3H), 4.86 (m, 1H), 5.67 (d, J=8.0 Hz, 1H), 7.06 (dd, J=8.0, 4.0 Hz, 1H), 7.12 (m, 1H), 7.25 (m, 1H), 7.29 (m, 1H).

### Step 3

To a solution of compound 8-3 (3.2 g, 13.66 mmol) in CH₂Cl₂ (40 ml) was added m-CPRA (6.74 g, 27.3 mmol) at 0° C. After stirring for 2 h at room temperature, the mixture was treated with 2 mol/L NaOH (30.6 ml) and stirred for 0.5 h. The aqueous layer was extracted with AcOEt, and the combined organic layers were washed with brine, dried over MgSO₄ and filtered. The filtrate was concentrated in vacuo. The crude product was used for next reaction without further purification.

¹H-NMR (400 MHz, CDCl₃) δ: 1.72 (s, 3H), 2.72 (d, J=8.0 Hz, 1H), 3.20 (d, J=8.0 Hz, 1H), 4.15 (m, 1H), 7.05 (m, 1H), 7.14 (m, 1H), 7.31 (m, 1H), 7.37 (m, 1H).

### Step 4

To a solution of compound 8-4 (3.42 g, 13.67 mmol) and Ti(OEt)₄ (18.71 g, 82 mmol) in DMF (30 ml) was added NaN₃ (3.55 mg, 54.7 mmol) at room temperature. After stirring for 20 h at the same temperature, the mixture was treated with saturated aqueous citric acid and stirred for 1 h. The aqueous layer was extracted with AcOEt, and the combined organic layers were washed with brine, dried over MgSO₄ and filtered. The filtrate was concentrated in vacuo. The crude product was added to a silica gel column and eluted with Hexane/EtOAc 20% to 60%. Collected fractions were evaporated to afford compound 8-5 (3.03 g, 11.26 mmol, 82%) as a yellow solid.

¹H-NMR (400 MHz, CDCl₃) δ: 1.93 (s, 3H), 2.85 (d, J=8.0 Hz, 1H), 3.19 (d, J=4.0 Hz, 1H), 3.75 (m, 1H), 4.47 (d, J=8.0 Hz, 1H), 7.11 (m, 1H), 7.22 (m, 1H), 7.37 (m, 1H), 7.58 (m, 1H).

### Step 5

To a solution of compound 8-5 (3.03 g, 11.25 mmol) in toluene (30 ml) and MeOH (30 ml) was added dibutyltin oxide (3.36 g, 13.51 mmol) at room temperature. After stirring for 3 h at 110° C. the reaction mixture was concentrated. Dry toluene (30 ml) was added to the residue, and the mixture was evaporated and dried in vacuo. The residue was dissolved in toluene (30 ml), and tetrabutylammonium bromide (0.726 g, 2.251 mmol) and benzyl bromide (3.54 mL 28.1 mmol) were added at room temperature. After being stirred for 20 h at 110° C. the reaction mixture was diluted with H₂O and extracted with AcOEt. The organic layers were dried over MgSO₄, filtered and concentrated. The crude product was added to a silica gel column and eluted with hexaneethyl acetate 0% to 30%. Collected fractions were evaporated to afford compound 8-6 (8.1 g, 8.09 mmol, 72%) as a colorless oil. ¹H NMR (400 MHz, CDCl₃) δ: 1.93 (s, 3H), 3.29 (d, J=12.0 Hz, 1H), 3.47 (q, J=8.0 Hz, 1H), 4.43 (d, J=8.0 Hz, 1H), 4.75 (m, 2H), 7.09 (m, 1H), 7.20 (m, 1H), 7.32-7.41 (m, 6H), 7.56 (m, 1H).

### Step 6

To a suspension of NaH (939 mg, 23.48 mmol) in THF (40 ml) was added compound 8-6 (3.0 g, 7.83 mmol) at 0° C. After stirring for 30 min at room temperature, to the mixture was added MeI (2.45 ml, 39.1 mmol), and the mixture was stirred for 30 min at room temperature. The reaction mixture was treated with saturated aqueous NH₄Cl, and the aqueous layer was extracted with AcOEt. The combined organic layers were washed with brine, dried over MgSO₄ and filtered. The filtrate was concentrated in vacuo. The crude product was added to a silica gel column and eluted with Hexane/EtOAc 0% to 30%. Collected fractions were evaporated to afford compound 8-7 (3.03 g, 7.63 mmol, 97%) as a colorless oil.

¹H-NMR (400 MHz, CDCl₃) δ: 1.91 (s, 2H), 3.54 (s, 3H), 3.61 (s, 3H), 4.08 (s, 1H), 4.72 (s, 2H), 7.07 (m, 1H), 7.15 (m, 1H), 7.29-7.41 (m, 6H), 7.51 (m, 1H).

### Step 7

A suspension of compound 8-7 (3.0 g, 7.55 mmol) and 10% Pd/C (600 mg) in MeOH (40 ml) was stirred under it hydrogen atmosphere at room temperature. After stirring for 24 h at the same temperature, the mixture was filtrated through a pad of Celite (Registered Trademark). The filtrate was concentrated under vacuum to give compound 8-8 (2.13 g, 7.57 mmol, 100%) as a white solid, which was used for the next step without purification.

¹H -MR (400 MHz, CDCl₃) δ: 1.65 (s, 3H), 3.17 (s, 1H), 3.61 (s, 3H), 3.65 (m, 1H), 3.99 (s, 1H), 7.07 (m, 1H), 7.20 (m, 1H), 7.31 (m, 1H), 7.56 (m, 1H).

### Step 8

To a stirred solution of compound 8-8 (2.13 g, 7.57 mmol) in CH₂Cl₂ (30 mL) was added benzoyl isothiocyanate (1.22 mL, 6.82 mmol) at 0° C. After being stirred for 2 h at room temperature, the reaction mixture was concentrated, and the resulting residue was added too silica gel column and eluted with hexane/ethyl acetate (0% to 50%. Collected fractions were evaporated to afford compound 8-9 (3.03 g, 6.82 mmol, 90%) as a yellow amorphous.

¹H-NMR (400 MHz, CDCl₃) δ: 2.32 (s, 3H), 3.48 (s, 1H), 3.56 (s, 3H), 3.95 (m, 1H), 7.09 (m, 1H), 7.18 (m, 1H), 7.32 (m, 1H), 7.41 (m, 1H), 7.52 (m, 2H), 7.63 (m, 1H), 7.87 (m, 2H), 8.88 (m, 1H), 11.66 (m, 1H).

### Step 9

To a stirred solution of compound 8-9 (8.03 g, 6.84 mmol) in acetonitrile (30 mL) was added EDC (3.93 g, 20.52 mmol) at room temperature. After being stirred for 20 h at the same temperature, the reaction mixture was diluted with H₂O and extracted wiih ethyl acetate. The organic layers were combined and washed with brine. The organic layer was dried over MgSO₄ filtered and concentrated. The crude product was used for next reaction without further purification.

¹H-NMR (400 MHz, CDCl₃) δ: 1.87 (s, 3H), 3.71 (s, 3H), 4.23 (s, 1H), 4.31 (m, 1H), 7.15 (m, 1H), 7.23 (m, 1H), 7.35-7.55 (m, 5H), 8.28 (m, 2H), 11.64 (m, 1H).

### Step 10

To a stirred solution of compound 8-10 (2.81 g, 6.85 mmol) in THF (40 mL) were added Boc₂O (2.385 mL, 10.27 mmol) and DMAP (251 mg, 2.05 mmol) at room temperature under nitrogen. After being stirred for 1 h, the reaction mixture was diluted with H₂O and extracted with ethyl acetate. The organic layers were combined and washed with brine. The organic layer was dried over MgSO₄, filtered and concentrated. The crude compound was dissolved in methanol (40 mL), and K₂CO₃ (2.50 g, 18.1 mmol) was added at 0° C. After being stirred for 2 h at room temperature. The reaction mixture was diluted with H₂O and extracted with ethyl acetate. The organic layer was dried over MgSO₄, filtered and concentrated. The crude product was added to a silica gel column and eluted with hexane/ethyl acetate 0% to 30%. Collected fractions were evaporated to afford the Boc-protected compound. The compound was dissolved in CH₂Cl₂ (15 mL), and TFA (4 ml) was added at 0° C. After being stirred for 2 h at r.t., the reaction mixture was quenched with 20% aq. Na₂CO₃ and extracted with ethyl acetate. The organic layer was dried over MgSO₄ and filtered. The filtrate was concentrated under vacuum to give compound 8-11 (1.16 g, 3.79 mmol, 55%) as a white amorphous, which was used for the next step without purification.

¹H-NMR (400 MHz, CDCl₃) δ: 1.69 (s, 3H), 3.66 (s, 3H), 3.99 (m, 1H), 4.16 (s, 1H), 7.07 (dd, J=12.0, 8.0 Hz, 1H), 7.17 (t, J=8.0 Hz, 1H), 7.30 (m, 1H), 7.39 (t, J=8.0 Hz, 1H).

### Step 11

To a solution of compound 8-11 (1.14 g, 3.72 mmol) in TFA (4.9 ml) was added sulfuric acid (1.25 ml, 23.5 mmol) at −10° C. After stirring for 5 min at −10° C., to the reaction mixture was added HNO₃ (0.36 ml, 5.58 mmol) at −10° C. After stirring for 30 min at −10° C., the reaction mixture was treated with aqueous K₂CO₃. The aqueous layer was extracted with AcOEt, and the organic layer was dried over MgSO₄, filtered and concentrated. The filtrate was concentrated under vacuum to give compound 8-12 (1.23 g, 3.50 mmol, 94%) as a white amorphous.

¹H-NMR (400 MHz, CDCl₃) δ: 3.67 (s, 3H), 3.67 (s, 3H), 3.93 (m, 1H), 4.13 (m, 1H), 7.07 (m, 1H), 8.21 (m, 1H), 8.39 (m, 1H).

### Step 12

To a solution of compound 8-12 (1.2 g, 3.42 mmol) and DMAP (125 mg, 1.027 mmol) in THF (10 ml) was added Boc₂O (2.38 ml, 10.3 mmol) at room temperature. After stirring for 2 h at the same temperature, the mixture was concentrated under vacuum. The crude product was added to a silica gel column and eluted with hexane/EtOAc 0% to 30%. Collected fractious were evaporated to afford compound 8-13 (1.73 g, 3.14 mmol, 92%) as a white amorphous.

¹H-NMR (400 MHz, CDCl₃) δ: 1.53 (s, 18H), 1.57 (s, 3H), 3.64 (s, 3H), 4.02 (m, 1H), 4.15 (s, 1H), 7.27 (m, 1H), 8.25 (m, 1H), 8.50 (m, 1H).

### Step 13

A suspension of compound 8-13 (1.73 g, 3.14 mmol) 10% Pd/C (300 mg) in MeOH (8 ml) and THF (6 ml) was stirred under a hydrogen atmosphere at room temperature. After stirring for 2 h at the same temperature, the mixture was filtrated through a pad of Celite (Registered trademark). The filtrate was concentrated under vacuum to give compound 8-14 (1.63 g, 3.18 mmol, 99%) as a white amorphous, which was used for the next step without purification.

¹H-NMR (400 MHz, CDCl₃) δ: 1.52 (s, 18H), 1.57 (s, 3H), 3.59 (s, 3H), 4.13 (m, 1H), 4.14 (m, 1H), 6.55 (m, 1H), 6.83 (m, 1H), 6.87 (m, 1H).

### Step 14

To a solution of compound 8-11 (201 mg, 0.385 mmol) in CH₂Cl₂ (2 ml) were added 5-cyanopicolinic acid hydrate (70.4 mg, 0.424 mmol), HATU (161 mg, 0.424 mmol) and DIPEA (0.101 ml, 0.578 mmol) at room temperature. After stirring for 18 h at the same temperature, the reaction mixture was treated with H₂O. The aqueous layer was extracted with AcOEt, and the organic layer was dried over MgSO₄, filtered and concentrated. The crude product was added to a silica gel column and eluted with hexane/EtOAc 0% to 50%. Collected fractions were evaporated to afford compound 8-15 (245 mg, 0.376 mmol, 98%) as a white amorphous.

¹H-NMR (400 MHz, CDCl₃) δ: 1-56 (s, 18H), 1.75 (s, 3H), 3.63 (s, 3H), 4.11 (m, 1H), 4.16 (m, 1H), 7.16 (dd, J=12.0, 8.0 Hz, 1H), 7.55 (dd, J=8.0, 4.0 Hz, 1H), 8.22 (dd, J=8.0, 4.0 Hz, 1H), 8.39 (m, 1H), 8.43 (d, J=8.0 Hz, 1H), 8.79 (m, 1H), 9.98 (s, 1H).

### Step 15

To a solution of compound 8-15 (44 mg, 0.073 mmol) in CH₂Cl₂ (1.5 ml) was added TFA (0.5 ml) at 0° C. After being stirred for 2 h at r.t., the reaction mixture was quenched with 20% aq. K₂CO₃. The aqueous layer was extracted with AcOEt and the organic layers were combined and washed with brine. The organic layer was dried over MgSO₄, filtered and concentrated. The crude product was purified by supercritical fluid chromatography (SFC) (Chiralpak (Registered trademark) IB: 5-40% ethyl alcohol with 0.1% diethylamine) to give compound I-34 (58 mg, 0.128 mmol, 34%) as a white solid.

### EXAMPLE 9

### Synthesis of Compound I-35

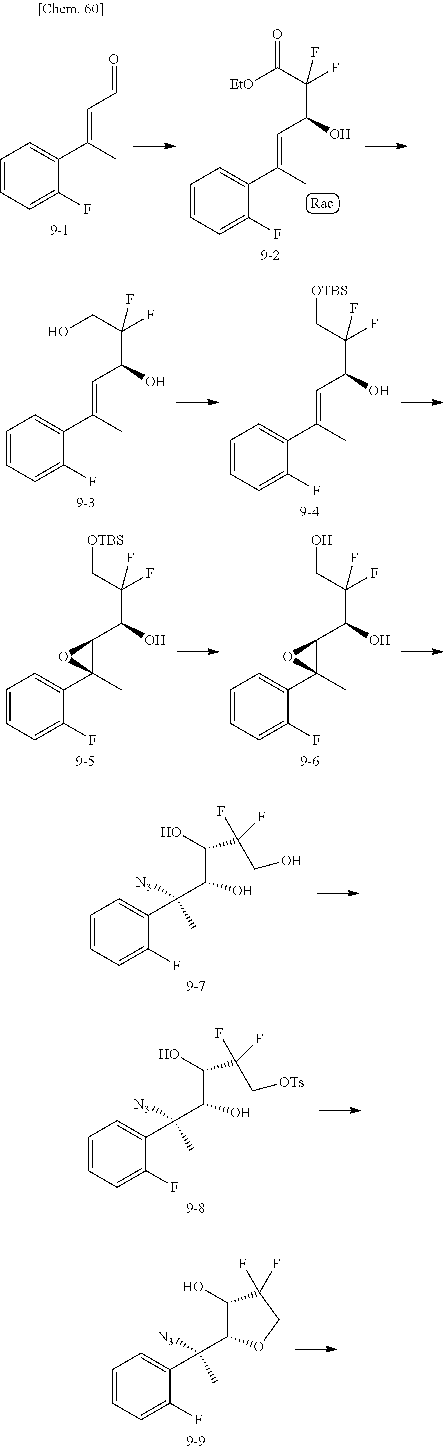

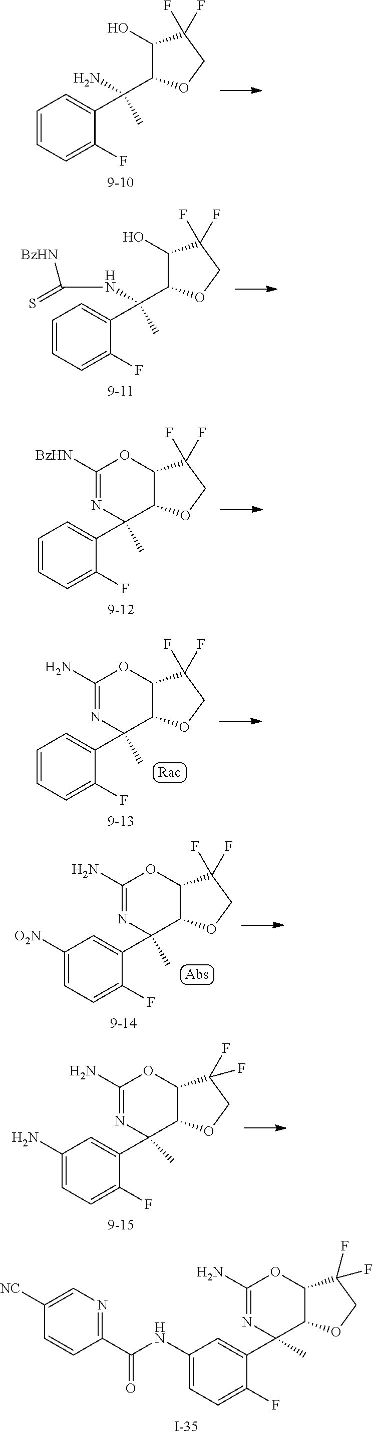

### Step 1

To a suspension of zinc (1.20 g, 18.27 mmol) in THF (10 ml) wire added a solution of ethylbromodifluoroacetate (5.19 g, 25.6 mmol) in THF (10 ml) and a solution of compound 9-1 (1.2 g, 7.31 mmol) in THF (10 ml) at room temperature. After stirring for 2 h at the same temperature, the reaction mixture was treated with saturated aqueous NH₄Cl, and the aqueous layer was extracted with AcOEt. The combined organic layers were washed with brine, dried over Na₂SO₄, filtered and concentrated. The crude product was added to a silica gel column and elided with Hexane/EtOAc 0% to 40%. Collected fractions were evaporated to afford compound 9-2 (1.91 g, 0.63 mmol, 91%) as a colorless oil.

¹H-NMR (400 MHz, CDCl₃) δ: 1.37 (t, J=8.0 Hz, 3H) 2.15 (s, 3H), 2.21 (d, J=4.0 Hz, 1H), 1.38 (q, J=8.0 Hz, 2H), 4.98 (m, 1H), 5.67 (d, J=8.0 Hz, 1H), 7.04 (m, 1H), 7.11 (t, J=8.0 Hz, 1H), 7.24 (m, 1H), 7.28 (m, 1H).

### Step 2

To a solution of compound 9-2 (2.09 g, 7.24 mmol) in MeOH (80 ml) was added NaBH4 (822 mg, 21.7 mmol) at 0° C. After stirring for 0.5 h at the same temperature, the mixture was treated with saturated aqueous NH4Cl and stirred for 0.5 h. The aqueous layer was extracted with AcOEt, and the combined organic layers were washed with brine. The organic layer was dried over MgSO₄ , filtered and concentrated. The crude product was added to a silica gel column and eluted with hexane/EtOAc 0% to 40%. Collected fractious were evaporated to afford compound 9-3 (1.70 g, 6.91 mmol, 95%) as a colorless oil.

¹H-NMR (400 MHz, CDCl₃) δ: 2.14 (s, 3H), 2.32 (t, J=8.0 Hz, 1H), 2.39 (d, J=8.0 Hz, 1H), 3.92 (m, 1H), 4.02 (m, 1H), 4.88 (m, 1H), 5.72 (d, J=12.0 Hz, 1H), 7.04 (m, 1H), 7.10 (t, J=8.0 Hz, 1H), 7.25 (m, 1H, 7.27 (m, 1H).

### Step 3

To a solution of compound 9-3 (1.71 g, 6.94 mmol) in CH₂Cl₂ (30 ml) were added TBSCl (2.00 g, 13.89 mmol) and imidazole (0.946 g, 13.89 mmol) at room temperature. After stirring for 0.5 h at the same temperature, the mixture was treated with water and stirred for 0.5 h. The aqueous layer was extracted with AcOEt, and the combined organic layers were washed with brine. The organic layer was dried over MgSO₄, filtered and concentrated. The crude product was added to a silica gel column and eluted with hexane/EtOAc 0% to 20%. Collected fractions were evaporated to afford compound 9-4 (1.95 g, 5.42 mmol, 78%) as a colorless oil.

¹H-NMR (400 MHz, CDCl₃) δ: 0.10 (s, 3H), 0.12 (s, 3H), 0.92 (s, 9H), 2.13 (s, 3H), 2.31 (d, J=4.0 Hz, 1H), 3.87 (m, 1H), 3.99 (m, 1H), 4.88 (m, 1H), 5.70 (d, J=12.0 Hz, 1H), 7.04 (dd, J=12.0, 8.0 Hz, 1H), 7.10 (t, J=8.0 Hz, 1H), 7.24 (m, 1H), 7.26 (m, 1H).

### Step 4

To a solution of compound 9-4 (195 g, 5.42 mmol) in CH₂Cl₂ (30 ml) was added m-CPBA (2.67 g, 10.8 mmol) at 0° C. After stirring for 2 h at room temperature, the mixture was treated with 2 mol/L NaOH (20.5 ml) and stirred for 0.5 h. The aqueous layer was extracted with AcOEt, and the rombined organic layer was washed with brine, dried aver MgSO₄ and filtered. The crude product was added to a silica gel column and eluted with Hexane/EtOAc 0% to 30%. Collected fractions were evaporated to afford compound 9-5 (1.92 g, 5.10 mmol, 94%) as a colorless oil.

¹H-NMR (490 MHz, CDCl₃) δ: 0.11 (s, 6H), 0.90 (s, 9H), 1.71 (s, 3H), 2.61 (d, J=8.0 Hz, 1H), 3.28 (d, J=8.0 Hz, 1H), 3.94 (m, 1H), 4.03 (m, 1H), 4.10 (m, 1H), 7.03 (m, 1H), 7.12 (t, J=8.0 Hz, 1H), 7.27 (m, 1H), 7.38 (m, 1H).

### Step 5

To a solution of compound 9-5 (1.92 g, 5.10 mmol) in THF (25 ml) was added 1.00 mol/L of TBAF in THF (5.61 ml, 5.61 mmol) at 0° C. After stirring for 2 h at the same temperature, the mixture was treated with saturated aqueous NaHCO₃ and stirred for 0.5 h. The aqueous layer was extracted with AcOEt, and the combined organic layer was washed with brine. The organic layer was dried over MgSO₄, filtered and concentrated. The crude product was added to a silica gel column and eluted with Hexane/EtOAc 10% to 50%. Collected fractions were evaporated to afford compound 9-6 (1.31 g, 5.00 mmol, 98%) as a colorless oil.

¹H-NMR (400 MHz, CDCl₃) δ: 1.72 (s, 3H), 2.09 (t, J=4.0 Hz, 1H) 2.63 (d, J=4.0 Hz, 1H), 3.31 (d, J=8.0 Hz, 1H), 3.97-4.18 (m, 3H), 7.05 (m, 1H), 7.13 (t, J=8.0 Hz, 1H), 7.29 (m, 1H), 7.39 (t, J=8.0 Hz, 1H).

### Step 6

To a solution of 9-6 (1.31 g, 5.00 mmol) and Ti(OEt)₄ (6.85 g, 30.0 mmol) in DMF (12 ml) was added NaN₃ (1.30 g, 20.0 mmol) at room temperature. After stirring for 20 h at the same temperature, the mixture was treated with saturated aqueous citric acid and stirred for 1 h. The aqueous layer was extracted with AcOEt, and the combined organic layers were washed with brine, dried over MgSO₄ and filtered. The filtrate was concentrated in vacuo. The crude product was added to a silica gel column and eluted with hexane/EtOAc 20% to 60%. Collected fractions were evaporated to afford compound 9-7 (1.38 g, 4.52 mmol 90%) as a white solid.

¹H NMH (400 MHz, CDCl₃) δ: 1.93 (s, 3H), 2.70 (br, 1H), 3.22 (d, J=8.0 Hz, 1H), 3.38 (br, 1H), 3.67 (m, 1H), 3.77 (m, 1H), 3.90 (m, 1H), 4.50 (s, 1H), 7.10 (dd, J=12.0, 8.0 Hz, 1H), 7.20 (t, J=8.0 Hz, 1H), 7.35 (m, 1H), 7.58 (t, J=8.0 Hz, 1H).

### Step 7

To a solution of compound 9-7 (1.38 g, 4.52 mmol) and p-toluenesulfonyl chloride (0.95 g, 4.97 mmol) in CH2Cl2 (26 ml) was added DMAP (1.11 g, 9.04 mmol) at 0° C. After stirring for 2 h at the same temperature, the mixture was treated with saturated aqueous NaHCO3 and stirred for 0.5 h. The aqueous layer was extracted with AcOEt, and the combined organic layers were washed with 0.1 mol/L aqueous HCl and brine, dried over MgSO₄ and filtered. The filtrate was concentrated in vacuo. The crude product was added to a silica gel column and eluted with hexanc/EtOAc 0% to 30%. Collected fractions were evaporated to afford compound 9-8 (1.92 g, 4.18 mmol, 92%) as a colorless oil.

¹H-NMR (400 MHz, CDCl₃) δ: 1.90 (s, 3H), 2.46 (s, 3H), 2.83 (d, J=8.0 Hz, 1H), 3.05 (d, J=8.0 Hz, 1H), 3.61 (s, 1H), 4.20 (m, 1H), 4.28 (m, 1H), 4.42 (d, J=8.0 Hz, 1H), 7.08 (dd, J=12.0, 8.0 Hz, 1H), 7.20 (t, J=8.0 Hz, 1H), 7.34 (m, 4H), 7.54 (m, 1H), 7.72 (m, 2H).

### Step 8

To a solution of compound 9-8 (1.92 g, 4.18 mmol) in MeOH (30 ml) was added K₂CO₃ (1.16 g, 8.36 mmol) at room temperature. After stirring for 20 h at the same temperature, the mixture waa treated with water and stirred for 0.5 h. The aqueous layer was extracted with AcOEt, and the combined organic layers were washed with brine, dried over MgSO₄ and filtered. The filtrate was concentrated in vacuo. The crude product was added to a silica gel column and eluted with hexane/EtOAc 0% to 30%. Collected fractions were evaporated to afford compound 9-9 (1.10 g, 3.84 mmol, 92%) as a colorless oil.

¹H-NMR (400 MHz, CDCl₃) δ: 1.92 (s, 3H), 3.72 (m, 1H), 3.78 (m, 1H), 4.04 (m, 1H), 4.28 (m, 1H), 4.74 (m, 1H), 7.09 (dd, J=12.0, 8.0 Hz, 1H), 7.19 (t, J=8.0 Hz, 1H), 7.33 (m, 1H), 7.67 (m, 1H).

### Step 9

A suspension of compound 9-9 (1.06 g, 3.66 mmol) in MeOH (25 ml) and 10% Pd/C (200 mg) was stirred under a hydrogen atmosphere at room temperature. After stirring for 24 h at the same temperature, the mixture was filtrated through a pad of Celite (Registered trademark). The filtrate was concentrated under vacuum to give compound 9-10 (0.9 g, 3.47 mmol, 95%) as a white solid, which was used for the next step without purification.

¹H-NMR (400 MHz, CDCl₃) δ: 1.70 (s, 3H), 3.67 (dd, J=8.0, 4.0 Hz, 1H), 4.04 (m, 1H), 4.25 (m, 1H), 4.54 (m, 1H), 7.09 (dd, J=12.0, 8.0 Hz, 1H), 7.20 (t, J=8.0 Hz, 1H), 7.83 (m, 1H), 7.55 (m, 1H).

### Step 10

To a stirred solution of compound 9-10 (750 mg, 2.87 mmol) in CH₂Cl₂ (15 mL) was added benzoyl isothiocyanate (0.579 mL, 4.31 mmol) at 0° C. After being stirred for 6 h at room temperature, the reaction mixture was concentrated, and the resulting residue was added in a silica gel column and eluted with hexane/ethyl acetate 0% to 35%. Collected fractions were evaporated to afford compound 9-11 (1.07 g, 2.51 mmol, 87%) as a colorless amorphous.

¹H-NMR (400 MHz, CDCl₃) δ: 2.28 (s, 3H), 3.96 (m, 1H), 1.14 (m, 1H), 4.46 (m, 1H), 4.80 (m, 1H), 7.06 (dd, J=12.0, 8.0 Hz, 1H), 7.14 (t, J=8.0 Hz, 1H), 7.29 (m, 1H), 7.52 (m, 3H), 7.62 (t, J=8.0 Hz, 1H), 7.87 (d, J=4.0 Hz, 1H), 8.86 (s, 1H), 11.81 (s, 1H).

### Step 11

To a stirred solution of compound 9-11 (1.156 g, 2.72 mmol) in acetonitrile (15 mL) was added EDC (1.04 g, 5.45 mmol) at room temperature. After being stirred for 20 h at the same temperature, the reaction mixture was diluted with H₂O and extracted with ethyl acetate. The organic layers were combined and washed with brine. The organic layer was dried over MgSO₄, filtered and concentrated. The crude product was added to a silica gel column and eluted with hexane/ethyl acetate 0% to 40%. Collected fractions were evaporated to afford compound 9-12 (853 mg, 2.19 mmol, 80%) aa a while solid.

¹H-NMR (400 MHz, CDCl₃) δ: 1.86 (s, 3H), 4.13-4.33 (m, 3H), 4.87 (s, 1H), 7.15 (m, 1H), 7.18 (m, 1H), 7.30 (m, 1H), 7.38 (m, 1H), 7.44 (t, J=8.0 Hz, 2H), 7.53 (t, J=8.0 Hz, 1H), 8.28 (d, J=8.0 Hz, 2H), 11.62 (s, 1H).

### Step 12

To a stirred solution of compound 9-12 (853 mg, 2.19 mmol) in THF (10 mL) were added Boc₂O (0.761 mL, 3.28 mmol) and DMAP (80 mg, 0.656 mmol) at room temperature. After being stirred for 1 h, the reaction mixture was diluted with H₂O and extracted with ethyl acetate. The organic layers were combined and wasbed with brine. The organic layer was dried over MgSO₄, filtered and concentrated. The crude compound was dissolved in methanol (15 mL), and K₂CO₃ (904 mg, 6.54 mmol) was added at 0° C. After being stirred for 2 h at room temperature, the reaction mixture was diluted with H₂O and extracted with ethyl acetate. The organic layer wee dried over MgSO₄, filtered and concentrated. The crude product was added to a silica gel column and eluted with hexane/ethyl acetate 0% to 30%. Collected fractions were evaporated to afford the Boc-protected compound. The compound was dissolved in CH₂Cl₂ (5 mL) and TFA (1.5 ml) was added at 0° C. After being stirred for 2 h at r.t., the reaction mixture was quenched with 20% aq. Na₂CO₃ and extracted with ethyl acetate. The organic layer was dried over MgSO₄ and filtered. The filtrate was concentrated under vacuum to give compound 9-13 (538 mg, 1.88 mmol, 86%) as a white amorphous, which was used for the next step without purification.

¹H-NMR (400 MHz, CDCl₃) δ: 1.65 (s, 3H), 4.00-4.23 (m, 3H), 4.70 (s, 1H), 7.06 (dd, J=12.0, 8.0 Hz, 1H), 7.14 (t, J=8.0 Hz, 1H), 7.28 (m, 1H), 7.36 (t, J=8.0 Hz, 1H).

### Step 13

To a solution of compound 9-13 (538 mg, 1.88 mmol) in TFA (2.2 ml) was added sulfuric acid (0.65 ml, 12.2 mmol) at −10° C. After stirring for 5 min at −10° C. to the reaction mixture was added HNO₃ (0.18 ml, 2.82 mmol) at −10° C. After stirring for 30 min at −10° C., the reaction mixture was treated with a aqueous K₂CO₂. The aqueous layer was extracted with AcOEt, and the organic layer was dried over MgSO₄, filtered and concentrated. The crude product was purified by supercritical fluid chromatography (SFC) (Chiralpak (Registered trademark) IC; 0-65% methyl alcohol with 0.1% diethylamine) to give compound 9-14 (300 mg, 0.91 mmol, 48%) as a white amorphous.

¹H-NMR (100 MHz, CDCl₃) δ: 1.66 (s, 3H), 4.04 (m, 1H), 4.05-4.24 (m, 2H), 4.68 (s, 1H), 7.23 (m, 1H), 8.21 (m, 1H), 8.31 (m, 1H).

### Step 14

A suspension of compound 9-14 (71 mg, 0.21 mmol) 10% Pd/C (20 mg) in MeOH (2 ml) was stirred under a hydrogen atmosphere at room temperature. After stirring for 2 h at the same temperature, the mixture was filtrated through a pad of Celite (Registered trademark). The filtrate was concentrated under vacuum to give compound 9-15 (63 mg, 0.21 mmol, 98%) as a white amorphous, which was used for the next step without purification.

¹H-NMR (400 MHz, CDCl₃) δ: 1.61 (s, 3H), 4.02-4.33 (m, 3H), 4.69 (s, 1H), 6.52 (m, 1), 6.64 (m, 1H), 6.84 (m, 1H).

### Step 15

To a solution of compound 9-15 (63 mg, 0.21 mmol) in CH₂Cl₂ (2 ml) were added 5-cyanopicolinic acid hydrate (38.2 mg, 0.23 mmol), EDC (48 mg, 0.25 mmol) and 2 mmol/L HCl (aqueous solution, 0.11 ml, 0.209 mmol) at room temperature. After stirring for 1 h at the same temperature, the reaction mixture was treated with H₂O. The aqueous layer was extracted with AcOEt, and the organic layer was dried over MgSO₄, filtered and concentrated. The crude product was added to a silica gel column and eluted with hexane/EtOAc 30% to 70%. Collected fractions were evaporated to afford compound I-35 (66 mg, 0.153 mmol, 73%) as a white solid.

### EXAMPLE 10

### Synthesis of Compound I-63

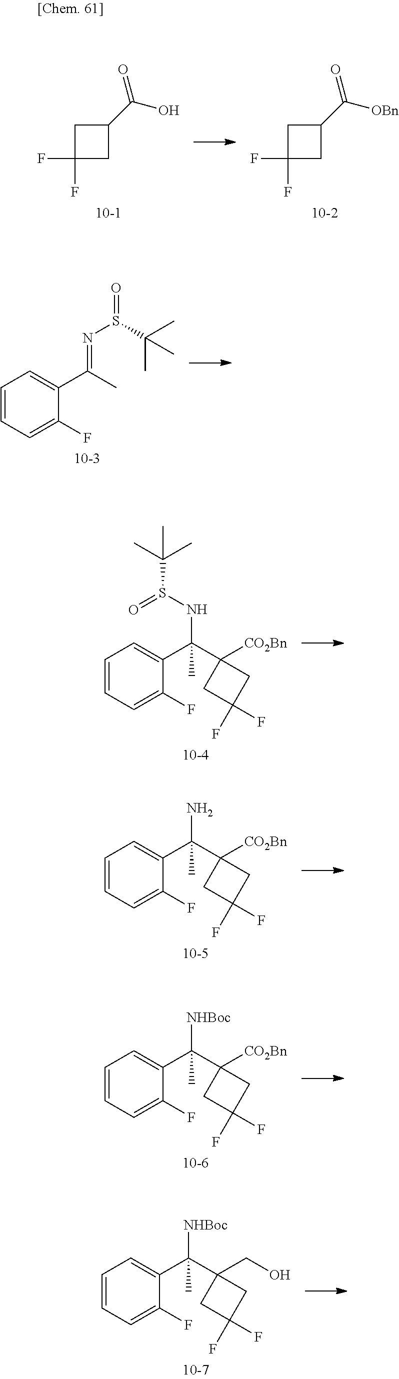

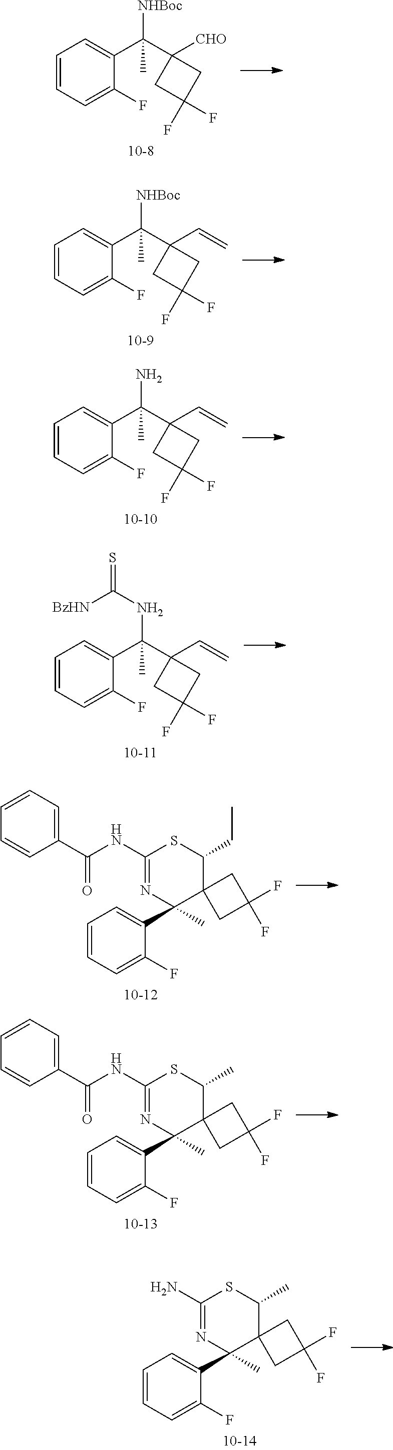

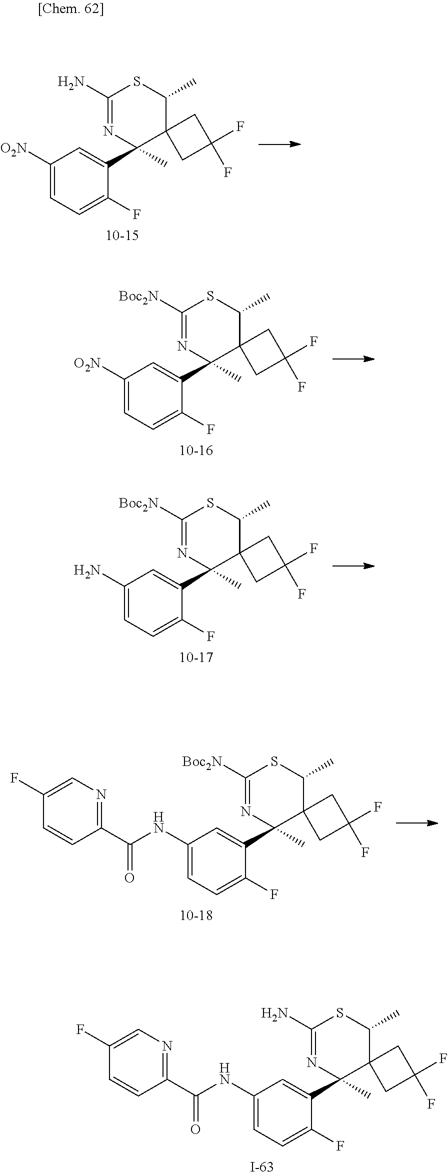

### Step 1: Synthesis of Compound 10-4

To a solution of 3,3-difluorocyclobutanecarboxylic acid (compound 10-1, 3.00 g, 22.0 mmol) in DMF (30 ml) were added Cs₂CO₃ (14.4 g, 44.1 mmol) and BnBr (2.62 ml, 22.0 mmol) at room temperature. After stirring for 20 min at 50° C., the mixture was treated with H₂O, and the aqueous layer was extracted with AcOEt. The combined organic layer was washed with H₂O and brine, dried over Na₂SO₄ and filtered. The filtrate was concentrated under vacuum to give compound 10-2 as a yellow oil, which was used for the next step without purifiration.

To a solution of diisopropylamine (2.91 ml, 20.5 mmol) in THF (25 ml) was added 1.6 mol/L of n-/BuLi (12.2 ml, 19.5 mmol) at −78° C. After stirring for 30 min at the same temperature, compound 10-2 (3.30 g, 14.6 mmol) in THF (10 ml) was added, and this was stirred for 1 h followed by addition of Ti(OiPr)₃Cl (4.89 ml, 20.5 mmol) in THF (10 ml). After stirring for 10 min at the same temperature, to the mixture was added 10-3 (2.35 g, 9.74 mmol) in THF (10 ml). The mixture was stirred for 1 h at the same temperature and was treated with saturated aqueous NH₄Cl. The aqueous layer was extracted with AcOEt, and the combined organic, layers were washed with brine, dried over Na₂SO₄ and filtered. The filtrate was concentrated in vacuo. The crude product was added in a silica gel column and eluted with hexane/EtOAc 30%. Collected fractions were evaporated to afford compound 10-4 (1.21 g, 2.65 mmol, 27%) as a yellow oil.

¹H-NMR (400 MHz/CDCl₃) δ: 1.24 (s, 9H) 1.94 (s, 3H), 2.87-3.33 (m, 4H), 4.79 (s, 2H), 5.15 (s, 1H), 6.94-7.01 (m, 1H), 7.03-7.08 (m, 1H), 7.09-7.14 (m, 2H), 7.22-7.39 (m, 5H).

### Step 2: Synthesis of Compound 10-6

To a solution of compound 10-4 (1.21 g, 2.65 mmol) in MeOH (8 ml) was added 4 mol/L of HCl in dioxane (0.995 ml, 3.98 mmol) at room temperature. After stirring for 1 h at the same temperature, the reaction mixture was treated with aqueous NaHCO₃, and the aqueoue layer was extracted with CHCl₃. This combined organic layer was washed with H₂O and brine, dried over Na₂SO₄ and filtered. The filtrate was concentrated under vacuum to give compound 10-5 as a brown oil, which was used for the next step without purification.

To a solution of compound 10-5 in MeOH (10 ml) was added Boc₂O (1.85 ml, 7.96 mmol) at room temperature. After stirring for 16 h at 60° C. the reaction mixture was concentrated. The resulting residue was added to a silica gel column and eluted with Hexane/EtOAc 0% to 10%. Collected fractions were evaporated to afford 10-6 (914 mg, 1.97 mmol, 74%) as a colorless oil.

¹H-NMR (400 MHz, CDCl₃) δ: 1.24 (s, 9H), 1.94 (s, 3H), 2.88-3.32 (m, 4H), 4.79 (s, 2H), 5.15 (s, 1H), 6.94-7.01 (m, 1H), 7.03-7.08 (m, 1H), 7.10-7.14 (m, 2H), 7.39-7.22 (m, 5H).

### Step 3: Synthesis of Compound 10-7

To a solution of compound 10-6 (914 mg, 1.97 mmol) in THF (9 ml) were added 3 mol/L of LiBH₄ in THF (1.97 ml, 5.92 mmol) and MeOH (0.240 ml, 5.92 mmol) at 0° C. After stirring for 30 min at room temperature, the mixture was treated with H₂O and ArOH. The aqueous layer was extracted with AcOEt, and the combined organic layers were washed with brine, dried over Na₂SO₄ and filtered. The filtrate was concentratec in vacuo. The crude product was added to a silica gel column and eluted with hexane/EtOAc 20%. Collected fractions were evaporated to afford compound 10-7 (693 mg, 1.93 mmol, 98%) as a white solid.

¹H-NMR (400 MHz, CDCl₃) δ: 1.38 (s, 9H), 1.99 (d, J=2.5 Hz, 3H), 2.04-2.19 (m, 1H), 2.46-2.60 (m, 1H), 2.78 (q, J=14.1 Hz, 1H), 2.92 (q, J=14.4 Hz, 1H), 3.60 (d, J=10.5 Hz, 1H), 3.77 (d, J=10.5 Hz, 1H), 6.51 (s, 1H), 6.99-7.06 (m, 1H), 7.11-7.16 (m, 1H), 7.30-7.23 (m, 1H), 7.35 (t, J=7.3 Hz, 1H).

### Step 4: Synthesis of Compound 10-8

To a solution of compound 10-7 (693 mg, 1.93 mmol) in CH₂Cl₂ (7 ml) was added DMP (2.13 g, 5.01 mmol) at room temperature. After stirring for 16 h at the same temperature, the mixture was treated with aqueous NaHCO₃ and aqueous NaHSO₃. The aqueous layer was extracted with CHCl₃, and the combined organic layers were washed with brine, dried over Na₂SO₄ and filtered. The fitrate was concentrated in vacuo. The crude product was added to a silica gel column and eluted with hexane/EtOAc 20%. Collected fractions were evaporated to afford compound 10-8 (350 mg, 0.996 mmol, 52%) as a white solid.

¹H-NMR (400 MHz, CDCl₃) δ: 1.38 (s, 9H), 1.88 (d, J=1.6 Hz, 3H), 2.70-3.01 (m, 4H), 5.11 (s, 1H), 7.03-7.11 (m, 1H), 7.13-7.20 (m, 1H), 7.34-7.22 (m, 2H), 9.57 (s, 1H).

### Step 5: Synthesis of Compound 10-9

To a solution of methyltriphenylphosphonium bromide (800 mg, 2.49 mmol) in toluene (8 ml) was added 1.00 mol/l of t-BuOK solution in THF (2.29 ml, 2.29 mmol) at room temperature. After stirring for 1 h at the same temperature, to the mixture was added a solution of compound 10-8 (356 mg, 0.996 mmol) in toluene (4 ml) at 10° C. and the mixture was stirred for 1 h at room temperature. The reaction mixture was treated with saturated aqueous NH₄Cl, and the aqueous layer was extracted with AcOEt. The combined organic layers were washed with brine, dried over Na₂SO₄ and filtered. The filtrate was concentrated in vacuo. The crude product whs added to a silica get column and eluted with Hexane/EtOAc 0% to 20%. Collected fractions were evaporated to afford compound 10-9 (304 mg, 0.855 mmol, 80%) as a colorless oil.

¹H-NMR (400 MHz, CDCl₃) δ: 1.35 (s, 9H), 1.90 (d, J=2.4 Hz, 3H), 2.41-2.59 (m, 2H), 2.82-3.01 (m, 2H), 4.99 (s, 1H), 5.23 (d, J=17.2 Hz, 1H), 5.35 (d, J=10.5 Hz, 1H), 5.76 (dd, J=17.2, 10.5 Hz, 1H), 7.02 (dd, J=13.1, 8.2 Hz, 1H), 7.11 (t, J=7.1 Hz, 1H), 7.29-7.19 (m, 2H).

### Step 6: Synthesis of Compound 10-11

A solution of compound 10-9 (304 mg, 0.855 mmol) in 4 mol/L HCl in dioxane (2.14 ml, 8.55 mmol) was stirred for 30 min at room temperature. The reaction mixture was treated with aqueous NaHCO₃ and the aqueous layer was extracted with AcOEt. The combined organic layers were washed with H₂O and brine, dried over Na₂SO₄ and filtered. The filtrate was concentrated under vacuum to give compound 10-10 as a brown oil that was used for the next step without purification.

To a solution of compound 10-10 in CH₂Cl₂ (2 ml) was added benzoyl isothiocyanate (0.176 ml, 1.28 mmol) at room temperature. After stirring for 15 h at the same temperature the reaction mixture was concentrated. The resulting reaidue was added to a silica gel column and eluted with Hexane/EtOAc 0% to 20%. Collected fractions were evaporated to afford compound 10-11 (347 mg, 0.829 mmol, 97%) as a white amorphous.

¹H-NMR (400 MHz, CDCl₃) δ: 1.56 (s, 9H), 2.16 (d, J=2.3 Hz, 3H), 2.56-2.73 (m, 2H), 2.85-3.01 (m, 2H), 5.42 (d, J=17.3 Hz, 3H), 5.46 (d, J=10.5 Hz, 1H), 5.89 (dd, J=17.3, 10.5 Hz, 1H), 7.02-7.08 (m, 1H), 7.14-7.19 (m, 1H), 7.21-7.34 (m, 2H), 7.52 (t, J=7.4 Hz, 2H), 7.63 (t, J=7.4 Hz, 1H), 7.86 (d, J=7.4 Hz, 2H), 8.85 (s, 1H), 11.55 (s, 1H).

### Step 7: Synthesis of Compound 10-12

To a solution of Iodine (421 mg, 1.66 mmol) in MeCN (9 ml) was added compound 10-11 (347 mg, 0.829 mmol) in MeCN (5 ml) at 0° C. After stirring for 2 h at the game temperature, the reaction mixture was treated with aqueous NaHCO₃ and Na₂S₂O₃. The aqueous layer was extruded with AcOEt. The combined organic layers were washed with brine, dried over Na₂SO₄, filtered and concentrated. The crude product was added to a silica gel column and eluted with hexane/EtOAc 0% to 15%. Collected fractions were evaporated to afford compound 10-12 (343 mg, 0.630 mmol, 76%) as a white amorphous.

¹H-NMR (400 MHz, CDCl₃) δ: 1.83 (s, 3H), 2.74-2.99 (m, 3H), 3.29 (t, J=11.1 Hz, 1H), 3.36-3.47 (m, 2H), 3.74 (dd, J=10.0, 2.5 Hz, 1H), 7.12 (dd, J=12.7, 8.2 Hz, 1H), 7.20 (t, J=7.7 Hz, 1H), 7.20-7.41 (m, 2H), 7.44 (t, J=7.5 Hz, 2H), 7.52 (t, J=7.5 Hz, 1H), 8.22 (d, J=7.5 Hz, 2H).

### Step 8: Synthesis of Compound 10-13

To a solution of compound 10-12 (313 mg, 0.630 mmol) in toluene (5 ml) were added Bu₃SnH (0.251 ml, 0.945 mmol) and AlBN (10.4 mg, 0.0630 mmol) at room temperature. After stirring for 1 h at 80° C. the reaction mixture was concentrated. The resulting residue was added to an amino silica gel column and eluted with Hexane/EtOAc 0% to 20%. Collected fractions were evaporated to afford compound 10-13 (212 mg, 0.507 mmol, 80%) as a white amorphous.

¹H-NMR (400 MHz, CDCl₃) δ: 1.33 (d, J=6.9 Hz, 3H), 1.85 (s, 3H), 2.81 (q, J=12.5 Hz, 1H), 2.88-3.01 (m, 1H), 3.23 (q, J=6.9 Hz, 1H), 3.35 (q, , J=12.5 Hz, 1H), 7.10 (dd, J=12.5, 8.2 Hz, 1H), 7.17 (t, J=7.6 Hz, 1H), 7.32-7.38 (m, 2H), 7.43 (t, J=7.5 Hz, 2H), 7.51 (t, J=7.5 Hz, 1H), 8.22 (d, J=7.5 Hz, 2H). 12.02 (br s, 1H).

### Step 9: Synthesis of Compound 10-14

To a solution of 1-5 (212 mg, 0.507 mmol) in MeOH (1 ml) and THF (1 ml) was added hydrazine hydrate (0.246 ml, 5.07 mmol) at room temperature. After stirring for 13 h at the same temperature, the reaction mixture was concentrated. The resulting residue was added to an amino silica gel column and eluted with Hexane/EtOAc 10% to 50%. Collected fractions were evaporated to afford compound 10-14 (150 mg, 0.177 mmol, 94%) as a white amorphous.

¹H-NMR (400 MHz, CDCl₃) δ: 1.21 (d, J=6.8 Hz, 3H), 1.72 (s, 3H), 2.67-2.81 (m, 3H), 3.20 (q, J=6.8 Hz, 1H), 3.27-3.42 (m, 1H), 7.02 (dd, J=12.7, 8.0 Hz, 1H), 7.11 (t, J=7.5 Hz, 1H), 7.22-7.28 (m, 1H), 7.32 (t, J=8.0 Hz, 1H).

### Step 10: Synthesis of Compound 10-16

To a solution of 1-6 in TFA (1.3 ml) was added sulfuric acid (0.318 ml, 5.96 mmol) at −20° C. After stirring for 5 min at 0° C., to the reaction mixture was added to HNO₃ (0.0320 ml, 0.716 mmol) at −20° C. After stirring for 20 min at 0° C. the reaction mixture was treated with aqueous K₂CO₃. The aqueous layer was extracted with AcOEt and the organic layer was dried over Na₂SO₄. The filtrate was concentrated under vacuum to give 1-15 as a yellow oil that was used for the next step without purification.

To a solution of compound 10-15 in THF (2 ml) were added Boc₃O (0.331 ml, 1.43 mmol) and DMAP (23.3 mg, 0.190 mmol) at room temperature. After stirring for 30 min at the same temperature, the mixture was concentrated under vacuum. The crude product was added to a silica gel column and eluted with hexane/EtOAc 0% to 20%. Collected fractions were evaporated to afford 10-16 (2.53 mg, 0.450 mmol, 95%) as a white amorphous.

¹H-NMR (400 MHz, CDCl₃) δ: 1.36 (d, J=6.9 Hz, 3H), 1.55 (s, 18H), 1.85 (d, J=1.6 Hz, 3H), 2.71-2.94 (m, 3H), 3.21 (q, J=15.7 Hz, 1H), 3.33 (q, J=6.9 Hz, 1H), 7.20 (dd, J=11.2, 9.0 Hz, 1H), 8.22-8.15 (m, 1H), 8.57 (dd, J=6.8, 2.8 Hz, 1H).

### Step 11: Synthesis of Compound 10-17

To a solution of compound 10-16 (252 mg, 0.450 mmol) in EtOH (2 ml), THF (1 ml) and H₂O (1 ml) were added NH₄Cl (289 mg, 5.40 mmol) and Fe (201 mg, 3.60 mmol) at room temperature. After stirring for 1 h at 60° C., the mixture was treated with H₂O and filtrated through a pad of Celite (Registered trademark). The aqueous layer was extracted with AcOEt, and the organic layer was dried over Na₂SO₄, filtered and concentrated. The crude product was added to a silica gel column and eluted with hexane/EtOAc 0% to 30%. Collected fractions were evaporated to afford compound 10-17 (177 mg, 0.334 mmol, 74%) as a white amorphous.

¹H-NMR (400 MHz, CDCl₃) δ: 1.27 (d, J=7.0 Hz, 3H), 1.54 (s, 18H), 1.86 (d, J=2.5 Hz, 3H), 2.63-2.87 (m, 3H), 3.28-3.47 (m, 2H), 3.61 (s, 2H), 6.66-6.50 (m, 1H), 6.82 (dd, J=12.5, 8.5 Hz, 1H), 7.03 (dd, J=7.0, 2.8 Hz, 1H).

### Step 10: Synthesis of Compound 10-18

To a solution of compound 10-17 (50.0 mg, 0.0940 mmol) in DMF (1 ml) were added 5-flouropicolinic acid (13.3 mg, 0.0940 mmol), HATU (43.1 mg, 0.113 mmol) and DIPEA (0.0330 ml, 0.189 mmol) at room temperature. After stirring for 30 min at the same temperature, the reaction mixture was treated with H₂O. The aqueous layer was extracted with AcOEt, and the organic layer was dried over Na₂SO₄, filtered and concentrated. The crude product was added to a silica gel column and eluted with hexane/EtOAc 0% to 30%. Collected fraction were evaporated to afford compound 10-18 (58.0 mg, 0.0890 mmol, 94%) as a colorless oil.

¹H-NMR (400 MHz, CDCl₃) δ: 1.29 (d, J=7.0 Hz, 3H), 1.60 (s, 18H), 1.92 (s, 3H), 2.70-2.93 (m, 3H), 3.33-3.49 (m, 2H), 7.08 (dd, J=12.2, 8.8 Hz, 1H), 7.58 (td, J=8.8, 2.8 Hz, 1H), 7.72 (dd, J=7.1, 2.8 Hz, 1H), 8.44-8.31 (m, 3H), 10.03 (s, 1H).

### Step 11: Synthesis of Compound I-63

To a solution of compound 10-18 (58.0 mg, 0.0890 mmol) in CH₂Cl₂ (0.7 ml) was added TFA (0.220 ml, 2.03 mmol) at room temperature. After stirring for 17 h at the same temperature, the reaction mixture was treated with aqueous K₂CO₃. The aqueous layer was extracted with AcOEt and the organic layer was dried over Na₂SO₄, filtered and concentrated to afford compound I-63 (34.0 mg, 0.0750 mmol, 85%) as a white solid.

¹H-NMR (400 MHz, CDCl₃) δ: 1.27 (d, J=7.0 Hz, 3H), 1.74 (s, 3H), 2.67-2.85 (m, 3H), 3.24-3.42 (m, 2H), 7.06 (dd, J=11.9, 8.7 Hz, 1H), 7.44 (dd, J=7.0, 2.6 Hz, 1H), 7.59 (td, J=8.7, 2.8 Hz, 1H), 7.93-7.87 (m, 1H), 8.33 (dd, J=8.7, 4.6 Hz, 1H), 8.46 (d, J=2.6 Hz, 1H), 9.70 (s, 1H).

### EXAMPLE 11

### Synthesis of Compound I-40

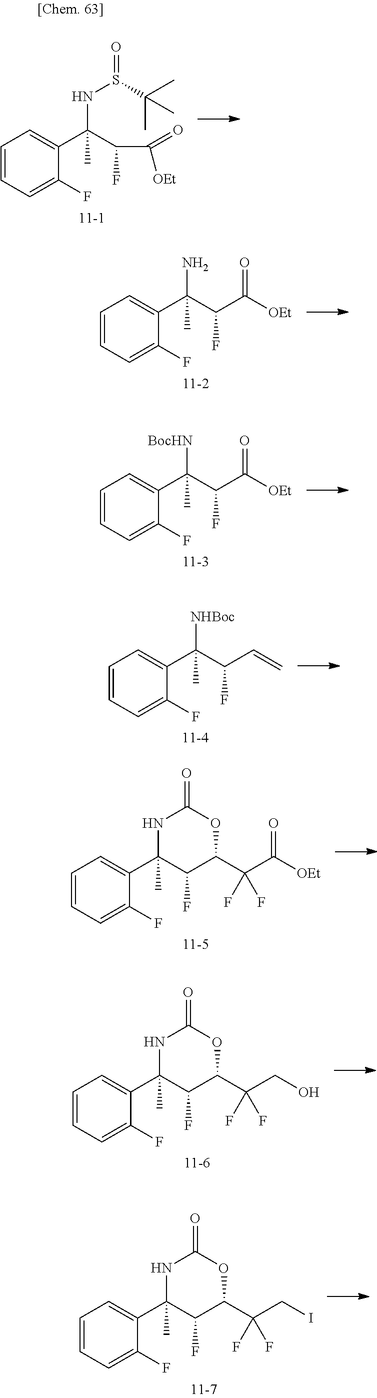

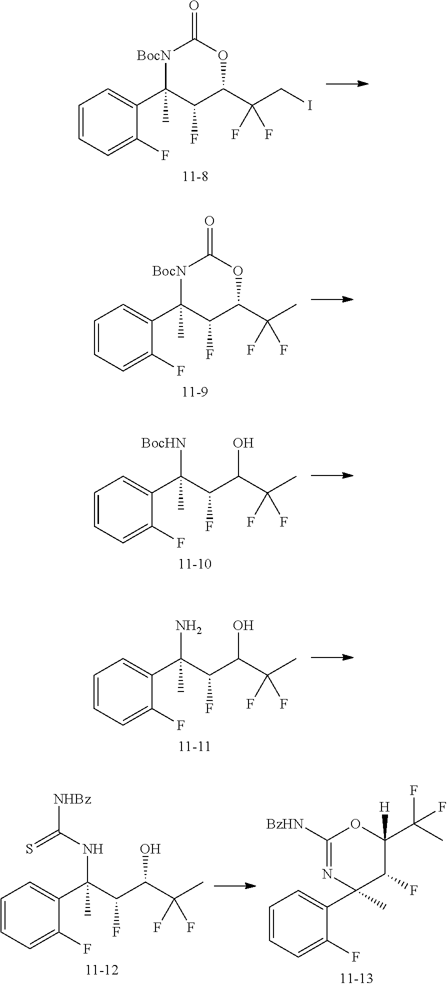

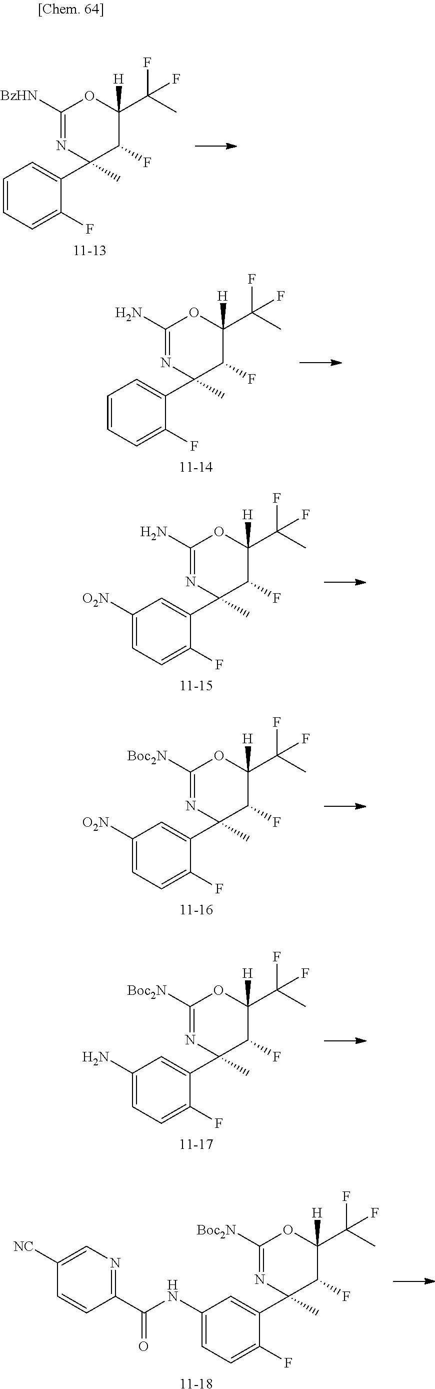

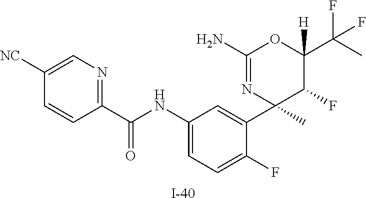

### Step 1

To a solution or compound 11-1 (16.1 g, 46.5 mmol) in methanol (160 ml) was added HCl-dioxane (4M, 16.3 ml, 65.0 mmol) at room temperature. After being stirred for 1.5 h at room temperature, the reaction mixture was concentrated and quenched with saturated aqueous NaHCO₃. The aqueous phase was extracted with AcOEt. The organic layer was dried over Na₂SO₄ and concentrated to afford compound 11-2 (12.8 g, quant). The obtained compound 11-2 was used in the next reaction without further purification.

LC/MS(Shimadzu): RT 0.80, MS calcd for 244.11 (M+H⁺), found 244.00.

### Step 2

To a solution of compound 11-2 (1.28 g, 48.5 mmol) in methanol (55 ml) was added Boc₂O (32.4 ml, 140 mmol) at room temperature. After being stirred for 4 h at (60° C., the reaction mixture was concentrated. The residue was purified by silica gel chromatography to afford compound 11-3 (21.35 g, quant., including Boc₂O, compound 3:Boc₂O=1:0.8).

¹H-NMR (CDCl₃) δ: 1.03 (brs, 3H), 1.39 (brs, 9H), 1.96 (brs, 3H), 4.06 (brs, 2H), 5.34 (d, J=47.2 Hz, 1H), 5.70 (brs, 1H), 7.04 (m, 1H), 7.11 (m, 1H), 7.25-7.35 (m, 2H).

### Step 3

To a solution of compound 11-3 (11.4 g, 24.7 mmol, including Boc₂O) in CH₂Cl₂ (110 ml) wan added DIBAL (1.02 M in toluene, 107 ml, 109 mmol) at −65° C. After being stirred for 50 min at −65° C. the reaction mixture was quenched with AcOEt and Rochelle's salt (93 g, 331 mmol) in H₂O. After being stirred for 1.5 h at room temperature, the aqueous phase was extracted with AcOEt. The organic layer was dried over Na₂SO₄ and concentrated to afford compound 11-4 (7.87 g, quant). The obtained compound 11-4 was used in the next reaction without further purification.

¹H-NMR (CDCl₃) δ: 1.39 (s, 9H), 1.71 (s, 3H), 5.07 (s, 1H), 5.53 (d, J=46.8 Hz, 1H), 7.08 (m, 1H), 7.16 (m, 1H), 7.30 (m, 1H), 7.37 (m, 1H), 9.55 (d, J=9.0 Hz, 1H).

### Step 4

To a solution of compound 11-4 (7.87 g, 24.7 mmol) and ethyl 2-bromo-2,2-difluoroacetate (15.0 g, 74.1 mmol) in THF (150 ml) was added zinc (4.84 g, 74.1 mmol) at room temperature. After being stirred for 1.5 h at 70° C. the reaction mixture was cooled to 0° C. and quenched with saturated aqueous NH₄Cl. The resulting mixture was filtered through a pad of Celite (Registered trademark) and washed with AcOEt. The aqueous phase was extracted with AcOEt. The organic layer was dried over Na₂SO₄ and concentrated. The residue was purified by silica gel chromatography to afford compound 11-5 (1.40 g, 4.01 mmol, 16%, 4 steps).

¹H-NMR (CDCl₃) δ: 1.34 (t, J=7.2 Hz, 3H), 1.78 (s, 3H), 4.34 (q, J=7.2 Hz, 2H), 4.40 (ddd, J=28.5, 13.7, 6.3 Hz, 1H), 5.49 (d, J=47.4 Hz, 1H), 5.83 (s, 1H), 7.15 (m, 1H), 7.28 (m, 1H), 7.42 (m, 1H), 7.51 (m, 1H).

### Step 5

To a solution of compound 11-6 (1.10 g, 4.01 mmol) in THF (28 ml) was added LiBH₄ (176 mg, 8.02 mmol) at room temperature. After being stirred for 45 min at room temperature, the reaction mixture was quenched with saturated aqueous NH₄Cl. The aqueous phase was extracted with AcOEt. The organic layer was dried over Na₂SO₄ and concentrated. The residue was purified by silica gel chromatography to afford compound 11-6 (740 mg, 2.41 mmol, 60%).

¹H-NMR (CDCl₃) δ: 1.79 (s, 3H), 1.97 (t, J=7.1 Hz, 1H), 3.84-4.11 (m, 2H), 4.28 (ddd, J=30.0, 13.2, 6.4 Hz, 1H), 5.47 (d, J=46.9 Hz, 1H), 5.69 (s, 1H), 7.15 (m, 1H), 7.27 (m, 1H), 7.41 (m, 1H), 7.50 (m, 1H).

### Step 6

To a solution of compound 11-6 (850 mg, 2.77 mmol), PPh₃ (2.90 g, 11.1 mmol) and imidazole (753 mg, 11.1 mmol) in THF (17 ml) was added iodine (2.81 g, 11.1 mmol) at room temperature. After being stirred for 16 h at 80° C., the reaction mixture was cooled to 0° C. and quenched with aqueous Na₂S₂O₃. The aqueous phase was extracted with AcOEt. The organic layer was dried over Na₂SO₄ and concentrated. The residue was purified by silica gel chromatography to afford compound 11-7 (1.07 g, 2.57 mmol, 93%).

¹H-NMR (CDCl₃) δ: 1.79 (g, 3H), 3.56-3.74 (m, 2H), 4.29 (dt, J=29.5, 7.6 Hz, 1H), 5.41 (d, J=47.2 Hz, 1H), 5.72 (s, 1H), 7.16 (m, 1H), 7.29 (m, 1H), 7.43 (m, 1H), 7.50 (m, 1H).

### Step 7

To a solution of compound 11-7 (1.07 g, 2.57 mmol) and Boc₂O (1.19 ml, 5.13 mmol) in CH₂Cl₂ (20 ml) was added DMAP (157 mg, 1.28 mmol) at room temperature. After being stirred for 45 min at room temperature, the reaction mixture was concentrated. The residue was purified by silica gel chromatography to afford compound 11-8 (1.33 g, 2.57 mmol, 100%).

¹H-NMR (CDCl₃) δ: 1.52 (s, 9H), 1.97 (s, 3H), 3.56-3.74 (m, 2H), 4.31 (dt, J=30.6, 7.6 Hz, 1H), 5.32 (d, J=46.9 Hz, 1H), 7.14 (m, 1H), 7.27 (m, 1H), 7.41 (m, 1H), 7.48 (m, 1H).

### Step 8

To a solution of compound 11-8 (1.33 g, 2.57 mmol) and n-Bu₃SnH (1.64 ml, 6.16 mmol) in toluene (20 ml) was added AlBN (63.2 mg, 385 μmol) at room temperature. After being stirred for 1 h at 80° C., the reaction mixture was cooled to room temperature and concentrated. The residue was purified by silica gel chromatography to afford compound 11-9 (717.1 mg 1.83 mmol, 71%).

¹H-NMR (CDCl₃) δ: 1.52 (s, 9H), 1.75 (t, J=19.8 Hz, 3H), 1.97 (s, 3H), 4.05 (ddd, J=30.7, 10.1, 4.8 Hz, 1H), 5.27 (d, J=46.9 Hz, 1H), 7.13 (m, 1H), 7.25 (m, 1H) 7.39 (m, 1H), 7.48 (m, 1H).

### Step 9

To a solution of compound 11-9 (717.1 mg 1.83 mmol) in EtOH (12 ml) and H₂O (6 ml) was added Ba(OH)₂ (1.73 g, 5.50 mmol) at room temperature. After being stirred for 1.5 h at room temperature, the reaction mixture was quenched with aqueous citric acid. The aqueous phase was extracted with AcOEt. The organic layer was dried over Na₂SO₄ and concentrated. The residue was washed with AeOEt and hexane and insoluble matter was filtered. The filtrate was concentrated and the residue was purified by silica gel chromatography to afford compound 11-10 (630.1 mg, 1.73 mmol, 94%).

¹H-NMR (CDCl₃) δ: 1.42 (s, 9H), 1.61 (t, J=19.3 Hz, 3H), 2.02 (s, 3H), 2.67 (dd, J=9.3, 3.8 Hz, 1H), 3.62 (brs, 1H), 5.14 (d, J=41.5 Hz, 1H), (6.27 (brs, 1H), 7.05 (m, 1H), 7.16 (m, 1H), 7.29 (m, 1H), 7.39 (m, 1H).

### Step 10

To a solution of compound 11-10 (630.1 mg, 1.73 mmol) and in MeOH (6 ml) was added HCl-dioxane (4M, 1.73 ml, 6.90 mmol) at room temperature. After being stirred for 2.5 h at 50° C., the reaction mixture was concentrated and quenched with saturated aqueous NaHCO₃. The aqueous phase was extracted with AcOEt. The organic layer was dried over Na₂SO₄ and concentrated. The obtained compound 11-11 was used in the next reaction without further purification.

¹H NMR (CDCl₃) δ: 1.64 (td, J=19.6, 1.5 Hz, 3H), 1.76 (s, 3H), 3.46 (dt, J=29.9, 9.5 Hz, 1H), 5.19 (d, J=45.7 Hz, 1H), 7.09 (m, 1H), 7.23 (m, 1H), 7.35 (m, 1H), 7.57 (m, 1H).

### Step 11

To a solution of compound 11-11 in CH₂Cl₂ (3 ml) was added BzNCS (348 μl, 2.59 mmol) at room temperature. After being stirred for 2.5 h at room temperature, the reaction mixture was concentrated. The residue was purified by silica gel chromatography to afford compound 11-12 (630.9 mg, 1.47 mmol, 85%, 2 steps).

¹H-NMR (CDCl₃) δ: 1.69 (t, J=19.2 Hz, 3H), 2.31 (s, 3H), 2.74 (dd, J=9.9, 2.9 Hz, 1H), 3.85 (m, 1H), 5.41 (d, J=44.4 Hz, 1H), 7.08 (m, 1H), 7.18 (m, 1H), 7.33 (m, 1H), 7.47 (m, 1H), 7.52 (m, 2H), 7.63 (m, 1H), 7.87 (m, 2H), 8.91 (s, 1H), 11.80 (s, 1H).

### Step 12

To a solution of compound 11-12 (630.9 mg, 1.47 mmol) in CH₃CN (12 ml) was added WSCD hydrochloride (565 mg, 2.95 mmol) at room temperature. After being stirred for 1.5 h at 50° C., the reaction mixture was cooled to room temperature and quenched with H₂O). The aqueous phase was extracted with AcOEt. The organic layer was dried over Na₂SO₄ and concentrated. The residue was purified by silica gel chromatography to afford compound 11-13 (564.0 mg, 1.43 mmol, 97%).

¹H-NMR (CDCl₃) δ: 1.83 (td, J=19.7, 1.9 Hz, 3H), 1.89 (s, 3H), 4.11 (ddd, J=29.1, 10.0, 5.0 Hz, 1H), 5.52 (d, J=47.2 Hz, 1H), 7.17 (m, 1H), 7.24 (m, 1H), 7.38-7.48 (m, 4H), 7.53 (m, 1H), 8.27 (m, 2H) 11.80 (s, 1H).

### Step 13

To a solution of compound 11-13 (554.0 mg, 1.40 mmol) in MeOH (22 ml) was added K₂CO₃ (1.17 g, 8.43 mmol) at room temperature. After being stirred for 3 h at 80° C., the reaction mixture was cooled to room temperature and quenched with H₂O. The aqueous phase was extracted with AcOEt. The organic layer was dried over Na₂SO₄ and concentrated. The residue was purified by silica gel chromatography to alford compound 11-14 (374.5 mg, 1.29 mmol, 92%).

¹H-NMR (CDCl₃) δ: 1.66 (s, 3H), 1.71 (t, J=19.7 Hz, 3H), 3.83 (ddd, J=30.0, 10.8, 4.4 Hz, 1H), 4.29 (brs, 2H), 5.35 (d, J=48.2 Hz, 1H), 7.05 (m, 1H), 7.17 (m, 1H), 7.29 (m, 1H), 7.41 (m, 1H).

### Step 14

To a solution of compound 11-14 (360.0 mg, 1.24 mmol) in TFA (4 ml) was added H₂SO₄ (1 ml) at −20° C. After being stirred for 5 min at 0° C. the reaction mixture was cooled to −20° C. and HNO₃ (83 μl, 1.86 mmol) was added. After being stirred for 15 min al 0° C., the reaction mixture was quenched with aqueous K₂CO₃. The aqueous phase was extracted with AcOEt. The organic layer was dried over Na₂SO₄ and concentrated. The residue was purified by silica gel chromatography to afford compound 11-15 (530 mg, quant.).

¹ H-NMR (CDCl₃) δ: 1.77 (t, J=19.7 Hz, 3H), 1.87 (s, 3H), 4.05 (m, 1H), 5.46 (d, J=46.6 Hz, 1H), 7.34 (m, 1H), 8.27-8.36 (m, 2H).

### Step 15

To a solution of compound 11-15 (530 mg, quant.) and Boc₂O (864 μl, 3.72 mmol) in CH₂Cl₂ (7 ml) was added DMAP (182 mg, 1.49 mmol) at room temperature. After being stirred for 1 h at room temperature, the reaction mixture was concentrated. The residue was purified by silica gel chromatography to afford compound 11-16 (603.8 mg, 1.13 mmol, 91%, 2 steps).

¹H-NMR (CDCl₃) δ: 1.52 (s, 18H), 1.73 (s, 3H), 1.74 (m, 3H), 3.88 (ddd, J=28.9, 10.3, 4.3 Hz, 1H), 5.39 (d, J=47.4 Hz, 1H), 7.20 (m, 1H), 8.27 (m, 1H), 8.51 (m, 1H).

### Step 16

To a solution of compound 11-16 (603.8 mg, 1.13 mmol) in THF (8 ml) and MeOH (4 ml) was added Pd/C (60.0 mg) at room temperature. After being stirred for 3.5 h under H₂ atmosphere at room temperature, the reaction mixture was filtered through a pad of Celite (Registered trademark), and the residue was washed with AcOEt. The filtrate was concentrated, and the residue was purified by silica gel chromatography to afford compound 11-17. The obtained compound 11-17 was further purified by trituration from AcOEt/hexane to give compound 11-17 (482.9 mg, 955 μmol, 85%).

¹H-NMR (CDCl₃) δ: 1.52 (s, 18H), 1.69 (s, 3H), 1.72 (t, J=19.6 Hz, 3H), 3.56 (brs, 2H), 4.02 (m, 1H), 5.37 (d, J=47.8 Hz, 1H), 6.57 (m, 1H), 6.81 (m, 1H), 6.87 (m, 1H).

### Step 17

To a solution of compound 11-17 (70.0 mg, 138 μmol), 5-cyanopicolinic acid hydrate (27.6 mg, 166 μmol) and diisopropylethylamine (48 μl, 277 μmol) in DMF (2 ml) was added HATU (63.2 mg, 166 μmol) at room temperature. After being stirred for 50 min at room temperature, the reaction mixture was quenched with saturated aqueous NH₄Cl. The aqueous phase was extracted with AcOEt. The organic layer was dried over Na₂SO₄ and concentrated. The residue was purified by silica gel chromatography to afford compound 11-18 (91.6 mg, quant.).

¹H NMR (CDCl₃) δ: 1.56 (s, 18H), 1.73 (t, J=19.7 Hz, 3H), 1.76 (s, 3H), 4.01 (ddd, J=29.6, 10.2, 1.4 Hz, 1H), 5.40 (d, J=47.6 Hz, 1H), 7.16 (m, 1H), 7.53 (m, 1H), 8.21 (d, J=8.2 Hz, 1H), 8.37 (m, 1H), 8.43 (d, J=8.2 Hz, 1H), 8.80 (s, 1H), 9.96 (s, 1H).

### Step 18

Compound 11-18 (91.6 mg) was solved in formic acid (1 ml, 26.1 mmol) at room temperature. After being stirred for 8 h at room temperature, the reaction, mixture was quenched with aqueous K₂CO₃. The aqueous phase was extracted with AcOEt. The organic layer was dried over Na₂SO₄ and concentrated. The residue waa purified by trituration from ArOEt/hexane to give pure compound I-40 (52.2 mg, 120 μmol, 2 steps).

¹H-NMR (CDCl₃) δ: 1.67 (s, 3H), 1.72 (t, J=19.7 Hz, 3H), 3.88 (ddd, J=29.9, 10.8, 4.4 Hz, 1H), 4.33 (brs, 2H), 5.38 (d, J=48.1 Hz, 1H), 7.12 (m, 1H), 7.55 (m, 1H), 8.02 (m, 1H), 8.21 (d, J=8.3 Hz, 1H), 8.43 (d, J=8.3 Hz, 1H), 8.90 (s, 1H), 9.87 (s, 1H).

### EXAMPLE 12

### Synthesis of Compound I-82

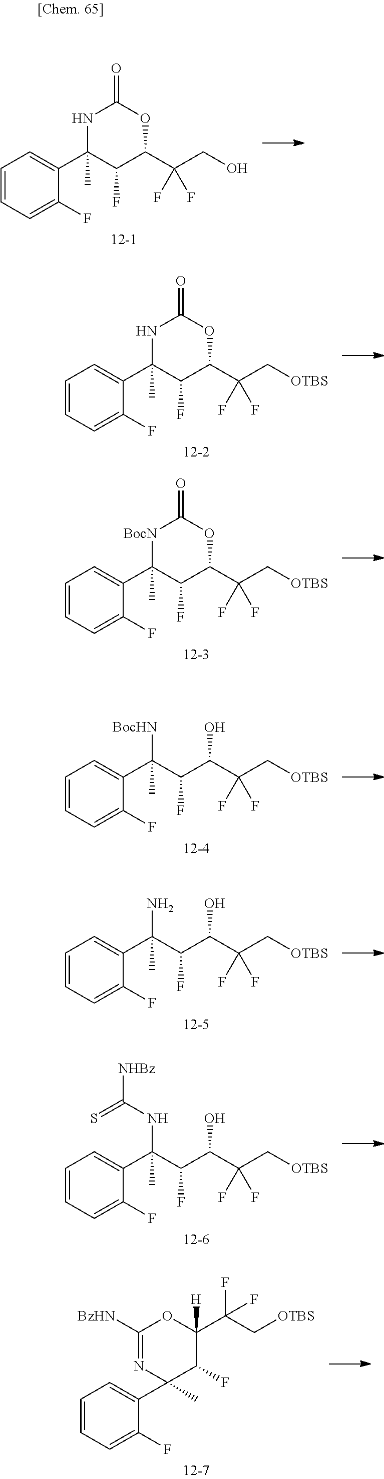

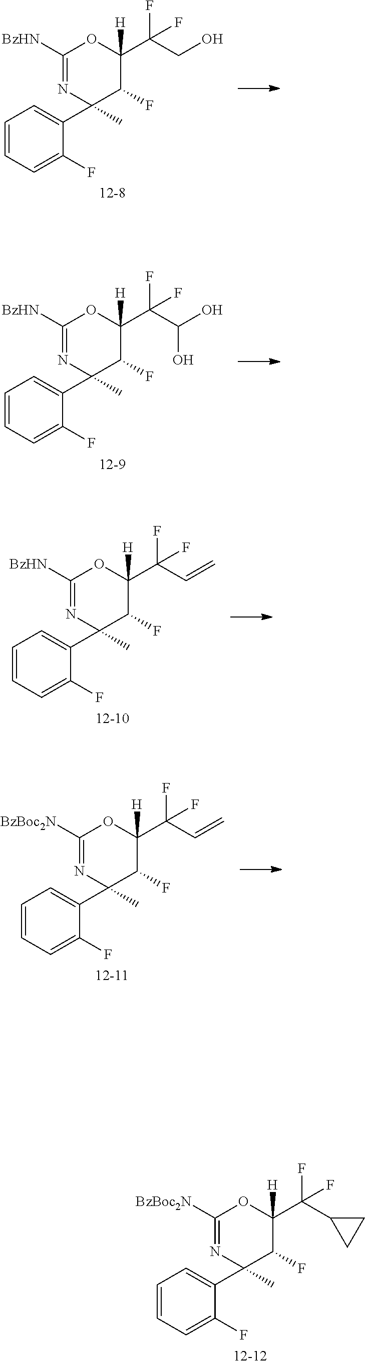

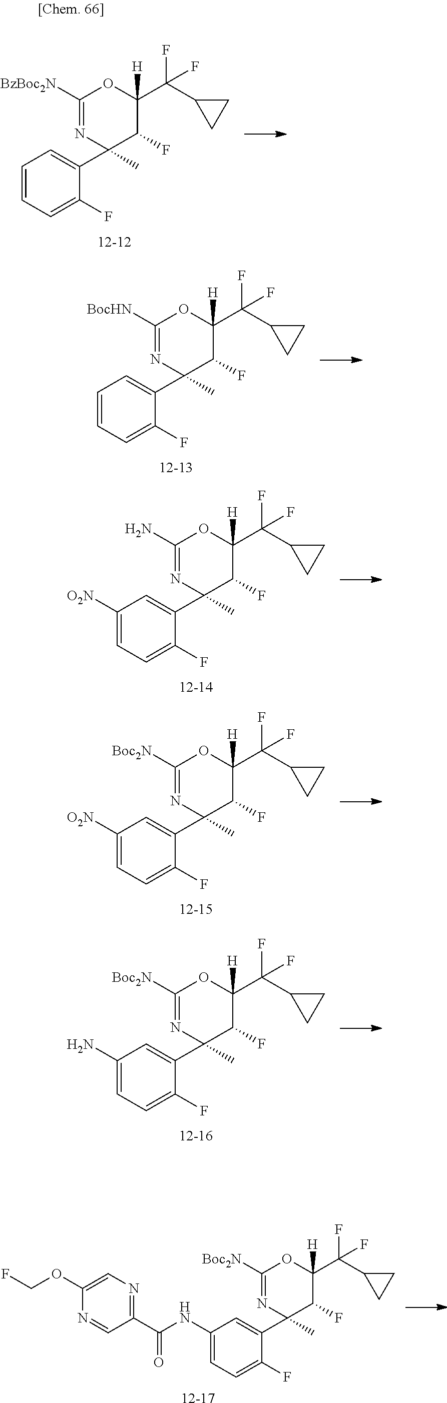

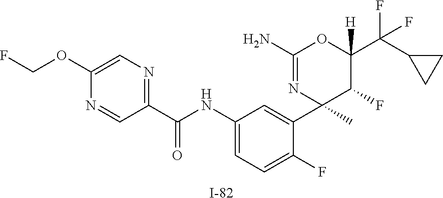

### Step 1

To a solution of compound 12-1 (1.50 g, 4.88 mmol) and imidazole (798 mg, 11.7 mmol) in DMF (15 ml) was added TBSCl (883 mg, 5.86 mmol) at room temperature. After stirring for 45 min at room temperature, the reaction mixture was quenched with saturated aqueous NaHCO₃. The aqueous phase was extracted with AcOEt. The organic layer was dried over Na₂SO₄ and concentrated to afford compound 12-2. The obtained compound 12-2 was used in the next reaction without further purification.

LC/MS(Shimadzu): RT 2.59, MS calcd for 422.18 (M°H⁺), found 422.00.

### Step 2

To a solution of compound 12-2 and Boc₂O (6.57 ml, 28.3 mmol) in CH₂Cl₂ (20 ml) was added DMAP (238 mg, 1.95 mmol) at room temperature. After being stirred for 1 h at room temperature, the reaction mixture was concentrated. The residue was purified by silica gel chromatography to afford compound 12-3 (2.60 g, quant.)

¹H-NMR (CDCl₃) δ: −0.04 (s, 3H), −0.02 (s, 3H), 0.69 (s, 9H), 1.52 (s, 9H), 1.98 (m, 3H), 3.76 (m, 1H), 3.94 (ddd, J=24.1, 11.8, 6.0 Hz, 1H), 4.34 (m, 1H), 5.38 (d, J=46.9 Hz, 1H), 7.09 (m, 1H), 7.23 (m, 1H), 7.36 (m, 1H), 7.48 (m, 1H).

### Step 3

To a solution of compound 12-3 (2.60 g) m MeOH (25 ml) was added K₂CO₃ (2.70 g, 19.5 mmol) at room temperature. After being stirred for 45 min at room temperature, the reaction mixture was quenched with saturated aqueous NH₄Cl. The aqueous phase was extracted with AcOEt. The organic layer was dried over Na₂SO₄ and concentrated to afford compound 12-4. The obtained compound 12-4 was used in the next reaction without, further purification.

LC/MS(Shimadzu): RT 2.94, MS calcd for 496.25 (M+H⁺), found 496.25.

### Step 4

To a solution of compound 12-4 in CH₂Cl₂ (50 ml) was added TFA (5 ml, 64.9 mmol) at room temperature. After being stirred for 1 h at room temperature, the reaction mixture was quenched with saturated aqueous NaHCO₃ and K₂CO₃. The aqueous phase was extracted with AcOEt. The organic layer was dried over Na₂SO₄ and concentrated. The residue was purified by silica gel chromatography to afford compound 12-5 (1.53 g, 3.87 mmol, 79%, 4 steps).

¹H-NMR (CDCl₃) δ: −0.05 (s, 3H), −0.02 (s, 3H), 0.74 (s, 9H), 1.74 (s, 3H), 3.61-3.77 (m, 2H), 3.91 (ddd, J=23.5, 11.5, 7.7 Hz, 1H), 5.29 (d, J=45.8 Hz, 1H), 7.06 (m, 1H), 7.20 (m, 1H), 7.32 (m, 1H), 7.59 (m, 1H).

### Step 5

To a solution of compound 12-5 (1.53 g, 3.87 mmol) in CH₂Cl₂ (6 ml) was added B₂NCS (780 μl, 5.80 mmol) at room temperature. After being stirred for 1 h at room temperature, the reaction mixture was concentrated. The residue was purified by silica gel chromatography to afford compound 12-6 (2.07 g, including compound 12-7).

¹H-NMR (CDCl₃) δ: 0.01 (s, 3H), 0.04 (s, 3H), 0.81 (s, 9H), 2.35 (brs, 3H), 3.22 (dd, J=11.0, 2.5 Hz, 1H), 3.76 (td, J=11.8, 5.5 Hz, 1H), 3.96-4.15 (m, 2H), 5.38 (d, J=44.2 Hz, 1H), 7.06 (m, 1H), 7.16 (m, 1H), 7.31 (m, 1H), 7.48 (m, 1H), 7.52 (m, 2H), 7.63 (m, 1H), 7.86 (m, 2H), 8.89 (s, 1H), 11.92 (s, 1H).

### Step 6

To a solution of compound 12-6 (2.07 g) in CH₃CH (21 ml) was added WSCD hydrochloride (1.42 g, 7.41 mmol) at room temperature. After being stirred for 1.5 h at 50° C., the reaction mixture was cooled to room temperature and quenched with H₂O. The aqueous phase was extracted with AcOEt. The organic layer was dried over Na₂SO₄ and concentrated to afford compound 12-7. The obtained compound 12-7 was used in the next reaction without further purification.

LC/MS(Shimadzu): RT 3.06, MS calcd for 525.22 (M+H⁺), found 525.20.

### Step 7

To a solution of compound 12-7 in THF (20 ml) was added TBAF (1 M in THF, 7.41 ml, 7.41 mmol) ut room temperature. After being stirred for 10 min at room temperature, the reaction mixture was quenched with saturated aqueous NH₄Cl. The aqueous phase was extracted with AcOEt. The organic layer was dried over Na₂SO₄ and concentrated. The residue was purified by silica gel chromatography to afford compound 12-8 (1.55 g, 3.78 mmol, 98%, 3 steps)

¹H-NMR (CDCl₃) δ: 1.90 (s, 3H), 3.96 (ddd, J=15.7, 13.3, 7.9 Hz, 1H), 4.14 (m, 1H), 4.35 (ddd, J=29.5, 12.9, 6.4 Hz, 1H), 5.62 (d, J=46.9 Hz, 1H), 7.17 (m, 1H), 7.24 (m, 1H), 7.38-7.48 (m, 4H), 7.53 (m, 1H), 8.24 (m, 2H), 11.77 (brs, 1H).

### Step 8

To a solution of compound 12-8 (500 mg, 1.22 mmol) in CH₂Ch₂ (10 ml) was added Dess-Martin periodinane (1.03 g, 2.44 mmol) at room temperature. After being stirred for 1 h at room temperature, the reaction mixture was quenched with saturated aqueous NaHCO₃ and Na₂S₂O₃. The aqueous phase was extracted with AcOEt. The organic layer was dried over Na₂SO₄ and concentrated to afford compound 12-9. The obtained compound 12-9 was used in the next reaction without further purification.

LC/MS(Shimadzu): RT 1.86, MS calcd for 427.13 (M+H⁺), found 427.10.

Step 9

To a suspension of methyltriphenylphosphonium bromide (1.39 g, 3.90 mmol) in THF (8 ml) was added KHMDS (0.5 M in toluene, 7.31 ml, 3.65 mmol) at 0° C. After being stirred for 20 min at 0° C., compound 12-9 in THF (8 ml) was added. After being stirred for 3 h at room temperature, the reaction mixture was quenched with H₂O. The aqueous phase was extracted with AcOEt. The organic layer was dried over Na₂SO₄ and concentrated. The residue was purified by silica gel chromatography to afford compound 12-10 (255.3 mg, 628 μmol, 52%).

¹H-NMR (CDCl₃) δ: 1.88 (s, 3H), 4.18 (ddd, J=28.9, 9.3, 6.0 Hz, 1H), 5.54 (d, J=47.2 Hz, 1H), 5.64 (d, J=11.0 Hz, 1H), 5.87 (m, 1H), 6.08 (m, 1H), 7.17 (m, 1H), 7.24 (m, 1H), 7.38-7.48 (m, 4H), 7.52 (m, 1H), 8.27 (m, 2H), 11.78 (brs, 1H).

Step 10

To a solution of compound 12-10 (225.3 mg, 554 μmol) and Boc₂O (257 μl, 1.11 mmol) in CH₂Cl₂ (4.6 ml) was added DMAP (13.6 mg, 111 μmol) at room temperature. After being stirred for 40 min al room temperature, the reaction mixture was concentrated. The residue was purified by silica gel chromatography to afford compound 12-11 (289.6 mg, quant).

¹H-NMR (CDCl₃) δ: 1.38 (m, 3H), 1.48 (s, 9H), 4.05 (ddd, J=28.6, 9.9, 6 3 Hz, 1H), 5.31 (d, J=47.8 Hz, 1H), 5.60 (d, J=11.0 Hz, 1H), 5.77 (m, 1H), 5.99 (m, 1H), 7.06 (m, 1H), 7.17 (m, 1H), 7.31 (m, 1H), 7.40-7.47 (m, 3H), 7.55 (m, 1H), 7.76 (m, 2H).

### Step 11

In a test tube, to a mixture of aqueous NaOH (30%, 2 ml) and Et₂O (6 ml) was added 1-methyl-1-nitrosourea (571 mg, 2.77 mmol) at 0° C. After being stirred for 20 min at 0° C., the color of organic phase was turned to yellow, which indicated that an Et₂O solution of diazomethane was able to be prepared. In a separate flask, to a suspension of compound 12-11 (289.6 mg) and Pd(OAc)₂ (24.9 mg, 111 μmol) in Et₂O (6 ml) was added the Et₂O solution of diazomethane at 0° C. After being stirred for 15 min at 0° C., the reaction mixture was quenched with H₂O and AcOH. Saturated aqueous NaHCO₃ was added, and the aqueous phase was extracted with AcOEt. For organic layer was dried over Na₂SO₄ and concentrated. The residue was purified by silica gel chromatography to afford compound 12-12 (275.7 mg, 530 μmol, 96%, 2 steps).

¹H-NMR (CDCl₃) δ: 0.59-0.78 (m, 4H), 1.33-1.44 (m, 4H), 1.48 (s, 9H), 4.02 (ddd, J=28.9, 9.5, 7.4 Hz, 1H), 5.34 (d, J=47.6 Hz, 1H), 7.05 (m, 1H), 7.17 (m, 1H), 7.30 (m, 1H), 7.40-7.49 (m, 3H), 7.54 (m, 1H), 7.79 (m, 2H).

### Step 12

To a solution of compound 12-12 (313.6 mg, 602 μmol) in MeOH (3 ml) was added K₂CO₂ (416 mg, 3.01 mmol) at room temperature. After being stirred for 40 min at room temperature, the reaction mixture was quenched with H₂O. The aqueous phase was extracted with AcOEt. The organic layer was dried over Na₂SO₄and concentrated. The residue was purified hy silica gel chromatugraphy to afford compound 12-13 (233.1 mg, 560 μmol, 93%).

¹H-NMR (CDCl₃) δ: 0.58-0.78 (m, 4H), 1.45 (m, 1H), 1.53 (s, 9H), 1.84 (s, 3H), 4.04 (dt, J=29.1, 8.0 Hz, 1H), 5.47 (d, J=47.2 Hz, 1H), 7.14 (m, 1H), 7.25 (m, 1H), 7.36-7.44 (m, 2H), 10.05 (brs, 1H).

### Step 13

Compound 12-13 (233.1 mg, 560 μmol) was solved in TKA (2 ml) at room temperature. After being stirred for 45 min at room temperature, the reaction mixture was cooled to −20° C. and H₂SO₄ (0.5 ml) was added. After being stirred for 5 min at 0° C. the reaction mixture was cooled to −20° C. and HNO₃ (75 μl, 1.68 mmol) was added. After being stirred for 50 min at 0° C., the reaction mixture was quenched with aqueous K₂CO₂. The aqueous phase was extracted with AcOEt. The organic layer was dried over Na₂SO₄ and concentrated to afford compound 12-14 (926.3 mg, quant.). The obtained compound 12-14 was used in the next reaction without further purification.

¹H-NMR (CDCl₃) δ: 0.60-0.78 (m, 1H), 1.39 (m, 1H), 1.69 (s, 3), 3.85 (ddd, J=29.6, 9.2, 7.4 Hz, 1H), 5.41 (d, J=47.4 Hz, 1H), 7.25 (dd, J=10.7, 8.9 Hz, 1H), 8.23 (ddd, J=8.9, 4.1, 2.9 Hz, 1H) 8.45 (dd, J=6.7, 2.9 Hz, 1H).

### Step 14

To a solution of compound 12-14 (326.3 mg, quant.) and Boc₂O (325 μl, 1.40 mmol) in CH₂Cl₂ (6 ml) was added DMAP (103 mg, 840 μmol) at room temperature. After being stirred for 40 min at room temperature, the reaction mixture was concentrated. The residue was purified by silica gel chromatography to afford compound 12-15 (270.1 mg, 492 μmol, 88%, 2 steps).

¹H-NMR (CDCl₃) δ: 0.59-0.80 (m, 4H), 1.40 (m, 1H), 1.52 (s, 18H), 1.74 (m, 3H), 3.93 (ddd, J=28.6. 8.9, 7.0 Hz, 1H), 5.44 (d, J=47.2 Hz, 1H), 7.29 (dd, J=10.8, 9.0 Hz, 1H), 8.27 (ddd, J=9.0, 4.2, 2.9 Hz, 1H), 8.53 (dd, J=6.7, 2.9 Hz, 1H).

### Step 15

To a solution of compound 12-15 (276.1 mg, 492 μmol) in THF (3 ml) and MeOH (1.5 ml) was added Pd/C (52.4 mg) at room temperature. After being stirred for 6.5 h under H₂ atmosphere at room temperature, the reaction mixture was filtered through a pad of Celite (Registered trademark), and the residue was washed with AcOEt. The filtrate was concentrated, and the residue was purified by silica gel chromatography to afford compound 12-16 (240.9 mg, 453 μmol, 92%).

¹ H-NMR (CDCl₃) δ: 0.58-0.77 (m, 4H), 1.39 (m, 1H), 1.52 (s, 18H), 1.71 (m, 3H), 3.57 (brs, 2H), 4.07 (ddd, J=28.7, 9.7, 6.9 Hz, 1H), 5.43 (d, J=47.7 Hz, 1H), 6.57 (ddd, J=8.7, 3.8, 3.0 Hz, 1H), 6.83 (dd, J=6.5, 3.0 Hz, 1H), 6.87 (dd, J=12.7, 8.7 Hz, 1H).

### Step 16

To a solution of compound 12-16 (70.0 mg, 132 μmol), 5-(fluoromethoxy)pyrazine-2-carboxylic acid (27.2 mg, 158 μmol) and diisopropylethylamine (46 μl, 263 μmol) in DMF (2 ml) was added HATU (60.1 mg, 158 μmol) at room temperature. After being stirred for 50 mm at room temperature, the reaction mixture was quenched with saturated aqueous NH₄Cl. The aqueous phase was extracted with AcOEt. The organic layer was dried over Na₂SO₄ and concentrated. The residue was purified by silica gel chromatography to afford compound 12-17 (86.6 mg, 126 μmol, 96%).

¹H-NMR (CDCl₃) δ: 0.59-0.77 (m, 4H), 1.38 (m, 1H), 1.54 (s, 18H), 1.76 (m, 3H), 4.05 (m, 1H), 5.45 (d, J=47.4 Hz, 1H), 6.15 (ddd, J=50.9, 11.5, 2.0 Hz, 2H), 7.15 (dd, J=11.5, 8.8 Hz, 1H), 7.48 (dd, J=6.8, 2.8 Hz, 1H), 8.20 (d, J=1.5 Hz, 1H), 8.37 (ddd, J=8.8, 4.0, 2.8 Hz, 1H), 9.08 (d, J=1.5 Hz, 1H), 9.63 (s, 1H).

### Step 17

Compound 12-17 (86.6 mg, 126 μmol) was solved in formic acid (1 ml, 26.1 mmol) at room temperature. After being stirred for 15 h at room temperature, the reaction mixture was quenched with aqueous K₂CO₂. The aqueous phase was extracted with AcOEt. The organic layer was dried over Na₂SO₄ and concentrated. The residue was purified by trituration from hexane to uive pure compound I-82 (52.8 mg, 109 μmol, 86%).

¹H-NMR (CDCl₃) δ: 0.64 (m, 2H), 0.73 (m, 2H), 1.38 (m, 1H), 1.67 (s, 3H), 3.95 (ddd, J=29.9, 10.0, 7.3 Hz, 1H), 4.37 (brs, 2H), 5.45 (d, J=48.1 Hz, 1H), 6.15 (m, 2H), 7.11 (dd, J=11.3, 8.9 Hz, 1H), 7.50 (dd, J=6.8, 2.8 Hz, 1H), 8.03 (ddd, J=8.9, 4.1, 2.8 Hz, 1H), 8.29 (d, J=1.4 Hz, 1H), 9.08 (d, J=1.4 Hz, 1H), 9.52 (s, 1H).

### EXAMPLE 13

### Synthesis of Compound I-109

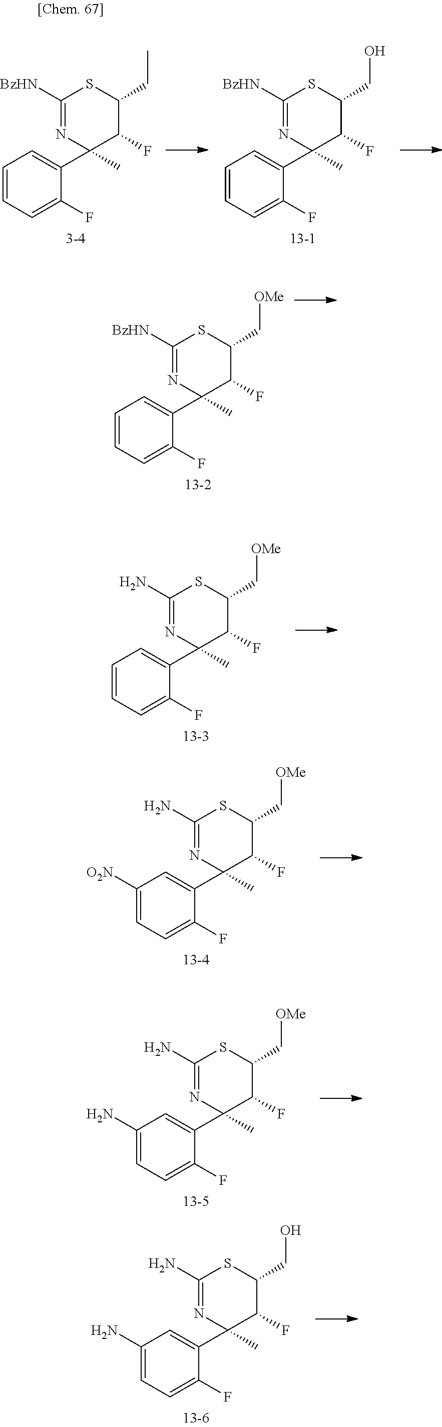

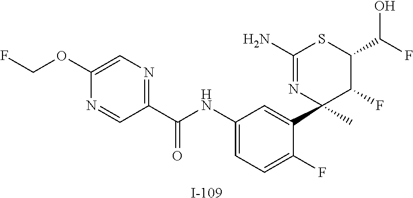

### Step 1

A solution of compound 3-4 (1.25 g, 2.57 mmol) and silver (I) tetrafluoroborate (1.00 g, 5.14 mmol) in DMSO (6.3 mL) and water (0.63 mL) was stirred for 3.5 h at room temperature. The reaction wan quenched with a saturated solution of sodium hydrogen carbonate. The resulting mixture was filtered through celite (Registered trademark) pad and the filtrate was extracted with ethyl acetate. The combined organic layers were washed with water and evaporated. The crude product was purified by flash column chromatography (silica gel, 2:1 hexane-ethyl acetate) to give compound 13-1 (560 mg, 58%) as a colorless amorphous.

¹H NMR (400 MHz, CDCl₃) δ: 1.89 (s, 3H), 3.17-3.29 (m, 1H), 3.80 (dd, J=10.8, 7.9 Hz, 1H), 4.04 (dd, J=10.8, 7.2 Hz, 1H), 5.60 (dd, J=47.3, 1.6 Hz, 1H), 7.12 (dd, J=12.2, 8.2 Hz, 1H), 7.20 (t, J=7.7 Hz, 1H), 7.35-7.54 (m, 1H), 8.23 (d, J=7.3 Hz, 2H).

### Step 2

To a stirred suspension of compound 13-1 (560 mg, 9.81 mmol) and sodium hydride (179 mg, 4.46 mmol, 60% in oil) in THF (6 mL) was added iodomethane (0.465 mL, 7.44 mmol) at 0° C. After being stirred for 2 h at 0° C., the reaction was quenched with a saturated solution of ammonium chloride. The mixture was extracted with ethyl acetate and the combined organic layers were washed with water. The solvent was evaporated and the erode product was purified by flash column chromatography (silica gel, gradient from 4:1 to 8:1 hexane-ethyl acetate) to give compound 13-2 (326 g, 56%) as a colorless amorphous.

¹H NMR (400 MHz, CDCl₃) δ: 1.88 (s, 3H), 3.21-3.33 (m, 1H), 3.35 (s, 3H), 3.44-3.48 (m, 1H), 3.81 (dd, J=9.3, 7.3 Hz, 1H), 5.52 (dd, J=47.2, 2.0 Hz, 1H), 7.12 (dd, J=12.3, 8.0 Hz, 1H), 7.19 (td, J=7.6, 1.2 Hz, 1H), 7.34-7.53 (m, 5H), 8.23 (d, J=7.0 Hz, 2H).

### Step 3

A solution of compound 13-2 (326 mg, 0.84 mmol) and hydrazine monohydrate (0.405 mL, 8.35 mmol) in ethanol (5 mL) was stirred for 18 h at room temperature. The mixture was evaporated, and the crude product, was purified by flash column chromatography (amino silica gel, 1:1 hexamethyl acetate) to give compound 13-3 (210 mg, 88%) as a colorless gum.

¹H NMR (400 MHz, CDCl₃) δ: 1.75 (t, J=1.5 Hz, 3H), 3.21-3.33 (m, 1H), 3.32 (s, 3H), 3.40-3.44 (m, 1H), 3.77 (dd, J=9.4, 6.7 Hz, 1H), 5.33 (dd, J=47.7, 1.8 Hz, 1H), 7.03 (ddd, J=12.4, 8.2, 1.3 Hz, 1H), 7.12 (td, J=7.5, 1.3 Hz, 1H), 7.23-7.30 (m, 2H).

### Step 4

To a stirred suspension of compound 13-3 (210 mg, 0.73 mmol) and sulfuric acid (0.520 mL, 9.76 mmol) in trifluoroacetic acid (2.1 mL) was added nitric acid (0.049 mL, 1.10 mmol) at −20° C. After being stirred for 30 min at between −20° C. and 10° C., the reaction was quenched with a solution of potassium carbonate. The mixture was extracted with ethyl acetate, and the combined organic layers were washed with water. The solvent was evaporated to give compound 13-4 (231 mg) as a crude product, which was used for the next reaction without further purification.

### Step 5

A suspension of compound 13-4 (231 mg), iron (311 mg, 5.58 mmol), and ammonium chloride (447 mg, 8.37 mmol) in toluene (2 mL) and water (2 mL) was stirred for 2 h at 80° C. After being cooled to room temperature, the reaction was quenched with a solution of potassium carbonate. The mixture was filtered through celite (Registered trademark) pad, and the filtrate was extracted with ethyl acetate. The combined organic layers were washed with water and evaporated to give compound 13-5 (206 mg) as a crude product, which was used for the next reaction without further purification.

### Step 6

To a stirred solution of compound 13-5 (55.6 mg) in dichloromethane (1.1 mL) was added boron tribromide (0.922 mL, 0.922 mmol, 1 mol/L in dichloromethane) at −78° C. After being stirred at 0° C. for 3 h, boron tribrornidc (0.553 mL, 0.553 mmol) was added to the mixture at −78° C. After being stirred at 0° C. for 1 h, the reaction was quenched with a saturated solution of sodium hydrogen carbonate. The mixture was extracted with ethyl acetate, and the combined organic layers were washed with water. The solvent was evaporated to give compound 13-6 (58.1 mg) as a crude product, which was used for the next reaction without further purification.

### Step 7

To a stirred solution of compound 13-6 (58.1 mg) and hydrogen chloride (0.092 mL, 0.184 mmol, 2 mol/L in water) were added 5-(fluoromethoxy)pyrazine-2-carboxylic acid (31.7 mg, 0.181 mmol) and WSCD (38.9 mg, 0.203 mmol) at room temperature. After being stirred for 45 min at room temperature, the reaction was quenched with a saturated solution of sodium hydrogen carbonate. The mixture was extracted with ethyl acetate. The combined organic layers were washed with brine, dried over sodium sulfate, and filtered. The solvent was evaporated, and the crude product was triturated with hexane to give I-109 (64.5 mg, 74% over 4 steps).

¹H NMR (400 MHz, CDCl₃) δ: 1.77 (s, 3H), 3.23-3.35 (m, 1H), 3.75 (dd, J=11.0, 7.3 Hz, 1H), 4.00 (dd, J=11.0, 7.3 Hz, 1H), 5.44 (d, J=46.7 Hz, 1H), 6.15 (d, J=50.2 Hz, 2H), 7.09 (dd, J=11.5, 8.8 Hz, 1H), 7.34 (dd, J=6.7, 2.9 Hz, 1H), 7.95-7.99 (m, 1H), 8.30 (s, 1H), 9.08 (s, 1H), 9.48 (s, 1H).

### EXAMPLE 14

### Synthesis of Compound I-94

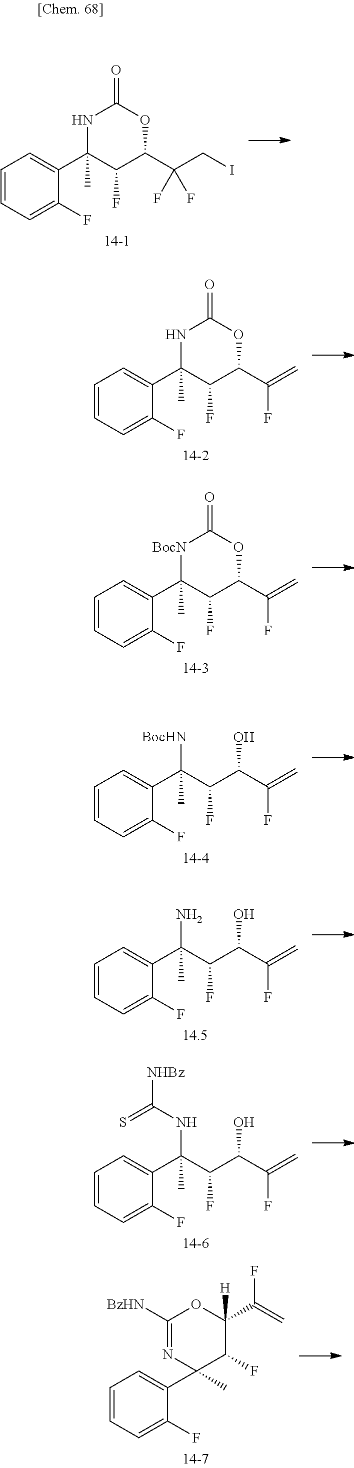

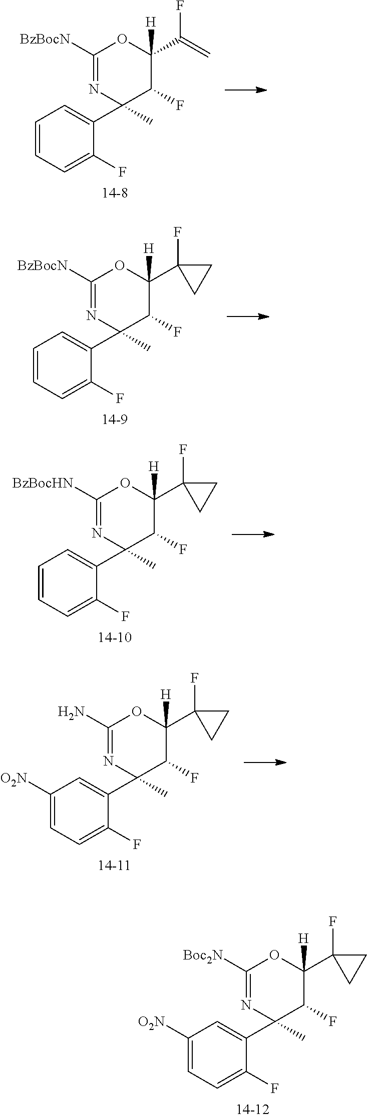

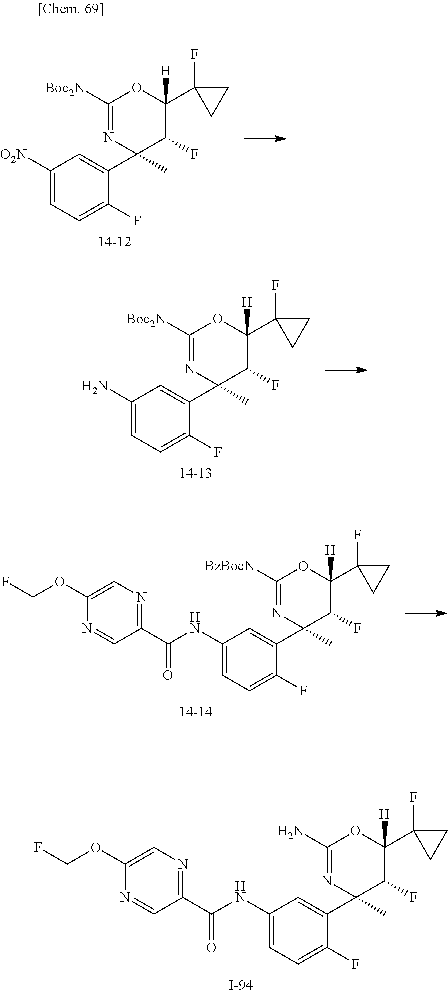

### Step 1

To a suspension of compound 14-1 (537.7 mg, 1.29 mmol) in dimethylacetamide (2.5 ml) and H₂O (2.5 ml) was added zinc (421 mg, 6.45 mmol) at room temperature. After being stirred for 2 h at 80° C., the reartion mixture was cooled to 0° C. and quenched with aqueous Na₂S₂O₃. The resulting mixture was filtered through a pad of Celite (Registered trademark), and the residue was washed with AcOEt. The aqueous phase was extracted with AcOEt. The organic phase was dried over Na₂SO₄ and concentrated to afford compound 14-2. The obtained compound 14-2 was used in the next reaction without further purification,

¹H-NMR (CDCl₃) δ: 1.80 (s, 3H), 4.45 (d, J=28.2 Hz, 1H), 4.91 (ddd, J=17.8, 3.9, 1.3 Hz, 1H), 5.00 (ddd, J=14.8, 3.9, 1.3 Hz, 1H) 5.29 (d, J=46.6 Hz, 1H), 6.02 (s, 1H), 7.15 (m, 1H), 7.27 (m, 1H), 7.41 (m, 1H), 7.53 (m, 1H).

### Step 2

To a solution of compound 14-2 and Boc₂O (748 μl, 3.22 mmol) in CH₂Cl₂ (3.5 ml) was added DMAP (79 mg, 645 μmol) at room temperature. After being stirred for 1.5 h at room temperature, the reaction mixture was concentrated. The residue was purified by silica gel chromatography to afford compound 14-3 (436.3 mg, 1.17 mmol, 91%, 2 steps)

¹H NMR (CDCl₃) δ: 1.52 (s, 9H), 1.97 (m, 3H), 4.46 (d, J=29.3 Hz, 1H), 4.91 (ddd, J=20.2, 4.0, 1.3 Hz, 1H), 4.99 (ddd, J=12.3, 4.0, 1.3 Hz, 1H), 5.21 (d, J=46.3 Hz, 1H), 7.13 (m, 1H), 7.25 (m, 1H), 7.39 (m, 1H), 7.50 (m, 1H).

### Step 3

To a notation of compound 14-3 (456.3 mg, 1.23 mmol) in MeOH (5 ml) was added K₂CO₃ (679 mg, 4.91 mmol) at room temperature. After being stirred for 15 min at room temperature, the reaction mixiure was quenched with saturated aqueous NH₄Cl. The aqueous phase was extracted with AeOEt. The organic layer was dried over Na₂SO₄ and rencentrated to afford compound 14-4. The obtained compound 14-4 was used in the next reaction without further purification.

LC/MS(Shimadzu): RT 2.15, MS calcd for 346.16 (M+H⁺), found 346.15.

### Step 4

To a solution of compound 14-4 in CH₂Cl₂ (5 ml) was added TFA (1 ml, 13.0 mmol) at room temperature. After being stirred for 1 h at room temperature, the reaction mixture was quenched with saturated aqueous NaHCO₃ and K₂CO₃ solutions. The aqueous phase was extracted with AcOEt. The organic phase was dried over Na₂SO₄ and concentrated. The residue was purified by silica gel chromatography to afford compound 14-5 (254.6 mg, 1.04 mmol, 84%, 2 steps).

¹H-NMR (CDCl₃) δ: 1.77 (s, 3H), 3.88 (d, J=31.0 Hz, 1H), 4.71 (ddd, J=22.2, 2.9, 1.4 Hz, 1H), 4.79 (ddd, J=10.9, 2.9, 1.4 Hz, 1H), 5.09 (dd, J=44.9, 1.1 Hz, 1H), 7.10 (m, 1H), 7.22 (m, 1H), 7.35 (m, 1H), 7.58 (m, 1H).

### Step 5

To a solution of compound 14-5 (254.6 mg, 1.04 mmol) in CH₂Cl₂ (2.5 ml) was added BzNCS (209 μl, 1.56 mmol) at room temperature. After being stirred for 2.5 h at room temperature, the reaction mixture was concentrated. The residue was purified by silica gel chromatography to afford compound 14-6 (375.5 mg, 919 μmol, 89%).

¹H-NMR (CDCl₃) δ: 2.30 (s, 3H), 2.56 (d, J=9.5 Hz, 1H), 4.36 (ddd, J=25.2, 9.5, 5.0 Hz, 1H), 4.76 (dd, J=48.9, 3.5 Hz, 1H), 4.85 (dd, J=17.7, 3.5 Hz, 1H), 5.37 (d, J=43.9 Hz, 1H), 7.08 (m, 1H), 7.18 (m, 1H), 7.33 (m, 1H), 7.40-7.56 (m, 3H), 7.63 (m, 1H), 7.87 (m, 2H), 8.89 (s, 1H), 11.85 (s, 1H).

### Step 6

To a solution of compound 14-6 (375.5 mg, 919 μmol) in CH₃CN (7 ml) was added WSCD hydrochloride (352 mg, 1.84 mmol) at room temperature. After being stirred for 1 h at 50° C., the reaction mixture was cooled to room temperature and quenched with H₂O. The aqueous phase was extracted with AcOEt. The organic phase was dried over Na₂SO₄ and concentrated to afford compound 14-7. Obtained compound 14-7 was used in the next reaction without further purification.

¹H-NMR (COCl₃) δ: 1.91 (s, 3H), 4.52 (d, J=27.7 Hz, 1H), 5.01 (dd, J=9.0, 4.0 Hz, 1H), 5.09 (dd, J=23.7, 4.0 Hz, 1H), 5.43 (d, J=46.4 Hz, 1H), 7.17 (m, 1H), 7.24 (m, 1H), 7.38-7.49 (m, 4H), 7.53 (m, 1H), 8.27 (m, 2H), 11.81 (s, 1H).

### Step 7

To a solution of compound 14-7 and Boc₂O (427 μl, 1.81 mmol) in CH₂Cl₂ (3.5 ml) was added DMAP (22.5 mg, 184 μmol) at room temperature. After being stirred for 30 min at room temperature,the reaction mixture was concentrated. The residue was purified by silica gel chromatography to afford compound 14-8 (416.5 mg, 878 μmol, 95%).

¹H-NMR (CDCl₃) δ: 1.47 (s, 9H), 1.55 (s, 3H), 4.40 (dd, J=27.5, 2.8 Hz, 1H), 4.80 (dd, J=49.4, 3.6 Hz, 1H), 4.95 (dd, J=17.8, 3.6 Hz, 1H) 5.23 (d, J=47.2 Hz, 1H), 7.06 (m, 1H), 7.17 (m, 1H), 7.30 (m, 1H), 7.41-7.53 (m, 3H), 7.55 (m, 1H), 7.78 (m, 2H).

### Step 8

In a test tube, to a mixture of aqueous NaOH (30%, 1 ml) and Et₂O (4 ml) was added 1-methyl-1-nitrosourea (905 mg, 4.39 mmol, 5 eq.) at 0° C. After being stirred for 20 min at 0° C., the color of organic phase was turned to yellow, which indicated that an Et₂O solution of diazomethane was able to be prepared. In a separate flask, to a suspension of compound 14-8 (416.5 mg, 878 μmol) and Pd(OAc)₂ (39.4 mg, 176 μmol, 0.2 eq.) in Et₂O (4 ml) was added the Et₂O solution of diazomethane at −30° C. The reaction mixture was stirred al −20° C. and the Et₂O solution of diazomethane (5×5 eq.) prepared above and Pd(OAc)₂ (0.2×2 eq.) were added in several batches til the compound 14-8 was consumed completely. After being stirred for 3 h at −20° C. from the first addition of the diazomethane solution, the reaction mixture was quenched with H₂O and AcOH. Saturated aqueous NaHCO₃ was added and the resulting mixture was filtered through a pad of Celite (Registered trademark). The filtrate was washed with AcOEt. The aqueous phase was extracted with AcOEt. The organic phase was dried over Na₂SO₄ and concentrated. The residue was purified by siliea gel chromatography to afford compound 14-9 (259.2 mg, 531 μmol, 60%).

¹H-NMR (CDCl₃) δ: 0.75-1.20 (m, 4H), 1.43 (s, 3H), 1.47 (s, 3H), 4.25 (dd, J=28.5, 10.7 Hz, 1H), 5.27 (d, J=47.6 Hz, 1H), 7.05 (m, 1H), 7.17 (m, 1H), 7.29 (m, 1H), 7.40-7.61 (m, 4H), 7.76 (m, 2H).

### Step 9

To a solution of compound 14-9 (259.2 mg, 531 μmol) in MeOH (5 ml) was added K₂CO₃ (367 mg, 2.65 mmol) at room temperature. After being stirred for 20 min at room temperature, the reaction mixture was quenched with H₂O. The aqueous phase was extracted with AcOEt. The organic phase was dried over Na₂SO₄ and concentrated. The residue was purified by silica gel chromatography to afford compound 14-10 (117.4 mg, 305 μmol, 58%).

¹H-NMR (CDCl₃) δ: 0.84-1.17 (m, 4H), 1.52 (s, 9H), 1.85 (s, 3H), 4.35 (dd, J=28.1, 5.3 Hz, 1H), 5.48 (d, J=46.9 Hz, 1H), 7.14 (m, 1H), 7.24 (m, 1H), 7.35-7.44 (m, 2H), 10.00 (brs, 1H).

### Step 10

Compound 14-10 (117.4 mg, 305 μmol) was solved in TFA (1 ml) at room temparature. After being stirred for 1 h at room temperature, the reaction mixture was cooled to −20° C. and then H₂SO₄ (250 μl) was added. After being stirred for 5 min at 0° C., the reaction mixture was cooled to −20° C. then HNO₃ (41 μl, 916 μmol) was added. After being stirred for 25 min at 0° C., the reaction mixture was quenched with aqueous K₂CO₃. The aqueous phase was extracted with AcOEt. The organic phase was dried over Na₂SO₄ and concentrated. The residue was purified by silica gel chronntography to afford compound 14-11 (82.8 mg, 251 μmol, 82%)

¹H NMR (CDCl₃) δ: 0.81 (m, 1H), 0.91 -1.07 (m, 2H), 1.13 (m, 1H), 1.66 (s, 3H), 4.05 (dd, J=29.1, 9.9 Hz, 1H), 4.35 (brs, 2H), 5.33 (d, J=47.6 Hz, 1H), 7.23 (dd, J=10.7, 9.0 Hz, 1H), 8.21 (ddd, J=9.0, 4.1, 2.9 Hz, 1H), 8.46 (dd, J=6.7, 2.9 Hz, 1H).

### Step 11

To a solution of compound 14-11 (82.8 mg, 251 μmol) and Boc₂O (175 μl, 754 μmol) in CH₂Cl₂ (1 ml) was added DMAP (30.7 mg, 251 μmol) at room temperature. After being stirred for 30 min at room temperature, the reaction mixture was concentrated. The residue was purified by silica gel chromatography to afford compound 14-12 (83.8 mg, 158 μmol, 63%).

¹H-NMR (CDCl₃) δ: 0.80 (m, 1H), 0.92-1.09 (m, 2H), 1.16 (m, 1H), 1.53 (s, 18H), 1.74 (s, 3H), 4.23 (dd, J=28.2, 8.2 Hz, 1H), 5.39 (d, J=47.1 Hz, 1H), 7.28 (m, 1H), 8.26 (m, 1H), 8.56 (dd, J=6.7, 2.9 Hz, 1H).

### Step 12

To a solution of compound 14-12 (83.8 mg, 158 μmol) in THF (0.5 ml) and MeOH (1 ml) wee added Pd/C (8.4 mg) at room temperature. After being stirred for 4.5 h under H₂ atmosphere at room temperature, the reaction mixture was filtered through a pad of Celite (Registered trademark), and the residue was washed with AcOEt. The filtrate was concentrated, and the residue was purified by silica gel chromatography to afford compound 14-13 (65.4 ) mg, 131 μmol, 83%).

¹H-NMH (CDCl₃) δ: 0.75-1.20 (m, 4H), 1.52 (s, 18H), 1.70 (m, 3H), 3.57 (brs, 2H), 4.31 (dd, J=28.6, 10.0 Hz, 1H), 5.35 (d, J=47.7 Hz, 1H), 6.56 (m, 1H), 6.83-6.00 (m, 2H).

### Step 13

To a solution of compound 14-13 (41.0 mg, 82 μmol), 5-(fluoromethoxy)pyrazine-2-carboxylic acid (17.0 mg, 98 μmol) and diisopropylethylamine 120 μl, 164 μmol) in DMF (2 ml) was added HATU (37.5 mg, 98 μmol) at room temperature. After being stirred for 1 h at room temperature, the reaction mixture was quenched with saturated aqueous NH₄Cl. The aqueous phase was extracted with AcOEt. The organic phase was dried over Ka₂SO₄ and concentrated. The residue was purified by silica gel chromatography to afford compound 14-14 (49.4 mg, 76 μmol, 92%).

¹H-NMR (CDCl₃) δ: 0.75-1.22 (m, 4H), 1.55 (s, 18H), 1.76 (m, 3H), 4.29 (dd, J=29.1, 9.9 Hz, 1H), 5.37 (d, J=47.4 Hz, 1H), 6.15 (ddd, J=51.1, 13.6, 2.0 Hz, 2H), 7.15 (dd, J=11.5, 9.0 Hz, 1H), 7.51 (dd, J=6.8, 2.8 Hz, 1H), 8.19 (d, J=1.3 Hz, 1H), 8.37 (ddd, J=9.0, 4.3, 2.8 Hz, 1H), 9.08 (d, J=1.3 Hz, 1H), 9.6 4 (s, 1H).

### Step 14

Compound 14-14 (49.4 mg, 76 μmol) was solved in formic acid (1 ml, 26.1 mmol) at room temperature. After being stirred for 16.5 h at room temperature, the reaction mixture was quenched with aqueous K₃CO₃. The aqueous phase was extracted with AcOEt. The organic phase was dried over Na₂SO₄ and concentrated. The residue was purified by solidification from hexane to give pure 1-94 (25.9 mg, 57 μmol, 76%).

¹H-NMR (CDCl₃) δ: 0.76-1.20 (m, 4H), 1.67 (s, 3H), 4.11 (dd, J=29.2, 11.8 Hz, 1H), 5.35 (d, J=47.9 Hz, 1H), 6.15 (m, 2H), 7.11 (dd, J=11.3, 8.9 Hz, 1H), 7.50 (dd, J=6.8, 2.0 Hz, 1H), 8.02 (m, 1H), 8.29 (s, 1H), 9.08 (s, 1H), 9.52 (s, 1H).

The following compounds are prepared in a manner similar to the above. In the tables, RT means LC/MS retention time (minute).

TABLE 1-1

| | | | MS-ESI | |
|---|---|---|---|---|
| | | NMR (solvent: shift value ascending | (m/z) | LC/MS |
| No. | Structure | order) | [M + H]+ | RT |
| I-1 | 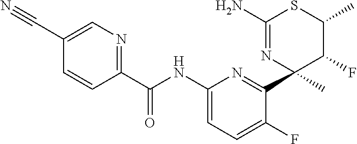 [SMILES: C[C@H]1SC(N)=N[C@](C)(c2nc(NC(=O)c3ccc(C#N)cn3)ccc2F)[C@H]1F] | 1H-NMR (CDCl3) δ: 1.39 (d, J = 6.9 Hz, 3H), 1.81 (s, 3H), 3.23-3.38 (m, 1H), 5.05 (dd, J = 47.6, 1.5 Hz, 1H), 7.51-7.46 (m, 1H), 6.22 (dd, J = 8.1, 1.9 Hz, 1H), 8.33 (dd, J = 9.0, 2.9 Hz, 1H), 8.43 (d, J = 8.1 Hz, 1H), 8.95 (d, J = 1.9 Hz, 1H), 10.25 (s, 1H). | 403 | 1.05 |
| I-2 | 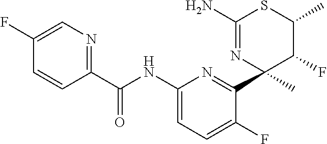 [SMILES: C[C@H]1SC(N)=N[C@](C)(c2nc(NC(=O)c3ccc(F)cn3)ccc2F)[C@H]1F] | 1H-NMR (CDCl3) δ: 1.39 (d, J = 7.0 Hz, 3H), 1.81 (s, 3H), 3.26-3.41 (m, 1H), 5.05 (dd, J = 47.7, 1.6 Hz, 1H), 7.46 (dd, J = 10.5, 8.9 Hz, 1H), 7.64-7.58 (m, 1H), 8.36-8.29 (m, 2H), 8.50 (d, J = 2.8 Hz, 1H), 10.20 (s, 1H). | 396 | 1.17 |
| I-3 | 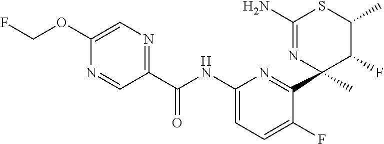 [SMILES: C[C@H]1SC(N)=N[C@](C)(c2nc(NC(=O)c3cnc(OCF)cn3)ccc2F)[C@H]1F] | 1H-NMR (CDCl3) δ: 1.39 (d, J = 7.0 Hz, 3H), 1.81 (s, 3H), 3.24-3.38 (m, 1H), 5.04 (dd, J = 1.5, 47.7 Hz, 1H), 6.16 (d, J = 50.9 Hz, 2H), 7.47 (dd, J = 10.5, 9.0 Hz, 1H), 8.34-8.30 (m, 2H), 9.09 (d, J = 1.0 Hz, 1H), 9.93 (s, 1H). | 427 | 1.15 |
| I-4 | 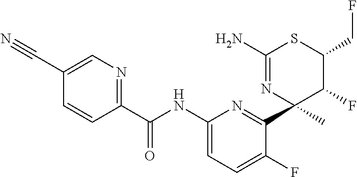 [SMILES: C[C@]1(c2nc(NC(=O)c3ccc(C#N)cn3)ccc2F)N=C(N)S[C@H](CF)[C@@H]1F] | 1H-NMR (CDCl3) δ: 1.81 (s, 3H), 3.76- 3.92 (m, 1H), 4.32-4.48 (m, 1H), 4.74- 4.91 (m, 1H), 5.45 (dd, J = 48.1, 2.7 Hz, 1H), 7.47-7.53 (m, 1H), 8.22 (dd, J = 8.2, 1.9 Hz, 1H), 8.35 (dd, J = 8.8, 3.0 Hz, 1H), 8.44 (d, J = 8.2 Hz, 1H), 8.95 (d, J = 1.1 Hz, 1H), 10.24 (s, 1H). | 421 | 1.06 |
| I-5 | 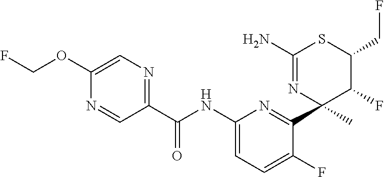 [SMILES: C[C@]1(c2nc(NC(=O)c3cnc(OCF)cn3)ccc2F)N=C(N)S[C@H](CF)[C@@H]1F] | 1H-NMR (CDCl3) δ: 1.81 (s, 3H), 3.78- 3.93 (m, 1H), 4.32-4.48 (m, 1H), 4.82 (dt, J = 47.1, 8.6 Hz, 1H), 5.44 (dd, J = 48.2, 2.5 Hz, 1H), 6.16 (dd, J = 50.9, 2.5 Hz, 2H), 7.52-7.46 (m, 1H), 8.36-8.32 (m, 2H), 9.09 (d, J = 1.3 Hz, 1H), 9.92 (s, 1H). | 445 | 1.14 |
| I-6 | 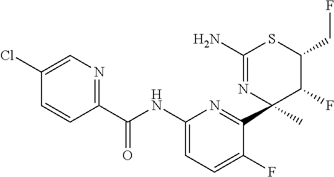 [SMILES: C[C@]1(c2nc(NC(=O)c3ccc(Cl)cn3)ccc2F)N=C(N)S[C@H](CF)[C@@H]1F] | 1H-NMR (CDCl3) δ: 1.81 (s, 3H), 4.74- 4.91 (m, 1H), 5.45 (dd, J = 48.1, 2.3 Hz, 1H), 7.45-7.53 (m, 1H), 7.90 (dd, J = 8.7, 2.3 Hz, 1H), 8.25 (d, J = 8.7 Hz, 1H), 6.34 (dd, J = 8.7, 3.1 Hz, 1H), 8.62 (s, 1H), 10.22 (s, 1H). | 430 | 129 |
| I-7 | 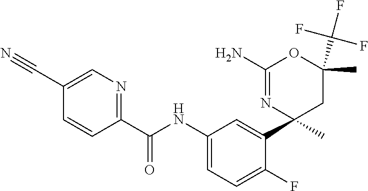 [SMILES: C[C@@]1(c2cc(NC(=O)c3ccc(C#N)cn3)ccc2F)C[C@@](C)(C(F)(F)F)OC(N)=N1] | 1H-NMR (CDCl3) δ: 1.05 (s, 3H), 1.64 (s, 3H), 2.14 (d, J = 14.1 Hz, 1H), 2.89 (d, J = 14.1 Hz, 1H), 7.11 (t, J = 10.0 Hz, 1H), 7.57 (d, J = 6.8 Hz, 1H), 8.05-8.00 (m, 1H), 8.21 (d, J = 8.5 Hz, 1H), 8.43 (d, J = 8.5 Hz, 1H), 8.91 (s, 1H), 9.88 (s, 1H). | 438 | 1.33 |

TABLE 1-2

| | | | | |
|---|---|---|---|---|
| I-8 | 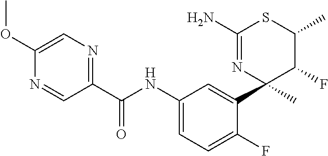 [SMILES: COc1cnc(C(=O)Nc2ccc(F)c([C@@]3(C)N=C(N)S[C@H](C)[C@@H]3F)c2)cn1] | 1H-NMR (CDCl3) δ: 1.35 (d, J = 6.9 Hz, 3H), 1.79 (s, 3H), 3.05-3.20 (m, 1H), 4.07 (s, 3H), 5.13 (d, J = 47.2 Hz, 1H), 7.08 (dd, J = 11.2, 9.2 Hz, 1H), 7.30 (dd, J = 6.7, 2.1 Hz, 1H), 7.97-8.02 (m, 1H), 8.16 (s, 1H), 9.01 (s, 1H), 9.51 (s, 1H). | 408 | 1.26 |
| I-9 | 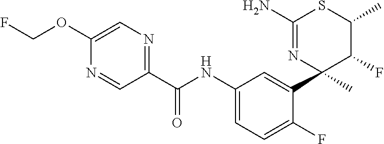 [SMILES: C[C@H]1SC(N)=N[C@](C)(c2cc(NC(=O)c3cnc(OCF)cn3)ccc2F)[C@H]1F] | 1H-NMR (CDCl3) δ: 1.35 (s, J = 6.9 Hz, 3H), 1.78 (s, 3H), 3.04-3.16 (m, 1H), 5.13 (d, J = 47.3 Hz, 1H), 6.15 (dd, J = 51.3, 3.1 Hz, 2H), 7.09 (dd, J = 9.0, 11.3 Hz, 1H), 7.29-7.32 (m, 1H), 8.02-7.97 (m, 1H), 8.30 (s, 1H), 9.08 (s, 1H), 9.49 (s, 1H). | 426 | 1.2 |
| I-10 | 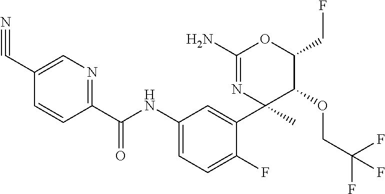 [SMILES: C[C@]1(c2cc(NC(=O)c3ccc(C#N)cn3)ccc2F)N=C(N)O[C@H](CF)[C@@H]1OCC(F)(F)F] | 1H-NMR (CDCl3) δ: 1.65 (s, 3H), 3.99- 4.11 (m, 2H), 4.18-4.32 (m, 4H), 4.50 (m, 1H), 4.62 (m, 1H), 7.11 (dd, J = 11.5, 8.9 Hz, 1H), 7.50 (dd, J = 6.9, 2.8 Hz, 1H), 8.03 (ddd, J = 8.9, 4.0, 2.8 Hz, 1H), 6.21 (dd, J = 8.2, 2.0 Hz, 1H), 8.43 (dd, J = 8.2, 0.8 Hz, 1H), 8.90 (dd, J = 2.0, 0.8 Hz, 1H), 9.87 (s, 1H). | 484 | 1.29 |
| I-11 | 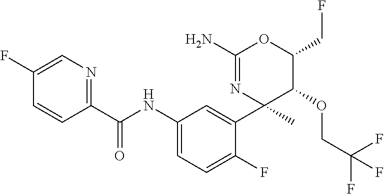 [SMILES: C[C@]1(c2cc(NC(=O)c3ccc(F)cn3)ccc2F)N=C(N)O[C@H](CF)[C@@H]1OCC(F)(F)F] | 1H-NMR (CDCl3) δ: 1.65 (s, 3H), 3.99- 4.12 (m, 2H), 4.17-4.37 (m, 4H), 4.50 (m, 1H), 4.61 (m, 1H), 7.09 (dd, J = 11.6, 8.9 Hz, 1H), 7.47 (dd, J = 6.9, 2.8 Hz, 1H), 7.60 (ddd, J = 8.7, 8.0, 2.8 Hz, 1H), 8.03 (ddd, J = 8.9, 4.2, 2.8 Hz, 1H), 8.32 (dd, J = 8.7, 4.5 Hz, 1H), 8.45 (d, J = 2.8 Hz, 1H), 9.82 (s, 1H). | 477 | 1.35 |
| I-12 | 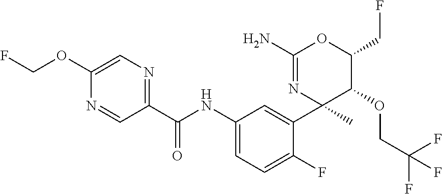 [SMILES: C[C@]1(c2cc(NC(=O)c3cnc(OCF)cn3)ccc2F)N=C(N)O[C@H](CF)[C@@H]1OCC(F)(F)F] | 1H-NMR (CDCl3) δ: 1.65 (s, 3H), 3.99- 4.12 (m, 2H), 4.16-4.35 (m, 4H), 4.50 (m, 1H), 4.61 (m, 1H), 6.15 (m, 2H), 7.10 (dd, J = 11.5, 8.8 Hz, 1H), 7.46 (dd, J = 6.9, 2.8 Hz, 1H), 8.02 (ddd, J = 6.8, 4.0, 2.8 Hz, 1H), 8.29 (brs, 1H), 9.08 (brs, 1H), 9.51 (s, 1H). | 508 | 1.34 |
| I-13 | 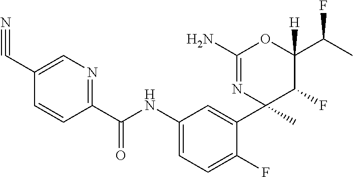 [SMILES: C[C@H](F)[C@H]1OC(N)=N[C@](C)(c2cc(NC(=O)c3ccc(C#N)cn3)ccc2F)[C@H]1F] | 1H-NMR (CDCl3) δ: 1.38 (d, J = 24.3, 6.4 Hz, 3H), 1.64 (s, 3H), 3.76 (ddd, J = 30.7, 13.5, 7.6 Hz, 1H), 4.37 (brs, 2H), 4.86 (ddq, J = 49.2, 7.6, 6.4 Hz, 1H), 5.18 (d, J = 48.1 Hz, 1H), 7.12 (m, 1H), 7.52 (m, 1H), 8.06 (m, 1H), 8.21 (dd, J = 8.2, 1.8 Hz, 1H), 8.43 (d, J = 8.2 Hz, 1H), 8.91 (s, 1H), 9.88 (s, 1H). | 418 | 1.22 |
| I-14 | 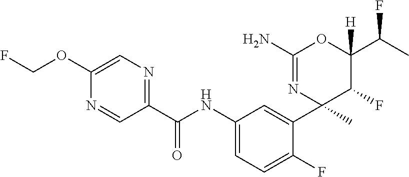 [SMILES: C[C@H](F)[C@H]1OC(N)=N[C@](C)(c2cc(NC(=O)c3cnc(OCF)cn3)ccc2F)[C@H]1F] | 1H-NMR (CDCl3) δ: 1.38 (dd, J = 24.2, 6.4 Hz, 3H), 1.65 (s, 3H), 3.77 (ddd, J = 30.9, 13.4, 7.7 Hz, 1H), 4.33 (brs, 2H), 4.85 (ddq, J = 49.1, 7.7, 6.4 Hz, 1H), 5.18 (d, J = 47.9 Hz, 1H), 6.15 (d, J = 51.1 Hz, 2H), 7.11 (dd, J = 11.4, 8.9 Hz, 1H), 7.46 (dd, J = 6.8, 2.5 Hz, 1H), 8.06 (m, 1H), 8.30 (s, 1H), 9.08 (s, 1H), 9.52 (s, 1H). | 442 | 1.23 |

TABLE 1-3

| | | | | |
|---|---|---|---|---|
| I-15 | 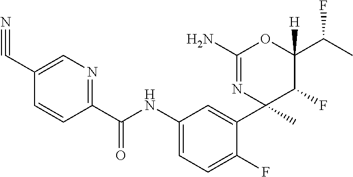 [SMILES: C[C@@H](F)[C@H]1OC(N)=N[C@](C)(c2cc(NC(=O)c3ccc(C#N)cn3)ccc2F)[C@H]1F] | 1H-NMR (CDCl3) δ: 1.39 (dd, J = 25.6, 6.0 Hz, 3H), 1.68 (s, 3H), 3.66 (m, 1H), 4.81 (m, 1H), 5.35 (d, J = 47.4 Hz, 1H), 7.11 (m, 1H), 7.56 (m, 1H), 8.00 (m, 1H), 8.21 (d, J = 8.3 Hz, 1H), 8.43 (d, J = 8.3 Hz, 1H), 8.90 (s, 1H), 9.88 (s, 1H). | 418 | 1.25 |
| I-16 | 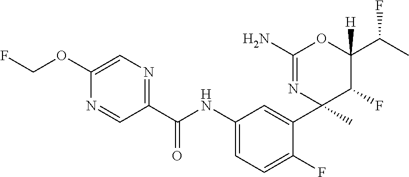 [SMILES: C[C@@H](F)[C@H]1OC(N)=N[C@](C)(c2cc(NC(=O)c3cnc(OCF)cn3)ccc2F)[C@H]1F] | 1H-NMR (CDCl3) δ: 1.39 (dd, J = 25.3, 6.1 Hz, 3H), 1.68 (s, 3H), 3.66 (m, 1H), 4.80 (m, 1H), 5.35 (d, J = 47.6 Hz, 1H), 6.15 (dd, J = 51.0, 5.9 Hz, 2H), 7.10 (m, 1H), 7.51 (m, 1H), 7.98 (m, 1H), 8.29 (s, 1H), 9.09 (s, 1H), 9.52 (s, 1H). | 442 | 1.3 |
| I-17 | 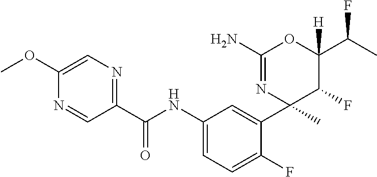 [SMILES: COc1cnc(C(=O)Nc2ccc(F)c([C@@]3(C)N=C(N)O[C@H]([C@H](C)F)[C@@H]3F)c2)cn1] | 1H-NMR (CDCl3) δ: 1.38 (dd, J = 24.2, 6.4 Hz, 3H), 1.65 (s, 3H), 3.77 (ddd, J = 30.7, 13.2, 7.5 Hz, 1H), 4.07 (s, 3H), 4.36 (brs, 2H), 4.85 (ddq, J = 49.1, 7.5, 6.4 Hz, 1H), 5.18 (d, J = 47.9 Hz, 1H), 7.10 (m, 1H), 7.43 (m, 1H), 8.08 (m, 1H), 8.16 (s, 1H), 9.02 (s, 1H), 9.53 (s, 1H). | 424 | 1.18 |
| I-18 | 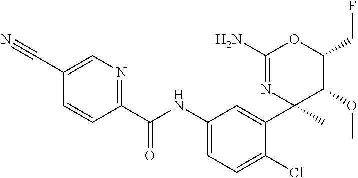 [SMILES: CO[C@H]1[C@@H](CF)OC(N)=N[C@]1(C)c1cc(NC(=O)c2ccc(C#N)cn2)ccc1Cl] | 1H-NMR (CDCl3) δ: 1.83 (s, 3H), 3.69 (s, 3H), 3.98-4.03 (m, 1H), 4.50-4.52 (br m, 2H), 4.58-4.66 (m, 1H), 7.42 (d, J = 8.5 Hz, 1H), 7.62 (s, 1H), 8.05 (d, J = 8.3 Hz, 1H), 8.21 (d, J = 8.0 Hz, 1H), 8.42 (d, J = 8.0 Hz, 1H), 8.91 (s, 1H), 9.91 (s, 1H). | 432 | 1.18 |
| I-19 | 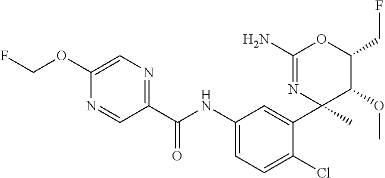 [SMILES: CO[C@H]1[C@@H](CF)OC(N)=N[C@]1(C)c1cc(NC(=O)c2cnc(OCF)cn2)ccc1Cl] | 1H-NMR (CDCl3) δ: 1.82 (s, 3H), 3.69 (s, 3H), 3.97-4.03 (m, 1H), 4.50-4.53 (br m, 2H), 4.61-4.63 (m, 1H), 6.09 (d, J = 4.5 Hz, 1H), 6.21 (d, J = 4.8 Hz, 1H), 7.40 (d, J = 8.5 Hz, 1H), 7.57 (s, 1H), 8.06 (d, J = 8.5 Hz, 1H), 8.29 (s, 1H), 9.07 (s, 1H), 9.57 (s, 1H). | 456 | 1.21 |
| I-20 | 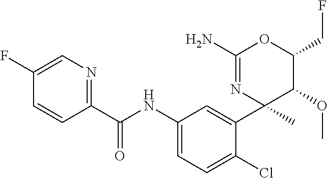 [SMILES: CO[C@H]1[C@@H](CF)OC(N)=N[C@]1(C)c1cc(NC(=O)c2ccc(F)cn2)ccc1Cl] | 1H-NMR (CDCl3) δ: 1.86 (s, 3H), 3.69 (s, 3H), 4.03-4.06 (m, 1H), 4.51-4.55 (m, 2H), 4.61-4.64 (m, 1H), 7.41 (d, J = 8.5 Hz, 1H), 7.58-7.62 (m, 2H), 8.08 (d, J = 8.3 Hz, 1H), 8.32 (dd, J = 8.0, 4.3 Hz, 1H), 8.47 (s, 1H), 9.90 (s, 1H). | 425 | 1.21 |
| I-21 | 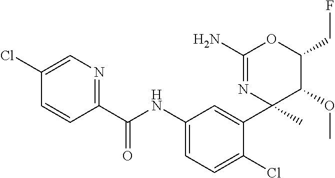 [SMILES: CO[C@H]1[C@@H](CF)OC(N)=N[C@]1(C)c1cc(NC(=O)c2ccc(Cl)cn2)ccc1Cl] | 1H-NMR (CDCl3) δ: 2.07 (s, 3H), 3.72 (s, 3H), 4.28-4.33 (m, 1H), 4.53-4.61 (m, 1H), 4.64-4.73 (m, 2H), 7.47 (d, J = 8.8 Hz, 1H), 7.59 (s, 1H), 7.89 (d, J = 8.3 Hz, 1H), 8.15-8.23 (m, 3H), 8.60 (s, 1H), 10.12 (s, 1H). | 441 | 1.3 |

TABLE 1-4

| | | | | |
|---|---|---|---|---|
| I-22 | 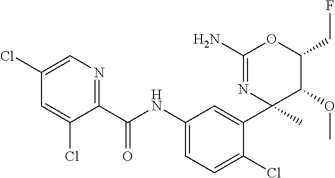 [SMILES: CO[C@H]1[C@@H](CF)OC(N)=N[C@]1(C)c1cc(NC(=O)c2ncc(Cl)cc2Cl)ccc1Cl] | 1H-NMR (CDCl3) δ: 2.06 (s, 3H), 3.72 (s, 3H), 4.27-4.33 (m, 1H), 4.53-4.62 (m, 1H), 4.64-4.74 (m, 2H), 7.43-7.46 (m, 2H), 7.89 (s, 1H), 8.16 (s, 1H), 8.32 (d, J = 8.5 Hz, 1H), 8.53 (s, 1H), 10.12 (s, 1H). | 475 | 1.32 |
| I-23 | 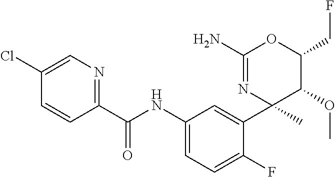 [SMILES: CO[C@H]1[C@@H](CF)OC(N)=N[C@]1(C)c1cc(NC(=O)c2ccc(Cl)cn2)ccc1F] | 1H-NMR (CDCl3) δ: 1.87 (s, 3H), 3.69 (s, 3H), 4.06 (s, 1H), 4.27-4.33 (m, 1H), 4.51-4.59 (m, 1H), 4.62-4.71 (m, 1H), 7.15 (t, J = 10.3 Hz, 1H), 7.39 (d, J = 6.5 Hz, 1H), 7.88 (d, J = 8.5 Hz, 1H), 8.12- 8.14 (m, 1H), 8.23 (d, J = 8.3 Hz, 1H), 8.58 (s, 1 H), 9.95 (s, 1H). | 425 | 1.14 |
| I-24 | 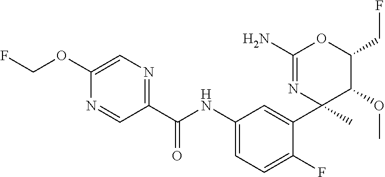 [SMILES: CO[C@H]1[C@@H](CF)OC(N)=N[C@]1(C)c1cc(NC(=O)c2cnc(OCF)cn2)ccc1F] | 1H-NMR (CDCl3) δ: 1.86 (s, 3H), 3.69 (s, 3H), 4.06 (s, 1H), 4.27-4.33 (m, 1H), 4.51-4.59 (m, 1H), 4.62-4.71 (m, 1H), 6.09 (d, J = 7.8 Hz, 1H), 6.21 (d, J = 7.8 Hz, 1H), 7.16 (t, J = 10.0 Hz, 1H), 7.34 (d, J = 6.8 Hz, 1H), 8.14-8.16 (m, 1H), 8.33 (s, 1H), 8.58 (s, 1H), 9.06 (s, 1H), 9.66 (s, 1H). | 440 | 1.06 |
| I-25 | 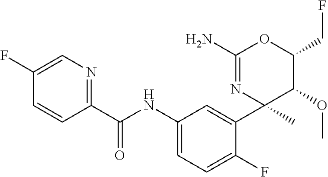 [SMILES: CO[C@H]1[C@@H](CF)OC(N)=N[C@]1(C)c1cc(NC(=O)c2ccc(F)cn2)ccc1F] | 1H-NMR (CDCl3) δ: 1.73 (s, 3H), 3.66 (s, 3H), 3.97 (s, 1H), 4.10-4.16 (m, 1H), 4.48-4.55 (m, 1H), 4.59-4.67 (m, 1H), 7.11 (t, J = 10.2 Hz, 1H), 7.42 (d, J = 6.8 Hz, 1H), 7.60 (t, J = 8.2 Hz, 1H), 8.07- 8.09 (m, 1H), 8.32 (dd, J = 8.5, 4.5 Hz, 1H), 8.46 (s, 1H), 9.86 (s, 1H). | 409 | 1.06 |
| I-26 | 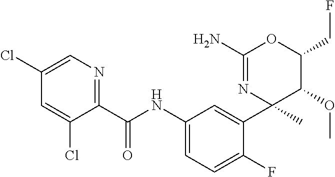 [SMILES: CO[C@H]1[C@@H](CF)OC(N)=N[C@]1(C)c1cc(NC(=O)c2ncc(Cl)cc2Cl)ccc1F] | 1H-NMR (CDCl3) δ: 1.66 (s, 3H), 3.65 (s, 3H), 3.94 (s, 1H), 4.04-4.10 (m, 1H), 4.47-4.54 (m, 1H), 4.58-4.65 (m, 1H), 7.09 (t, J = 10.2 Hz, 1H), 7.26-7.28 (m, 2H), 7.90 (s, 1H), 8.15-8.16 (m, 1H), 8.48 (s, 1H), 9.61 (s, 1H). | 459 | 1.16 |
| I-27 | 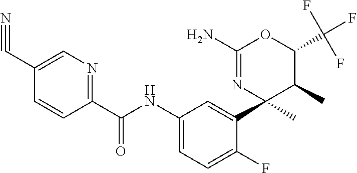 [SMILES: C[C@@H]1[C@@H](C(F)(F)F)OC(N)=N[C@]1(C)c1cc(NC(=O)c2ccc(C#N)cn2)ccc1F] | 1H-NMR (400 MHz, CDCl3) δ: 1.01 (d, J = 5.8 Hz, 3H), 1.77 (s, 3H), 2.09-2.16 (m, 1H), 4.04-4.06 (m, 1H), 7.09 (dd, J = 11.2, 9.7 Hz, 1H), 7.50-7.52 (m, 1H), 7.78-7.80 (m, 1H), 8.21 (d, J = 7.8 Hz, 1H), 8.44 (d, J = 7.6 Hz, 1H), 8.91 (s, 1H), 9.84 (s, 1H). | 438 | 1.27 |
| I-28 | 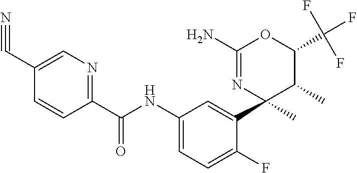 [SMILES: C[C@H]1[C@@H](C(F)(F)F)OC(N)=N[C@]1(C)c1cc(NC(=O)c2ccc(C#N)cn2)ccc1F] | 1H-NMR (400 MHz, CDCl3) δ: 1.17 (d, J = 6.8 Hz, 3H), 1.57 (s, 3H), 2.78 (q, J = 6.8 Hz, 1H), 4.12-4.18 (m, 1H), 7.11 (dd, J = 11.3, 8.8 Hz, 1H), 7.46 (dd, J = 7.1, 2.7 Hz, 1H), 7.99-8.01 (m, 1H), 8.21 (d, J = 8.0 Hz, 1H), 8.43 (d, J = 8.0 Hz, 1H), 8.90 (s, 1H), 9.86 (s, 1H). | 436 | 1.27 |

TABLE 1-5

| | | | | |
|---|---|---|---|---|
| I-29 | 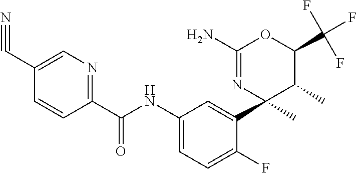 [SMILES: C[C@H]1[C@H](C(F)(F)F)OC(N)=N[C@]1(C)c1cc(NC(=O)c2ccc(C#N)cn2)ccc1F] | 1H-NMR (400 MHz, CDCl3) δ: 0.98 (d, J = 5.7 Hz, 3H), 1.59 (s, 3H), 2.31-2.39 (m, 1H), 4.33-4.40 (m, 1H), 7.08 (dd, J = 11.9, 8.7 Hz, 1H), 7.73-7.80 (m, 2H), 8.21 (dd, J = 8.1, 1.9 Hz, 1H), 6.43 (d, J = 8.1 Hz, 1H), 8.90 (d, J = 1.0 Hz, 1H), 9.85 (s, 1H). | 436 | 1.27 |
| I-30 | 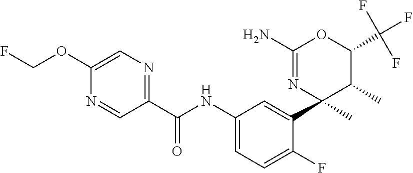 [SMILES: C[C@H]1[C@@H](C(F)(F)F)OC(N)=N[C@]1(C)c1cc(NC(=O)c2cnc(OCF)cn2)ccc1F] | 1H-NMR (400 MHz, CDCl3) δ: 1.17 (d, J = 7.0 Hz, 3H), 1.56 (s, 3H), 2.77 (q, J = 7.0 Hz, 1H), 4.16 (q, J = 7.0 Hz, 1H), 6.15 (d, J = 50.9 Hz, 1H), 7.10 (dd, J = 10.7, 9.4 Hz, 1H), 7.42 (d, J = 6.8 Hz, 1H), 7.96-7.96 (m, 1H), 8.29 (s, 1H), 9.08 (s, 1H), 9.50 (s, 1H). | 460 | 1.33 |
| I-31 | 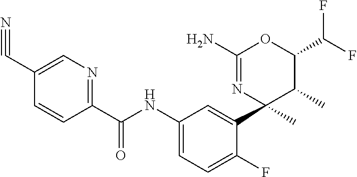 [SMILES: C[C@H]1[C@@H](C(F)F)OC(N)=N[C@]1(C)c1cc(NC(=O)c2ccc(C#N)cn2)ccc1F] | 1H-NMR (400 MHz, CDCl3) δ: 1.12 (d, J = 6.9 Hz, 3H), 1.56 (s, 3H), 2.66-2.72 (m, 1H), 3.88-3.93 (m, 1H), 5.75 (ddt, J = 55.2, 6.5, 2.3 Hz, 1H), 7.10 (dd, J = 11.2, 9.0 Hz, 1H), 7.46 (dd, J = 8.9, 2.3 Hz, 1H), 7.97-7.99 (m, 1H), 8.21 (d, J = 8.0 Hz, 1H), 8.43 (d, J = 8.0 Hz, 1H), 8.90 (s, 1H), 9.86 (s, 1H). | 418 | 1.25 |
| I-32 | 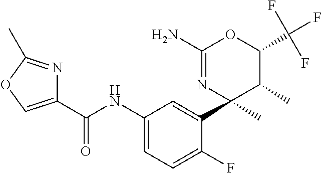 [SMILES: Cc1nc(C(=O)Nc2ccc(F)c([C@@]3(C)N=C(N)O[C@H](C(F)(F)F)[C@@H]3C)c2)co1] | 1H-NMR (400 MHz, CDCl3) δ: 1.16 (d, J = 6.8 Hz, 3H), 1.56 (s, 3H), 2.25 (s, 3H), 2.75 (q, J = 6.8 Hz, 1H), 4.13-4.18 (m, 1H), 7.07 (dd, J = 11.3, 9.3 Hz, 1H), 7.36-7.38 (m, 1H), 7.89-7.91 (m, 1H), 6.17 (s, 1H), 6.67 (s, 1H). | 415 | 1.13 |
| I-33 | 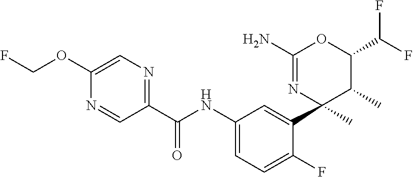 [SMILES: C[C@H]1[C@@H](C(F)F)OC(N)=N[C@]1(C)c1cc(NC(=O)c2cnc(OCF)cn2)ccc1F] | 1H-NMR (400 MHz, CDCl₃) δ: 1.12 (d, J = 6.9 Hz, 3H), 1.55 (s, 3H), 2.67-2.72 (m, 1H), 3.90-3.94 (m, 1H), 5.75 (ddt, J = 55.4, 8.1, 2.4 Hz, 1H), 8.15 (d, J = 50.9 Hz, 2H), 7.09 (dd, J = 11.4, 9.0 Hz, 1H), 7.40 (dd, J = 6.9, 2.4 Hz, 1H), 7.98-8.01 (m, 1H), 8.29 (s, 1H), 9.08 (s, 1H), 9.50 (s, 1H). | 442 | 1.28 |
| I-34 | 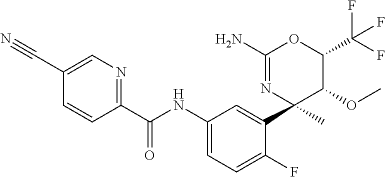 [SMILES: CO[C@H]1[C@@H](C(F)(F)F)OC(N)=N[C@]1(C)c1cc(NC(=O)c2ccc(C#N)cn2)ccc1F] | 1H-NMR (400 MHz, CDCl3) δ: 1.66 (s, 3H), 3.66 (s, 3H), 4.03 (m, 1H), 4.18 (s, 1H), 7.13 (dd, J = 12.0, 8.0 Hz, 1H), 7.48 (dd, J = 8.0, 4.0 Hz, 1H), 8.03 (m, 1H), 8.21 (dd, J = 8.0, 4.0 Hz, 1H), 8.42 (d, J = 8.0 Hz, 1H), 6.90 (m, 1H), 9.67 (s, 1H). | | |
| I-35 | 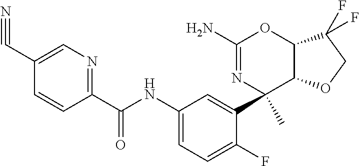 [SMILES: C[C@]1(c2cc(NC(=O)c3ccc(C#N)cn3)ccc2F)N=C(N)O[C@H]2[C@@H]1OCC2(F)F] | 1H-NMR (400 MHz, CDCl3) δ: 1.67 (s, 3H), 4.05-4.24 (m, 3H), 4.74 (s, 1H), 7.11 (t, J = 8.0 Hz, 1H), 7.53 (d, J = 8.0 Hz, 1H), 7.93 (m, 1H), 8.21 (d, J = 8.0 Hz, 1H), 8.42 (d, J = 8.0 Hz, 1H), 8.89 (m, 1H), 9.84 (s, 1H). | | |

TABLE 1-6

| I-36 | 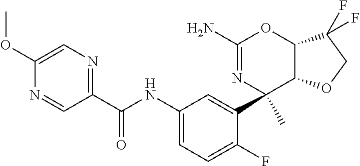 [SMILES: COc1cnc(C(=O)Nc2ccc(F)c([C@@]3(C)N=C(N)O[C@H]4[C@@H]3OCC4(F)F)c2)cn1] | 1H-NMR (400 MHz, CDCl3) δ: 1.66 (s, 3H), 4.05-4.21 (m, 3H), 4.07 (s, 3H), 4.73 (s, 1H), 7.09 (t, J = 8.0 Hz, 1H), 7.46 (d, J = 8.0 Hz, 1H), 7.93 (m, 1H), 8.15 (s, 1H), 9.01 (s, 1H), 9.49 (s, 1H). | | |
|---|---|---|---|---|
| I-37 | 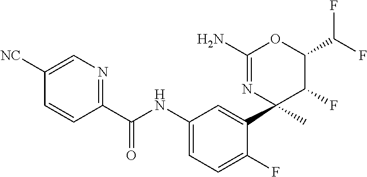 [SMILES: C[C@]1(c2cc(NC(=O)c3ccc(C#N)cn3)ccc2F)N=C(N)O[C@H](C(F)F)[C@@H]1F] | | | |
| I-38 | 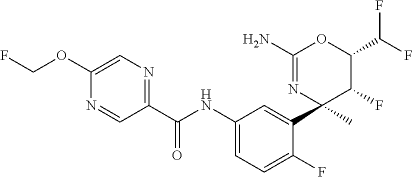 [SMILES: C[C@]1(c2cc(NC(=O)c3cnc(OCF)cn3)ccc2F)N=C(N)O[C@H](C(F)F)[C@@H]1F] | | | |
| I-39 | 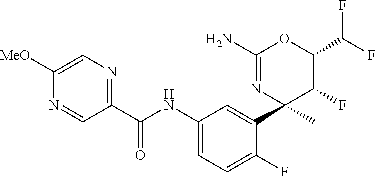 [SMILES: COc1cnc(C(=O)Nc2ccc(F)c([C@@]3(C)N=C(N)O[C@H](C(F)F)[C@@H]3F)c2)cn1] | | | |
| I-40 | 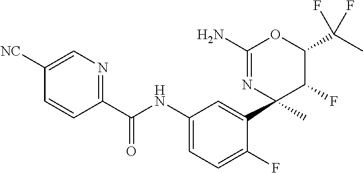 [SMILES: CC(F)(F)[C@H]1OC(N)=N[C@](C)(c2cc(NC(=O)c3ccc(C#N)cn3)ccc2F)[C@H]1F] | 1H-NMR (400 MHz, CDCl3) δ: 1.67 (s, 3H), 1.72 (t, J = 19.7 Hz, 3H), 3.88 (ddd, J = 29.9, 10.8, 4.4 Hz, 1H), 4.33 (brs, 2H), 5.38 (d, J = 48.1 Hz, 1H), 7.12 (m, 1H), 7.55 (m, 1H), 8.02 (m, 1H), 8.21 (d, J = 8.3 Hz, 1H), 8.43 (d, J = 8.3 Hz, 1H), 6.90 (s, 1H), 9.87 (s, 1H). | 436 | 1.28 |
| I-41 | 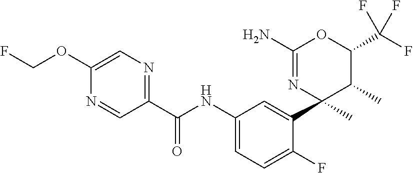 [SMILES: C[C@H]1[C@@H](C(F)(F)F)OC(N)=N[C@]1(C)c1cc(NC(=O)c2cnc(OCF)cn2)ccc1F] | 1H-NMR (400 MHz, CDCl3) δ: 1.66 (s, 3H), 1.72 (t, J = 19.6 Hz, 3H), 3.89 (ddd, J = 29.7, 10.3, 4.5 Hz, 1H), 4.38 (brs, 2H), 5.38 (d, J = 48.1 Hz, 1H), 6.15 (d, J = 51.1 Hz, 2H), 7.11 (m, 1H), 7.51 (m, 1H), 8.01 (m, 1H), 8.29 (s, 1H), 9.08 (s, 1H), 9.51 (s, 1H). | 460 | 1.33 |
| I-42 | 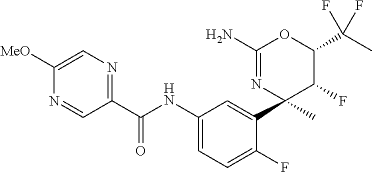 [SMILES: COc1cnc(C(=O)Nc2ccc(F)c([C@@]3(C)N=C(N)O[C@H](C(C)(F)F)[C@@H]3F)c2)cn1] | 1H-NMR (400 MHz, CDCl3) δ: 1.66 (s, 3H), 1.72 (t, J = 19.6 Hz, 3H), 3.89 (ddd, J = 29.8, 10.7, 4.6 Hz, 1H), 4.07 (s, 3H), 4.33 (brs, 2H), 5.38 (d, J = 48.2 Hz, 1H), 7.10 (m, 1H), 7.49 (m, 1H), 8.01 (m, 1H), 6.15 (s, 1H), 9.02 (s, 1H), 9.51 (s, 1H). | 442 | 1.3 |

TABLE 1-7

| | | | | |
|---|---|---|---|---|
| I-43 | 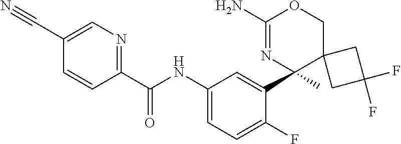 [SMILES: C[C@]1(c2cc(NC(=O)c3ccc(C#N)cn3)ccc2F)N=C(N)OCC12CC(F)(F)C2] | 1H-NMR (400 MHz, CDCl3) δ: 1.73 (d, J = 3.1 Hz, 3H), 2.10-2.20 (m, 1H), 2.30-2.42 (m, 1H), 2.74 (d, J = 29.4, 14.6 Hz, 1H), 2.94 (dd, J = 29.4, 14.6 Hz, 1H), 4.03 (d, J = 10.8 Hz, 1H), 4.19 (d, J = 10.8 Hz, 1H), 7.11 (dd, J = 11.9, 8.8 Hz, 1H), 7.63 (dd, J = 7.2, 2.6 Hz, 1H), 7.85-7.90 (m, 1H), 8.21 (dd, J = 8.1, 1.9 Hz, 1H), 8.43 (d, J = 8.1 Hz, 1H), 8.90 (s, 1H), 9.86 (s, 1H). | 430 | 1.17 |
| I-44 | 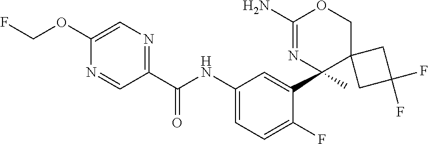 [SMILES: C[C@]1(c2cc(NC(=O)c3cnc(OCF)cn3)ccc2F)N=C(N)OCC12CC(F)(F)C2] | 1H-NMR (400 MHz, CDCl3) δ: 1.76 (s, 3H), 2.08-2.21 (m, 1H), 2.31-2.45 (m, 1H), 2.73-3.02 (m, 2H), 4.08 (d, J = 11.0 Hz, 1H), 4.21 (d, J = 11.0 Hz, 1H), 6.15 (d, J = 51.2 Hz, 2H), 7.06- 7.14 (m, 1H), 7.61 (d, J = 7.0 Hz, 1H), 7.85-7.79 (m, 1H), 8.30 (s, 1H), 9.08 (s, 1H), 9.52 (s, 1H). | 454 | 1.25 |
| I-45 | 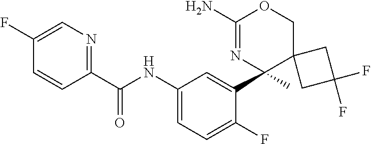 [SMILES: C[C@]1(c2cc(NC(=O)c3ccc(F)cn3)ccc2F)N=C(N)OCC12CC(F)(F)C2] | 1H-NMR (400 MHz, CDCl3) δ: 1.80 (s, 3H), 2.09-2.21 (m, 1H), 2.34-2.46 (m, 1H), 2.78-3.03 (m, 2H), 4.12 (d, J = 10.8 Hz, 1H), 4.23 (d, J = 10.8 Hz, 1H), 7.07-7.14 (m, 1H), 7.57-7.67 (m, 2H), 7.84-7.77 (m, 1H), 8.33 (dd, J = 8.9, 4.6 Hz, 1H), 8.47 (s, 1H), 9.84 (s, 1H). | 423 | 1.24 |
| I-46 | 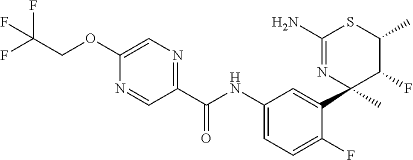 [SMILES: C[C@H]1SC(N)=N[C@](C)(c2cc(NC(=O)c3cnc(OCC(F)(F)F)cn3)ccc2F)[C@H]1F] | 1H-NMR (400 MHz, CDCl3) δ: 1.34 (d, J = 7.0 Hz, 3H), 1.76 (s, 3H), 3.09 (dq, J = 31.0, 7.0 Hz, 1H), 4.87 (q, J = 8.2 Hz, 2H), 5.11 (d, J = 47.4 Hz, 1H), 7.08 (dd, J = 11.4, 8.9 Hz, 1H), 7.30 (dd, J = 6.7, 2.6 Hz, 1H), 8.01- 7.95 (m, 1H), 8.31 (s, 1H), 9.02 (s, 1H), 9.45 (s, 1H). | 476 | 1.48 |
| I-47 | 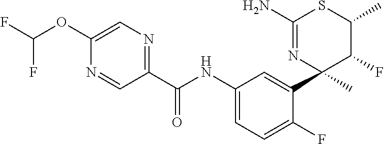 [SMILES: C[C@H]1SC(N)=N[C@](C)(c2cc(NC(=O)c3cnc(OC(F)F)cn3)ccc2F)[C@H]1F] | 1H-NMR (400 MHz, CDCl3) δ: 1.35 (d, J = 7.0 Hz, 3H), 1.78 (s, 3H), 3.10 (dq, J = 30.7, 7.0 Hz, 1H), 5.12 (d, J = 47.3 Hz, 1H), 7.10 (dd, J = 11.4, 8.8 Hz, 1H), 7.34-7.29 (m, 1H), 7.60 (d, J = 71.4 Hz, 1H), 7.96-8.00 (m, 1H), 8.34 (s, 1H), 9.07 (s, 1H), 9.45 (s, 1H). | 444 | 1.33 |

TABLE 1-8

| | | | | |
|---|---|---|---|---|
| I-48 | 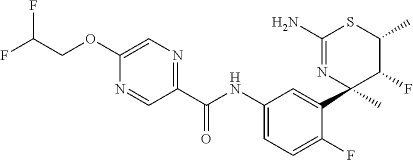 [SMILES: C[C@H]1SC(N)=N[C@](C)(c2cc(NC(=O)c3cnc(OCC(F)F)cn3)ccc2F)[C@H]1F] | 1H-NMR (400 MHz, CDCl3) δ: 1.45 (d, J = 7.0 Hz, 3H), 1.93 (s, 3H), 3.30 (dq, J = 30.0, 7.0 Hz, 1H), 4.65 (td, J = 13.3, 4.0 Hz, 2H), 5.25 (d, J = 46.4 Hz, 1H), 6.16 (tt, J = 55.0, 4.0 Hz, 1H), 7.14 (dd, J = 11.5, 9.0 Hz, 1H), 7.39 (dd, J = 6.6, 2.4 Hz, 1H), 8.04- 7.98 (m, 1H), 8.28 (s, 1H), 8.99 (s, 1H), 9.59 (s, 1H). | 458 | 1.32 |
| I-49 | 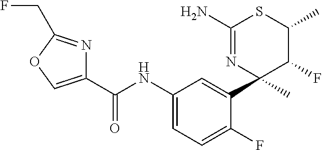 [SMILES: C[C@H]1SC(N)=N[C@](C)(c2cc(NC(=O)c3coc(CF)n3)ccc2F)[C@H]1F] | 1H-NMR (400 MHz, CDCl3) δ: 1.34 (d, J = 6.9 Hz, 3H), 1.76 (s, 3H), 3.09 (dq, J = 30.9, 6.9 Hz, 1H), 5.10 (d, J = 47.4 Hz, 1H), 5.44 (d, J = 47.1 Hz, 2H), 7.07 (dd, J = 11.4, 8.9 Hz, 1H), 7.24-7.30 (m, 1H), 7.85-7.91 (m, 1H), 8.34 (s, 1H), 8.66 (s, 1H). | 399 | 1.07 |
| I-50 | 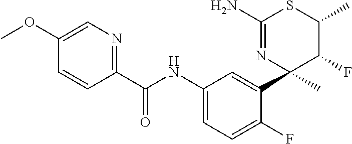 | 1H-NMR (400 MHz, CDCl3) δ: 1.34 (d, J = 7.0 Hz, 3H), 1.77 (s, 3H), 3.11 (dq, J = 31.2, 7.0 Hz, 1H), 3.94 (s, 3H), 5.11 (d, J = 48.2 Hz, 1H), 7.06 (dd, J = 11.7, 8.9 Hz, 1H), 7.25-7.36 (m, 2H), 7.98-8.05 (m, 1H), 8.30-8.20 (m, 2H), 9.80 (s, 1H). | 407 | 1.19 |
| I-51 | 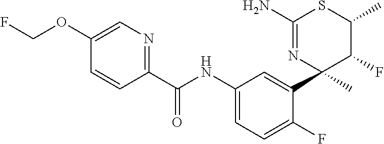 [SMILES: C[C@H]1SC(N)=N[C@](C)(c2cc(NC(=O)c3ccc(OCF)cn3)ccc2F)[C@H]1F] | 1H-NMR (400 MHz, CDCl3) δ: 1.34 (d, J = 6.8 Hz, 3H), 1.77 (s, 3H), 3.11 (dq, J = 31.4, 6.8 Hz, 1H), 5.12 (d, J = 48.4 Hz, 1H), 5.81 (d, J = 53.5 Hz, 2H), 7.07 (dd, J = 11.5, 8.7 Hz, 1H), 7.24-7.33 (m, 1H), 7.57 (dd, J = 8.7, 2.6 Hz, 1H), 7.98-8.04 (m, 1H), 8.29 (d, J = 8.7 Hz, 1H), 8.42 (d, J = 2.6 Hz, 1H), 9.81 (s, 1H). | 425 | 1.23 |
| I-52 | 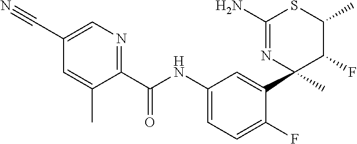 [SMILES: Cc1cc(C#N)cnc1C(=O)Nc1ccc(F)c([C@@]2(C)N=C(N)S[C@H](C)[C@@H]2F)c1] | 1H-NMR (400 MHz, CDCl3) δ: 1.34 (d, J = 6.9 Hz, 3H), 1.76 (s, 3H), 2.65 (s, 3H), 3.10 (dq, J = 30.3, 6.9 Hz, 1H), 5.12 (dd, J = 47.4, 1.4 Hz, 1H), 7.08 (dd, J = 11.5, 8.9 Hz, 1H), 7.19 (dd, J = 6.7, 2.7 Hz, 1H), 7.94 (s, 1H), 8.07-8.02 (m, 1H), 8.73 (d, J = 1.4 Hz, 1H), 9.97 (s, 1H). | 416 | 1.24 |

TABLE 1-9

| | | | | |
|---|---|---|---|---|
| I-53 | 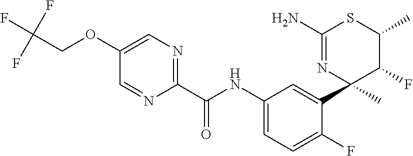 [SMILES: C[C@H]1SC(N)=N[C@](C)(c2cc(NC(=O)c3ncc(OCC(F)(F)F)cn3)ccc2F)[C@H]1F] | 1H-NMR (400 MHz, CDCl3) δ: 1.34 (d, J = 7.0 Hz, 3H), 1.77 (s, 3H), 3.10 (dq, J = 30.9, 7.0 Hz, 1H), 4.58 (q, J = 7.6 Hz, 2H), 5.11 (d, J = 48.4 Hz, 1H). 7.09 (dd, J = 11.5, 8.8 Hz, 1H), 7.38 (dd, J = 6.8, 2.8 Hz, 1H), 7.99- 8.03 (m, 1H), 8.62 (s, 2H), 9.72 (s, 1H). | 476 | 1.21 |
| I-54 | 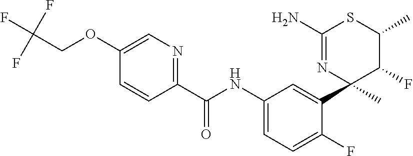 [SMILES: C[C@H]1SC(N)=N[C@](C)(c2cc(NC(=O)c3ccc(OCC(F)(F)F)cn3)ccc2F)[C@H]1F] | 1H-NMR (400 MHz, CDCl3) δ: 1.34 (d, J = 7.0 Hz, 3H), 1.77 (s, 3H), 3.10 (dq, J = 31.6, 7.0 Hz, 1H), 4.49 (q, J = 7.8 Hz, 2H), 5.12 (d, J = 47.4 Hz, 1H), 7.07 (dd, J = 11.7, 8.9 Hz, 1H), 7.31 (dd, J = 7.0, 2.9 Hz, 1H), 7.40 (dd, J = 8.9, 2.9 Hz, 1H), 7.97-8.02 (m, 1H), 8.28 (d, J = 8.9 Hz, 1H), 8.35 (d, J = 2.9 Hz, 1H), 9.78 (s, 1H). | 475 | 1.42 |
| I-55 | 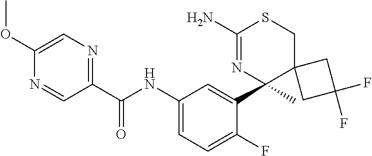 [SMILES: COc1cnc(C(=O)Nc2ccc(F)c([C@@]3(C)N=C(N)SCC34CC(F)(F)C4)c2)cn1] | 1H-NMR (400 MHz, CDCl3) δ: 1.68 (d, J = 2.8 Hz, 3H), 2.30-2.42 (m, 1H), 2.44-2.55 (m, 1H), 2.82 (q, J = 14.6 Hz, 1H), 2.93-3.07 (m, 2H), 3.17 (d, J = 12.8 Hz, 1H), 4.07 (s, 3H), 7.07 (dd, J = 11.8, 8.8 Hz, 1H), 7.56 (dd, J = 7.0, 2.8 Hz, 1H), 7.88-7.83 (m, 1H), 8.16 (d, J = 1.3 Hz, 1H), 9.02 (d, J = 1.3 Hz, 1H), 9.49(s, 1H). | 452 | 1.24 |
| I-56 | 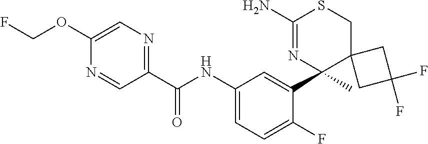 [SMILES: C[C@]1(c2cc(NC(=O)c3cnc(OCF)cn3)ccc2F)N=C(N)SCC12CC(F)(F)C2] | 1H-NMR (400 MHz, CDCl3) δ: 1.68 (d, J = 2.8 Hz, 3H), 2.31-2.42 (m, 1H), 2.44-2.55 (m, 1H), 2.82 (q, J = 14.1 Hz, 1H), 2.92-3.07 (m, 2H), 3.18 (d, J = 12.3 Hz, 1H), 6.15 (d, J = 51.2 Hz, 2H), 7.08 (dd, J = 11.9, 8.7 Hz, 1H), 7.57 (dd, J = 7.0, 2.8 Hz, 1H), 7.90-7.81 (m, 1H), 8.23 (d, J = 1.3 Hz, 1H), 9.09 (d, J = 1.3 Hz, 1H), 9.48 (s, 1H). | 470 | 1.28 |
| I-57 | 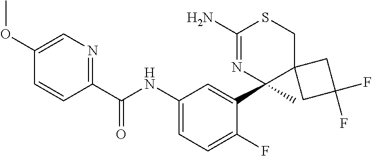 [SMILES: COc1ccc(C(=O)Nc2ccc(F)c([C@@]3(C)N=C(N)SCC34CC(F)(F)C4)c2)nc1] | 1H-NMR (400 MHz, CDCl3) δ: 1.68 (d, J = 2.8 Hz, 3H), 2.30-2.41 (m, 1H), 2.43-2.56 (m, 1H), 2.82 (q, J = 15.1 Hz, 1H), 2.94-3.09 (m, 2H), 3.16 (d, J = 13.1 Hz, 1H), 3.94 (s, 3H), 7.06 (dd, J = 11.9, 8.9 Hz, 1H), 7.34 (dd, J = 8.9, 2.8 Hz, 1H), 7.57 (dd, J = 7.2, 2.8 Hz, 1H), 7.85-7.91 (m, 1H), 8.24 (d, J = 8.9 Hz, 1H), 8.27 (d, J = 2 8 Hz, 1H), 9.82 (s, 1H). | 451 | 1.33 |

TABLE 1-10

| | | | | |
|---|---|---|---|---|
| I-58 | 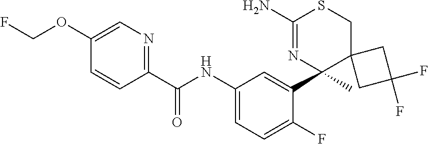 [SMILES: C[C@]1(c2cc(NC(=O)c3ccc(OCF)cn3)ccc2F)N=C(N)SCC12CC(F)(F)C2] | 1H-NMR (400 MHz, CDCl3) δ: 1.69 (d, J = 2.8 Hz, 3H), 2.30-2.43 (m, 1H), 2.43-2.55 (m, 1H), 2.82 (q, J = 14.8 Hz, 1H), 3.08-2.92 (m, 2H), 3.17 (d, J = 12.5 Hz, 1H), 5.81 (d, J = 53.5 Hz, 2H), 7.07 (dd, J = 12.0, 8.8 Hz, 1H), 7.62-7.55 (m, 2H), 7.91-7.85 (m, 1H), 8.29 (d, J = 8.8 Hz, 1H), 8.42 (d, J = 2.8 Hz, 1H), 9.82 (s, 1H). | 469 | 1.34 |
| I-59 | 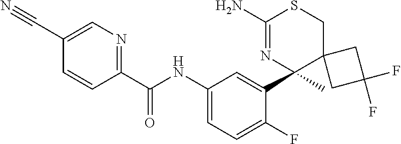 [SMILES: C[C@]1(c2cc(NC(=O)c3ccc(C#N)cn3)ccc2F)N=C(N)SCC12CC(F)(F)C2] | 1H-NMR (400 MHz, CDCl3) δ: 1.69 (d, J = 2.8 Hz, 3H), 2.32-2.44 (m, 1H), 2.44-2.56 (m, 1H), 2.83 (q, J = 1.38 Hz, 1H), 3.06-2.92 (m, 2H), 3.19 (d, J = 12.6 Hz, 1H), 7.10 (dd, J = 11.8, 8.8 Hz, 1H), 7.61 (dd, J = 7.0, 2.8 Hz, 1H), 7.92-7.85 (m, 1H), 8.20 (dd, J = 8.3, 2.0 Hz, 1H), 8.43 (d, J = 8.3 Hz, 1H), 8.90 (s, 1H), 9.84 (s, 1H). | 446 | 1.27 |
| I-60 | 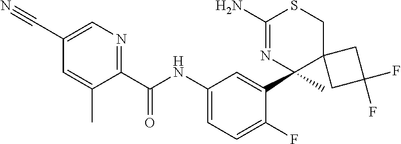 [SMILES: Cc1cc(C#N)cnc1C(=O)Nc1ccc(F)c([C@@]2(C)N=C(N)SCC23CC(F)(F)C3)c1] | 1H-HMR (400 MHz, CDCl3) δ: 1.69 (d, J = 2.5 Hz, 3H), 2.32-2.43 (m, 1H), 2.43-2.55 (m, 1H), 2.75-2.89 (m, 4H), 2.91-3.05 (m, 2H), 3.19 (d, J = 12.3 Hz, 1H), 7.09 (dd, J = 11.6, 8.8 Hz, 1H), 7.44 (dd, J = 2.3, 6.8 Hz, 1H), 7.98-7.90 (m, 2H), 8.73 (s, 1H), 9.98 (s, 1H). | 460 | 1.33 |
| I-61 | 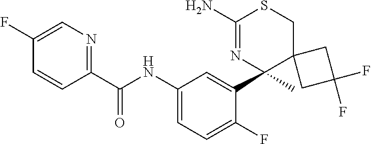 [SMILES: C[C@]1(c2cc(NC(=O)c3ccc(F)cn3)ccc2F)N=C(N)SCC12CC(F)(F)C2] | 1H-NMR (400 MHz, CDCl3) δ: 1.68 (d, J = 2.8 Hz, 3H), 2.31-2.43 (m, 1H), 2.43-2.55 (m, 1H), 2.82 (dd, J = 30.1, 13.8 Hz, 1H), 2.92-3.07 (m, 2H), 3.17 (d, J = 12.5 Hz, 1H), 7.08 (dd, J = 12.0, 6.6 Hz, 1H), 7.56-7.63 (m, 2H), 7.90-7.84 (m, 1H), 8.33 (dd, J = 8.5, 4.5 Hz, 1H), 8.46 (d, J = 2.5 Hz, 1H), 9.79 (s, 1H). | 439 | 1.27 |
| I-62 | 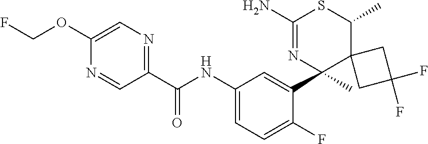 [SMILES: C[C@H]1SC(N)=N[C@](C)(c2cc(NC(=O)c3cnc(OCF)cn3)ccc2F)C12CC(F)(F)C2] | 1H-NMR (400 MHz, CDCl3) δ: 1.27 (d, J = 6.9 Hz, 3H), 1.73 (s, 3M), 2.68-2.86 (m, 3H), 3.22-3.42 (m, 2H), 6.15 (dd, J = 51.1, 1.8 Hz, 2H), 7.07 (dd, J = 11.9, 8.7 Hz, 1H), 7.43 (dd, J = 7.0, 2.6 Hz, 1H), 7.91-7.86 (m, 1H), 8.29 (s, 1H), 9.08 (s, 1H), 9.45 (s, 1H). | 484 | 1.38 |

TABLE 1-11

| | | | | |
|---|---|---|---|---|
| I-63 | 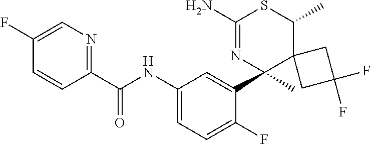 [SMILES: C[C@H]1SC(N)=N[C@](C)(c2cc(NC(=O)c3ccc(F)cn3)ccc2F)C12CC(F)(F)C2] | 1H-NMR (400 MHz, CDCl3) δ: 1.27 (d, J = 7.0 Hz, 3H), 1.74 (s, 3H), 2.67-2.85 (m, 3H), 3.24-3.42 (m, 2H), 7.06 (dd, J = 11.9, 8.7 Hz, 1H), 7.44 (dd, J = 7.0, 2.6 Hz, 1H), 7.59 (td, J = 8.7, 2.6 Hz, 1H), 7.93-7.87 (m, 1H), 8.33 (dd, J = 6.7, 4.6 Hz, 1H), 8.46 (d, J = 2.6 Hz, 1H), 9.76 (s, 1H). | 453 | 1.39 |
| I-64 | 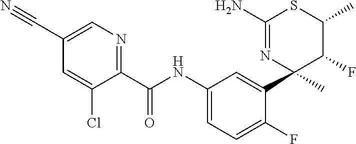 [SMILES: C[C@H]1SC(N)=N[C@](C)(c2cc(NC(=O)c3ncc(C#N)cc3Cl)ccc2F)[C@H]1F] | 1H-NMR (400 MHz, CDCl3) δ: 1.35 (d, J = 7.0 Hz, 3H), 1.75 (s, 3H), 3.09 (dq, J = 29.7, 7.0 Hz, 1H), 5.12 (d, J = 47.4 Hz, 1H), 7.06-7.18 (m, 2H), 8.13-8.08 (m, 1H), 8.17 (d, J = 1.8 Hz, 1H), 8.79 (d, J = 1.8 Hz, 1H). | 436 | 1.28 |
| I-65 | 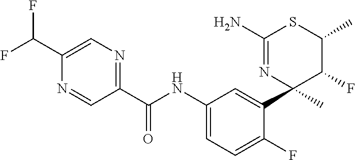 [SMILES: C[C@H]1SC(N)=N[C@](C)(c2cc(NC(=O)c3cnc(C(F)F)cn3)ccc2F)[C@H]1F] | 1H-NMR (400 MHz, CDCl3) δ: 1.35 (d, J = 6.9 Hz, 3H), 1.77 (s, 3H), 3.10 (dq, J = 30.7, 6.9 Hz, 1H), 5.12 (d, J = 48.4 Hz, 1H), 6.80 (t, J = 54.4 Hz, 1H), 7.11 (dd, J = 11.5, 8.8 Hz, 1H), 7.35 (dd, J = 6.7, 2.7 Hz, 1H), 8.02- 7.96 (m, 1H), 8.93 (s, 1H), 9.53 (s, 1H), 9.62 (s, 1H). | 428 | 1.22 |
| I-66 | 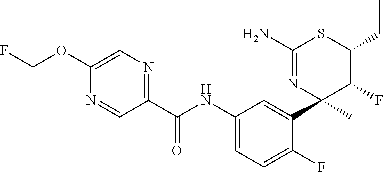 [SMILES: CC[C@H]1SC(N)=N[C@](C)(c2cc(NC(=O)c3cnc(OCF)cn3)ccc2F)[C@H]1F] | 1H-NMR (400 MHz, CDCl3) δ: 0.94 (t, J = 7.4 Hz, 3H), 1.61-1.70 (m, 1H), 1.76 (s, 3H), 1.82-1.91 (m, 1H), 2.93 (dt, J = 31.1, 8.0 Hz, 1H), 5.25 (d, J = 48.7 Hz, 1H), 6.15 (dd, J = 51.1, 2.9 Hz, 2H), 7.10 (dd, J = 11.4, 8.9 Hz, 1H), 7.26-7.30 (m, 1H), 8.05-7.99 (m, 1H), 8.30 (s, 1H), 9.08 (s, 1H), 9.48 (s, 1H). | 440 | 1.36 |
| I-67 | 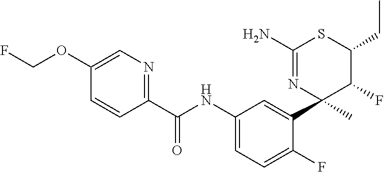 [SMILES: CC[C@H]1SC(N)=N[C@](C)(c2cc(NC(=O)c3ccc(OCF)cn3)ccc2F)[C@H]1F] | 1H-NMR (400 MHz, CDCl3) δ: 0.94 (t, J = 7.4 Hz, 3H), 1.61-1.70 (m, 1H), 1.76 (s, 3H), 1.82-1.89 (m, 1H), 2.94 (dt, J = 31.4, 7.5 Hz, 1H), 5.25 (d, J = 48.9 Hz, 1H), 5.81 (d, J = 53.5 Hz, 2H), 7.08 (dd, J = 11.7, 8.9 Hz, 1H), 7.27-7.31 (m, 12H), 7.57 (dd, J = 8.4, 2.9 Hz, 1H), 8.01-8.06 (m, 1H), 6.29 (d, J = 8.4 Hz, 1H), 8.42 (d, J = 2.9 Hz, 1H), 9.82 (s, 1H). | 439 | 1.40 |

TABLE 1-12

| | | | | |
|---|---|---|---|---|
| I-68 | 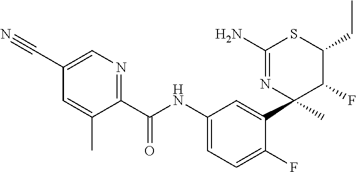 [SMILES: CC[C@H]1SC(N)=N[C@](C)(c2cc(NC(=O)c3ncc(C#N)cc3C)ccc2F)[C@H]1F] | 1H-NMR (400 MHz, CDCl3 δ: 0.94 (t, J = 7.4 Hz, 3H), 1.62-1.71 (m, 1H), 1.75 (s, 3H), 1.83-1.90 (m, 1H), 3.00- 2.34 (m, 4H), 5.26 (d, J = 47.2 Hz, 1H), 7.09 (dd, J = 11.7, 8.7 Hz, 1H), 7.19 (dd, J = 6.5, 2.8 Hz, 1H), 7.95 (s, 1H), 6.05-6.09 (m, 1H), 8.73 (s, 1H), 9.98 (s, 1H). | 430 | 1.42 |
| I-69 | 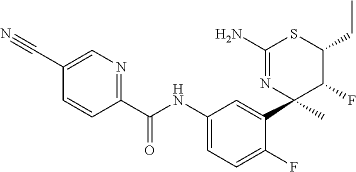 [SMILES: CC[C@H]1SC(N)=N[C@](C)(c2cc(NC(=O)c3ccc(C#N)cn3)ccc2F)[C@H]1F] | 1H-NMR (400 MHz, CDCl3) δ: 0.94 (t, J = 7.5 Hz, 3H), 1.61-1.70 (m, 1H), 1.76 (s, 3H), 1.81-1.92 (m, 1H), 2.93 (dt, J = 31.4, 7.1 Hz, 1H), 5.25 (d, J = 47.3 Hz, 1H), 7.11 (dd, J = 11.5, 8.8 Hz, 1H), 7.34 (dd, J = 6.7, 2.8 Hz, 1H), 7.99-8.05 (m, 1H), 8.21 (dd, J = 8.1, 1.9 Hz, 1H), 8.43 (d, J = 8.1 Hz, 1H), 8.91 (s, 1H), 9.84 (s, 1H). | 416 | 1.31 |
| I-70 | 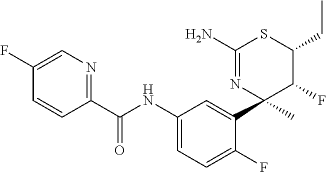 [SMILES: CC[C@H]1SC(N)=N[C@](C)(c2cc(NC(=O)c3ccc(F)cn3)ccc2F)[C@H]1F] | 1H-NMR (400 MHz, CDCl3) δ: 0.94 (t, J =7.4 Hz, 3H), 1.61-1.72 (m, 1H), 1.76 s, 3H), 1.81-1.91 (m, 1H), 2.94 (dt, J = 30.9, 7.2 Hz, 1H), 5.25 (d, J = 47.6 Hz, 1H), 7.09 (dd, J = 11.5, 8.9 Hz, 1H), 7.30 (dd, J = 6.7, 2.8 Hz, 1H), 7.63-7.57 (m, 1H), 7.99-8.05 (m, 1H), 8.33 (dd, J = 8.7, 4.6 Hz, 1H), 8.46 (d, J = 2.8 Hz, 1H), 9.79 (s, 1H). | 409 | 1.34 |
| I-71 | 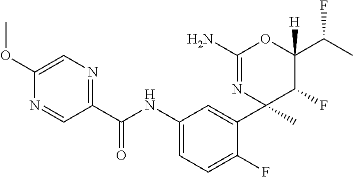 [SMILES: COc1cnc(C(=O)Nc2ccc(F)c([C@@]3(C)N=C(N)O[C@H]([C@@H](C)F)[C@@H]3F)c2)cn1] | 1H-NMR (400 MHz, CDCl3) δ: 1.39 (dd, J = 25.6, 6.3 Hz, 3H), 1.68 (s, 3H), 3.67 (m, 1H), 4.07 (s, 3H), 4.80 (m, 1H), 5.35 (d, J = 47.7 Hz, 1H), 7.09 (dd, J = 11.3, 8.8 Hz, 1H), 7.48 (dd, J = 7.0, 2.8 Hz, 1H), 8.00 (m, 1H), 8.16 (d, J = 1.3 Hz, 1H), 9.02 (d, J = 1.3 Hz, 1H), 9.52 (s, 1H). | 424 | 1.3 |
| I-75 | 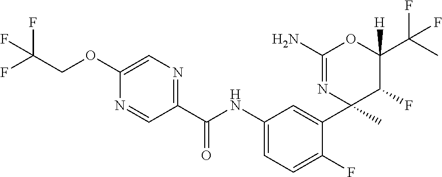 [SMILES: CC(F)(F)[C@H]1OC(N)=N[C@](C)(c2cc(NC(=O)c3cnc(OCC(F)(F)F)cn3)ccc2F)[C@H]1F] | 1H-NMR (400 MHz, CDCl3) δ: 1.66 (s, 3H), 1.72 (t, J = 19.7 Hz, 3H), 3.89 (ddd, J = 29.7, 10.8, 4.8 Hz, 1H), 4.32 (brs, 2H), 4.85 (d, J = 8.3 Hz, 1H), 4.89 (d, J = 8.3 Hz, 1H), 5.38 (d, J = 48.1 Hz, 1H), 7.11 (dd, J = 11.3, 8.9 Hz, 1H), 7.52 (dd, J = 6.7, 2.6 Hz, 1H), 7.99 (m, 1H), 8.31 (brs, 1H), 9.02 (brs, 1H), 9.50 (s, 1H). | 510 | 1.62 |

TABLE 1-13

| | | | | |
|---|---|---|---|---|
| I-76 | 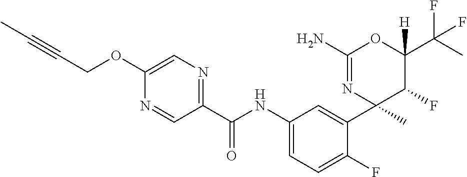 [SMILES: CC#CCOc1cnc(C(=O)Nc2ccc(F)c([C@@]3(C)N=C(N)O[C@H](C(C)(F)F)[C@@H]3F)c2)cn1] | 1H-NMR (400 MHz, CDCl3) δ: 1.67 (s, 3H), 1.72 (t, J = 19.6 Hz, 3H), 1.89 (t, J = 2.3 Hz, 3H), 3.89 (ddd, J = 29.7, 10.7, 4.8 Hz, 1H), 4.38 (brs, 2H), 5.05 (q, J = 2.3 Hz, 2H), 5.38 (d, J = 47.9 Hz, 1H), 7.10 (dd, J = 11.4, 8.9 Hz, 1H), 7.49 (dd, J = 6.8, 2.7 Hz, 1H), 8.00 (m, 1H), 8.20 (brs, 1H), 9.03 (brs, 1H), 9.51 (s, 1H). | 480 | 1.58 |
| I-77 | 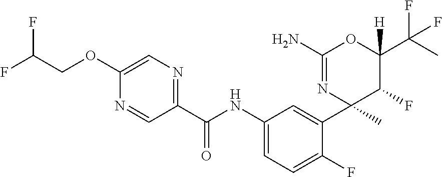 [SMILES: CC(F)(F)[C@H]1OC(N)=N[C@](C)(c2cc(NC(=O)c3cnc(OCC(F)F)cn3)ccc2F)[C@H]1F] | 1H-NMR (400 MHz, CDCl3) δ: 1.66 (s, 3H), 1.72 (t, J = 19.6 Hz, 3H), 3.89 (ddd, J = 29.6, 10.8, 4.9 Hz, 1H), 4.38 (brs, 2H), 4.66 (td, J = 13.4, 4.0 Hz, 2H), 5.38 (d, J = 48.2 Hz, 1H), 6.16 (tt, J = 55.1, 4.0 Hz, 1H), 7.11 (dd, J = 11.4, 8.8 Hz, 1H), 7.50 (dd, J = 6.8, 2.8 Hz, 1H), 8.00 (ddd, J = 8.8, 4.3, 2.8 Hz, 1H), 8.27 (d, J = 1.3 Hz, 1H), 9.02 (d, J = 1.3 Hz, 1H), 9.50 (s, 1H). | 492 | 1.49 |
| I-78 | 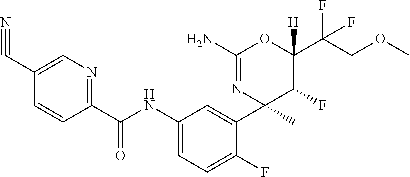 [SMILES: COCC(F)(F)[C@H]1OC(N)=N[C@](C)(c2cc(NC(=O)c3ccc(C#N)cn3)ccc2F)[C@H]1F] | 1H-NMR (400 MHz, CDCl3) δ: 1.67 (s, 3H), 3.39 (s, 3H), 3.74 (m, 2H), 4.07 (m, 1H), 4.37 (brs, 2H), 5.48 (d, J = 48.1 Hz, 1H), 7.12 (dd, J = 11.2, 9.0 Hz, 1H), 7.54 (m, 1H), 8.04 (m, 1H), 8.21 (dd, J = 8.2, 2.0 Hz, 1H), 8.43 (dd, J = 8.2, 0.8 Hz, 1H), 8.90 (brs, 1H), 9.88 (s, 1H). | 466 | 1.31 |
| I-79 | 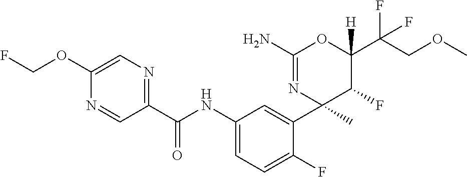 [SMILES: COCC(F)(F)[C@H]1OC(N)=N[C@](C)(c2cc(NC(=O)c3cnc(OCF)cn3)ccc2F)[C@H]1F] | 1H-NMR (400 MHz, CDCl3) δ: 1.66 (s, 3H), 3.39 (s, 3H), 3.74 (m, 2H), 4.08 (m, 1H), 4.36 (brs, 2H), 5.47 (d, J = 48.1 Hz, 1H), 6.15 (m, 2H), 7.11 (dd, J = 11.4, 8.9 Hz, 1H), 7.50 (dd, J = 6.7, 2.6 Hz, 1H), 8.03 (m, 1H), 8.29 (d, J = 1.3 Hz, 1H), 9.08 (d, J = 1.3 Hz, 1H), 9.52 (s, 1H). | 490 | 1.33 |
| I-80 | 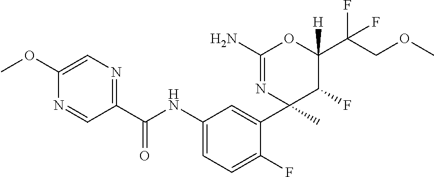 | 1H-NMR (400 MHz, CDCl3) δ: 1.66 (s, 3H), 3.39 (s, 3H), 3.74 (m, 2H), 4.07 (s, 3H), 4.07 (m, 1H), 4.35 (brs, 2H), 5.47 (d, J = 48.1 Hz, 1H), 7.10 (dd, J = 11.4, 8.9 Hz, 1H), 7.48 (dd, J = 6.8, 2.6 Hz, 1H), 8.03 (ddd, J = 8.9, 4.4, 2.6 Hz, 1H), 8.16 (d, J = 1.3 Hz, 1H), 9.02 (d, J = 1.3 Hz, 1H), 9.52 (s, 1H). | 472 | 1.3 |

TABLE 1-14

| | | | | |
|---|---|---|---|---|
| I-81 | 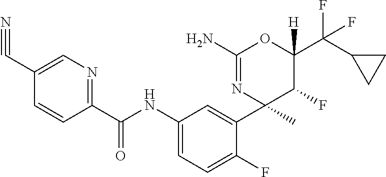 [SMILES: C[C@]1(c2cc(NC(=O)c3ccc(C#N)cn3)ccc2F)N=C(N)O[C@H](C(F)(F)C2CC2)[C@@H]1F] | 1H-NMR (400 MHz, CDCl3) δ: 0.64 (m, 2H), 0.73 (m, 2H), 1.38 (m, 1H), 1.68 (s, 3H), 3.94 (ddd, J = 29.9, 10.0, 7.2 Hz, 1H), 4.38 (brs, 2H), 5.45 (d, J = 48.1 Hz, 1H) 7.12 (dd, J = 11.3, 8.9 Hz, 1H), 7.55 (dd, J = 6.7, 2.6 Hz, 1H), 8.04 (ddd, J = 8.9, 4.1, 2.6 Hz, 1H), 8.21 (dd, J = 8.2, 2.0 Hz, 1H), 8.43 (dd, J = 8.2, 0.8 Hz, 1H), 8.90 (dd, J = 2.0, 0.8 Hz, 1H), 9.88 (s, 1H). | 462 | 1.44 |
| I-82 | 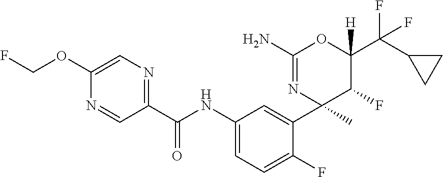 [SMILES: C[C@]1(c2cc(NC(=O)c3cnc(OCF)cn3)ccc2F)N=C(N)O[C@H](C(F)(F)C2CC2)[C@@H]1F] | 1H-NMR (400 MHz, CDCl3) δ: 0.64 (m, 2H), 0.73 (m, 2H), 1.38 (m, 1H), 1.67 (s, 3H), 3.95 (ddd, J = 29.9, 10.0, 7.3 Hz, 1H), 4.37 (brs, 2H), 5.45 (d, J = 48.1 Hz, 1H), 6.15 (m, 2H), 7.11 (dd, J = 11.3, 8.9 Hz, 1H), 7.50 (dd, J = 6.8, 2.8 Hz, 1H), 8.03 (ddd, J = 8.9, 4.1, 2.8 Hz, 1H), 8.29 (d, J = 1.4 Hz, 1H), 9.08 (d, J = 1.4 Hz, 1H), 9.52 (s, 1H), | 486 | 1.48 |
| I-83 | 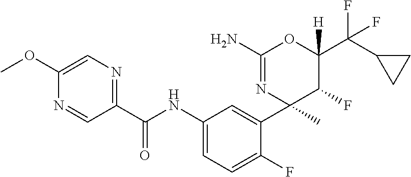 [SMILES: COc1cnc(C(=O)Nc2ccc(F)c([C@@]3(C)N=C(N)O[C@H](C(F)(F)C4CC4)[C@@H]3F)c2)cn1] | 1H-NMR (400 MHz, CDCl3) δ: 0.64 (m, 2H), 0.72 (m, 2H), 1.38 (m, 1H), 1.67 (s, 3H), 3.95 (ddd, J = 29.9, 10.0, 7.4 Hz, 1H), 4.07 (s, 3H), 4.36 (brs, 2H), 5.45 (d, J = 48.1 Hz, 1H), 7.10 (dd, J = 11.3, 8.9 Hz, 1H), 7.49 (dd, J = 6.7, 2.6 Hz, 1H), 8.04 (m, 1H), 8.16 (brs, 1H), 9.02 (brs, 1H), 9.53 (s, 1H). | 468 | 1.45 |
| I-84 | 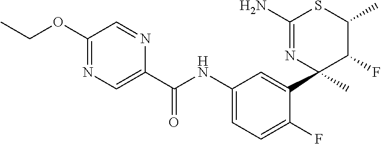 [SMILES: CCOc1cnc(C(=O)Nc2ccc(F)c([C@@]3(C)N=C(N)S[C@H](C)[C@@H]3F)c2)cn1] | 1H-NMR (CDCl3) δ: 1.34 (d, J = 6.9 Hz, 3H), 1.46 (t, J = 7.0 Hz, 3H), 1.77 (s, 3H), 3.10 (dq, J = 30.5, 6.8 Hz, 1H), 4.49 (q, J = 7.1 Hz, 2H), 5.12 (d, J = 47.4 Hz, 1H), 7.07 (dd, J = 11.5, 8.8 Hz, 1H), 7.26-7.30 (m, 1H), 7.97- 8.01 (m, 1H), 8.12 (d, J = 1.1 Hz, 1H), 8.99 (d, J = 1.1 Hz, 1H), 9.48 (s, 1H). | 422 | 1.28 |
| I-85 | 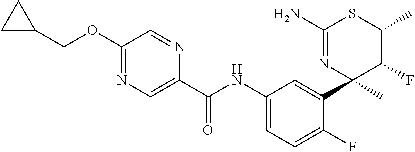 [SMILES: C[C@H]1SC(N)=N[C@](C)(c2cc(NC(=O)c3cnc(OCC4CC4)cn3)ccc2F)[C@H]1F] | 1H-NMR (CDCl3) δ: 0.40 (dd, J = 10.5, 4.8 Hz, 2H), 0.65-0.68 (m, 2H), 1.29-1.36 (m, 3H), 1.76 (s, 3H), 3.10 (dq, J = 30.5, 6.7 Hz, 1H), 4.27 (d, J = 7.3 Hz, 2H), 5.11 (dd, J = 47.6, 1.1 Hz, 1H), 7.07 (dd, J = 11.5, 8.9 Hz, 1H), 7.28 (dd, J = 6.8, 2.7 Hz, 1H), 7.96-8.00 (m, 1H), 8.16 (d, J = 1.3 Hz, 1H), 8.97 (d, J = 1.3 Hz, 1H), 9.47 (s, 1H). | 448 | 1.48 |

TABLE 1-15

| | | | | |
|---|---|---|---|---|
| I-86 | 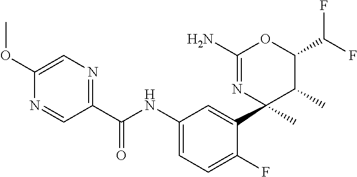 [SMILES: COc1cnc(C(=O)Nc2ccc(F)c([C@@]3(C)N=C(N)O[C@H](C(F)F)[C@@H]3C)c2)cn1] | 1H-NMR (400 MHz, CDCl3) δ 1.16 (d, J = 6.8 Hz, 3H), 1.65 (s, 3H), 2.76-2.78 (m, 1H), 4.01-4.07 (m, 4H), 5.79 (td, J = 56.4, 6.4 Hz, 1H), 7.09- 7.14 (m, 1H), 7.36-7.38 (m, 1H), 8.02-8.05 (m, 1H), 8.18 (s, 1H), 8.56 (s, 1H), 9.02 (s, 1H), 9.54 (s, 1H). | 424 | 1.28 |
| I-87 | 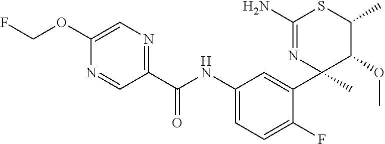 [SMILES: CO[C@H]1[C@@H](C)SC(N)=N[C@]1(C)c1cc(NC(=O)c2cnc(OCF)cn2)ccc1F] | 1H-NMR (400 MHz, CDCl3) δ 1.29 (d, J = 6.9 Hz, 3H), 1.75 (s, 3H), 3.03-3.07 (m, 1H), 3.71 (s, 3H), 3.88 (d, J = 1.6 Hz, 1H), 6.15 (dd, J = 51.5, 2.5 Hz, 2H), 7.08 (dd, J = 11.7, 8.8 Hz, 1H), 7.18 (dd, J = 6.8, 2.8 Hz, 1H), 8.02-8.06 (m, 1H), 8.29 (d, J = 1.3 Hz, 1H), 9.08 (d, J = 1.3 Hz, 1H), 9.46 (s, 1H). | 438 | 1.19 |
| I-88 | 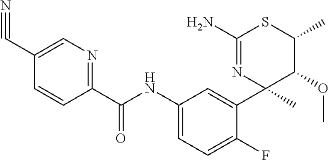 [SMILES: CO[C@H]1[C@@H](C)SC(N)=N[C@]1(C)c1cc(NC(=O)c2ccc(C#N)cn2)ccc1F] | 1H-NMR (400 MHz, CDCl3) δ 1.30 (d, J = 6.8 Hz, 3H), 1.76 (s, 3H), 3.02-3.07 (m, 1H), 3.72 (s, 3H), 3.88 (d, J = 1.8 Hz, 1H), 7.09 (dd, J = 11.5, 8.8 Hz, 1H), 7.24 (dd, J = 6.9, 2.8 Hz, 1H), 8.01-8.05 (m, 1H), 8.20 (dd, J = 8.1, 1.9 Hz, 1H), 8.42 (d, J = 8.2 Hz, 1H), 8.90 (d, J = 1.1 Hz, 1H), 9.82 (s, 1H). | 414 | 1.15 |
| I-89 | 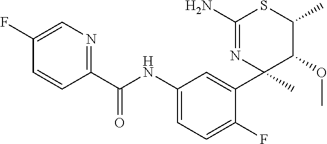 [SMILES: CO[C@H]1[C@@H](C)SC(N)=N[C@]1(C)c1cc(NC(=O)c2ccc(F)cn2)ccc1F] | 1H-NMR (400 MHz, CDCl3) δ 1.29 (d, J = 6.8 Hz, 3H), 1.75 (s, 3H), 3.04-3.06 (m, 1H), 3.72 (s, 3H), 3.87 (d, J = 2.0 Hz, 1H), 7.07 (dd, J = 11.7, 8.9 Hz, 1H), 7.20 (dd, J = 6.9, 2.9 Hz, 1H), 7.59 (td, J = 8.3, 2.8 Hz, 1H), 8.01-8.05 (m, 1H), 6.32 (dd, J = 8.8, 4.5 Hz, 1H), 8.46 (d, J = 2.8 Hz, 1H), 9.77 (s, 1H). | 407 | 1.24 |
| I-90 | 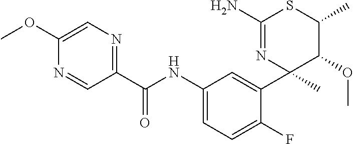 | 1H-NMR (400 MHz, CDCl3) δ 1.29 (d, J = 6.9 Hz, 3H), 1.75 (s, 3H), 3.03-3.08 (m, 1H), 3.72 (s, 3H), 3.87 (d, J = 1.6 Hz, 1H), 4.07 (s, 3H), 7.07 (dd, J = 11.7, 8.8 Hz, 1H), 7.17 (dd, J = 6.9, 2.6 Hz, 1H), 8.03 (ddd, J = 8.7, 4.0, 3.0 Hz, 1H), 8.16 (d, J = 1.1 Hz, 1H), 9.01 (d, J = 1.1 Hz, 1H), 9.47 (s, 1H). | 420 | 1.23 |

TABLE 1-16

| | | | | |
|---|---|---|---|---|
| I-91 | 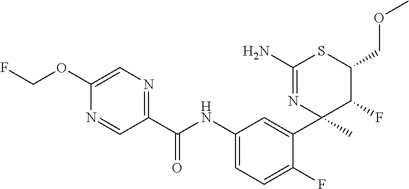 [SMILES: COC[C@H]1SC(N)=N[C@](C)(c2cc(NC(=O)c3cnc(OCF)cn3)ccc2F)[C@H]1F] | 1H-NMR (400 MHz, CDCl3) δ 1.76 (s, 3H), 3.29-3.45 (m, 1H), 3.33 (s, 3H), 3.70 (dd, J = 9.3, 6.5 Hz, 1H), 5.36 (dd, J = 47.8, 1.6 Hz, 1H), 6.16 (dd, J = 51.2, 0.8 Hz, 2H), 7.09 (dd, J = 11.3, 8.8 Hz, 1H), 7.32 (dd, J = 6.7, 2.6 Hz, 1H), 7.98 (ddd, J = 6.8, 4.1, 2.9 Hz, 1H), 8.29 (d, J = 1.3 Hz, 1H), 9.08 (d, J = 1.3 Hz, 1H), 9.47 (s, 1H). | 456 | 1.20 |
| I-92 | 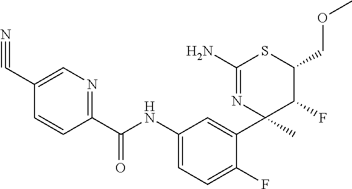 [SMILES: COC[C@H]1SC(N)=N[C@](C)(c2cc(NC(=O)c3ccc(C#N)cn3)ccc2F)[C@H]1F] | 1H-NMR (400 MHz, CDCl3) δ 1.76 (s, 3H), 3.28-3.45 (m, 1H), 3.33 (s, 3H), 3.78 (dd, J = 9.2, 6.5 Hz, 1H), 5.36 (dd, J = 47.8, 1.5 Hz, 1H), 7.10 (dd, J = 11.4, 8.8 Hz, 1H), 7.36 (dd, J = 6.7, 2.8 Hz, 1H), 8.00 (dt, J = 8.8, 3.5 Hz, 1H), 8.20 (dd, J = 8.2, 2.0 Hz, 1H), 8.43 (d, J = 8.2 Hz, 1H), 8.90 (d, J = 1.1 Hz, 1H), 9.82 (s, 1H). | 432 | 1.25 |
| I-93 | 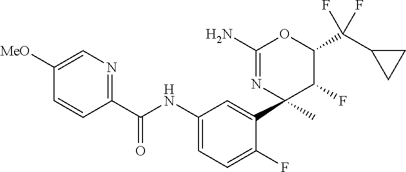 [SMILES: COc1ccc(C(=O)Nc2ccc(F)c([C@@]3(C)N=C(N)O[C@H](C(F)(F)C4CC4)[C@@H]3F)c2)nc1] | 1H-NMR (CDCl3) δ: 0.64 (m, 2H), 0.72 (m, 2H), 1.38 (m, 1H), 1.67 (s, 3H), 3.94 (s, 3H), 3.95 (m, 1H), 4.36 (brs, 2H), 5.45 (d, J = 48.2 Hz, 1H), 7.09 (dd, J = 11.4, 8.8 Hz, 1H), 7.34 (dd, J = 8.7, 2.8 Hz, 1H), 7.50 (dd, J = 6.8, 2.8 Hz, 1H), 8.05 (ddd, J = 8.8, 4.3, 2.6 Hz, 1H), 8.24 (d, J = 8.7 Hz, 1H), 8.27 (d, J = 2.8 Hz, 1H), 9.86 (s, 1H). | 467 | 1.52 |
| I-94 | 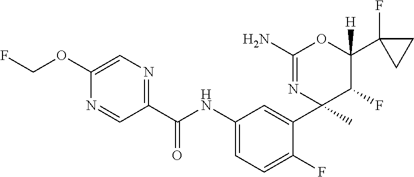 [SMILES: C[C@]1(c2cc(NC(=O)c3cnc(OCF)cn3)ccc2F)N=C(N)O[C@H](C2(F)CC2)[C@@H]1F] | 1H-NMR (CDCl3) δ: 0.76-1.20 (m, 4H), 1.67 (s, 3H), 4.11 (dd, J = 29.2, 11.8 Hz, 1H), 5.35 (d, J = 47.9 Hz, 1H), 6.15 (m, 2H), 7.11 (dd, J = 11.3, 8.9 Hz, 1H), 7.50 (dd, J = 6.8, 2.6 Hz, 1H), 8.02 (m, 1H), 8.29 (s, 1H), 9.08 (s, 1H), 9.52 (s, 1H). | 454 | 1.39 |
| I-95 | 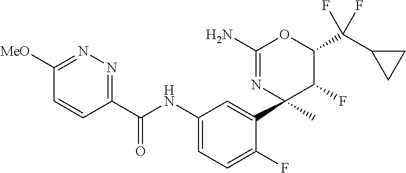 [SMILES: COc1ccc(C(=O)Nc2ccc(F)c([C@@]3(C)N=C(N)O[C@H](C(F)(F)C4CC4)[C@@H]3F)c2)nn1] | 1H-NMR (CDCl3) δ: 0.64 (m, 2H), 0.73 (m, 2H), 1.39 (m, 1H), 1.68 (s, 3H), 3.94 (ddd, J = 30.0, 9.9, 7.0 Hz, 1H), 4.24 (s, 3H), 5.43 (d, J = 48.2 Hz, 1H), 7.11 (dd, J = 11.4, 8.9 Hz, 1H), 7.17 (d, J = 9.1 Hz, 1H), 7.51 (dd, J = 6.8, 2.8 Hz, 1H), 8.05 (m, 1H), 6.28 (d, J = 9.1 Hz, 1H), 9.91 (s, m | 468 | 1.45 |

TABLE 1-17

| | | | | |
|---|---|---|---|---|
| I-96 | 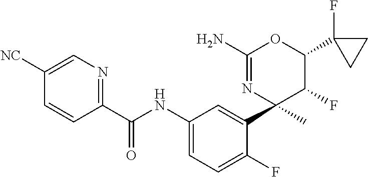 [SMILES: C[C@]1(c2cc(NC(=O)c3ccc(C#N)cn3)ccc2F)N=C(N)O[C@H](C2(F)CC2)[C@@H]1F] | 1H-NMR (CDCl3) δ: 0.76-1.20 (m, 4H), 1.67 (s, 3H), 4.12 (dd, J = 29.2, 11.5 Hz, 1H), 5.35 (d, J = 47.9 Hz, 1H), 7.12 (dd, J = 11.3, 8.9 Hz, 1H), 7.55 (dd, J = 6.8, 2.8 Hz, 1H), 8.03 (m, 1H), 8.21 (dd, J = 8.2, 2.0 Hz, 1H), 6.43 (d, J = 8.2 Hz, 1H), 8.91 (d, J = 2.0 Hz, 1H), 9.88 (s, 1H). | 430 | 1.32 |
| I-97 | 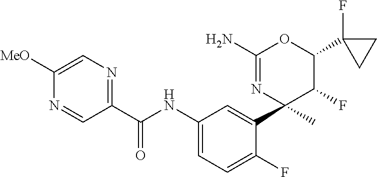 [SMILES: COc1cnc(C(=O)Nc2ccc(F)c([C@@]3(C)N=C(N)O[C@H](C4(F)CC4)[C@@H]3F)c2)cn1] | 1H-NMR (CDCl3) δ: 0.75-1.20 (m, 4H), 1.67 (s, 3H), 4.07 (s, 3H), 4.11 (dd, J = 29.5, 11.9 Hz, 1H), 5.35 (d, J = 47.9 Hz, 1H), 7.10 (dd, J = 11.3, 8.9 Hz, 1H), 7.48 (dd, J = 6.3, 2.6 Hz, 1H), 8.03 (m, 1H), 8.16 (d, J = 1.1 Hz, 1H), 9.02 (d, J = 1.1 Hz, 1H), 9.53 (s, 1H). | 436 | 1.33 |
| I-99 | 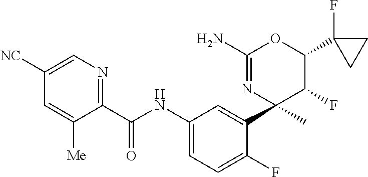 [SMILES: Cc1cc(C#N)cnc1C(=O)Nc1ccc(F)c([C@@]2(C)N=C(N)O[C@H](C3(F)CC3)[C@@H]2F)c1] | 1H-NMR (CDCl3) δ: 0.76-1.20 (m, 4H) 1.66 (s, 3H), 2.86 (s, 3H), 4.12 (dd, J = 29.4, 11.8 Hz, 1H), 5.35 (d, J = 47.9 Hz, 1H), 7.10 (dd, J = 11.3, 8.8 Hz, 1H), 7.43 (dd, J = 6.8, 2.5 Hz, 1H), 7.95 (d, J = 1.5 Hz, 1H), 8.06 (m, 1H), 8.73 (d, J = 1.5 Hz, 1H), 10.03 (s, 1H). | 444 | 1.40 |
| I-100 | 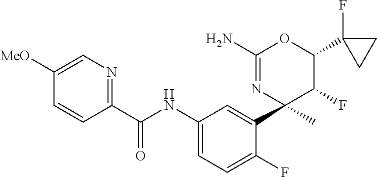 [SMILES: COc1ccc(C(=O)Nc2ccc(F)c([C@@]3(C)N=C(N)O[C@H](C4(F)CC4)[C@@H]3F)c2)nc1] | 1H-NMR (CDCl3) δ: 0.74-1.20 (m, 4H), 1.68 (s, 3H), 3.94 (s, 3H), 4.11 (dd, J = 29.4, 11.9 Hz, 1H), 5.35 (d, J = 47.9 Hz, 1H), 7.09 (dd, J = 11.4, 8.9 Hz, 1H), 7.34 (dd, J = 8.7, 2.8 Hz, 1H), 7.49 (dd, J = 6.8, 2.6 Hz, 1H), 8.05 (m, 1H), 8.24 (d, J = 8.7 Hz, 1H), 8.27 (d, J = 2.8 Hz, 1H), 9.86 (s, 1H). | 435 | 1.38 |

TABLE 1-18

| | | | | |
|---|---|---|---|---|
| I-101 | 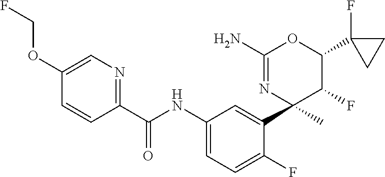 [SMILES: C[C@]1(c2cc(NC(=O)c3ccc(OCF)cn3)ccc2F)N=C(N)O[C@H](C2(F)CC2)[C@@H]1F] | 1H-NMR (CDCl3) δ: 0.75-1.20 (m, 4H), 1.67 (s, 3H), 4.11 (dd, J = 29.4, 11.9 Hz, 1H), 5.35 (d, J = 47.9 Hz, 1H), 5.81 (d, J = 53.5 Hz, 2H), 7.10 (dd, J = 11.4, 8.9 Hz, 1H), 7.51 (dd, J = 6.7, 2.6 Hz, 1H), 7.58 (dd, J = 8.7, 2.6 Hz, 1H), 8.04 (m, 1H), 8.29 (d, J = 8.7 Hz, 1H), 8.42 (d, J = 2.6 Hz, 1H), 9.86 (s, 1H). | 453 | 1.39 |
| I-102 | 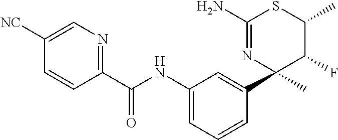 [SMILES: C[C@H]1SC(N)=N[C@](C)(c2cccc(NC(=O)c3ccc(C#N)cn3)c2)[C@H]1F] | 1H-NMR (CDCl3) δ: 1.29 (d, J = 6.9 Hz, 3H), 1.74 (d, J = 2.1 Hz, 3H), 3.02-3.14 (m, 1H), 4.65 (dd, J = 47.9, 1.1 Hz, 1H), 7.10 (d, J = 8.0 Hz, 1H), 7.40 (t, J = 8.0 Hz, 1H), 7.60 (s, 1H), 7.76 (dd, J = 6.0, 1.1 Hz, 1H), 8.22 (dd, J = 8.2, 2.0 Hz, 1H), 8.44 (d, J = 8.2 Hz, 1H), 6.91 (d, J = 1.1 Hz, 1H), 9.88 (s, 1H). | 384 | 1.08 |
| I-103 | 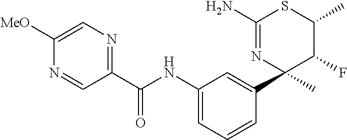 [SMILES: COc1cnc(C(=O)Nc2cccc([C@@]3(C)N=C(N)S[C@H](C)[C@@H]3F)c2)cn1] | 1H-NMR (CDCl3) δ: 1.29 (d, J = 7.0 Hz, 3H), 1.74 (d, J = 2.0 Hz, 4H), 3.09 (dq, J = 30.5, 7.0 Hz, 1H), 4.07 (s, 3H), 4.65 (d, J = 47.9 Hz, 1H), 7.05 (d, J = 7.9 Hz, 1H), 7.37 (t, J = 7.9 Hz, 1H), 7.59 (s, 1H), 7.72 (d, J = 7.9 Hz, 1H), 6.17 (d, J = 1.1 Hz, 1H), 9.03 (d, J = 1.1 Hz, 1H), 9.53 (s, 1H). | 390 | 1.11 |
| I-104 | 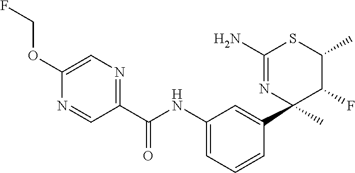 [SMILES: C[C@H]1SC(N)=N[C@](C)(c2cccc(NC(=O)c3cnc(OCF)cn3)c2)[C@H]1F] | 1H-NMR (CDCl3) δ: 1.29 (d, J = 6.9 Hz, 3H), 1.74 (d, J = 2.0 Hz, 3H), 3.09 (dq, J = 30.5, 6.9 Hz, 1H), 4.65 (dd, J = 47.9, 0.9 Hz, 1H), 6.16 (dd, J = 51.1, 1.3 Hz, 2H), 7.07 (d, J = 8.0 Hz, 1H), 7.38 (t, J = 8.0 Hz, 1H), 7.58 (s, 1H), 7.73 (d, J = 8.0 Hz, 1H), 8.30 (d, J = 1.1 Hz, 1H), 9.09 (d, J = 1.1 Hz, 1H), 9.53 (s, 1H). | 408 | 1.10 |
| I-105 | 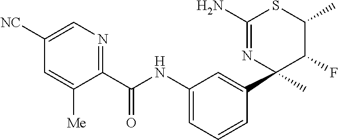 [SMILES: Cc1cc(C#N)cnc1C(=O)Nc1cccc([C@@]2(C)N=C(N)S[C@H](C)[C@@H]2F)c1] | 1H-NMR (CDCl3) δ: 1.29 (d, J = 7.0 Hz, 3H), 1.74 (d, J = 2.1 Hz, 4H), 2.87 (s, 3H), 3.08 (ddd, J = 30.2, 13.7, 7.0 Hz, 1H), 4.48 (s, 2H), 4.66 (d, J = 48.8 Hz, 1H), 7.07 (d, J = 8.0 Hz, 1H), 7.38 (t, J = 8.0 Hz, 1H), 7.49 (s, 1H), 7.78 (d, J = 8.0 Hz, 1H), 7.95 (s, 1H), 8.74 (d, J = 1.1 Hz, 1H), 10.03 (s, 1H). | 398 | 1.17 |

TABLE 1-19

| | | | | |
|---|---|---|---|---|
| I-106 | 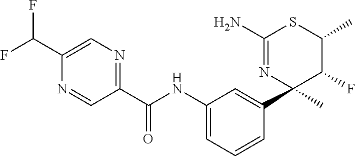 [SMILES: C[C@H]1SC(N)=N[C@](C)(c2cccc(NC(=O)c3cnc(C(F)F)cn3)c2)[C@H]1F] | 1H-NMR (CDCl3) δ: 1.30 (d, J = 7.0 Hz, 3H), 1.74 (s, 3H), 3.09 (dq, J = 30.3, 7.0 Hz, 1H), 4.65 (d, J = 47.9 Hz, 1H), 6.80 (t, J = 54.5 Hz, 1H), 7.11 (d, J = 7.9 Hz, 1H), 7.41 (t, J = 7.9 Hz, 1H), 7.60 (s, 1H), 7.76 (d, J = 7.9 Hz, 1H), 8.94 (s, 1H), 9.54 (s, 1H), 9.66 (s, 1H). | 410 | 1.11 |
| I-107 | 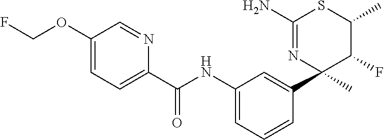 [SMILES: C[C@H]1SC(N)=N[C@](C)(c2cccc(NC(=O)c3ccc(OCF)cn3)c2)[C@H]1F] | 1H-NMR (CDCl3) δ: 1.29 (d, J = 6.9 Hz, 3H), 1.74 (d, J = 2.1 Hz, 3H), 3.09 (dq, J = 30.4, 6.9 Hz, 1H), 4.66 (d, J = 47.9 Hz, 1H), 5.82 (d, J = 53.5 Hz, 2H), 7.05 (d, J = 7.9 Hz, 1H), 7.37 (t, J = 7.9 Hz, 1H), 7.57-7.61 (m, 2H), 7.74 (d, J = 8.0 Hz, 1H), 8.30 (d, J = 8.7 Hz, 1H), 8.43 (d, J = 2.6 Hz, 1H), 9.87 (s, 1H). | 407 | 1.15 |
| I-108 | 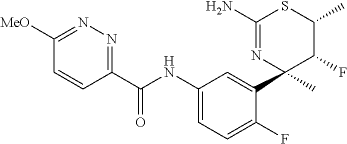 [SMILES: COc1ccc(C(=O)Nc2ccc(F)c([C@@]3(C)N=C(N)S[C@H](C)[C@@H]3F)c2)nn1] | 1H-NMR (400 MHz, CDCl3) δ: 1.33 (d, J = 7.0 Hz, 3H), 1.77 (s, 3H), 3.07 (dq, J = 31.1, 7.0 Hz, 1H), 4.24 (s, 3H), 5.09 (d, J = 48.7 Hz, 1H), 7.09 (dd, J = 11.5, 9.0 Hz 1H), 7.17 (d, J = 9.0 Hz, 1H), 7.26-7.30 (m, 1H), 8.07-8.01 (m, 1H), 8.28 (d, J = 9.2 Hz, 1H), 9.86 (s, 1H). | 406 | 1.18 |
| I-109 | 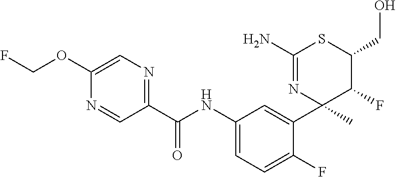 [SMILES: C[C@]1(c2cc(NC(=O)c3cnc(OCF)cn3)ccc2F)N=C(N)S[C@H](CO)[C@@H]1F] | 1H-NMR (400 MHz, CDCl3) δ: 1.77 (s, 3H), 3.23-3.35 (m, 1H), 3.75 (dd, J = 11.0, 7.3 Hz, 1H), 4.00 (dd, J = 11.0, 7.3 Hz, 1H), 5.44 (d, J = 46.7 Hz, 1H), 6.15 (d, J = 50.2 Hz, 2H), 7.09 (dd, J = 11.5, 8.8 Hz, 1H), 7.34 (dd, J = 6.7, 2.9 Hz, 1H), 7.95-7.99 (m, 1H), 8.30 (s, 1H), 9.08 (s, 1H), 9.48 (s, 1H). | 442 | 1 |
| I-110 | 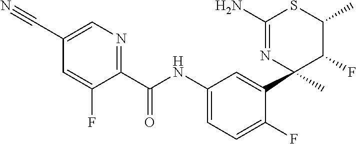 [SMILES: C[C@H]1SC(N)=N[C@](C)(c2cc(NC(=O)c3ncc(C#N)cc3F)ccc2F)[C@H]1F] | 1H-NMR (400 MHz, CDCl3) δ: 1.34 (d, J = 6.9 Hz, 3H), 1.76 (s, 3H), 3.09 (dq, J = 31.2, 6.9 Hz, 1H), 5.12 (d, J = 47.6 Hz, 1H), 7.10 (dd, J = 11.4, 8.9 Hz, 1H), 7.23 (dd, J = 6.8, 2.8 Hz, 1H), 7.91 (dd, J = 9.7, 1.5 Hz, 1H), 8.08-8.03 (m, 1H), 8.72 (s, 1H), 9.62 (s, 1H). | 420 | 1.16 |

TABLE 1-20

| | | | | |
|---|---|---|---|---|
| I-111 | 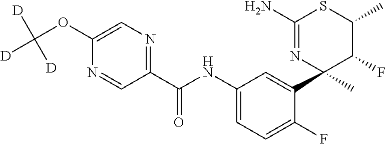 [SMILES: [2H]C([2H])([2H])Oc1cnc(C(=O)Nc2ccc(F)c([C@@]3(C)N=C(N)S[C@H](C)[C@@H]3F)c2)cn1] | 1H-NMR (400 MHz, CDCl3) δ: 1.34 (d, J = 7.0 Hz, 3H), 1.76 (s, 3H), 3.10 (dq, J = 29.9, 7.0 Hz, 1H), 5.11 (d, J = 48.7 Hz, 1H), 7.08 (dd, J = 11.5, 8.9 Hz, 1H), 7.25-7.29 (m, 1H), 8.03- 7.98 (m, 1H), 8.16 (d, J = 1.3 Hz, 1H), 9.02 (d, J = 1.3 Hz, 1H), 9.47 (s, 1H). | 411 | 1.20 |
| I-112 | 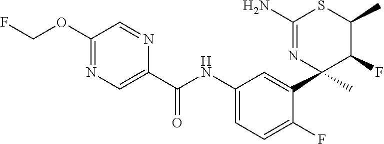 [SMILES: C[C@@H]1SC(N)=N[C@](C)(c2cc(NC(=O)c3cnc(OCF)cn3)ccc2F)[C@@H]1F] | 1H-NMR (COCl3) δ: 1.46 (d, J = 7.0 Hz, 3H), 1.52 (s, 3H), 3.68-3.83 (m, 1H), 4.42 (s, 2H), 5.01 (dd, J = 46.9, 1.8 Hz, 1H), 6.15 (dq, J = 51.2, 2.0 Hz, 2H), 7.09 (dd, J = 12.0, 8.8 Hz, 1H), 7.77 (dd, J = 7.0, 2.8 Hz, 1H), 8.09-8.13 (m, 1H), 8.28 (d, J = 1.5 Hz, 1H), 9.08 (d, J = 1.3 Hz, 1H), 9.53 (s, 1H). | 426 | 1.18 |
| I-113 | 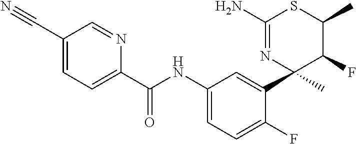 [SMILES: C[C@@H]1SC(N)=N[C@](C)(c2cc(NC(=O)c3ccc(C#N)cn3)ccc2F)[C@@H]1F] | 1H-NMR (CDCl3) δ: 1.46 (d, J = 6.8 Hz, 3H), 1.53 (s, 3H), 3.69-3.83 (m, 1H), 5.01 (dd, J = 46.9, 1.8 Hz, 1H), 7.10 (dd, J = 12.0, 8.8 Hz, 1H), 7.82 (dd, J = 7.0, 2.8 Hz, 1H), 8.08-8.13 (m, 1H), 8.19 (dd, J = 8.0, 2.0 Hz, 1H), 8.42 (dd, J = 8.2, 0.8 Hz, 1H), 8.87 (dd, J = 2.0, 0.8 Hz, 1H), 9.89 (s, 1H). | 402 | 1.13 |
| I-114 | 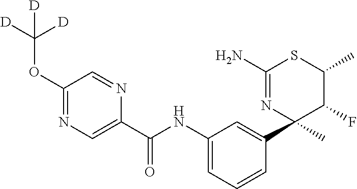 [SMILES: [2H]C([2H])([2H])Oc1cnc(C(=O)Nc2cccc([C@@]3(C)N=C(N)S[C@H](C)[C@@H]3F)c2)cn1] | 1H-NMR (CDCl3) δ: 1.29 (d, J = 7.0 Hz, 3H), 1.74 (d, J = 2.1 Hz, 3H), 3.09 (dq, J = 29.9, 7.0 Hz, 1H), 4.65 (d, J = 48.7 Hz, 1H), 7.05 (d, J = 7.9 Hz, 1H), 7.37 (t, J = 7.9 Hz, 1H), 7.59 (s, 1H), 7.72 (d, J = 7.9 Hz, 1H), 8.17 (d, J = 1.0 Hz, 1H), 9.03 (d, J = 1.0 Hz, 1H), 9.53 (s, 1H). | 393 | 1.1 |
| I-115 | 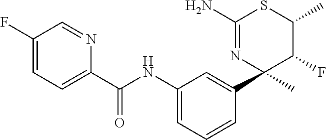 [SMILES: C[C@H]1SC(N)=N[C@](C)(c2cccc(NC(=O)c3ccc(F)cn3)c2)[C@H]1F] | 1H-NMR (CDCl3) δ: 1.29 (d, J = 7.0 Hz, 3H), 1.74 (d, J = 2.1 Hz, 3H), 3.03-3.15 (m, 1H), 4.65 (dd, J = 47.9, 2.1 Hz, 1H), 7.06 (d, J = 8.0 Hz, 1H), 7.38 (t, J = 8.0 Hz, 1H), 7.58-7.64 (m, 2H), 7.74 (dd, J = 8.0, 1.1 Hz, 1H), 8.34 (dd, J = 8.7, 4.6 Hz, 1H), 8.47 (d, J = 2.6 Hz, 1H), 9.83 (s, 1H). | 377 | 1.14 |
| I-116 | 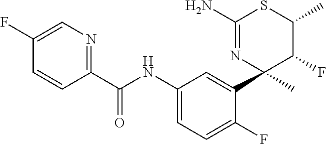 [SMILES: C[C@H]1SC(N)=N[C@](C)(c2cc(NC(=O)c3ccc(F)cn3)ccc2F)[C@H]1F] | 1H-NMR (400 MHz, CDCl3) δ: 1.34 (d, J = 7.0 Hz, 3H), 1.77 (s, 3H), 3.10 (dq, J = 29.7, 7.0 Hz, 1H), 5.12 (dd, J = 47.4, 1.5 Hz, 1H), 7.08 (dd, J = 11.5, 8.8 Hz, 1H), 7.30 (dd, J = 6.8, 2.8 Hz, 1H), 7.62-7.57 (m, 1H), 7.98- 8.03 (m, 1H), 8.33 (dd, J = 8.8, 4.5 Hz, 1H), 8.46 (d, J = 2.8 Hz, 1H), 9.78 (s, 1H). | 395 | 1.2 |

Test Examples for the compounds of the present invention are mentioned below.

### EXAMPLE 15

### Test Example 1-1: Assay of BACE1 Inhibitory Activity: 96-Well

48.5 μL of substrate peptide solution (Biotin-XSEVNLDAEFRHDSGC-Eu: X=ε-amino-n-capronic acid. Eu=Europium cryplate) was added to each well of 96-hole half-area plate (a black plate: Costar), and after addition of 0.5 μl of the compound of the present invention (DMSO solution) and 1 μl of Recombinant human BACE1(R&D Systems), the reaction mixture was incubated at 30° C. for 3.5 hours. The substrate peptide was synthesized by reacting Cryptate TBPCOOH mono SMP (CIS bio international) with Biotin-XSEVNLDAEFRHDSGC (Peptide Institute, Inc.). The final concentrations of the substrate peptide and Recombinant human BACE1 were adjusted to 9.7 nmol/L and 500 nmol/L, respectively, and the reaction was performed in sodium acetate buffer (50 mmol/L sodium actuate, pH 5.0, 0.008% Triton X-100).

After the incubation for reaction, 50 μl of 8.0 μg/ml Streptavidin-XL665 (CIS bio international) dissolved in phosphate buffer (150 mmol/L K₃HPO₄—KH₂PO₄, pH 7.0, 0.008% Triton X-100, 0.8 mol/L KF) was added to each well and left stand at 30° C. for 45 minutes. After then, fluorescence intensity was measured (excitation wavelength: 320 nm, measuring wavelength: 620 nm and 665 nm) using ARVO-X4 2030 Multilabel Reader (Perkin Elmer life sciences). Enzymatic activity was determined from counting ratio of each wavelength (10,000×Count 665/Count 620) and 50% inhibitory concentration against the enzymatic activity (IC₅₀). was calculated.

### Test Example 1-2: Assay of BACE1 Inhibitory Activity: 384-Well

5 μL of substrate peptide solution (Biotin-XSEVNLDAEFRHDSGC-Eu: X=ε-amino-n-capronic acid, Eu=Europium eryplate) was added to each well of 384-well plate (a black plate: Corning), and after addition of 0.1 μl of the compound of the present invention (DMSO solution) and 5 μl of Recombinant human BACE1 (R&D Systems), the reaction mixture was incubated at 25° C. for 2 hours. The substrate peptide was synthesized by reacting Cryptate TBPCOOH mono SMP (CIS bio international) with Biotin-XSEVNLDAEFRHDSGC (Peptide Institute, Inc.). The final concentrations of the substrate peptide and Recombinant human BACE1 were adjusted to 9.7 nmol/L and 500 nmol/L, respectively, and the reaction was performed in sodium acetate buffer (50 mmol/L sodium acetate, pH 5.0, 0.008% Triton X-100).

After the incubation for reaction, 10 μl of 8.0 μg/ml Streptavidin-XL665 (CIS bio International) dissolved in phosphate buffer (150 mmol/L K2HPO4-KH2PO4, pH 7.0, 0.008% Triton X-100, 0.8 mol/L KF) was added to each well and left stand at 25° C. for 30 minutes. After then, fluorescence intensity was measured (excitation wavelength: 320 nm, measuring wavelength: 620 nm and 665 nm) using RUBYstar (BMG LABTECH). Enzymatic activity was determined from counting ratio of each wavelength (10,000×Count 665/Count 620) and 50% inhibitory concentration against the enzymatic activity (IC₅₀) was calculated.

TABLE 2

| | IC₅₀ value | |
|---|---|---|
| No. | nmol/L | Assay format |
| I-1 | 13.3 | 96-well |
| I-2 | 67.9 | 96-well |
| I-3 | 61.3 | 96-well |
| I-4 | 11.5 | 96-well |
| I-5 | 29.9 | 96-well |
| I-6 | 15 | 96-well |
| I-7 | 47.5 | 96-well |
| I-8 | 11.8 | 96-well |
| I-9 | 10.3 | 96-well |
| I-10 | 25.8 | 96-well |
| I-11 | 102 | 96-well |
| I-12 | 56.8 | 96-well |
| I-13 | 32 | 96-well |
| I-14 | 52.2 | 96-well |
| I-15 | 15.2 | 96-well |
| I-16 | 30.3 | 96-well |
| I-17 | 59.7 | 96-well |
| I-18 | 18 | 96-well |
| I-19 | 46.5 | 96-well |
| I-20 | 72.7 | 96-well |
| I-21 | 26.9 | 96-well |
| I-22 | 23 | 96-well |
| I-23 | 34.4 | 96-well |
| I-24 | 97 | 96-well |
| I-25 | 156 | 96-well |
| I-26 | 33.9 | 96-well |
| I-27 | 40.5 | 96-well |
| I-28 | 22.6 | 96-well |
| I-29 | 1560 | 96-well |
| I-30 | 52.1 | 96-well |
| I-31 | 22.3 | 96-well |
| I-32 | 43.6 | 96-well |
| I-33 | 49.2 | 96-well |
| I-34 | 16.5 | 96-well |
| I-35 | 13.1 | 96-well |
| I-36 | 22 | 96-well |
| I-40 | 21.1 | 384-well |
| I-41 | 14.6 | 384-well |
| I-42 | 14.7 | 384-well |
| I-43 | 21.8 | 96-well |
| I-44 | 69.6 | 96-well |
| I-45 | 134 | 96-well |
| I-46 | 23.6 | 384-well |
| I-47 | 4.77 | 384-well |
| I-48 | 7.49 | 384-well |
| I-49 | 3.13 | 384-well |
| I-50 | 7.77 | 384-well |
| I-51 | 17.5 | 384-well |
| I-52 | 7.62 | 384-well |
| I-53 | 14.5 | 384-well |
| I-54 | 13 | 384-well |
| I-55 | 19.8 | 384-well |
| I-56 | 11.9 | 384-well |
| I-57 | 16.1 | 384-well |
| I-58 | 13.7 | 384-well |
| I-59 | 10.9 | 384-well |
| I-60 | 6.62 | 384-well |
| I-61 | 15.4 | 384-well |
| I-62 | 7.74 | 384-well |
| I-63 | 11.9 | 384-well |
| I-64 | 7.06 | 384-well |
| I-65 | 21.9 | 384-well |
| I-66 | 5.62 | 384-well |
| I-67 | 5.47 | 384-well |
| I-68 | 11.6 | 384-well |
| I-69 | 9.24 | 384-well |
| I-70 | 10.6 | 384-well |
| I-71 | 12 | 384-well |
| I-75 | 15.6 | 384-well |
| I-76 | 3.77 | 384-well |
| I-77 | 45.8 | 384-well |
| I-78 | 7.35 | 384-well |
| I-79 | 13.2 | 384-well |
| I-80 | 35.5 | 384-well |
| I-81 | 16.9 | 384-well |
| I-82 | 25.1 | 384-well |
| I-83 | 30.3 | 384-well |
| I-84 | 12.9 | 384-well |
| I-85 | 15.4 | 384-well |
| I-86 | 101 | 384-well |
| I-87 | 21.4 | 384-well |
| I-88 | 14.4 | 384-well |
| I-89 | 15.1 | 384-well |
| I-90 | 15.3 | 384-well |
| I-91 | 6.37 | 384-well |
| I-92 | 8.27 | 384-well |
| I-93 | 44.5 | 384-well |
| I-94 | 22.4 | 384-well |
| I-95 | 23.5 | 384-well |
| I-96 | 21.2 | 384-well |
| I-97 | 10.5 | 384-well |
| I-99 | 24.5 | 384-well |
| I-100 | 26 | 384-well |
| I-101 | 25.3 | 384-well |
| I-102 | 23.4 | 384-well |
| I-103 | 17.3 | 384-well |
| I-104 | 10.8 | 384-well |
| I-105 | 30.5 | 384-well |
| I-106 | 19.4 | 384-well |
| I-107 | 23.5 | 384-well |
| I-108 | 136 | 384-well |
| I-109 | 10.8 | 384-well |
| I-110 | 22.5 | 384-well |
| I-111 | 10.3 | 384-well |
| I-112 | 141 | 384-well |
| I-113 | 164 | 384-well |
| I-114 | 41.5 | 384-well |
| I-115 | 34.3 | 384-well |
| I-116 | 7.84 | 384-well |

### Test Example 1-8: Assay or BACE2 Inhibitory Activity

89 μL of substrate peptide solution (SEVNLDAEFRHDSGYEK-biotin) is added to each well of 96-well plate (a black plate: Costar), and after addition of 1 μl of the compound of the present invention (DMSO rotation) and 10 μl of the human BACE2 whirh purified FreeStyle TM293-F cells condition medium that expression human BACE2 ectodomain, the reaction mixture is incubated al 37° C. for 1 hours. The final concentrations of the substrate peptide and human BACE2 are adjusted to 1000 nmol/L and 20 ng/mL, respectively, and the reaction is performed in stadium acetate buffer (50 mmol/L sodium acetate, pH 4.5, 0.25 mg/mL bovine serum albumin).

After the intubation for reaction, 30 μl of 1 M Tris-HCL (pH 7.6) is added to reaction mixtures. The reaction mixtures added to each well with 82E1 (anti-amyloid β antibody: Immuno-Biological Laboratories) and incubated overnight at 4° C. After the incubation and five wash, Neutravidin-Horseradish Peroxidase conjugated (Thermo Fisher) is added to each wells and incubated for 1 hour at room temperature. After five washes, 45 μL of mixture of Supersignal pico solution A and B (Thermo Fishers) is added to each wells. The count of chemi-luminescence in each well is measured by ARVO MX 1420 Multilabel Reader (Perkin Elmer life sciences). Enzymatic activity is determined from counting ratio of each wavelength (10,000×Count 665/Count 620) and 50% inhibitory concentration against the enzymatic activity (IC₅₀ is calculated.

### EXAMPLE 16

### Test Example 2-1 Measurement of β-Amyloid (Aβ) Production Inhibitory Effect in Call: 96-Well

Neuroblastoma SH-SY5V cells (SH/APPwt) with human wild-type R-APP excessively expressed therein were prepared at 8×10⁵ cells/mL, and 150 μl portions thereof were inoculated into each well of a 96-well culture plate (Falcon). The cells were cultured for 2 hours at 17° C. in a 5% gaseous carbon dioxide incubator. Then, a solution which have been preliminarily prepared by adding and suspending the compound of the present invention (DMSO (dimethyl sulfoxide) solution) so as to be 2 μl/50 μl medium was added lo the cell sap. Namely, the final DMSO concentration was 1%, and the amount of the cell culture was 200 μl.

After the incubation was performed for 24 hours from the addition of the test compound, 100 μl of the culture supernatant was collected from each fraction. The amount of the Aβ in each fraction was measured.

The Aβ amount was measured as follows. 10 μl of a homogeneous time resolved fluorescence (HTRF) measurement reagent (Amyloid β1-40 peptide: CIS bio international) and 10 μof the culture supernatant were put into a 384-well half area microplate (black microplate, Costar) and mixed with each other, and then left standing overnight at 4° C. while the light was shielded. Then, the fluoreccence intensity (excitation wavelength: 337 nm, measurement wavelength: 620 nm and 665 nm) was measured with a micro plate reader (Artemis K-101; FU RUNO ELECTRIC). The Aβ amount was determined from the count rate at each measurement wavelength (10000×Count 665/Count 620), and the amount needed to inhibit Aβ production by 50% (IC₅₆) was calculated from at least six different dosages.

### Test Example 2-2: Measurement of β-Amyloid (Aβ) Production Inhibitory Effect in Cell: 384 Well

Neuroblastoma SH-SY5Y cells (SH/APPwt) with human wild-type β-APT excessively expressed therein were prepared at 4×10⁵ cells/mL, and 50 μl portions thereof were inoculated into each well of a 384-well culture plate (Corning) added 0.5 μl of the test compound of the present invention (DMSO solution). The final DMSO concentration was 1%, and the amount of the cell culture was 50 μl. After the incubation was performed for 21 hours from the cell seeding, 5 μl of the culture supernatant was collected from each fraction. The amount of the Aβ in each fraction was measured.

The Aβ amount was measured as follows. 5 μl of a homogeneous time resolved fluoresrenee (HTRF) measurement reagent (Amyloid β1-40 peptide; CIS bio international) and 5 μl of the culture supernatant were put into a 384′well plate (a black plate: Corning) and mixed with each other, and then left standing overnight at 4° C. while the light was shielded. Then, the fluorescence intensity (620 nm and 665 nm) was measured with EnViston (Perkin Elmer life sciences). The Aβ amount was determined from the count rate at each measurement wave length (Count 665/Count 620), and the amount needed to inhibit Aβ production by 50 % (IC₅₀) was calculated from at leaal six different dosages.

TABLE 3

| | IC₅₀ value | |
|---|---|---|
| No. | nmol/L | Assay format |
| I-1 | 0.504 | 96-well |
| I-2 | 3.73 | 96-well |
| I-3 | 3.59 | 96-well |
| I-4 | 0.697 | 96-well |
| I-5 | 2.88 | 96-well |
| I-6 | 1.29 | 96-well |
| I-7 | 2.52 | 96-well |
| I-8 | 0.26 | 96-well |
| I-9 | 0.235 | 96-well |
| I-10 | 0.708 | 96-well |
| I-11 | 4.21 | 96-well |
| I-12 | 1.88 | 96-well |
| I-13 | 1.12 | 96-well |
| I-14 | 2.09 | 96-well |
| I-15 | 0.65 | 96-well |
| I-16 | 1.12 | 96-well |
| I-17 | 3.23 | 96-well |
| I-18 | 0.573 | 96-well |
| I-19 | 1.2 | 96-well |
| I-20 | 4.19 | 96-well |
| I-21 | 2.93 | 96-well |
| I-22 | 4.22 | 96-well |
| I-23 | 1.53 | 96-well |
| I-24 | 2.28 | 96-well |
| I-25 | 4.31 | 96-well |
| I-26 | 1.76 | 96-well |
| I-27 | 6.7 | 96-well |
| I-28 | 1.5 | 96-well |
| I-29 | 93.5 | 96-well |
| I-30 | 2.33 | 96-well |
| I-31 | 0.668 | 96-well |
| I-32 | 2.39 | 96-well |
| I-33 | 1.07 | 96-well |
| I-34 | 1.85 | 96-well |
| I-35 | 0.391 | 96-well |
| I-36 | 2.29 | 96-well |
| I-40 | 6 | 384-well |
| I-41 | 15 | 384-well |
| I-42 | 21 | 384-well |
| I-43 | 0.615 | 96-well |
| I-44 | 2.11 | 96-well |
| I-45 | 5.71 | 96-well |
| I-46 | 2.4 | 384-well |
| I-47 | 1.2 | 384-well |
| I-48 | 5.4 | 384-well |
| I-49 | 0.025 | 384-well |
| I-50 | 0.39 | 384-well |
| I-51 | 0.41 | 384-well |
| I-52 | 0.42 | 384-well |
| I-53 | 0.89 | 384-well |
| I-54 | 7.2 | 384-well |
| I-55 | 0.3 | 384-well |
| I-56 | 0.53 | 384-well |
| I-57 | 0.68 | 384-well |
| I-58 | 0.68 | 384-well |
| I-59 | 0.058 | 384-well |
| I-60 | 0.29 | 384-well |
| I-61 | 1.3 | 384-well |
| I-62 | 0.25 | 384-well |
| I-63 | 5.4 | 384-well |
| I-64 | 0.22 | 384-well |
| I-65 | 1.1 | 384-well |
| I-66 | 0.13 | 384-well |
| I-67 | 0.56 | 384-well |
| I-68 | 0.41 | 384-well |
| I-69 | 0.42 | 384-well |
| I-70 | 2 | 384-well |
| I-71 | 0.54 | 384-well |
| I-75 | 8.3 | 384-well |
| I-76 | 1.6 | 384-well |
| I-77 | 79 | 384-well |
| I-78 | 6.7 | 384-well |
| I-79 | 4.5 | 384-well |
| I-80 | 18 | 384-well |
| I-81 | 21 | 384-well |
| I-82 | 17 | 384-well |
| I-83 | 14 | 384-well |
| I-84 | 2.3 | 384-well |
| I-85 | 4.8 | 384-well |
| I-86 | 2.21 | 384-well |
| I-87 | 0.66 | 384-well |
| I-88 | 0.13 | 384-well |
| I-89 | 1.5 | 384-well |
| I-90 | 0.51 | 384-well |
| I-91 | 0.25 | 384-well |
| I-92 | 1 | 384-well |
| I-93 | 28 | 384-well |
| I-94 | 3.5 | 384-well |
| I-95 | 450 | 384-well |
| I-96 | 1.8 | 384-well |
| I-97 | 2.5 | 384-well |
| I-99 | 0.99 | 384-well |
| I-100 | 13 | 384-well |
| I-101 | 11 | 384-well |
| I-102 | 1.6 | 384-well |
| I-103 | 2.1 | 384-well |
| I-104 | 0.49 | 384-well |
| I-105 | 0.74 | 384-well |
| I-106 | 2.6 | 384-well |
| I-107 | 0.42 | 384-well |
| I-108 | 8.8 | 384-well |
| I-109 | 0.45 | 384-well |
| I-110 | 1.1 | 384-well |
| I-111 | 0.97 | 384-well |
| I-112 | 50 | 384-well |
| I-113 | 40 | 384-well |
| I-114 | 5.1 | 384-well |
| I-115 | 7.2 | 384-well |
| I-116 | 0.99 | 384-well |

### EXAMPLE 17

### Test Example 3-1: Lowering Effect on the Brain β Amyloid in Rats

Compound of the present invenlion is suspended in 0.5% methylcellulose, the final concentration is adjusted to 2 mg/mL, and this is orally administered to male Crl:SD rat (7 to 9 weeks old) at 10 mg/kg. In a vehicle control group, only 0.5% methylcellulose is administered, and an administration test is performed at 3 to 8 animals per group. A brain is isolated 3 hours after administration, a cerebral hemisphere is isolated, a weight thereof is measured, the hemisphere is rapidly frozen in liquid nitrogen, and stored at −80° C. until extraction date. The frozen cerebral hemisphere is transferred to a homogenizer manufactured by Teflon (Registered trademark) under ice cooling, a 4-fold volume of a weight of an extraction buffer (containing 1% CHAPS ({3-[(3-chloroamidopropyl)dimethylammonio]-1-propanesulfonate}). 20 mmol/L Tris-HCl (pH 8.0), 150 mmol/L NaCl. Complete (Roche) protease inhibitor) is added, up and down movement is repeated, and this is homogenized to solubilize for 2 minutes. The suspension is transferred to a centrifugation tube, allowed to stand on an ice for 3 hours or more and, thereafter centrifuged at 100,000×g, 4° C. for 20 minutes. After centrifugation, the supernatant is transferred to an ELISA plate (product No. 294-62501, Wako Junyaku Kogyo) for measuring β amyloid 40. ELISA measurement is performed according to the attached instruction. The lowering effect in calculated as a ratio compared to the brain β amyloid 40 level of vehicle control group of each test.

### Test Example 3-2: Lowering Effect on the Brain β Amyloid in Mice

Compound of the present invention is dissolved in 20% hydroxyl-beta-cyclodextrin, the final concentration is adjusted to 2 mg/mL, and this is orally administered to male Crl:CD1 (ICR) mouse (6 to 8 weeks old) at 1 to 10 mg/kg. In a vehicle control group, only 20% hydroxyl-beta-cyclodextrin is administered, and an administration test is performed at 3 to 6 animals per group. A brain is isolated 1 to 6 hours after administration, a cerebral hemisphere is isolated, a weight thereof is measured, the hemisphere is rapidly frozen in liquid nitrogen, and stored at −80° C. until extraction date.

The frozen cerebral hemisphere is transferred to a homogenize tube containing ceramic beads in a 8-fold volume of a weight of an extraction buffer (containing 0.4% DEA (diethylamme), 50 mmol/L NaCl. Complete protease inhibitor (Roche)) and incubated on an ice for 20 minutes. Thereafter, the homogenization is done using MP BIO Fast Prep (Registrred trademark)-24 with Lysing matrix D 1.4 mm ceramic beads (20 seconds at 6 m/s). Then, the tube spins down for 1 minute, the supernatant is transferred to a centrifugation tube, and ceutrifuged at 221,000×g, 4° C. for 50 minutes. After centrifugation, the supernatant is transferred to Nunc Maxisorp (Registered trademark) plate (Thermo Fisher Scientific) coating with antibody against N-terminal of β amyloid for measuring total β amyloid, and the plate is incubated overnight at 4° C. The plate is washed with TBS-T (Tris buffered saline containing 0.05% Triton X-100), and HRP-conjugated 4G8 dissolved in PBS (pH 7.4) containing 0.1% casein is added in the plate and incubated at 4° C. for 1 hour. After it is washed with TBS-T, SuperSignal ELISA Pico Chemilominescent Substrate (Thermo Scientific) is added in the plate. Then, the chemi-luminescence counting is measured by ARVO (Registered trademark) MX 1420 Muitilabel Counter (Perkin Elmer) as soon as possible. The lowering effect is calculated as a ratio compared to the brain total β amyloid level of vehicle control group of each test.

### EXAMPLE 18

### Test Example 4-1: CYP3A4 Fluorescent MBI Test

The CYP3A4 fluorescent MBI test is a test of investigating enhancement of CYP3A4 inhibition of a compound by a metabolism reaction. 7-benzyloxytrifluoromethylcoumarin (7-BFC) is debenzylated by the CYP3A4 enzyme (enzyme expressed in Escherhia coli) and 7-hydroxytrifluoromethylcoumarin (7-HFC) is produced as a fluorescing metabolite. The test is performed using 7-HFC production reaction as an index.

The reaction conditions were as follows: substrate, 5.6 μmol/L 7-BFC: pre-reaction time, 0 or 30 minutes; substrate reaction time, 15 minutes: reaction temperature, 25° C. (room temperature); CYP3A4 content (expressed in Eschericia coli), at pre-reaction time 62.5 pmol/mL, at reaction time 6.25 pmol/mL (at 10-fold dilution): concentrations of the compound of the present invention, 0.625, 1.25, 2.5, 5, 10, 20 μmol/L (six points).

An enzyme in a K-Pi buffer (pH 7.4) and a compound of the present invention solution as a pre-reaction solution were added to a 96-well plate at the composition of the pre-reaction. A part of pre-reaction solution was transferred to another 96-well plate, and 1/10 diluted by a substrate in a K-Pi buffer. NADPH as a co-factor was added to initiate a reaction as an index (without preincubation). After a predetermined time of a reaction, acetonitrile/0.5 mol/L Tris (trishydroxyaminomethane)=4/1 (v/v) solution was added to stop the reaction. On the other hand, NADPH was also added to a remaining pre-reaction solution in order to initiate a preincubation (with prnncubation). After a predetermined time of a preincubation, a part was transferred to another 96-well plate, and 1/10 diluted by a substrate in a K-Pi buffer in order to initiate a reaction as an index. After a predetermined time of a reaction, acetibutruke/0.5 mol/L Tris (trishydroxyaminomethnne)=4/1 (v/v) solution was added go stop the reaction. Fluorescent values of 7-HFC at a metabolite were measured in each index reaction plate with a fluorescent plate reader (Ex=420 nm, Em=535 nm).

The sample adding DMSO to a reaction system instead of compound of the present invention solution was adopted its a control (100%) because DMSO was used as a solvent to dissolve a compound of the present invention. Remaining activity (%) was calculated at each concentration of the compound of the present invention added as the solution, and IC₅₀ value was calculated by reverse presumption by a logistic model using a concentration and an inhibition rate. When a difference subtracting IC₅₀ value with preincubation from that without IC₅₀ value was 5 μM or more, this was defined as positive (+). When the difference was 3 μM or less, this was defined as negative (−).

The following compounds were defined as negative,

1-2, 3, 4, 5, 6, 7, 9, 10, 11, 12, 13, 14, 15, 16, 17, 18, 19, 20, 21, 22, 23, 24, 25, 26, 27, 28, 29, 30, 31, 32, 33, 34, 36, 40, 41, 42, 43, 44, 45, 71 and 86.

### Test Example 4-2 CYP3A4(MDZ) MBI Test

CYP3A4(MDZ) MBI test is a test of investigating mechanism based inhibition (MBI) ability on CYP3A4 inhibition of a compound by enhancement of a metabolism reaction. CYP3A4 inhibition is evaluated using hydroxylation reaction of midazolam (MDZ) by pooled human liver microsomes as an index.

The reaction conditions were at follows: substrate, 10 μmol/L, MDZ: pre-reaction time, 0 or 30 minutes; substrate reaction time, 2 minutes: reaction temperature, 37° C.; protein content of pooled human liver microsomes, at pre-reaction time 0.5 mg/mL, at reaction time 0.05 pmg/mL (at 10-fold dilution): concentrations of the compound of the present invention, 1, 5, 10, 20 μmol/L (four points).

Pooled human liver microsomes in a K-Pi buffer (pH 7.1) and a compound of the present invention solution as a pre reaction solution were added to a 96-well plate at the composition of the pre-reaction. A part of pre-reaction solution was transferred to another 96-well plate, and 1/10 diluted by a substrate in a K-Pi buffer. NADPH as a co-factor was added to initiate a reaction as an index (without preincubation). After a predetermined time of a reaction, methanol/acetonitrile-=1/1 (v/v) solution was added to stop the reaction. On the other hand, NADPH was also added to a remaining pre-reaction solution in order to initiate a preincubation (with preincubation). After a predetermined time of a preincubation, a part was transferred to another 96-well plate, and 1/10 diluted by a substrate in a K-Pi buffer in order to initiate a reaction as an index. After a predetermined time of a reaction, methanol/acetonitrile=1/1 (v/v) solution was added to stop the reaction. After centrifuged at 3000 rpm for 15 minutes, 1-hydroxymidazolam in the supernatant was quantified by LC/MS/MS.

The sample addiiui DMSO to a reaction system instead of compound of the present invention solution wa adopted as a control (100%) because DMSO was used as a solvent to dissolve a compound of the present invention. Remaining activity (%) was calculated at each concentration of the compound of the present invention added as the solution, and IC₅₀ value was calculated by reverse-presumption by a logistic model using a concentration and an inhibition rate. Shifted IC value was calculated as “IC of preincubation at 0 min/ IC of preincubation at 30 min”. When a shifted IC was 1.5 or more, this was defined as positive. When a shifted IC was 1.0 or less, this was defined as negative.

The following compounds were defined as negative.

### I-50, 52, 54, 56, 64, 65, 75, 77, 81, 82, 88, 89, 90, 91, 94, 97, 99, 100, 101, 102, 103, 104, 105, 106, 107, 108, 109, 111, 114 and 115.

### EXAMPLE 19

### Test Example 5: CYP Inhibition Test

The CYP inhibition test is a test to assess the inhibitory effect of a compound of the present invention towards typical substrate metabolism reactions on CYP enzymes in human liver microsomes. The marker reactions on human main five CYP enzymes (CYP1A2, 2C9, 2C19, 2D6, and 3A4) are used as follows: 7-ethoxyresorufin O-deethylation (CYP1A2), tolbutamide methyl-hydroxylation (CYP2C9), mephenytoin 4′-hydroxylation (CYP2C19), dextromethorphan O-demethylation (CYP2D6), and terfenadine hydroxylation (CYP3A4). The commercially available pooled human liver microsomes are used as an enzyme resource.

The reaction conditions were as follows: substrate, 0.5 μmol/L ethoxyresorufin (CYP1A2), 100 μmol/L tolbutamide (CYP2C9), 50 μmol/L, S-mephenytoin (CYP2C19), 5 μmol/L dextromethorphan (CYP2D6), 1 μmol/L terfenadine (CYP3A4): reaction time, 15 minutes: reaction temperature, 37° C.; enzyme, pooled human liver microsomes 0.2 mg protein/mL; concentrations of the compound of the present invention, 1, 5, 10, 20 μmol/L (four points).

Five kinds of substrates, human liver microsomes, and a compound solution of the present invention in 50 μmol/L Hepes buffer were added to a 96-well plate at the composition as described above as a reaction solution. NADPH as a cofactor was added to this 96-well plate in order to initiate metabolism reactions. After the incubation at 37° C.for 5 minutes, a methanol/acetonitrile=1/1 (v/v) solution was added to stop the reaction. After the centrifugation at 3000 rpm for 15 minutes, resorufin (CYP1A2 metabolite) in the supernatant was quantified by a fluorescent plate reader, and hydroxytolbutamide (CYP2C9 metabolite), 4′-hydroxymephenytoin (CYP2C19 metabolite), dextrorphan (CYP2D6 metabolite), and terfenadine alcohol metabolite (CYP3A4 metabolite) in the supernatant were quantified by LC/MS/MS.

The sample adding DMSO to a reaction system instead of compound of the present invention solution was adopted as a control (100%) because DMSO was used as a solvent to dissolve a compound of the present invention. Remaining activity (%) was calculated at each concentration of a compound of the present invention, and IC₅₀ value was calculated by reverse presumption by a logistic model using a concentration and an inhibition rate.

CYP1A2 20 μM or more: Compound I-1, 2, 3, 4, 5, 6, 8, 9, 10 11, 12, 13, 14, 15, 16, 17, 18, 19, 20, 21, 22, 23, 24, 25, 20, 27, 28, 30, 33, 32, 33, 34, 35, 36, 40, 41, 42, 43, 44, 45, 46, 47, 48, 49, 50, 51, 52, 53, 55, 56, 57, 58, 59, 60, 61, 62, 63, 61, 66, 66, 67, 68, 60, 70, 71, 75, 76, 77, 78, 79, 80, 81, 82, 83, 84, 85, 86, 87, 88, 89, 90, 91, 92, 93, 94, 95, 96, 97, 99, 100, 101, 102, 103, 104, 105, 106, 107, 108, 109, 110, 111, 112, 113, 114 and 116
CYP1A2 10 μM or more: Compound I-29 and 115
CYP2P9 20 μM or more: Compound I-1, 10, 12, 13, 14, 15, 17, 18, 19, 20, 21, 22, 23, 24, 25, 26, 27, 28, 29, 30, 31, 32, 33, 34, 35, 36, 40, 41, 12, 43, 44, 45, 55, 56, 57, 58, 59, 60, 61, 71, 78, 86, 87, 88, 89, 90, 102, 105, 106, 112, 113 and 115
CYP2C9 10 μM or more: Compound I-2, 3, 4, 5, 11, 16, 49, 62, 63, 75, 77, 79, 80, 81, 94, 95, 96, 99, 103, 104, 107, 110 and 114
CYP2C19 20 μM or more: Compound I-1, 2, 3, 4, 5, 8, 9, 10, 11, 12, 13, 14, 15, 16, 17, 18, 19, 20, 21, 22, 23, 24, 25, 26, 27, 28, 29, 30, 31, 32, 33, 34, 35, 36, 40, 41, 42, 43, 44, 45, 46, 47, 48, 49, 50, 51, 52, 53, 55, 56, 57, 58, 59, 60, 61, 62, 63, 64, 65, 66, 67, 68, 70, 71, 75, 77, 78, 79, 80, 81, 82, 83, 84, 85, 86, 87, 88, 89, 90, 91, 92, 93, 94, 95, 96, 97, 99, 100, 101, 102, 103, 104, 105, 106, 107, 108, 109, 110, 111, 112, 113, 114, 115 and 116
CYP2C19 10 μM or more: Compound I-6 and 76
CYP2D6 20 μM or more: Compound I-1, 4, 10, 13, 15, 18, 20, 23, 25, 26, 27, 28, 29, 31, 34, 35, 36, 40, 43, 45, 49, 52, 53, 64, 85, 87, 88, 89, 90, 96, 102, 103, 104, 105, 106, 107, 112, 113, 114, 115 and 116
CYP2D6 10 μM or more: Compound I-11, 12, 22, 24, 32, 44, 46, 48, 70, 78, 99, 108 and 110
CYP3A4 20 μM or more: Compound I-1, 2, 3, 4, 5, 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 40, 41, 42, 43, 44, 45, 47, 48, 49, 50, 52, 53, 64, 65, 71, 86, 87, 88, 89, 90, 91, 92, 94, 96, 97, 99, 100, 101, 102, 103, 104, 105, 106, 107, 108, 109, 110, 111, 112, 113, 114, 115 and 116
CYP3A4 10 μM or more: Compound I-51, 55, 56, 57, 61, 66, 68, 69, 70, 78, 79, 80, 81 and 95

### EXAMPLE 20

### Test Example 6—Flucluation Ames Test

Each 20 μL, of freeze-stored Salmonella typhimurium (TA98 and TA100 strain) is inoculated in 10 mL of liquid nutrient medium (2.58 % Oxoid nutrient broth No. 2) and the cultures are incubated at 37° C. under shaking for 10 hours. 7.70 mL of TA98 culture in centrifuged (2000×g, 10 minutes) to remove medium, and the bacteria is suspended in 7.70 mL of Micro F buffer (K₂HPO₄: 3.5 g/L, KH₂PO₄: 1 g/L, (NH₄)₂SO₄: 1 g/L, trisodium citrate dihydrate 0.25 g/L, MgSO₄ 7H₂O: 0.1 g/L), and the suspension is added to 120 mL of Exposure medium (Micro F buffer containing Biotin: 8 μg/mL, histidine: 0.2 μg/mL, glucose: 8 mg/mL), 3.42 mL of TA100 culture is added to 130 mL of Exposure medium to prepare the test bacterial solution. 588 μL of the test bacterial solution (or mixed solution of 498 μl of the test bacterial solution and 90 μl, of the S9 mix in the case with metabolic activation system) are mixed with each 12 μL of the following solution: DMSO solution of the compuomd of the present invention (several stage dilution from maximum dose 50 mg/mL at 2 to 3-fold ratio): DMSO as negative control: 50 μg/mL of 4-nitroquinoline-1-oxide DMSO solution as positive control for TA98 without metabolic activation system: 0.25 μg/mL of 2-(2-fury1)-3-(5-nitro-2-furyl)acrylamide DMSO solution as positive control for TA100 without metabolic activation system: 40 μg/mL of 2-aminoanthracene DMSO solution as positive control for TA98 with metabolic activation system: or 20 μg/mL of 2-aminoanthracene DMSO solution as positive control for TA100 with metabolic activation system. A mixed solution is incubated at 37° C. under shaking for 90 minutes. 460 μL of the bacterial solution exposed to the compound of the present invention is mixed with 2300 μL of Indicator medium (Micro F buffer containing biotin: 8 μg/mL, histidine 0.8 μg/mL, glucose: 8 mg/mL, Bromo Cresol Purple: 37.5 μg/mL), each 50 μL is dispensed into 48 wells/dose in the microwell plates, and is subjected to stationary cultivation at 37° C. for 3 days. A well containing the bacteria, which has obtained the ability of proliferation by mutation in the gene coding amino acid (histidine) synthetase, turns the color from purple to yellow due to pH change. The number of the yellow wells among the 48 total wells per dose is counted, and evaluate the mutagenicity by comparing with the negative control group. (−) means that mutagenicity is negative and (+) means positive.

### EXAMPLE 21

### Test Example 7: Solubility Test

The solubility of each compound of the present invention was determined under 1% DMSO addition conditions. A 10 mmol/L solution of the compound was prepared with DMSO, and 2 μL of the compound of the present invention solution was added, respectively, to 198 μL of JP 1st fluid (water was added to 2.0 g of sodium chloride and 7.0 ml, of hydrochloric acid to reach 1000 mL) and JP 2nd fluid (See Table 4). The mixture was left standing for 16 hours at 25° C. or shaken for 1 hour at ruom temperature, and the mixture was vacuum-filtered. The nitrate was ten or one hundred-fold diluted with metbanol/water=1/1 (v/v) or MeCN/MeOH/H₂O(=1/1/2), and the compound concent ration in the filtrate was measured with LC/MS or solid phase extraction (SPE)/MS by the absolute calibration method.

TABLE 4

| No. | Condition | JP2 fluid | JP1 μM | JP2_μM |
|---|---|---|---|---|
| I-1 | *1 | A | >50 | >50 |
| I-2 | *1 | A | 23.3 | >50 |
| I-3 | *1 | A | >50 | >50 |
| I-4 | *1 | A | 35.8 | >50 |
| I-5 | *1 | A | 34.3 | >50 |
| I-7 | *1 | A | >50 | >50 |
| I-8 | *1 | A | 46.4 | >50 |
| I-9 | *2 | A | >50 | >50 |
| I-10 | *1 | A | 40 | >50 |
| I-11 | *1 | A | 42.7 | >50 |
| I-12 | *1 | A | 36.1 | >50 |
| I-13 | *1 | A | 39.6 | >50 |
| I-14 | *1 | A | 38.3 | >50 |
| I-15 | *2 | A | 31.9 | >50 |
| I-16 | *2 | A | 30.1 | >50 |
| I-17 | *2 | A | 33.9 | >50 |
| I-18 | *1 | A | 36 | >50 |
| I-19 | *1 | A | 37.1 | >50 |
| I-20 | *1 | A | 38.9 | >50 |
| I-21 | *1 | A | 28.8 | >50 |
| I-22 | *1 | A | 31.2 | >50 |
| I-23 | *1 | A | 46.7 | >50 |
| I-24 | *1 | A | 38.8 | >50 |
| I-25 | *1 | A | 46.7 | >50 |
| I-26 | *1 | A | 41.9 | >50 |
| I-27 | *1 | A | 39.7 | >50 |
| I-28 | *1 | A | 40.4 | >50 |
| I-29 | *1 | A | 35 | >50 |
| I-30 | *1 | A | 39.2 | >50 |
| I-31 | *1 | A | 38.8 | >50 |
| I-32 | *1 | A | >50 | >50 |
| I-33 | *2 | A | 39.4 | >50 |
| I-34 | *1 | A | 44.4 | >50 |
| I-35 | *1 | A | >50 | >50 |
| I-36 | *2 | A | 37.6 | 15.9 |
| I-40 | *2 | A | >50 | >50 |
| I-41 | *2 | A | >50 | >50 |
| I-42 | *2 | A | >50 | >50 |
| I-43 | *1 | A | 29.9 | >50 |
| I-44 | *1 | A | 34 | >50 |
| I-45 | *1 | A | 35.8 | >50 |
| I-46 | *2 | A | >50 | 8.3 |
| I-47 | *2 | A | >50 | 30.5 |
| I-48 | *2 | A | >50 | 39.5 |
| I-49 | *2 | A | >50 | >50 |
| I-50 | *2 | B | >50 | >50 |
| I-51 | *2 | B | >50 | >50 |
| I-52 | *2 | B | >50 | 13.9 |
| I-53 | *2 | B | >50 | >50 |
| I-54 | *2 | B | >50 | 38.6 |
| I-55 | *2 | B | >50 | >50 |
| I-56 | *2 | B | >50 | >50 |
| I-57 | *2 | B | >50 | >50 |
| I-58 | *2 | B | >50 | >50 |
| I-59 | *2 | B | >50 | >50 |
| I-60 | *2 | B | >50 | >50 |
| I-61 | *2 | B | >50 | >50 |
| I-62 | *2 | B | >50 | >50 |
| I-63 | *2 | B | >50 | >50 |
| I-64 | *2 | B | >50 | >50 |
| I-65 | *2 | B | >50 | >50 |
| I-66 | *2 | B | >50 | >50 |
| I-67 | *2 | B | >50 | >50 |
| I-68 | *2 | B | >50 | 9.1 |
| I-69 | *2 | B | >50 | >50 |
| I-70 | *2 | B | >50 | >50 |
| I-71 | *2 | A | >50 | >50 |
| I-75 | *2 | B | >50 | 3 |
| I-76 | *2 | B | >50 | 8.2 |
| I-77 | *2 | B | >50 | 11.1 |
| I-78 | *2 | B | >50 | >50 |
| I-79 | *2 | B | >50 | >50 |
| I-80 | *2 | B | >50 | >50 |
| I-81 | *2 | B | >50 | 19.3 |
| I-82 | *2 | B | >50 | >50 |
| I-83 | *2 | B | >50 | 29.6 |
| I-84 | *2 | B | >50 | 49.4 |
| I-85 | *2 | B | 47.2 | 0.6 |
| I-86 | *2 | A | >50 | >50 |
| I-87 | *2 | B | >50 | >50 |
| I-88 | *2 | B | >50 | >50 |
| I-89 | *2 | B | >50 | >50 |
| I-90 | *2 | B | >50 | >50 |
| I-91 | *2 | B | >50 | >50 |
| I-92 | *2 | B | >50 | >50 |
| I-93 | *2 | B | >50 | 6.1 |
| I-94 | *2 | B | >50 | >50 |
| I-95 | *2 | B | >50 | >50.0 |
| I-96 | *2 | B | 45.8 | >50.0 |
| I-97 | *2 | B | >50 | >50.0 |
| I-99 | *2 | B | >50 | >50.0 |
| I-100 | *2 | B | >50 | >50.0 |
| I-101 | *2 | B | >50 | >50.0 |
| I-102 | *2 | B | >50 | >50.0 |
| I-103 | *2 | B | 49.4 | >50.0 |
| I-104 | *2 | B | 40.6 | >50.0 |
| I-105 | *2 | B | 47.3 | >50.0 |
| I-106 | *2 | B | 42 | >50.0 |
| I-107 | *2 | B | 33.5 | >50.0 |
| I-108 | *2 | B | >50 | >50 |
| I-109 | *2 | B | >50 | 19 |
| I-110 | *2 | B | 42.1 | >50.0 |
| I-111 | *2 | B | 48.4 | >50.0 |
| I-112 | *2 | B | >50.0 | >50.0 |
| I-113 | *2 | B | >50.0 | 48.6 |
| I-114 | *2 | B | >50.0 | >50.0 |
| I-115 | *2 | B | >50.0 | >50.0 |
| I-116 | *1 | A | >50 | >50 |

*1: standing for 16 hours at 25° C.
*2: shaken for 1 hour at room temperature
A: 0.2 mol/L potassium dihydrogen phosphate solution adjusted to pH 6.8 with 0.2 mol/L sodium hydroxide solution/water = 1/1.5)
B: Dissolve 3.40 g of potassium dihydrogen phosphate and 3.55 g of anhydrous disodium hydrogen phosphate in water to make 1000 mL

### EXAMPLE 22

### Test Example 8: Metabolic Stability Test

Using a commercially available pooled human liver microsomes, a compound of the present invention was reacted for a constant time, a remaining rate was calculated by comparing a reacted sample and an unreacted sample, thereby, a degree of metabolism in liver was assessed.

A reaction was performed (oxidative reaction) at 37° C. for 0 minute or 30 minutes in the presence of 1 mmol/L NADPH in 0.2 mL of a buffer (60 mmol/L Tris-HCl pH 7.4, 150 mmol/L potassium chloride, 10 mmol/L magnesium chloride) containing 0.5 mg protein/mL of human liver microsomes. After the reaction, 50 μL of the reaction solution was added to 100 μL of a methanol/acetonltrile=1/1 (v/v), mixed and centrifuged at 3000 rpm for 15 minutes. The compound of the present invention in the supernatant was quantified by LC/MS/MS or solid phase extraction (SPE)/MS, and a remaining amount of the compound of the present invention after the reaction area calculated, letting a compound amount at 0 minute reaction time to be 100%.

TABLE 5

| No. | remaining % |
|---|---|
| I-1 | 82.4 |
| I-2 | 88 |
| I-3 | 81.4 |
| I-7 | 82 |
| I-9 | 95.7 |
| I-10 | 95.6 |
| I-11 | 104 |
| I-12 | 97.8 |
| I-13 | 101 |
| I-14 | 103 |
| I-15 | 104 |
| I-16 | 97.6 |
| I-18 | 71.4 |
| I-19 | 69.9 |
| I-20 | 75.4 |
| I-23 | 86.6 |
| I-24 | 95.5 |
| I-25 | 89.1 |
| I-26 | 76 |
| I-27 | 84.7 |
| I-29 | 98.2 |
| I-31 | 85 |
| I-32 | 94.6 |
| I-33 | 87.5 |
| I-40 | 106 |
| I-41 | 105 |
| I-42 | 68.2 |
| I-43 | 84.5 |
| I-44 | 86.4 |
| I-45 | 93.5 |
| I-46 | 97.8 |
| I-47 | 96.9 |
| I-48 | 92.9 |
| I-49 | 95.4 |
| I-51 | 76.5 |
| I-52 | 60.3 |
| I-53 | 109 |
| I-54 | 91 |
| I-59 | 66.8 |
| I-61 | 71.5 |
| I-63 | 75.8 |
| I-64 | 80.3 |
| I-65 | 96.5 |
| I-66 | 67 |
| I-67 | 69 |
| I-69 | 77.6 |
| I-70 | 74.3 |
| I-71 | 63 |
| I-75 | 85.2 |
| I-77 | 93.1 |
| I-81 | 101 |
| I-82 | 96.5 |
| I-83 | 55.3 |
| I-85 | 93.2 |
| I-86 | 67.5 |
| I-87 | 81 |
| I-88 | 83.5 |
| I-89 | 86.7 |
| I-90 | 83.1 |
| I-91 | 74.4 |
| I-92 | 81 |
| I-93 | 82.2 |
| I-94 | 96.8 |
| I-95 | 105 |
| I-96 | 101 |
| I-99 | 89.4 |
| I-100 | 60.2 |
| I-101 | 91.8 |
| I-102 | 78.1 |
| I-103 | 78.3 |
| I-104 | 77.7 |
| I-105 | 56.8 |
| I-106 | 84.1 |
| I-107 | 64.2 |
| I-108 | 92.1 |
| I-109 | 86.8 |
| I-110 | 81.1 |
| I-111 | 64.9 |
| I-114 | 80.5 |
| I-115 | 78.9 |
| I-116 | 106 |

### EXAMPLE 23

### Test Example 9: hERG Test

For the purpose of assessing risk of an electrocardiogram QT interval prolongation, effects on delayed rectifier K+ current (I_{Kr}), which plays an important role in the ventricular repolarization process of the compound of the present invention, was studied using CHO cells expressing human ether-a-go-go related gene (hERG) channel.

A cell was retained at a membrane potential of =80 mV by whole cell patch clamp method using an automated patch clamp system (QPatch:Sophion Bioscience A/S). After application of leak potential at −50 mV, I_{Kr} induced by depolarization pulse stimulation at +20 mV for 2 seconds and, further, repolarization pulse stimulation at −50 mV for 2 seconds was recorded.

After the generated current was stabilised, extracellular solution (NaCl: 145 mmol/L, KCl: 4 mmol/L, CaCl₂:2 mmol/L MgCl₂:1 mmol/L, 1 mmol/L, HEPES(4-(2-hydroxyethyl)-1-piperazineethanesulfonic acid: 10 mmol/L, glucose-10 mmol/L pH=7.4) in which the compound of the present invention have been dissolved at an objective concentration was applied to the cell under the room temperature condition for 10 minutes. From the recording I_{Kr}, an absolute value of the tail peak current was measured baaed on the current value at the resting membrane potential using an analysis software (Falster Patch: Sophion Bioscience A/S). Further, the % inhibition relative to the tail peak current before application of the compound of the present invention was calculated, and compared with the vehicle-applied group (0.1% dimethyl sulfoxide solution) to assess influence of the compound of the present invention on I_{Kr}.

TABLE 6

| No. | hERG_3 uM_% |
|---|---|
| I-2 | 38.7 |
| I-3 | 45.5 |
| I-4 | 47.9 |
| I-5 | 27.7 |
| I-7 | 47.4 |
| I-8 | 16.7 |
| I-9 | 30.6 |
| I-11 | 42.5 |
| I-12 | 35.5 |
| I-13 | 52.8 |
| I-14 | 16.1 |
| I-15 | 57 |
| I-16 | 31.5 |
| I-17 | 19.5 |
| I-18 | 44.8 |
| I-19 | 18.8 |
| I-20 | 30.4 |
| I-21 | 51.1 |
| I-22 | 14.2 |
| I-24 | 6.7 |
| I-25 | 41.9 |
| I-26 | 29.6 |
| I-27 | 28.8 |
| I-28 | 36.2 |
| I-29 | 45.4 |
| I-30 | 15.1 |
| I-31 | 27.4 |
| I-32 | 15.2 |
| I-33 | 21.9 |
| I-34 | 15.5 |
| I-35 | 41.1 |
| I-36 | 29.9 |
| I-40 | 48.4 |
| I-41 | 24.8 |
| I-42 | 15.5 |
| I-44 | 28.9 |
| I-45 | 51.6 |
| I-46 | 33.4 |
| I-47 | 40.4 |
| I-49 | 27.2 |
| I-50 | 34.3 |
| I-51 | 39.4 |
| I-52 | 55.7 |
| I-53 | 47.9 |
| I-54 | 37.7 |
| I-55 | 39.5 |
| I-56 | 37.6 |
| I-58 | 58.6 |
| I-62 | 29.6 |
| I-63 | 53.2 |
| I-64 | 41.3 |
| I-65 | 28 |
| I-66 | 27.1 |
| I-67 | 56.5 |
| I-70 | 58.6 |
| I-71 | 17 |
| I-75 | 21.3 |
| I-76 | 5.8 |
| I-77 | 44.1 |
| I-78 | 39.6 |
| I-79 | 7.8 |
| I-80 | 8.9 |
| I-81 | 49.3 |
| I-82 | 17.5 |
| I-83 | 18.1 |
| I-84 | 28.8 |
| I-85 | 41.4 |
| I-86 | 23.8 |
| I-87 | 13.8 |
| I-88 | 45.6 |
| I-89 | 42.7 |
| I-90 | 11.4 |
| I-91 | 11.3 |
| I-94 | 25.7 |
| I-108 | 39.5 |
| I-109 | 11 |
| I-116 | 45.5 |

### EXAMPLE 24

### Test Example 10: Powder Solubility Test

Appropriate amounts of the compound of the present invention are put in to appropriate containers, 200 μL of JP 1st fluid (water is added to 2.0 g of sodium chloride and 7.0 mL of hydrochloric acid to reach 1000 mL), 200 μL of JP 2nd fluid (1 volume of wafer is added to 1 volume of the solution which 3.40 g of potassium dihydrogen phosphate and 3.55 g of anhydrous disodium hydrogen phosphate dissolve in water to reach 1000 mL), and 200 μL of JP 2nd fluid containing 20 mmol/L of sodium taurocholate (TCA) (TCA 1.08 g and JP 2nd fluid to make 100 mL) are added to the respective containers. When total amount or the compound of the present invention is dissolved after the addition of the test fluid, the compound is added as appropriate. The containers are sealed, and shaken for 1 hour al 37° C. The mixtures are filtered, and 100 μL of methanol is added to each of the filtrate (100 μL) so that the filtrates are two-fold diluted. The dilution ratio may be changed if necessary. After confirming that there is no bubbles and precipitates in the diluted solution, the containers are sealed and shaken. Quantification is performed by HPLC with an absolute calibration method.

### EXAMPLE 25

### Test Example 11: Pharmacokinetic Study

### Materials and Methods for Studies on Oral Absorption

(1l) Animal: mouse or rat
(2) Breeding conditions: mouse or rat was allowed free access to the tap water and the solid food.
(3) Dose and grouping: orally or intravenously administered at a predetermined dose: grouping was as follows (Dose depends on the compound)
Oral administration: 1 to 30 mg/kg (n=2 to 3)
Intravenous administration: 0.5 to 10 mg/kg (n=2 to 3)
(4) Dosing formulation: for oral administration, in a solution or a suspension state; for intravenous administration, in a solubilized state
(5) Dosing method: in oral administration, forcedly administer using a syringe attached a flexible feeding tube; in intravenous administration, administer from caudal vein using a syringe attached with a needle.
(6) Evaluation items: blood was collected at the scheduled time, and the plasma concentration of the compound of the present invention was measured by LC/MS/MS
(7) Statistical analysis: regarding the transition of the plasma concentration of the compound of the present invention, the area under the plasma concentration-time curve (AUC) was calculated by non-linear least squares program WinNonlin (Registered trademark), and the bioavailability (BA) of the compound of the present invention was calculated from the AUCs of the oral administration group and intravenous administration group

TABLE 7

| No. | BA_% |
|---|---|
| I-1 | 66.7 |
| I-2 | 51.3 |
| I-3 | 38.8 |
| I-4 | 83.2 |
| I-7 | 32.8 |
| I-8 | 27.4 |
| I-9 | 31.4 |
| I-10 | 46.3 |
| I-12 | 47.4 |
| I-13 | 47.3 |
| I-14 | 40.5 |
| I-15 | 37.4 |
| I-19 | 39.1 |
| I-24 | 44 |
| I-40 | 36.2 |
| I-41 | 35.2 |
| I-42 | 28.7 |
| I-46 | 22.2 |
| I-52 | 39.1 |
| I-116 | 78.7 |

### EXAMPLE 26

### Test Example 12: Brain Distribution Studies

Compound of the present invention was intravenously administered to a rat at 0.5 mg/mL/kg dosage. 30 minutes later, all blood was drawn from the abdominal aorta under isoflurane anesthesia for death from exsanguination.

The brain was enucleated and 20 tn 25% of homogenate thereof was prepared with distilled water.

The obtained blood was used as plasma after centrifuging. The control plasma was added to the brain sample at 1:1. The control brain homogenate was added to the plasma sample at 1:1. Each sample was measured using LC/MS/MS. The obtained area ratio (a brain/plasma) was used for the brain Kp value.

TABLE 8

| No. | Kp Brain |
|---|---|
| I-1 | 2.0 |
| I-2 | 2.9 |
| I-3 | 2.3 |
| I-4 | 2.0 |
| I-7 | 2.0 |
| I-8 | 1.8 |
| I-9 | 3.3 |
| I-10 | 1.1 |
| I-12 | 1.0 |
| I-13 | 0.56 |
| I-14 | 1.1 |
| I-15 | 0.79 |
| I-19 | 0.51 |
| I-30 | 3.2 |
| I-40 | 0.74 |
| I-41 | 1.9 |
| I-42 | 2.0 |
| I-46 | 1.9 |
| I-47 | 1.1 |
| I-48 | 2.1 |
| I-52 | 0.63 |
| I-62 | 4.8 |
| I-65 | 1.7 |
| I-91 | 1.0 |
| I-116 | 1.8 |

### EXAMPLE 27

### Test Example 18: Ames Test

Ames test is performed by using Salmonellas (Salmonella typhimnrium) TA 98, TA100, TA1535 and TA1537 and Escherichia coli WP2uvrA as test, strains with or without metabolic activation in the pro-incubation method to check the presence or absence of gene mutagenicity of compounds of the present invention.

### EXAMPLE 28

### Test Example 14: P-gp Substrate Test

1. Cell line:
   a. MDR/LLC-PK1 (Becton Dickinson)
   b. LLC-PK1 (Becton Dickinson)
2. Reference substrates:
   a Digoxin (2 μM)

Methods and Procedures

1. MPR1 expressing LLC-PK1 cells and its parent cells were routinely cultured in Medium A (Medium 199 (invitrogen) supplemented with 10% PBS (Invitrogen), gentamycin (0.05 mg/mL, Invitrogen) and hygromycin B (100 μg/mL, Invitrogen)) at 37° C. under 5% CO2/95% O2 gasses. For the transport experiments, these cells were seeded on Transwell (Registered trademark) insert (96-well, pore size: 0.4 μm, Coaster) at a density of 1.4×10⁴ cells/insert and added Medium B (Medium 199 supplemented with 10% FBS and gentamycin at 0.05 mg/mL) to the feeder tray. These cells were incubated in a CO2 incubator (5% CO2/95% O2 gasses, 37° C. and replace apical and basolateral culture medium every 48-72 hr after seeding. These cells were used between 4 and 6 days after seeding.
2. The medium in the culture insert seeded with MDK1 expressing cells or parent cells were removed by aspiration and rinsed by HBSS. The apical side (140 μL) or homolateral side (175 μL) was replaced with transport buffer containing reference substrates and the present invention and then an aliquot (50 μL) of transport buffer in the donor side was collected to estimate initial concentration of reference substrate and the present invention. After incubation for designed time at 37° C., an aliquot (50 μL) of transport buffer in the donor and receiver side were collected. Assay was performed by duplicate or triplicate.
3. Reference substrate and the present invention in the aliquot was quantified by LC/MS/MS.

Calculations

Permeated amounts across monolayers of MDR1 expressing and parent cells were determined, and permeation coefficients (Pc) were calculated using Excel 2003 from the following equitation:

Pc(cm/sec)−Permeated amount (pmol)/area of cell membrane (cm2)/initial concentration (nM)/incubation time (sec)

Where, permeated amount was calculated from permeation concentration (nM, concentration of the receiver side) of the substance after incubation for the defined time (sec) multiplied by volume, (mL) and area of cell membrane was used 0.1433 (cm²).
The efflux ratio was calculated using the following equation:

Efflux Ratio=Basolateral-to-Apical Pe/Apical-to-Basolateral Pe

The net flux was calculated using the following equation

Net flux=Efflux Ratio in MDK1 expressing cells/Efflux Ratio in parent cells

TABLE 9

| | ER ratio |
|---|---|
| No. | (Cell: MDR1/LLC-PK1) |
| I-1 | 1.2 |
| I-8 | 1.3 |
| I-9 | 1.1 |
| I-10 | 4 |
| I-13 | 2.4 |
| I-14 | 2.2 |
| I-15 | 2.7 |
| I-16 | 1.8 |
| I-17 | 0.73 |
| I-28 | 1.2 |
| I-31 | 2.9 |
| I-33 | 3.2 |
| I-35 | 2.1 |
| I-40 | 1.1 |
| I-41 | 1.4 |
| I-42 | 1.5 |
| I-44 | 4.6 |
| I-46 | 1.5 |
| I-47 | 1.3 |
| I-48 | 1.1 |
| I-50 | 1.1 |
| I-51 | 0.56 |
| I-52 | 2.1 |
| I-54 | 1.9 |
| I-55 | 0.9 |
| I-56 | 1.4 |
| I-57 | 0.6 |
| I-59 | 1.2 |
| I-60 | 1.4 |
| I-62 | 0.7 |
| I-64 | 3.2 |
| I-65 | 1.1 |
| I-66 | 0.6 |
| I-67 | 0.9 |
| I-71 | 0.92 |
| I-75 | 0.8 |
| I-76 | 1 |
| I-77 | 1.2 |
| I-79 | 1.7 |
| I-82 | 0.96 |
| I-86 | 1.5 |
| I-87 | 3.4 |
| I-90 | 3.1 |
| I-91 | 1.5 |
| I-94 | 0.79 |
| I-96 | 2.0 |
| I-101 | 1.1 |
| I-103 | 1.4 |
| I-104 | 1.4 |
| I-106 | 1.9 |
| I-108 | 1.4 |
| I-109 | 8.5 |
| I-116 | 1.1 |

### EXAMPLE 29

### Test Example 15: Inhibitory Effects on P-gp Transport Materials

1. Cell line:
   a. MDR1/LLC-PK1 (Becton Dickinson)
   b. LLC-PK1 (Becton Dickinson)
2: Reference substrates:
   a. [⁹H]Digoxin (1 μM)
   b. [¹⁴C]Mannitol (5 μM)
3. Reference inhibitor:
   Cyclosporin A (10 μM)

Methods and Procedures

1. MDR1 expressing LLC-PK1 cells and its parent cells are routinely cultured in Medium A (Medium 199 (Invitrogen) supplemented with 10% FBS (Invitrogen), gentamycin (0.05 mg/mL, Invitrogen) and hygromycin B (100 μg/mL, Invitrogen)) at 37° C. under 5% CO₂/95% O₂ gasses. For the transport experiments, these cells are seeded on Transwell (Registered trademark) insert (24-well, pore size: 0.4 μm, Coaster) at a density of 1×10⁴ cells/insert and added Medium B (Medium 199 supplemented with 10% FBS and gentamycin at 0.05 mg/mL) to the feeder tray. These cells are incubated in a CO₂ incubator (5% CO₂/95% O₂ gasses, 37° C.) and replace apical and basolateral culture medium every 48-72 hr after seeding. These cells are used between 6 and 9 days after seeding.
2. The medium in the culture insert seeded with MDR1 expressing cells or parent cells are removed by aspiration and rinsed by HBSS. The apical side (250 μL) or basolateral side (850 μL) is replaced with transport buffer containing reference substrates with or without the compound of the present invention and then an aliquot (50 μL) of transport buffer in the donor side is collected to estimate initial concentration of reference substrate. After incubation for designed time at 37° C., an aliquot (50 μL) of transport buffer in the donor and receiver side are collected. Assay is performed by triplicate.
3. An aliquot (50 μL) of the transport buffer is mixed with 5 mL of a scinfillation cocktail, and the radioactivity is measured using a liquid scintillation counter.

Calculations

Permeated amounts across monolayers of MDR1 expressing and patent cells are determined, and permeation coefficients (Pc) are calculated using Excel 2003 from the following equitation:

Pc(cm/sec)−Permeated amount (pmol)/area of cell membrane (cm²)/initial concern ration (nM)/incubation time (sec)

Where, permeated amount is calculated from permeation concentration time, concentration of the receiver side) of the substance after incubation for the defined time (sec) multiplied by volume.(mL) and area of cell membrane is used 0.33 (cm²).
The efflux ratio will be calculated using the following equation:

Efflux Ratio=Basolateral-to-Apical Pe/Apical-to-Basolateral Pe

The net flux is calculated using the following equation:

Net flux=Efflux Ratio in MDR1 expressing cells/Efflux Ratio in parent cells

The percent of control is calculated as the net efflux ratio of reference compounds in the presence of the compound of the present invention to that in the absence of the compound of the present invention.
IC₅₀ values are calculated using WinNonlin (Registered trademark) pharmacokinetic software modeling propram.

### EXAMPLE 30

### Test Example 16: P-gp Substrate Test using mdrla (−/−) B6 Mice

Materials

Animal: mdrla (−/−) B6 mice (KO mouse) or C67BL/6J mice (Wild mouse)

Methods and Procedures

1. Animals may be fed prior to dosing of the compounds of the present invention.
2. The compounds of the present invention were dosed to three animals for each time point and blood and brain samples were removed at selected time points (e.g. 15 min, 30 min, 1 hr, 2 hr, 4 hr, 6 hr, 8 hr, or 24 hr) after dosing. Blood (0.3-0.7 mL) was collected via trunk blood collection with syringe containing anticoagulants (EDTA and heparin). Blood and tissue (e.g. brain) samples were immediately placed on melting ice.
3. Blood samples were centrifuged (1780×g for 10 minutes) for cell removal to obtain plasma. Then, plasma samples were transferred to a clean tube and stored in a −70° C. freezer until analysis.
4. Tissue (e.g. brain) samples were homogenized at a 1:3 ratio of tissue weight to ml of stilled water and transferred to a clean tube and stored in a −70° C. freezer until analysis.
5. Plasma and tissue (e.g. brain) samples were prepared using protein prceipitation and analyzed by LC/MS/MS. The analytical method was calibrated by including a standard curve constructed with blank plasma or brain samples and known quantities of analyte. Quality control samples were included to monitor the accuracy and precision of the methodology.
6. Plasma and brain concentration values (ng/mL and ng/g) were introduced into an appropriate tool used for calculating the pharmacokinetic parameters. A common platform was the WinNonlin (Registered trademark) pharmacokinetic software modeling program.

Calculations

Kp: Tissue to Plasma concentration ratio
Kp ratio=Kp in KO mouse/Kp in Wild mouse
KO/Wild ratio of AUC Tissue/AUC Plasma
={AUC Tissue/AUC Plasma (KO mouse)}/{AUC Tissue/AUC Plasma (Wild mouse)}

TABLE 10

| | Kp ratio |
|---|---|
| No. | (2 mg/kg, po, 2 hr) |
| I-1 | 4.12 |
| I-2 | 0.959 |
| I-3 | 5.16 |
| I-4 | 2.92 |
| I-7 | 4.21 |
| I-8 | 2.22 |
| I-9 | 3.13 |
| I-14 | 6.32 |
| I-16 | 8.13 |
| I-28 | 5.41 |
| I-30 | 3.63 |
| I-35 | 5.15 |
| I-40 | 6.97 |
| I-41 | 2.66 |
| I-42 | 2.07 |
| I-46 | 6.13 |
| I-47 | 2.12 |
| I-48 | 5.57 |
| I-116 | 4.54 |

### EXAMPLE 31

### Test Example 17: Anesthetized Guinea Pig Cardiovascular Study

Animal species: Guinea pig (Slc:Hartley, 4-6 weeks old, male), N=4
Study design:
Dosage: 3, 10, and 30 mg/kg (in principle)
(The compounds of the present invention are administered cumulatively)

### Formulation:

Composition of Vehicle: Dimethylacetamide (DMA): Polyethylene glycol 400 (PEG 400): Distilled water (D.W.)=1:7:2 (in principle).
The compounds of the present invention are dissolved with DMA and then added PEG400 and D.W. Finally, 1.5, 5, and 15 mg/mL solutions are prepared.
Dosing route and schedule:
Intravenous infusion for 10 min (2 mL/Kg).
0 to 10 min: 3 mg/kg, 30 to 10 min: 10 mg/kg, 60 to 70 min: 30 mg/kg
Vehicle is administered by the same schedule as the above.
Group composition:
Vehicle group and the compound of the present invention group (4 guinea pigs per group).
Evaluation method:
Evaluation items:
Mean blood pressure [mmHg], Heart rate (derived from blood pressure waveform [heats/min]), QTc (ms), and Toxicokinetics.

### Experimental Procedure:

Guinea pigs are anesthetized by urethane (1.4 g/kg, i.p.), and inserted polyethylene tubes into carotid artery (for measuring blood pressure and sampling bloud) and jugular vein (for infusion test compounds). Electrodes are attached subcutaneously (Lead 2). Blood pressure, heart rate and electrocardiogram (ECG) are measured using PowerLab IKegistered trademark) system (ADInstruments).

### Toxicokinetics:

Approximately 0.3 mL of blood (approximately 150 μL as plasma) is drawn from carotid artery with a syringe containing heparin sodium and cooled with ice immediately at each evaluation point. Plasma samples are obtained by centrifugation (4° C., 10000 rpm, 9300×g, 2 minutes). The procedure for separation of plasma is conducted on ice or at 4° C. The obtained plasma (TK samples) is stored in a deep freezer (set temperature: −80° C.).

Analysis methods: Mean blood pressure and heart rate are averaged a 30-second period at each evaluation time point. ECG parameters (QT Interval [ms] and QTc are derived as the average waveform of a 10-second consecutive beats in the evaluation time points. QTc [Fridericia's formula; QTc=QT/(RR)1/3)] is calculated using the PowerLab (Registered trademark) system. The incidence of arrhythmia is visually evaluated for all ECG recordings (from 0.5 hours before dosing to end of experiment) for all four animals.
Evaluation time points:
Before (pre dosing), and 10, 25, 40, 55, 70, and 85 min after the first dosing.
Data analysis of QTc:
Percentage changes (%) in QTc from no pro-dose value are calculated (the pre-dose value is regarded as 100%). Relative QTc is compared with vehicle value at the same evaluation point.

### FORMULATION EXAMPLES

The following Formulation Examples are only exemplified and not intended to limit the scope of the present invention.

### Formulation Example 1: Tablet

| | | |
|---|---|---|
| Compound of the present invention | 15 | mg |
| Lactose | 15 | mg |
| Calcium stearate | 3 | mg |

All of the above ingredients except for calcium stearate are uniformly mixed. Then the mixture is crushed, granulated and dried to obtain a suitable size of granules. Then, calcium stearate is added to the granules. Finally, tableting is performed under a compression force.

### Formulation Example 2: Capsules

| | | |
|---|---|---|
| Compound of the present invention | 10 | mg |
| Magnesium stearate | 10 | mg |
| Lactose | 80 | mg |

The above ingredients are mixed uniformly to obtain powders or fine granules, and then the obtained mixture is filled in capsules.

### Formulation Example 3: Granules

| | | |
|---|---|---|
| Compound of the present invention | 30 | g |
| Lactose | 265 | g |
| Magnesium stearate | 5 | g |

After the above ingredients, are mixed uniformly, the mixture is compressed. The compressed matters are crushed, granulated and sieved to obtain suitable size of granules.

### Formulation Example 4: Orally Disintegrated Tablets

The compounds of the present invention and crystalline cellulose are mixed, granulated and tablets are made to give orally disintegrated tablets.

### Formulation Example 5: Dry Syrups

The compounds of the present invention and lactose are mixed, crushed, granulated and sieved to give suitable sizes of dry syrups.

### Formulation Example 6: Injections

The compounds of the present invention and phosphate buffer are mixed to give injection.

### Formulation Example 7: Infusions

The compounds of the present invention and phosphate buffer are mixed to give injection.

### Formulation Example 8: Inhalations

The compound of the present invention and lactose are mixed and crushed finely to give inhalations.

### Formulation Example 9: Ointments

The compounds of the present invention and petrolatum are mixed to give ointments.

### Formulation Example 10: Patches

The compounds of the present invention and base such as adhesive plaster or the like are mixed to give patches.

### INDUSTRIAL APPLICABILITY

The compounds of the present invention can be a medicament useful as an agent for treating or preventing a disease induced by production, secretion and/or deposition of amyloid β proteins.

1. A compound of formula (I): 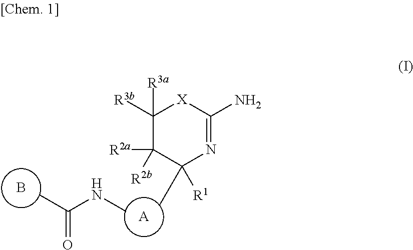
wherein
X is —S—or —O—,
(i) when X is —S—, then
R^{3a} is alkyl, haloalkyl, hydroxyalkyl, or alkyloxyalkyl,
R^{2a} is halogen, alkyloxy or haloalkyloxy and
R^{2a} may be alkyl when R^{3a} is haloalkyl,
R^{2b} is H,
R^{2a} and R^{2b} together with the carbon atom to which they are attached may form substituted cycloalkane,
R^{3a} may be H when R^{2a} and R^{2b} together with the carbon atom to which they are attached may form substituted cycloalkane,
(ii) when X is —O—, then
R^{3a} is haloalkyl optionally substituted with one or more selected from alkyloxy and cycloalkyl, or cycloalkyl substituted with one or more selected from halogen,
R^{2a} is H, halogen, alkyl, alkyloxy or haloalkyloxy,
R^{2b} is H,
R^{2a} and R^{2b} together with the carbon atom to which they are attached may form substituted cycloalkane,
R^{3a} may be H or alkyl when R^{2a} and R^{2b} together with the carbon atom to which they are attached may form substituted cycloalkane,
R^{3b} is H or alkyl, 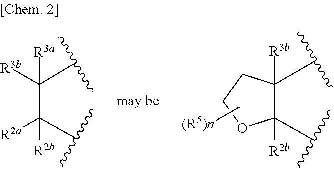
ring A is a substituted or unsubstituted aromatic carbocycle, a substituted or unsubstituted non-aromatic carbocycle, a substituted or unsubstituted aromatic heterocycle or a substituted or unsubstituted non-aromatic heterocycle,
ring B is a substituted or unsubstituted aromatic carbocycle, a substituted or unsubstituted non-aromatic carbocycle, a substituted or unsubstituted aromatic heterocycle or a substituted or unsubstituted non-aromatic heterocycle,
R¹ is substituted or unsubstituted alkyl, substituted or unsubstituted alkenyl, substituted or unsubstituted alkynyl or substituted or unsubstituted cycloalkyl,
R⁵ is halogen or substituted or unsubstituted alkyl,
n is an integer of 0 to 2,
provided that the following compounds are excluded:
(1) a compound wherein X is —O—, R^{3a} is CH₂F or CF₃, R^{3b} is H, R^{2a} is H or F, and R^{2b} is H,
(2) a compound wherein X is —O—, R^{3a} is CHF₂, R^{3b} is H, R^{2a} is OMe and R^{2b} is H, and
(3) the following compound: 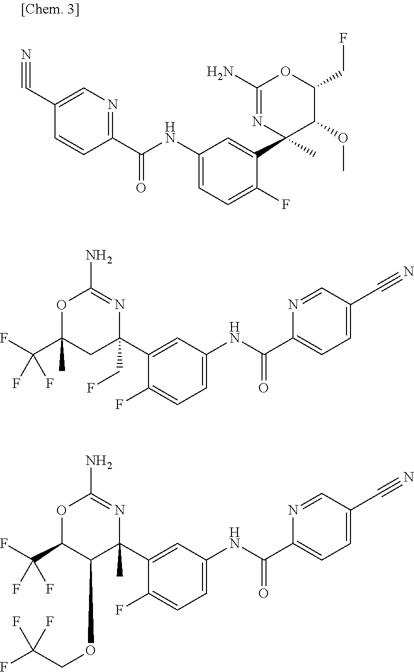 [SMILES: CO[C@H]1[C@@H](CF)OC(N)=N[C@]1(C)c1cc(NC(=O)c2ccc(C#N)cn2)ccc1F ; C[C@]1(C(F)(F)F)C[C@](CF)(c2cc(NC(=O)c3ccc(C#N)cn3)ccc2F)N=C(N)O1 ; C[C@]1(c2cc(NC(=O)c3ccc(C#N)cn3)ccc2F)N=C(N)O[C@H](C(F)(F)F)[C@@H]1OCC(F)(F)F]
or a pharmaceutically acceptable salt thereof.

2. The compound according to claim 1 wherein X is —O—, or a pharmaceutically acceptable salt thereof.

3. The compound according to claim 2 wherein R^{3a} is CH₂F, CHF₂, CF₃, CH(F)CH₃ or CF₂CH₃, and R^{3b} is H or CH₃, or a pharmaceutically acceptable salt thereof.

4. The compound according to claim 2 wherein R^{2a} is H, F, CH₃, OCH₃ or OCH₂CF₃, or a pharmaceutically acceptable salt thereof.

5. The compound according to claim 2 wherein R^{2a} is H, halogen or alkyl, R^{2b} is H, and R^{3a} is CHF₂, CH(F)CH₃ or CF₂CH₃, or a pharmaceutically acceptable salt thereof.

6. The compound according to claim 2 wherein R^{2a} is H or halogen, R^{2b} is H, R^{3a} is CH₂F or CF₃, R_{3b} is alkyl, and R¹ is unsubstituted alkyl, or a pharmaceutically acceptable salt thereof.

7. The compound according to claims 2 wherein R^{2a} is alkyl, alkyloxy or haloalkyloxy, or a pharmaceutically acceptable salt thereof.

8. The compound according to claim 2 wherein 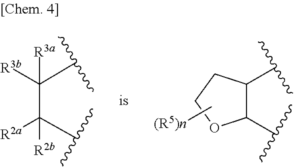
R⁵ is halogen and n is 1 or 2, or a pharmaceutically acceptable salt thereof.

9. The compound according to claim 2 wherein R^{3a} is haloalkyl substituted with alkyloxy or cycloalkyl, or a pharmaceutically acceptable salt thereof.

10. The compound according to claim 1 wherein X is —S—, R^{2a} is halogen or alkyloxy, R^{2b} is H, R^{3a} is alkyl, haloalkyl, hydroxyalkyl or alkyloxyalkyl, and R^{3b} is H, or a pharmaceutically acceptable salt thereof.

11. The compound according to claim 1 wherein X is —S—, R^{2a} is F, R^{2b} is H, R^{3a} is CH₃ or CH₂F, and R^{3b} is H, or a pharmaceutically acceptable salt thereof.

12. The compound according to claim 1 wherein R^{2a} and R^{2b} together with the carbon atom to which they are attached form cycloalkane substituted with halogen, R^{3a} is H or alkyl, or a pharmaceutically acceptable salt thereof.

13. The compound according to claim 1 wherein R¹ is alkyl, or a pharmaceutically acceptable salt thereof.

14. The compound according to claim 1 wherein ring A is 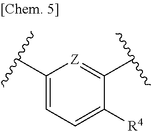
wherein R⁴ is H or halogen, and —Z═ is —CH═ or —N═, or a pharmaceutically acceptable salt thereof.

15. The compound according to claim 14 wherein R⁴ is halogen and —Z═ is —CH═, or a pharmaceutically acceptable salt thereof.

16. The compound according to claim 1 wherein ring B is substituted or unsubstituted pyridine, substituted or unsubstituted pyrazine, substituted or unsubstituted pyrimidine, substituted or unsubstituted pyridazine or substituted or unsubstituted oxazole, or a pharmaceutically acceptable salt thereof.

17. A pharmaceutical composition comprising the compound according to claim 1, or a pharmaceutically acceptable salt thereof.

18. A pharmaceutical composition having BACE1 inhibitory activity comprising the compound according to claim 1, or a pharmaceutically acceptable salt thereof.

19. A method for inhibiting BACE1 activity comprising administering the compound according to claim 1, or a pharmaceutically acceptable salt thereof.

20. The compound according to claim 1, or a pharmaceutically acceptable salt thereof for use in a method for inhibiting BACE1 activity.

21. The pharmaceutical composition according to claim 17 for treating or preventing Alzheimer dementia, mild cognitive impairment or prodromal Alzheimer's disease, for preventing the progression of Alzheimer dementia, mild cognitive impairment, or prodromal Alzheimer's disease, or for preventing the progression in a patient asymptomatic at risk for Alzheimer dementia.

22. A method for treating or preventing Alzheimer dementia, mild cognitive impairment or prodromal Alzheimer's disease, for preventing the progression of Alzheimer dementia, mild cognitive impairment, or prodromal Alzheimer's disease, or for preventing the progression in a patient asymptomatic at risk for Alzheimer dementia comprising administering the compound according to claim 1, or a pharmaceutically acceptable salt thereof.

23. A compound according to claim 1, or a pharmaceutically acceptable salt thereof for use in treating or preventing Alzheimer dementia, mild cognitive impairment or prodromal Alzheimer's disease, for use in preventing the progression of Alzheimer dementia, mild cognitive impairment or prodromal Alzheimer's disease, or for use in preventing the progression in a patient asymptomatic at risk for Alzheimer dementia.
